SEQUENCE LISTING (130 sequences)

SEQ ID NO: 1 - DNA, 1413 bp, Homo sapiens
  source 1..1413: mol_type = unassigned DNA
ttcctgttgcagataagcccagcttagcccagctgaccccagaccctctcccctcactccccccatgtcgcaggatcgagaccctgaggcagacagcccgttcaccaagccccccgccccgcccccatcaccccgtaaacttctcccagcctccgccctgccctcacccagcccgctgttccccaagcctcgctccaagcccacgccacccctgcagcagggcagccccagaggccagcacctatccccgaggctggggtcgaggctcggccccgcccctgcctctgcaacttgagcctggctgcgacccctgctctgacgtctcggaaaattcccccttgcccaggcccttgggggagggggtgcatggtatgaaatggggctgagacccccggctgggggcagaggaacccgccagagaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaataaaaattcccggtcaaacctcaaaaaaaaaaaaaaa

SEQ ID NO: 2 - DNA, 1275 bp, synthetic construct
  misc_feature 1..1275: note = Description of Artificial Sequence: Synthetic polynucleotide
  source 1..1275: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgggatcaggtagcggtagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 3 - DNA, 1287 bp, synthetic construct
  misc_feature 1..1287: note = Description of Artificial Sequence: Synthetic polynucleotide
  source 1..1287: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcggatcaggtagcggtctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 4 - DNA, 1305 bp, synthetic construct
  misc_feature 1..1305: note = Description of Artificial Sequence: Synthetic polynucleotide
  source 1..1305: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatggatcaggtagcggtagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 5 - DNA, 1290 bp, synthetic construct
  misc_feature 1..1290: note = Description of Artificial Sequence: Synthetic polynucleotide
  source 1..1290: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcggatcaggtagcggtgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 6 - DNA, 1206 bp, synthetic construct
  misc_feature 1..1206: note = Description of Artificial Sequence: Synthetic polynucleotide
  source 1..1206: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacggatcaggtagcggtcaaaagctcatttctgaagaggacttgttgcggaaacgacgagaacagttgaaacacaaacttgaacagctacggaactcttgtgcg

SEQ ID NO: 7 - DNA, 1230 bp, synthetic construct
  misc_feature 1..1230: note = Description of Artificial Sequence: Synthetic polynucleotide
  source 1..1230: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggag

SEQ ID NO: 8 - DNA, 906 bp, synthetic construct
  misc_feature 1..906: note = Description of Artificial Sequence: Synthetic polynucleotide
  source 1..906: mol_type = other DNA
atgggatcaggtagcggtctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 9 - DNA, 1062 bp, synthetic construct
  misc_feature 1..1062: note = Description of Artificial Sequence: Synthetic polynucleotide
  source 1..1062: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtc

SEQ ID NO: 10 - DNA, 1317 bp, synthetic construct
  misc_feature 1..1317: note = Description of Artificial Sequence: Synthetic polynucleotide
  source 1..1317: mol_type = other DNA
atgcccctcaacgttagcttcaccaacaggaactatgacctcgactacgactcggtgcagccgtatttctactgcgacgaggaggagaacttctaccagcagcagcagcagagcgcgctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 11 - DNA, 1317 bp, synthetic construct
  misc_feature 1..1317: note = Description of Artificial Sequence: Synthetic polynucleotide
  source 1..1317: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccgccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 12 - DNA, 1317 bp, synthetic construct
  misc_feature 1..1317: note = Description of Artificial Sequence: Synthetic polynucleotide
  source 1..1317: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctggcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 13 - DNA, 1086 bp, synthetic construct
  misc_feature 1..1086: note = Description of Artificial Sequence: Synthetic polynucleotide
  source 1..1086: mol_type = other DNA
atggcgactgcggagacagcacttccatcaatctcaacactcactgcactggggccatttccagatacccaggacgatttccttaagtggtggcggtccgaagaggctcaagacatgggacctggtccgccggatcccaccgaacctcctctgcatgtcaaaagtgaagatcagcctggcgaggaagaggatgacgaaaggggtgccgacgccacttgggacttggatcttctccttaccaatttctctggtccggaacctggcggggcaccacagacgtgcgctctcgctccctcagaagcgagcggggctcagtacccaccccctcccgaaactctgggagcctatgctgggggtcctggactggtggctgggttgcttggtagtgaggaccattctggctgggtacgccccgctttgagggcccgcgctccggacgcctttgtgggaccggcgctcgctcctgcaccggctccggaaccaaaagccctcgcgctgcagcccgtgtaccccggacccggagccggatcctcagggggatacttcccacggaccggactcagcgttccagcggcttccggggcgccatacggattgttgagcggctacccggctatgtatcccgctccccagtaccaaggacacttccaattgttccggggtcttcaagggcctgcgcccgggcctgctaccagtcccagtttcctcagttgtctgggaccgggaactgttggcactggacttggcgggactgcagaggacccaggcgttatagcagagacagcgccaagtaaaaggggccgacgaagctgggccaggaaacgccaagctgcgcacacttgtgcccatccaggttgcggtaaatcctacacgaagagcagtcatcttaaagcacatcttcgcacacacacgggcgagaagccctacgcctgtacttgggaaggttgcggctggagattcgctagatctgacgagctcacccggcattatcgaaaacacactggccagcgaccgttccggtgccaactctgcccaagggcgttcagtcgctcagatcatctggctttgcatatgaagcgacacctt

SEQ ID NO: 14 - DNA, 1065 bp, synthetic construct
  misc_feature 1..1065: note = Description of Artificial Sequence: Synthetic polynucleotide
  source 1..1065: mol_type = other DNA
atggcccttagtgaacccattcttcccagcttttccacgttcgcgtctccttgccgagagagaggccttcaggaaaggtggccgagggctgaacccgagtctggaggtacggatgatgatcttaacagtgtgctcgatttcatactctcaatgggactggacgggctgggagcggaggcagctcctgaaccaccaccaccccctccgcccccagcgttttactacccggagccaggtgcgccgccgccatattcagccccggcgggtggcttggtgtccgagctcctccggcctgaattggatgccccgctcggcccggcgctgcatggtagatttctgctcgcgcctccgggtcgactcgttaaggctgaacctcctgaggctgatggtggaggtggctacggatgtgcccccgggcttacccgaggaccgagaggtcttaagcgggaaggggcacctggcccggctgcaagctgtatgcgggggcccggtgggaggcctcccccgccccctgatacacccccccttagtccagatggaccagctcgacttcccgcacctggccccagagcgagtttcccccctccatttggaggaccggggtttggcgccccaggtcctggacttcactacgcccctcctgcccccccagcttttggtcttttcgacgatgctgctgctgccgcagcagccttgggccttgcgccgcccgcagccaggggactgctcacgccaccggcaagccccctggagctccttgaagccaagccgaagcgaggacgcagatcatggccgcgcaagcggacagctacgcatacctgctcatatgcgggctgcggaaaaacctacacaaagagttcacaccttaaagcgcaccttcgcacacacacaggcgagaaaccatatcattgtaactgggacggatgtggatggaaatttgctcggtctgatgagcttacgagacattatcgaaagcataccggacatcggccctttcaatgccatctttgtgacagagctttttcccggtctgaccacctcgctctgcacatgaagaggcacatg

SEQ ID NO: 15 - DNA, 1035 bp, synthetic construct
  misc_feature 1..1035: note = Description of Artificial Sequence: Synthetic polynucleotide
  source 1..1035: mol_type = other DNA
atgctcatgtttgacccagttcctgtcaagcaagaggccatggaccctgtctcagtgtcatacccatctaattacatggaatccatgaagcctaacaagtatggggtcatctactccacaccattgcctgagaagttctttcagaccccagaaggtctgtcgcacggaatacagatggagccagtggacctcacggtgaacaagcggagttcacccccttcggctgggaattcgccctcctctctgaagttcccgtcctcacaccggagagcctcgcctgggttgagcatgccttcttccagcccaccgataaaaaaatactcacccccttctccaggcgtgcagcccttcggcgtgccgctgtccatgccaccagtgatggcagctgccctctcgcggcatggaatacggagcccggggatcctgcccgtcatccagccggtggtggtgcagcccgtcccctttatgtacacaagtcacctccagcagcctctcatggtctccttatcggaggagatggaaaattccagtagtagcatgcaagtacctgtaattgaatcatatgagaagcctatatcacagaaaaaaattaaaatagaacctgggatcgaaccacagaggacagattattatcctgaagaaatgtcaccccccttaatgaactcagtgtcccccccgcaagcattgttgcaagagaatcacccttcggtcatcgtgcagcctgggaagagacctttacctgtggaatccccggatactcaaaggaagcggaggatacacagatgtgattatgatggatgcaacaaagtgtacactaaaagctcccacttgaaagcacacagaagaacacacacaggagaaaaaccctacaaatgtacatgggaagggtgcacatggaagtttgctcggtctgatgaactaacaagacatttccgaaaacatactggaatcaaacctttccagtgcccggactgtgaccgcagcttctcccgttctgaccatcttgccctccataggaaacgccacatgctagtc

SEQ ID NO: 16 - DNA, 1371 bp, synthetic construct
  misc_feature 1..1371: note = Description of Artificial Sequence: Synthetic polynucleotide
  source 1..1371: mol_type = other DNA
atggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaac

SEQ ID NO: 17 - DNA, 849 bp, synthetic construct
  misc_feature 1..849: note = Description of Artificial Sequence: Synthetic polynucleotide
  source 1..849: mol_type = other DNA
atggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctc

SEQ ID NO: 18 - DNA, 906 bp, synthetic construct
  misc_feature 1..906: note = Description of Artificial Sequence: Synthetic polynucleotide
  source 1..906: mol_type = other DNA
atggacgtgttggctagttatagtatattccaggagctacaacttgtccacgacaccggctacttctcagctttaccatccctggaggagacctggcagcagacatgccttgaattggaacgctacctacagacggagccccggaggatctcagagacctttggtgaggacttggactgtttcctccacgcttcccctcccccgtgcattgaggaaagcttccgtcgcttagaccccctgctgctccccgtggaagcggccatctgtgagaagagctcggcagtggacatcttgctctctcgggacaagttgctatctgagacctgcctcagcctccagccggccagctcttctctagacagctacacagccgtcaaccaggcccagctcaacgcagtgacctcattaacgcccccatcgtcccctgagctcagccgccatctggtcaaaacctcacaaactctctctgccgtggatggcacggtgacgttgaaactggtggccaagaaggctgctctcagctccgtaaaggtgggaggggtcgcaacagctgcagcagccgtgacggctgcgggggccgttaagagtggacagagcgacagtgaccaaggagggctaggggctgaagcatgtcccgaaaacaagaagagggttcaccgctgtcagtttaacgggtgccggaaagtttatacaaaaagctcccacttaaaggcccaccagaggactcacacaggtgagaagccttataagtgctcatgggagggatgtgagtggcgttttgcacgaagcgatgagctcacgaggcactacaggaaacacacaggtgcaaagcccttcaaatgcaaccactgcgacaggtgtttttccaggtctgaccatcttgccctccacatgaagagacatatc

SEQ ID NO: 19 - DNA, 1077 bp, synthetic construct
  misc_feature 1..1077: note = Description of Artificial Sequence: Synthetic polynucleotide
  source 1..1077: mol_type = other DNA
atggtcgatatggataaactcataaacaacttggaggtccaacttaattcagaaggtggctcaatgcaggtattcaagcaggtcactgcttctgttcggaacagagatccccctgagatagaatacagaagtaatatgacttctccaacactcctggatgccaaccccatggagaacccagcactgtttaatgacatcaagattgagcccccagaagaacttttggctagtgatttcagcctgccccaagtggaaccagttgacctctcctttcacaagcccaaggctcctctccagcctgctagcatgctacaagctccaatacgtccccccaagccacagtcttctccccagacccttgtggtgtccacgtcaacatctgacatgagcacttcagcaaacattcctactgttctgaccccaggctctgtcctgacctcctctcagagcactggtagccagcagatcttacatgtcattcacactatcccctcagtcagtctgccaaataagatgggtggcctgaagaccatcccagtggtagtgcagtctctgcccatggtgtatactactttgcctgcagatgggggccctgcagccattacagtcccactcattggaggagatggtaaaaatgctggatcagtgaaagttgaccccacctccatgtctccactggaaattccaagtgacagtgaggagagtacaattgagagtggatcctcagccttgcagagtctgcagggactacagcaagaaccagcagcaatggcccaaatgcagggagaagagtcgcttgacttgaagagaagacggattcaccaatgtgactttgcaggatgcagcaaagtgtacaccaaaagctctcacctgaaagctcaccgcagaatccatacaggagagaagccttataaatgcacctgggatggctgctcctggaaatttgctcgctcagatgagctcactcgccatttccgcaagcacacaggcatcaagccttttcggtgcacagactgcaaccgcagcttttctcgttctgaccacctgtccctgcatcgccgtcgccatgacaccatg

SEQ ID NO: 20 - DNA, 732 bp, synthetic construct
  misc_feature 1..732: note = Description of Artificial Sequence: Synthetic polynucleotide
  source 1..732: mol_type = other DNA
atgtccgcggccgcctacatggacttcgtggctgcccagtgtctggtttccatttcgaaccgcgctgcggtgccggagcatggggtcgctccggacgccgagcggctgcgactacctgagcgcgaggtgaccaaggagcacggtgacccgggggacacctggaaggattactgcacactggtcaccatcgccaagagcttgttggacctgaacaagtaccgacccatccagaccccctccgtgtgcagcgacagtctggaaagtccagatgaggatatgggatccgacagcgacgtgaccaccgaatctgggtcgagtccttcccacagcccggaggagagacaggatcctggcagcgcgcccagcccgctctccctcctccatcctggagtggctgcgaaggggaaacacgcctccgaaaagaggcacaagtgcccctacagtggctgtgggaaagtctatggaaaatcctcccatctcaaagcccattacagagtgcatacaggtgaacggccctttccctgcacgtggccagactgccttaaaaagttctcccgctcagacgagctgacccgccactaccggacccacactggggaaaagcagttccgctgtccgctgtgtgagaagcgcttcatgaggagtgaccacctcacaaagcacgcccggcggcacaccgagttccaccccagcatgatcaagcgatcgaaaaaggcgctggccaacgctttg

SEQ ID NO: 21 - DNA, 1440 bp, synthetic construct
  misc_feature 1..1440: note = Description of Artificial Sequence: Synthetic polynucleotide
  source 1..1440: mol_type = other DNA
atgctcaacttcggtgcctctctccagcagactgcggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcattttgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacag

SEQ ID NO: 22 - DNA, 1536 bp, synthetic construct
  misc_feature 1..1536: note = Description of Artificial Sequence: Synthetic polynucleotide
  source 1..1536: mol_type = other DNA
atgcatactcctgatttcgctggacctgacgacgcccgagccgtggacattatggacatttgtgaatctatactcgaaagaaagagacatgattcagagcgaagtacatgctctatcctcgagcaaacagacatggaggcggtagaagctctggtgtgcatgtccagttggggtcagagatcccagaagggggacttgcttagaatccgaccgcttactccagtttccgatagcggcgacgtaacaactactgttcatatggacgcagccacgcctgagctgcccaaagactttcacagcctctcaactctttgcatcactccaccacagtcccccgatcttgtcgaaccatcaacccggacccctgttagcccgcaagttacagattcaaaggcgtgtaccgcgaccgatgttctgcagagttcagcggttgtagcgcgggcattgagcggaggggctgaacgaggtctgttgggtcttgaacccgtaccgagttctccttgtagagccaagggtactagtgttattcggcataccggcgagagtccggcagcttgtttccccaccatacaaaccccagactgtcgccttagtgattcccgggaaggggaggaacagctgttgggccacttcgagacacttcaagatacacacttgacagatagcttgctgtccaccaacctggtgtcatgtcaaccttgtttgcacaagtccgggggtctccttctgactgacaaaggtcaacaagcgggatggcctggcgctgtccaaacatgcagtcctaaaaactacgaaaatgatttgcctaggaaaaccacgccgcttatcagtgtgagtgttcccgctccacctgtcctgtgccagatgatccctgtaaccgggcaatcatctatgttgcctgcgttcttgaagccccccccacaactgtccgttggtactgttcgcccgatccttgcgcaagcagcgcccgccccgcaacccgtgttcgtggggcccgctgtcccgcagggtgcagtcatgttggttcttccccagggggccctcccgccaccagctccgtgtgcagcgaatgtcatggctgccggaaacacgaaattgttgccccttgcacccgctccagttttcataacgagctcacagaattgtgtgccacaagtcgacttctcacgaagacggaactatgtgtgctctttcccaggttgcagaaaaacatatttcaaatcctctcatctgaaagcacatcttcggacccatacaggagagaagccttttaattgtagctgggatggctgtgataaaaaattcgcaagaagtgatgagctcagtcgacatcgcaggacgcataccggggaaaaaaaattcgtttgtccagtttgtgacagaagatttatgaggtccgaccatctcaccaagcacgcgcgacgccacatgactacaaagaaaattcctggctggcaagccgaggtgggaaaactcaaccgaatcgcttccgctgaatcccccggcagcccgctggtaagtatgcctgccagtgcc

SEQ ID NO: 23 - DNA, 1206 bp, synthetic construct
  misc_feature 1..1206: note = Description of Artificial Sequence: Synthetic polynucleotide
  source 1..1206: mol_type = other DNA
atgaacattcacatgaagcgcaagacgataaagaacatcaatacattcgagaaccgaatgttgatgttggatggcatgcccgctgtacgggtaaaaaccgagctcctggagtctgaacaaggatccccaaacgtccacaactacccggatatggaggcagtgccgctcttgctcaacaatgtgaagggagagccgcctgaggactctctctccgtagatcatttccagacacagactgagcccgtagatctttcaattaacaaagccagaacatctcctactgcggtaagttcttctcccgtaagtatgacagcaagtgcatctagtccaagttctacgagcactagcagttcttcatctagtagacttgctagttcaccaacggtgatcacaagtgtttctagcgccagcagcagctcaacggtactgactcccggtccactcgtggcaagcgctagtggcgtgggtggccaacaatttctccatattattcaccccgtgcctccgtctagtccgatgaatctccagagcaacaagcttagtcacgtacataggatccccgtcgtcgtccagtcagttcccgtcgtctacacagctgtgcgatcccctgggaatgtcaataatactatagttgttcctttgcttgaggatggtaggggccatgggaaagcacagatggacccccgcggcttgtcaccgagacagtctaaatccgatagtgacgacgatgatttgcctaacgtaacactggactctgtgaacgagaccgggagtaccgctctgtcaatcgctagggccgtacaggaggtccacccaagccctgtgtcacgagtccgaggtaacaggatgaataatcagaaatttccctgtagcatcagcccattttctatagagtccactcggagacagcgacgaagtgaatcacccgactccagaaaaaggaggatacatcgctgtgactttgagggctgtaacaaggtctacacaaaaagttcacacctcaaggcgcatcgacggacgcatactggggaaaaaccgtacaaatgcacctgggagggatgcacgtggaaatttgcacgctctgacgagttgacacgccactatcgaaagcatacgggcgtaaagccgtttaaatgcgctgattgcgacaggagttttagccgctctgatcaccttgctcttcaccggaggcgacacatgcttgtt

SEQ ID NO: 24 - DNA, 864 bp, synthetic construct
  misc_feature 1..864: note = Description of Artificial Sequence: Synthetic polynucleotide
  source 1..864: mol_type = other DNA
atggctgcggctgcatatgtggatcattttgcggctgagtgcctggtgtcaatgtctagtagagcggtggtacacggtcccagagaaggcccagaatcacgcccagagggcgccgccgtcgctgcaacaccgacgctgcctcgggtcgaggagcgccgcgacgggaaggacagtgcgtcacttttcgtagtagcgagaatattggcagatctgaatcaacaggctccagcacctgcgcccgctgaacgccgggagggcgccgctgccagaaaggccagaacaccatgccgcttgccgccacctgcgccagaacccacaagtccaggtgccgaaggtgcggcggctgcccctccttcaccggcctggtctgaaccagaaccagaggcaggtcttgaacctgagcgcgaacccggccctgcaggctctggggaacctggcctgaggcagcgggtgaggcgcggccggagcagggccgacctggaatcaccgcaaaggaaacataaatgccattatgctggttgcgaaaaggtttatggaaagtcatcccacctgaaagcacacctccgcactcacacgggtgagcgaccttttgcgtgttcctggcaagactgcaataaaaagtttgctagatctgatgaacttgcacggcattatcgaactcataccggtgaaaagaagttctcatgccctatatgtgagaaacggttcatgcgctctgaccacttgacgaaacatgcaagacgacatgctaattttcatccggggatgttgcagagacggggagggggaagtaggactggaagtctctccgactattcccgatccgacgcttcctcaccaacgattagccccgcaagcagtccc

SEQ ID NO: 25 - DNA, 969 bp, synthetic construct
  misc_feature 1..969: note = Description of Artificial Sequence: Synthetic polynucleotide
  source 1..969: mol_type = other DNA
atgtcagccgcagtcgcatgccttgattacttcgcggccgagtgtcttgtttccatgtcagcgggggctgtcgttcacagaagaccaccagacccggagggagcgggaggggcagctggatctgaagtcggcgcggctccacctgaatcagcgcttcccggccctggtcctccaggtcccgctagcgtgccccaactcccacaagtgcctgctccgagtcctggagcgggcggagcagccccgcatctccttgcagcatcagtgtgggccgatcttcgcggaagctccggggagggctcctgggaaaacagcggagaggccccgcgagcttcaagcggcttttccgatccaatcccttgcagtgttcaaaccccatgctccgagctcgcgcccgcgtccggagctgcggcagtgtgcgcacctgaaagctcatccgatgcgccggccgttccatctgcgccagctgctcccggtgcacccgcagcatctggcggctttagtggtggagctcttggggcgggtcccgcccctgcggcggatcaagctcctcgcaggcgcagtgttacgcccgcagcaaaacggcatcaatgcccctttcctggttgtacaaaagcatactataagtcatcccatctcaagagtcaccagaggacgcatacaggtgagagaccttttagctgtgactggctcgattgcgacaagaaatttacgcggagcgacgaacttgcgcggcactaccgcactcacactggagaaaagaggttctcttgtcccctgtgtcccaagcagttctcacgcagtgatcacttgacaaaacatgctaggagacatccaacataccatcccgacatgatagagtatcgaggtaggcgacgcacacctagaattgatcctccgctgactagtgaagtcgagtcaagtgccagtggaagcggaccgggtcccgcgccctcatttacaacctgtctt

SEQ ID NO: 26 - DNA, 1248 bp, synthetic construct
  misc_feature 1..1248: note = Description of Artificial Sequence: Synthetic polynucleotide
  source 1..1248: mol_type = other DNA
atggtggaccacttacttccagtggacgagaacttctcgtcgccaaaatgcccagttgggtatctgggtgataggctggttggccggcgggcatatcacatgctgccctcacccgtctctgaagatgacagcgatgcctccagcccctgctcctgttccagtcccgactctcaagccctctgctcctgctatggtggaggcctgggcaccgagagccaggacagcatcttggacttcctattgtcccaggccacgctgggcagtggcgggggcagcggcagtagcattggggccagcagtggccccgtggcctgggggccctggcgaagggcagcggcccctgtgaagggggagcatttctgcttgcccgagtttcctttgggtgatcctgatgacgtcccacggcccttccagcctaccctggaggagattgaagagtttctggaggagaacatggagcctggagtcaaggaggtccctgagggcaacagcaaggacttggatgcctgcagccagctctcagctgggccacacaagagccacctccatcctgggtccagcgggagagagcgctgttcccctccaccaggtggtgccagtgcaggaggtgcccagggcccaggtgggggccccacgcctgatggccccatcccagtgttgctgcagatccagcccgtgcctgtgaagcaggaatcgggcacagggcctgcctcccctgggcaagccccagagaatgtcaaggttgcccagctcctggtcaacatccaggggcagaccttcgcactcgtgccccaggtggtaccctcctccaacttgaacctgccctccaagtttgtgcgcattgcccctgtgcccattgccgccaagcctgttggatcgggacccctggggcctggccctgccggtctcctcatgggccagaagttccccaagaacccagccgcagaactcatcaaaatgcacaaatgtactttccctggctgcagcaagatgtacaccaaaagcagccacctcaaggcccacctgcgccggcacacgggtgagaagcccttcgcctgcacctggccaggctgcggctggaggttctcgcgctctgacgagctgtcgcggcacaggcgctcgcactcaggtgtgaagccgtaccagtgtcctgtgtgcgagaagaagttcgcgcggagcgaccacctctccaagcacatcaaggtgcaccgcttcccgcggagcagccgctccgtgcgctccgtgaac

SEQ ID NO: 27 - DNA, 756 bp, synthetic construct
  misc_feature 1..756: note = Description of Artificial Sequence: Synthetic polynucleotide
  source 1..756: mol_type = other DNA
atgtcagccgcggtcgcgtgcgtggattattttgcagcagatgtgctgatggcaatttcatccggtgcagtagttcatcgcggaagaccaggtcctgagggtgcggggcctgcggccgggttggatgttcgcgccgcgcgcagggaagccgcttctcccggaacacctggccctcctcctcctccgccggcggcatcaggcccgggtcctggtgcagctgcggctcctcacctgttggcagcctccatactggctgacctgcgaggggggccaggcgctgcacctggtggcgcgagtccagcaagttccagctccgcggcgtcctccccgagtagtgggcgagctccgggcgcggcaccttctgctgccgctaaatcacaccgatgccctttcccagactgcgcgaaggcgtattataagtccagtcatttgaaatcacacttgaggacacataccggcgagagaccttttgcgtgcgactggcagggttgtgataagaaatttgcgagaagcgacgaactggcccgccatcaccgcacccacacaggggaaaaaagattctcatgcccactctgttctaagcgcttcacgcgaagcgaccatcttgcaaagcacgctaggagacaccctgggttccaccccgacctcttgcgacgacctggcgcccggtctactagcccgtctgactcattgccgtgctctctcgcagggtcccctgctccgagccccgcaccgtccccagctcctgccgggctt

SEQ ID NO: 28 - DNA, 1167 bp, synthetic construct
  misc_feature 1..1167: note = Description of Artificial Sequence: Synthetic polynucleotide
  source 1..1167: mol_type = other DNA
atgtacggccgaccgcaggctgagatggaacaggaggctggggagctgagccggtggcaggcggcgcaccaggctgcccaggataacgagaactcagcgcccatcttgaacatgtcttcatcttctggaagctctggagtgcacacctcttggaaccaaggcctaccaagcattcagcactttcctcacagcgcagagatgctggggtcccctttggtgtctgttgaggcgccggggcagaatgtgaatgaaggggggccacagttcagtatgccactgcctgagcgtggtatgagctactgcccccaagcgactctcactccttcccggatgatttactgtcagagaatgtctccccctcagcaagagatgacgattttcagtgggccccaactaatgcccgtaggagagcccaatattccaagggtagccaggcccttcggtgggaatctaaggatgccccccaatgggctgccagtctcggcttccactggaatcccaataatgtcccacactgggaaccctccagtgccttaccctggcctctcgacagtaccttctgacgaaacattgttgggcccgactgtgccttccactgaggcccaggcagtgctcccctccatggctcagatgttgcccccgcaagatgcccatgaccttgggatgcccccagctgagtcccagtcattgctggttttaggatctcaggactctcttgtcagtcagccagactctcaagaaggcccatttctaccagagcagcccggacctgctccacagacagtagagaagaactccaggcctcaggaagggactggtagaaggggctcctcagaggcaaggccttactgctgcaactacgagaactgcggaaaagcttataccaaacgctcccacctcgtgagccaccagcgcaagcacacaggtgagaggccatattcttgcaactgggaaagttgttcatggtctttcttccgttctgatgagcttagacgacatatgcgggtacacaccagatatcgaccatataaatgtgatcagtgcagccgggagttcatgaggtctgaccatctcaagcaacaccagaagactcatcggccgggaccctcagacccacaggccaacaacaacaatggagagcaggacagtcctcctgctgctggtcct

SEQ ID NO: 29 - protein, 6 aa, synthetic construct
  REGION 1..6: note = Description of Artificial Sequence: Synthetic peptide
GSGSGS

SEQ ID NO: 30 - DNA, 59 bp, synthetic construct
  misc_feature 1..59: note = Description of Artificial Sequence: Synthetic primer
  source 1..59: mol_type = other DNA
gtctcgtgggctcggagatgtgtataagagacagagaactatttcctggctgttacgcg

SEQ ID NO: 31 - DNA, 58 bp, synthetic construct
  misc_feature 1..58: note = Description of Artificial Sequence: Synthetic primer
  source 1..58: mol_type = other DNA
acactctttccctacacgacgctcttccgatctagaactatttcctggctgttacgcg

SEQ ID NO: 32 - DNA, 56 bp, synthetic construct
  misc_feature 1..56: note = Description of Artificial Sequence: Synthetic primer
  source 1..56: mol_type = other DNA
gactggagttcagacgtgtgctcttccgatcttgtcttcgttgggagtgaattagc

SEQ ID NO: 33 - DNA, 708 bp, synthetic construct
  misc_feature 1..708: note = Description of Artificial Sequence: Synthetic polynucleotide
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 34 - DNA, 708 bp, synthetic construct
  misc_feature 1..708: note = Description of Artificial Sequence: Synthetic polynucleotide
  source 1..708: mol_type = other DNA
atggagtcttctgctaaaatggagtccggaggcgcgggacaacaaccacaaccgcaaccacaacaacccttcctgccgccggccgcatgttttttcgcgaccgctgctgctgctgcagcggcggcggctgctgccgccgcgcaatccgcccaacagcaacaacaacaacagcagcagcagcaacaagcgcctcaacttcgacccgctgcagacgggcagccctcagggggagggcacaagagcgctccgaagcaggttaaaaggcagaggagcagtagtcccgaactgatgcgatgtaagaggcgcctcaattttagcggttttggttactctttgccccagcagcagccggctgccgtagctcgccgaaatgagcgggaaaggaaccgcgttaaacttgtgaatctcggtttcgcgacacttcgagagcacgtaccaaatggggcagctaacaagaaaatgagtaaagttgagacactgcggtctgcagtggagtatattagagctcttcaacaattgcttgacgagcacgatgccgtatcagccgcatttcaagccggggtgctgtccccaacaatatctccgaactacagcaatgatcttaatagcatggcgggaagtcccgtttcctcctactcctctgatgagggcagctacgaccctctcagtcccgaggagcaagagcttcttgacttcactaactggttc

SEQ ID NO: 35 - DNA, 573 bp, synthetic construct
  misc_feature 1..573: note = Description of Artificial Sequence: Synthetic polynucleotide
  source 1..573: mol_type = other DNA
atgatggacaacagaggcaactctagtctacctgacaaacttcctatcttccctgattctgcccgcttgccacttaccaggtccttctatctggagcccatggtcactttccacgtgcacccagaggccccggtgtcatctccttactctgaggagctgccacggctgccttttcccagcgactctcttatcctgggaaattacagtgaaccctgccccttctctttcccgatgccttatccaaattacagagggtgcgagtactcctacgggccagccttcacccggaaaaggaatgagcgggaaaggcagcgggtgaaatgtgtcaatgaaggctacgcccagctccgacatcatctgccagaggagtatttggagaagcgactcagcaaagtggaaaccctcagagctgcgatcaagtacattaactacctgcagtctcttctgtaccctgataaagctgagacaaagaataaccctggaaaagtttcctccatgatagcaaccaccagccaccatgctgaccctatgttcagaattgtttgcccaactttcttgtacaaagttgtcccc

SEQ ID NO: 36 - DNA, 516 bp, synthetic construct
  misc_feature 1..516: note = Description of Artificial Sequence: Synthetic polynucleotide
  source 1..516: mol_type = other DNA
atggagacgcgtaaaccggcggaacggctggccttgccatactcgctgcgcaccgcgcccctgggcgttccggggaccctgcccggactcccgcggagggaccccctcagggtcgccctgcgtctggacgccgcgtgctgggagtgggcgcgcagcggctgcgcacggggatggcagtacttgcccgtgccgctggacagcgccttcgagcccgccttcctccgcaagcgcaacgagcgcgagcggcagcgggtgcgctgcgtgaacgagggctatgcgcgcctccgagaccacctgccccgggagctggcagacaagcgcctcagcaaagtggagacgctccgcgctgccatcgactacatcaagcacctgcaggagctgctggagcgccaggcctgggggctcgagggcgcggccggcgccgtcccccagcgcagggcggaatgcaacagcgacggggagtccaaggcctcttcggcgccttcgcccagcagcgagcccgaggaggggggcagc

SEQ ID NO: 37 - DNA, 833 bp, synthetic construct
  misc_feature 1..833: note = Description of Artificial Sequence: Synthetic polynucleotide
  source 1..833: mol_type = other DNA
atgccgatgggggcagcagaaagaggtgctgggccccaatcatctgcagcaccatgggctggttcagaaaaggcggcaaagagagggccatcaaaaagctggtacccaagagctgctgcatctgatgtcacgtgcccgactggtggtgatggagctgacccaaaacctggaccttttggaggtggtttagctttagggcctgcgcccagaggaacaatgaataataatttctgcagggcccttgttgacagaaggcctttaggacccccttcatgtatgcaattaggtgtaatgccaccgccaagacaagcgcccctcccgccggctgaaccccttggaaatgtacctttcctcctataccctggcccagctgaaccaccatattatgatgcatatgctggtgttttcccatatgtgcctttccctggtgcttttggtgtatatgaatacccttttgagccggcttttatccaaaagaggaatgaaagagagagacagagagtgaagtgtgtgaatgaaggatacgccagattgagaggccatttgcctggtgccctggcagaaaagagattatcaaaagttgaaaccctgagggcggcaatcagatatataaaatacctccaagaactcctttcatcagcacctgatggatcgacaccaccggcttcaagaggtttacctggaactggaccatgccctgcaccgcctgctacaccaaggccagacagacctggagatggagaagcaagagcaccttcttcccttgtccctgaatcttctgaatcatcatgtttttcgccttccccttttttagaaagtgaagaatcctggca

SEQ ID NO: 38 - DNA, 1482 bp, synthetic construct
  misc_feature 1..1482: note = Description of Artificial Sequence: Synthetic polynucleotide
  source 1..1482: mol_type = other DNA
atgggagacgacagaccgtttgtgtgcaatgccccgggctgtggacagagatttacaaacgaggaccacctggcagttcataaacacaagcatgagatgacattgaaatttggcccagcccgaactgactcagtcatcattgcagatcaaacgcctactccaactagattcctgaagaactgtgaggaggtgggactcttcaatgaactagctagctcctttgaacatgaattcaagaaagctgcagatgaggatgagaaaaaggcaagaagcaggactgttgccaaaaaactggtggctgctgctgggccccttgacatgtctctgccttccacaccagacatcaaaatcaaagaagaagagccagtggaggtagactcatccccacctgatagccctgcctctagtccctgttccccaccactgaaggagaaggaggttaccccaaagcctgttctgatctctacccccacacccaccattgtacgtcctggctccctgcctctccacttgggctatgatccacttcatccaacccttccctccccaacctctgtcatcacacaggctccaccatccaacaggcaaatggggtctcccactggctccctccctcttgtcatgcatcttgctaatggacagaccatgcctgtgttgccagggcctccagtacagatgccgtctgttatatcgctggccagacctgtgtccatggtgcccaacattcctggtatccctggcccaccagttaacagtagtggctccatttctccctctggccaccctataccatcagaagccaagatgagactgaaagccaccctaactcaccaagtctcctcaatcaatggtggttgtggaatggtggtgggtactgccagcaccatggtgacagcccgcccagagcagagccagattctcatccagcaccctgatgccccatcccctgcccagccacaggtctcaccagctcagcccacccctagtactggggggcgacggcggcgcacagtagatgaagatccagatgagcgacggcagcgctttctggagcgcaaccgggctgcagcctcccgctgccgccaaaagcgaaagctgtgggtgtcctccctagagaagaaggccgaagaactcacttctcagaacattcagctgagtaatgaagtcacattactacgcaatgaggtggcccagttgaaacagctactgttagctcataaagactgcccagtcactgcactacagaaaaagactcaaggctatttagaaagccccaaggaaagctcagagccaacgggttctccagcccctgtgattcagcacagctcagcaacagcccctagcaatggcctcagtgttcgctctgcagctgaagctgtggccacctcggtcctcactcagatggccagccaaaggacagaactgagcatgccgatacaatcgcatgtaatcatgaccccacagtcccagtctgcgggcaga

SEQ ID NO: 39 - DNA, 939 bp, synthetic construct
  misc_feature 1..939: note = Description of Artificial Sequence: Synthetic polynucleotide
  source 1..939: mol_type = other DNA
atgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcggcagcgaacttggacagcgcgcagtccccggggccatcctggccggcagcgtatggcgccccactccgggaggactggaatggctacgcgcccggaggcgccgcggccgccgccaacgccgtggctcacggcctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgtctggctctgtccctggggttctggggccaactgggggggtgctaaaccccaccgtcacccag

SEQ ID NO: 40 - DNA, 897 bp, synthetic construct
  misc_feature 1..897: note = Description of Artificial Sequence: Synthetic polynucleotide
  source 1..897: mol_type = other DNA
atgatggcgtatatgaacccggggccccactattctgtcaacgccttggccctaagtggccccagtgtggatctgatgcaccaggctgtgccctacccaagcgcccccaggaagcagcggcgggagcgcaccaccttcacccggagccaactggaggagctggaggcactgtttgccaagacccagtacccagacgtctatgcccgtgaggaggtggctctgaagatcaatctgcctgagtccagggttcaggtttggttcaagaaccggagggctaaatgcaggcagcagcgacagcagcagaaacagcagcagcagcccccagggggccaggccaaggcccggcctgccaagaggaaggcgggcacgtccccaagaccctccacagatgtgtgtccagaccctctgggcatctcagattcctacagtccccctctgcccggcccctcaggctccccaaccacggcagtggccactgtgtccatctggagcccagcctcagagtcccctttgcctgaggcgcagcgggctgggctggtggcctcagggccgtctctgacctccgccccctatgccatgacctacgccccggcctccgctttctgctcttccccctccgcctatgggtctccgagctcctatttcagcggcctagacccctacctttctcccatggtgccccagctagggggcccggctcttagccccctctctggcccctccgtgggaccttccctggcccagtcccccacctccctatcaggccagagctatggcgcctacagccccgtggatagcttggaattcaaggaccccacgggcacctggaaattcacctacaatcccatggaccctctggactacaaggatcagagtgcctggaagtttcagatcttg

SEQ ID NO: 41 - DNA, 1437 bp, synthetic construct
  misc_feature 1..1437: note = Description of Artificial Sequence: Synthetic polynucleotide
  source 1..1437: mol_type = other DNA
atggccagcactattaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaatgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactac

SEQ ID NO: 42 - DNA, 1326 bp, synthetic construct
  misc_feature 1..1326: note = Description of Artificial Sequence: Synthetic polynucleotide
  source 1..1326: mol_type = other DNA
atgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccattgctctggtctgatcctgcagataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtc

SEQ ID NO: 43 - DNA, 1110 bp, synthetic construct
  misc_feature 1..1110: note = Description of Artificial Sequence: Synthetic polynucleotide
  source 1..1110: mol_type = other DNA
atggatctttggaactgggatgaagcttcccctcaagaagttccccccggaaataaactcgcggggcttggaagactccctcgccttccgcaacgcgtctggggcggatgccctggtggagcctcagcggacccaaaccctttgtctccagcggagggggcaaagttgggtttctgcttcccggatcttgctttgcaaggcgatactccaacggcgacggcagagacctgttggaaaggcaccagtagctccctggccagctttccgcagctcgattgggggtcagcccttctccatcccgaagttccctggggggcggaacccgactcccaagcccttccctggagtggtgattggacagatatggcatgcacagcctgggacagttggtccggggcgtcacagacattgggaccagccccacttggaccggggcctatccccgcagcaggaagcgaaggagctgctggtcagaactgtgtgcccgtggctggtgaggctaccagttggtccagggcccaggcagcaggcagtaacaccagctgggattgctcagtggggcctgacggggatacttattggggctctggtcttggtggagaaccgagaacggactgtacgataagttggggcggtccagctgggcctgattgtactacgtcatggaatcctggcttgcacgccggcggcacgacaagccttaagagatatcaaagttcagcccttacagtttgctcagaaccttccccgcaaagtgaccgagcgtcactggcgcgatgtcctaaaactaatcatcgagggccgatccagttgtggcagtttttgcttgaactccttcacgatggcgcgaggagcagttgcatcagatggaccggtaacagcagggagttccaattgtgtgaccccaaggaagtggctcgactgtggggtgagcgcaaacggaagcctggtatgaattacgaaaagttgagtaggggtttgcgatattactataggcgcgacatcgttcgaaagtccggtggtcgaaagtacacatacagattcggcggtcgcgtaccatctcttgcataccctgattgcgcaggcgggggtaggggtgcggaaacacaa

SEQ ID NO: 44 - DNA, 1356 bp, synthetic construct
  misc_feature 1..1356: note = Description of Artificial Sequence: Synthetic polynucleotide
  source 1..1356: mol_type = other DNA
atggacgggactattaaggaggctctgtcggtggtgagcgacgaccagtccctctttgactcagcgtacggagcggcagcccatctccccaaggccgacatgactgcctcggggagtcctgactacgggcagccccacaagatcaaccccctcccaccacagcaggagtggatcaatcagccagtgagggtcaacgtcaagcgggagtatgaccacatgaatggatccagggagtctccggtggactgcagcgttagcaaatgcagcaagctggtgggcggaggcgagtccaaccccatgaactacaacagctatatggacgagaagaatggcccccctcctcccaacatgaccaccaacgagaggagagtcatcgtccccgcagaccccacactgtggacacaggagcatgtgaggcaatggctggagtgggccataaaggagtacagcttgatggagatcgacacatcctttttccagaacatggatggcaaggaactgtgtaaaatgaacaaggaggacttcctccgcgccaccaccctctacaacacggaagtgctgttgtcacacctcagttacctcagggaaagttcactgctggcctataatacaacctcccacaccgaccaatcctcacgattgagtgtcaaagaagacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactac

SEQ ID NO: 45 - DNA, 1416 bp, synthetic construct
  misc_feature 1..1416: note = Description of Artificial Sequence: Synthetic polynucleotide
  source 1..1416: mol_type = other DNA
atgttgggcaccgtgaagatggaggggcatgagacaagcgactggaattcctactacgcggatacccaagaagcgtattcttcagttcccgtaagcaatatgaactccggattggggagcatgaatagtatgaacacgtatatgacaatgaatacgatgaccaccagcggcaacatgacaccggcctcctttaatatgtcatatgcgaaccctggtcttggcgctggcctctcaccaggtgcggtcgctggaatgcccggggggagcgccggagcgatgaactccatgaccgctgcgggcgtgacggccatgggtacggccctgtcacccagtggaatgggagctatgggggcccagcaagccgcctcaatgaatggattggggccctatgccgcggcgatgaatccctgcatgtcccctatggcttatgcccccagcaatttgggtcgcagtagagccggcggtggtggcgatgccaaaaccttcaagcgaagttatcctcatgcgaagcctccttattcatatatatccttgattacgatggcgatacagcaggccccgtctaagatgctgactctgagtgagatataccagtggatcatggacctttttccttactaccggcaaaaccaacagagatggcaaaactcaatacgccatagcctttccttcaatgattgctttgtcaaagtcgctcggagccctgacaagcccggtaaagggtcctattggacccttcatccagatagcggcaatatgttcgagaatggttgttatcttagacggcagaaacgattcaaatgtgagaaacagccaggtgccggcggtggtggcggcagcggttcaggcggaagtggtgccaagggtgggcctgagtctagaaaagaccccagcggagcaagcaatccaagcgcggactctcccctgcaccgcggtgttcatggtaagacaggtcagcttgagggggcgcctgctccaggcccggctgcgtcaccgcaaacactggaccatagtggagctacagcgaccggaggtgcttcagaactcaagacgcctgcgtcctccactgcgcctccgatctccagtggtcccggtgcacttgcctctgttcctgcatctcatccagcacacggactcgcgccgcacgagtcccagctccatttgaaaggggacccacactacagctttaaccacccattctctattaacaatttgatgtcatcctcagaacagcagcataaactcgacttcaaagcctatgaacaggccctgcagtattctccatatggctctacacttcctgcttctcttccattggggtctgcaagtgtgacaacgcgctccccaatcgagccaagtgccctcgagcctgcttattatcaaggagtatattcccgaccagttttgaatacaagt

SEQ ID NO: 46 - DNA, 1374 bp, synthetic construct
  misc_feature 1..1374: note = Description of Artificial Sequence: Synthetic polynucleotide
  source 1..1374: mol_type = other DNA
atgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggctggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggggcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccagccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctctttg

SEQ ID NO: 47 - DNA, 1050 bp, synthetic construct
  misc_feature 1..1050: note = Description of Artificial Sequence: Synthetic polynucleotide
  source 1..1050: mol_type = other DNA
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcc

SEQ ID NO: 48 - DNA, 342 bp, synthetic construct
  misc_feature 1..342: note = Description of Artificial Sequence: Synthetic polynucleotide
  source 1..342: mol_type = other DNA
atgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacagtggcatctcataaaccatcagccctctaggagtcccagcagttggcttaagagactaatttcaagcccttgggagttggaagtcctgcaggtccccttgtggggagcagttgctgagacgaagatgagtggacctgtgtgtcagcctaacccttccccattt

SEQ ID NO: 49 - DNA, 1005 bp, synthetic construct
  misc_feature 1..1005: note = Description of Artificial Sequence: Synthetic polynucleotide
  source 1..1005: mol_type = other DNA
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccagtctttcaggtgtacccattgctcaactgtatggaggggatcccagggggctcaccatatgccggctgggcctacggcaagacggggctctaccctgcctcaactgtgtgtcccacccgcgaggactctcctccccaggccgtggaagatctggatggaaaaggcagcaccagcttcctggagactttgaagacagagcggctgagcccagacctcctgaccctgggacctgcactgccttcatcactccctgtccccaatagtgcttatgggggccctgacttttccagtaccttcttttctcccaccgggagccccctcaattcagcagcctattcctctcccaagcttcgtggaactctccccctgcctccctgtgaggccagggagtgtgtgaactgcggagcaacagccactccactgtggcggagggacaggacaggccactacctatgcaacgcctgcggcctctatcacaagatgaatgggcagaacaggcccctcatccggcccaagaagcgcctgattgtcagtaaacgggcaggtactcagtgcaccaactgccagacgaccaccacgacactgtggcggagaaatgccagtggggatcccgtgtgcaatgcctgcggcctctactacaagctacaccaccagcactactgtggtggctccgctcagctcatgagggcacagagcatggcctccagaggaggggtggtgtccttctcctcttgtagccagaattctggacaacccaagtctctgggccccaggcaccccctggct

SEQ ID NO: 50 - DNA, 1440 bp, synthetic construct
  misc_feature 1..1440: note = Description of Artificial Sequence: Synthetic polynucleotide
  source 1..1440: mol_type = other DNA
atggaggtggcgccggagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcaacccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggc

SEQ ID NO: 51 - DNA, 1326 bp, synthetic construct
  misc_feature 1..1326: note = Description of Artificial Sequence: Synthetic polynucleotide
  source 1..1326: mol_type = other DNA
atgtaccagagcctggctatggctgctaatcatggacctccccctggagcctatgaagccggaggacctggcgcttttatgcatggagctggcgccgcttcttctcccgtgtatgtgcctacacctagagtgcccagcagcgtgctgggcctttcttatcttcagggaggaggagcaggatctgcttctggcggagcttcaggcggatcttctggaggcgctgcttcaggtgctggacctggaactcaacagggatctcctggatggtcacaggcaggagctgatggagccgcttatacccctcctcctgtgagccccaggtttagctttcctggcacaacaggctctttagctgccgctgctgctgcagccgcagctagagaagcagctgcatattctagtggcggaggagctgctggagccggcttagctggaagagagcagtacggaagagccggatttgccggaagctatagcagcccttaccctgcctatatggccgatgttggcgcatcttgggcagccgccgcagcagcttctgcaggaccttttgactcacctgtgcttcactctctgcctggcagagctaatcctgccgccagacatcccaacctggacatgttcgacgacttcagcgagggcagagaatgcgtgaactgcggagccatgagcacccccctttggagaagagacggcaccggccactacctttgcaatgcctgtggcctgtaccacaagatgaacggcatcaacagacccctgatcaagccccagagaagactgagcgctagcagaagagtgggcctgtcctgcgccaattgccagaccacaaccaccacactgtggaggagaaatgccgagggcgagcctgtgtgtaacgcctgtggactgtacatgaagctgcacggcgtgcccagacctctggccatgagaaaggagggcatccagaccagaaagagaaagcccaagaacctgaacaagagcaagacccccgctgctccttctggaagcgagagcctgcctccagcctctggagccagcagcaatagctctaacgccaccacatcttcttctgaggagatgaggcccatcaaaaccgagccaggcctgagcagccactacggccacagctctagcgtgagccagacttttagcgtgtctgccatgtcaggccacggacctagcattcaccctgtgctgagcgccctgaagttgagcccacagggctatgcttctcctgtgtctcagagccctcagacctccagcaagcaggactcctggaattctctggtgctggccgacagccacggcgatatcatcaccgcc

SEQ ID NO: 52 - DNA, 1785 bp, synthetic construct
  misc_feature 1..1785: note = Description of Artificial Sequence: Synthetic polynucleotide
  source 1..1785: mol_type = other DNA
atggccctgaccgacggcggatggtgtctccctaaaagattcggcgccgctggcgctgatgcttctgacagcagagccttccccgctagggaacccagcacaccacctagccccatcagcagctcaagctctagctgtagcagaggcggagagagaggacctggaggcgcttctaactgcggcacacctcagctggatacagaagccgccgccggaccaccagccagatctcttttacttagcagctacgccagccacccttttggcgctcctcatggaccctctgctcctggtgtggccggacctggcggaaacctgagctcttgggaggaccttctgctgtttaccgacctggaccaggctgccaccgctagcaagcttctgtggagcagcaggggcgctaagctgagcccttttgcccctgagcagcccgaggagatgtaccagaccctggctgctttaagctctcagggacctgccgcttatgacggagcccctggtggatttgttcactcagcggcagcagccgcagctgctgcagccgctgccagctcacctgtgtatgtgcctaccacaagagtgggcagcatgttacctggacttccttaccatctgcagggcagcggaagcggccctgctaaccatgccggaggagctggagctcaccccggatggcctcaggcttctgcagattctcctccttatggatctggaggaggagcagctggagggggagctgcaggaccaggtggagccggaagcgcagcagcacatgtgtctgccagatttccctatagccctagccctcctatggccaatggcgctgctagagaacccggaggatatgctgcggcaggctctggcggcgctggcggagtttctggaggtggatcttcactggccgctatgggaggaagagagcctcagtactcttctctgagcgccgctagaccactgaacggcacctatcatcaccaccaccatcaccatcatcatcaccccagcccttactccccttatgtgggagccccccttacacccgcttggcctgccggccctttcgagacacctgtgctgcacagccttcagtctagagctggcgcacctttaccagtgcctagaggcccctctgccgacttgctggaggatctgagcgagagcagagagtgcgtgaactgtggcagcatccagacacccctgtggagaagagacggcaccggccactacctgtgcaacgcttgcggcctgtacagcaagatgaatgggctgagcagacccctgatcaagccccagaagagggtgcccagcagcagacggctgggactgagctgcgccaactgtcataccacaacaaccacactgtggcggagaaacgccgagggcgagcccgtgtgtaacgcctgcggcctttacatgaagctgcacggcgtgcccagacctctggccatgaagaaggagggaatccagaccagaaagagaaagcccaagaacatcaacaagagcaagacctgcagcggcaacagcaacaacagcatccccatgacccccaccagcacatctagcaacagcgacgactgtagcaagaacacatcacctaccacccagcccacagctagcggagccggcgcccccgtgatgacaggcgccggagagtccacaaatcccgagaatagcgaactgaagtactctggacaggacggactgtatatcggcgtgagcctggcttctcccgccgaggtgaccagctctgtcagacctgactcttggtgtgccctcgccctggcc

SEQ ID NO: 53 - DNA, 3318 bp, synthetic construct
  misc_feature 1..3318: note = Description of Artificial Sequence: Synthetic polynucleotide
  source 1..3318: mol_type = other DNA
atgttcaactcgatgaccccaccaccaatcagtagctatggcgagccctgctgtctccggcccctccccagtcagggggcccccagtgtggggacagaaggactgtctggcccgcccttctgccaccaagctaacctcatgtccggcccccacagttatgggccagccagagagaccaacagctgcaccgagggcccactcttttcttctccccggagtgcagtcaagttgaccaagaagcgggcactgtccatctcacctctgtcggatgccagcctggacctgcagacggttatccgcacctcacccagctccctcgtagctttcatcaactcgcgatgcacatctccaggaggctcctacggtcatctctccattggcaccatgagcccatctctgggattcccagcccagatgaatcaccaaaaagggccctcgccttcctttggggtccagccttgtggtccccatgactctgcccggggtgggatgatcccacatcctcagtcccggggacccttcccaacttgccagctgaagtctgagctggacatgctggttggcaagtgccgggaggaacccttggaaggtgatatgtccagccccaactccacaggcatacaggatcccctgttggggatgctggatgggcgggaggacctcgagagagaggagaagcgtgagcctgaatctgtgtatgaaactgactgccgttgggatggctgcagccaggaatttgactcccaagagcagctggtgcaccacatcaacagcgagcacatccacggggagcggaaggagttcgtgtgccactgggggggctgctccagggagctgaggcccttcaaagcccagtacatgctggtggttcacatgcgcagacacactggcgagaagccacacaagtgcacgtttgaagggtgccggaagtcatactcacgcctcgaaaacctgaagacgcacctgcggtcacacacgggtgagaagccatacatgtgtgagcacgagggctgcagtaaagccttcagcaatgccagtgaccgagccaagcaccagaatcggacccattccaatgagaagccgtatgtatgtaagctccctggctgcaccaaacgctatacagatcctagctcgctgcgaaaacatgtcaagacagtgcatggtcctgacgcccatgtgaccaaacggcaccgtggggatggccccctgcctcgggcaccatccatttctacagtggagcccaagagggagcgggaaggaggtcccatcagggaggaaagcagactgactgtgccagagggtgccatgaagccacagccaagccctggggcccagtcatcctgcagcagtgaccactccccggcagggagtgcagccaatacagacagtggtgtggaaatgactggcaatgcagggggcagcactgaagacctctccagcttggacgagggaccttgcattgctggcactggtctgtccactcttcgccgccttgagaacctcaggctggaccagctacatcaactccggccaatagggacccggggtctcaaactgcccagcttgtcccacaccggtaccactgtgtcccgccgcgtgggccccccagtctctcttgaacgccgcagcagcagctccagcagcatcagctctgcctatactgtcagccgccgctcctccctggcctctcctttcccccctggctccccaccagagaatggagcatcctccctgcctggccttatgcctgcccagcactacctgcttcgggcaagatatgcttcagccagagggggtggtacttcgcccactgcagcatccagcctggatcggataggtggtcttcccatgcctccttggagaagccgagccgagtatccaggatacaaccccaatgcaggggtcacccggagggccagtgacccagcccaggctgctgaccgtcctgctccagctagagtccagaggttcaagagcctgggctgtgtccataccccacccactgtggcagggggaggacagaactttgatccttacctcccaacctctgtctactcaccacagccccccagcatcactgagaatgctgccatggatgctagagggctacaggaagagccagaagttgggacctccatggtgggcagtggtctgaacccctatatggacttcccacctactgatactctgggatatgggggacctgaaggggcagcagctgagccttatggagcgaggggtccaggctctctgcctcttgggcctggtccacccaccaactatggccccaacccctgtccccagcaggcctcatatcctgaccccacccaagaaacatggggtgagttcccttcccactctgggctgtacccaggccccaaggctctaggtggaacctacagccagtgtcctcgacttgaacattatggacaagtgcaagtcaagccagaacaggggtgcccagtggggtctgactccacaggactggcaccctgcctcaatgcccaccccagtgaggggcccccacatccacagcctctcttttcccattacccccagccctctcctccccaatatctccagtcaggcccctatacccagccaccccctgattatcttccttcagaacccaggccttgcctggactttgattcccccacccattccacagggcagctcaaggctcagcttgtgtgtaattatgttcaatctcaacaggagctactgtgggagggtgggggcagggaagatgcccccgcccaggaaccttcctaccagagtcccaagtttctggggggttcccaggttagcccaagccgtgctaaagctccagtgaacacatatggacctggctttggacccaacttgcccaatcacaagtcaggttcctatcccaccccttcaccatgccatgaaaattttgtagtgggggcaaatagggcttcacatagggcagcagcaccacctcgacttctgcccccattgcccacttgctatgggcctctcaaagtgggaggcacaaaccccagctgtggtcatcctgaggtgggcaggctaggagggggtcctgccttgtaccctcctcccgaaggacaggtatgtaaccccctggactctcttgatcttgacaacactcagctggactttgtggctattctggatgagccccaggggctgagtcctcctccttcccatgatcagcggggcagctctggacataccccacctccctctgggccccccaacatggctgtgggcaacatgagtgtcttactgagatccctacctggggaaacagaattcctcaactctagtgcc

SEQ ID NO: 54 - DNA, 540 bp, synthetic construct
  misc_feature 1..540: note = Description of Artificial Sequence: Synthetic polynucleotide
  source 1..540: mol_type = other DNA
atgagtctggtaggtggttttccccaccacccggtggtgcaccacgagggctacccgtttgccgccgccgccgccgccagccgctgcagccatgaggagaacccctacttccatggctggctcatcggccaccccgagatgtcgccccccgactacagcatggccctgtcctacagccccgagtatgccagcggcaccgccaaccgcaaggagcggcgcaggactcagagcatcaacagcgccttcgccgaactgcgcgagtgcatccccaacgtacccgccgacaccaaactctccaaaatcaagaccctgcgcctggccaccagctacatcgcctacctcatggacctgctggccaaggacgaccagaatggcgaggcggaggccttcaaggcagagatcaagaagaccgacgtgaaagaggagaagaggaagaaggagctgaacgaaatcttgaaaagcacagtgagcagcaacgacaagaaaaccaaaggccggacgggctggccgcagcacgtctgggccctggagctcaagcag

SEQ ID NO: 55 - DNA, 1896 bp, synthetic construct
  misc_feature 1..1896: note = Description of Artificial Sequence: Synthetic polynucleotide
  source 1..1896: mol_type = other DNA
atggtttctaaactgagccagctgcagacggagctcctggcggccctgctggagtcagggctgagcaaagaggcactgctccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacaggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagttg

SEQ ID NO: 56 - DNA, 1671 bp, synthetic construct
  misc_feature 1..1671: note = Description of Artificial Sequence: Synthetic polynucleotide
  source 1..1671: mol_type = other DNA
atggttagcaaactgacatccctccagcaggaacttctttctgccctcctctccagtggggtaaccaaagaggtactggtccaggctttggaggagttgctcccctcaccgaattttggtgtaaagttggagactctccccctctcccctggttctggagcagagccggatactaaaccggtatttcatacgcttacaaacggacacgcaaagggtcggctttcaggtgacgaagggtctgaggacggcgatgattatgacaccccgcccatcctcaaagaactgcaggcccttaatacagaggaagcggcggagcagcgagctgaagttgacagaatgctctcagaagatccgtggagagctgcgaaaatgattaagggatatatgcagcaacataacattccccagagagaggtagttgatgttaccggccttaaccagagccacctgtctcagcatctcaataagggtactcctatgaaaacacagaagcgagcggccctttacacatggtacgtgcggaagcaacgagaaattctccgacagttcaatcagacagtacaatcttcagggaacatgacggataaaagctcacaggatcagctcttgtttctcttccccgagttcagccaacagtcccacggtccaggtcaatctgatgatgcttgcagtgaacctacaaacaaaaaaatgaggaggaacaggtttaaatggggaccggcctctcagcagatactgtaccaagcgtacgatcggcagaaaaacccaagcaaagaggagcgcgaggcattggtcgaggagtgtaatcgggccgagtgcttgcaacggggtgtaagtcctagcaaagcccatggtctcggctcaaacttggtcacggaggtgagggtatataattggtttgccaacaggcggaaggaggaagcattccggcaaaagctggcgatggatgcctactcaagcaaccagacacatagcctcaaccctctgttgtcacacgggtcccctcatcaccaaccttcttcctctccacccaacaaactttctggtgtccgatattcccagcaggggaacaacgagataacatcttcctctactataagtcatcacggaaattctgcaatggtaacgtcacagagtgtgttgcaacaggtatcacccgcgtctcttgatccaggccacaatctgttgagccctgacggaaagatgatctctgtttctggtggcggactcccgccggtctccacacttaccaacatacatagtctcagtcatcataatcctcagcagagccaaaacctgattatgactcctcttagcggagtgatggctattgcgcaatctttgaacacctcacaagcacaatctgtacccgtcataaacagcgtagcgggctcattggcggcgctccaaccagtgcagttctcccagcagctccattcaccccatcaacagcctctgatgcagcagagccctggtagtcacatggctcaacagccgttcatggcagctgtcactcagctccagaactcccatatgtatgcccacaagcaagaaccaccacaatacagtcacacatcaagattccccagtgctatggttgttactgacacatcctctatctcaactctgacgaacatgtccagtagtaaacaatgtcctctgcaagcatgg

SEQ ID NO: 57 - DNA, 1251 bp, synthetic construct
  misc_feature 1..1251: note = Description of Artificial Sequence: Synthetic polynucleotide
  source 1..1251: mol_type = other DNA
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgct

SEQ ID NO: 58 - DNA, 1005 bp, synthetic construct
  misc_feature 1..1005: note = Description of Artificial Sequence: Synthetic polynucleotide
  source 1..1005: mol_type = other DNA
atggacaacgcgcggatgaattccttcctcgagtacccaattttgtctagtggagacagtggcacttgcagtgcccgagcctatccatcagaccacagaattacaacattccaaagctgtgcggtgtcagccaacagttgcggcggagacgaccgcttcctggtcggaagaggggttcaaattggatcacctcaccatcaccatcaccaccaccatcaccacccccaaccggcgacttaccaaaccagcggcaatttgggcgtgagctatagccattcctcatgtggaccttcctatgggtctcagaatttctccgccccttatagcccatacgccctgaaccaagaggccgatgtatcaggaggctatccccagtgcgcgccagcggtttactcaggtaatctttctagcccgatggtccagcaccaccatcaccatcaaggttatgccggcggtgcagtcggatccccacaatacatacaccatagttacggccaagagcaccaatccctggccctcgctacatataacaactcactgtctccgcttcatgcttcccaccaagaagcttgtcggagtcccgcctcagaaacttcctctccagctcagacttttgattggatgaaggtcaagcggaatccgcctaaaacgggcaaagtaggtgaatatggctatttgggacagcctaatgctgtccgcaccaatttcacaacaaaacagcttactgaactcgagaaggaatttcattttaataagtatttgactcgagcgagacgagtcgaaatcgccgctagtcttcaacttaacgagacccaggttaagatatggttccagaacagaagaatgaaacaaaaaaagcgggagaaggaaggactcctccctatatcaccagccacacccccaggtaacgacgagaaggcggaggaatcttcagagaagagttccagctccccttgtgttccttctcctggtagctcaaccagcgataccctcacgacgagtcac

SEQ ID NO: 59 - DNA, 282 bp, synthetic construct
  misc_feature 1..282: note = Description of Artificial Sequence: Synthetic polynucleotide
  source 1..282: mol_type = other DNA
atgtgtcaaggcaattccaaaggtgaaaacgcagccaactggctcacggcaaagagtggtcggaagaagcgctgcccctacacgaagcaccagacactggagctggagaaggagtttctgttcaatatgtaccttactcgagagcggcgcctagagattagccgcagcgtccacctcacggacagacaagtgaaaatctggtttcagaaccgcaggatgaaactgaagaaaatgaatcgagaaaaccggatccgggagctcacagccaactttaatttttcc

SEQ ID NO: 60 - DNA, 942 bp, synthetic construct
  misc_feature 1..942: note = Description of Artificial Sequence: Synthetic polynucleotide
  source 1..942: mol_type = other DNA
atggattttgatgagcgtggtccctgctcctctaacatgtatttgccaagttgtacttactacgtctcgggtccagatttctccagcctcccttcttttctgccccagaccccgtcttcgcgcccaatgacatactcctactcctccaacctgccccaggtccaacccgtgcgcgaagtgaccttcagagagtacgccattgagcccgccactaaatggcacccccgcggcaatctggcccactgctactccgcggaggagctcgtgcacagagactgcctgcaggcgcccagcgcggccggcgtgcctggcgacgtgctggccaagagctcggccaacgtctaccaccaccccacccccgcagtctcgtccaatttctatagcaccgtgggcaggaacggcgtcctgccacaggctttcgaccagtttttcgagacagcctacggcaccccggaaaacctcgcctcctccgactaccccggggacaagagcgccgagaaggggcccccggcggccacggcgacctccgcggcggcggcggcggctgcaacgggcgcgccggcaacttcaagttcggacagcggcggcggcggcggctgccgggagatggcggcggcagcagaggagaaagagcggcggcggcgccccgagagcagcagcagccccgagtcgtcttccggccacactgaggacaaggccggcggctccagtggccaacgcacccgcaaaaagcgctgcccctataccaagtaccagatccgagagctggaacgggagttcttcttcagcgtctacattaacaaagagaagcgcctgcaactgtcccgcatgctcaacctcactgatcgtcaagtcaaaatctggtttcagaacaggagaatgaaggaaaaaaaaattaacagagaccgtttacagtactactcagcaaatccactcctcttg

SEQ ID NO: 61 - DNA, 672 bp, synthetic construct
  misc_feature 1..672: note = Description of Artificial Sequence: Synthetic polynucleotide
  source 1..672: mol_type = other DNA
atgagttcctatttcgtgaactccaccttccccgtcactctggccagcgggcaggagtccttcctgggccagctaccgctctattcgtcgggctatgcggacccgctgagacattaccccgcgccctacgggccagggccgggccaggacaagggctttgccacttcctcctattacccgccggcgggcggtggctacggccgagcggcgccctgcgactacgggccggcgccggccttctaccgcgagaaagagtcggcctgcgcactctccggcgccgacgagcagcccccgttccaccccgagccgcggaagtcggactgcgcgcaggacaagagcgtgttcggcgagacagaagagcagaagtgctccactccggtctacccgtggatgcagcggatgaattcgtgcaacagttcctcctttgggcccagcggccggcgaggccgccagacatacacacgttaccagacgctggagctggagaaggagtttcactacaatcgctacctgacgcggcggcggcgcatcgagatcgcgcacgccctgtgcctgacggagaggcagatcaagatatggttccagaaccgacgcatgaagtggaaaaaggagagcaaactgctcagcgcgtctcagctcagtgccgaggaggaggaagaaaaacaggccgag

SEQ ID NO: 62 - DNA, 1410 bp, synthetic construct
  misc_feature 1..1410: note = Description of Artificial Sequence: Synthetic polynucleotide
  source 1..1410: mol_type = other DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 63 - DNA, 1236 bp, synthetic construct
  misc_feature 1..1236: note = Description of Artificial Sequence: Synthetic polynucleotide
  source 1..1236: mol_type = other DNA
atggaggcgcgcggggagctgggcccggcccgggagtcggcgggaggcgacctgctgctagcactgctggcgcggagggcggacctgcgccgagagatcccgctgtgcgctggctgtgaccagcacatcctggaccgcttcatcctcaaggctctggaccgccactggcacagcaagtgtctcaagtgcagcgactgccacacgccactggccgagcgctgcttcagccgaggggagagcgtttactgcaaggacgactttttcaagcgcttcgggaccaagtgcgccgcgtgccagctgggcatcccgcccacgcaggtggtgcgccgcgcccaggacttcgtgtaccacctgcactgctttgcctgcgtcgtgtgcaagcggcagctggccacgggcgacgagttctacctcatggaggacagccggctcgtgtgcaaggcggactacgaaaccgccaagcagcgagaggccgaggccacggccaagcggccgcgcacgaccatcaccgccaagcagctggagacgctgaagagcgcttacaacacctcgcccaagccggcgcgccacgtgcgcgagcagctctcgtccgagacgggcctggacatgcgcgtggtgcaggtttggttccagaaccgccgggccaaggagaagaggctgaagaaggacgccggccggcagcgctgggggcagtatttccgcaacatgaagcgctcccgcggcggctccaagtcggacaaggacagcgttcaggaggggcaggacagcgacgctgaggtctccttccccgatgagccttccttggcggaaatgggcccggccaatggcctctacgggagcttgggggaacccacccaggccttgggccggccctcgggagccctgggcaacttctccctggagcatggaggcctggcaggcccagagcagtaccgagagctgcgtcccggcagcccctacggtgtccccccatcccccgccgccccgcagagcctccctggcccccagcccctcctctccagcctggtgtacccagacaccagcttgggccttgtgccctcgggagcccccggcgggcccccacccatgagggtgctggcagggaacggacccagttctgacctatccacggggagcagcgggggttaccccgacttccctgccagccccgcctcctggctggatgaggtagaccacgctcagttctcaggcctcatgggcccagctttcttgtac

SEQ ID NO: 64 - DNA, 300 bp, synthetic construct
  misc_feature 1..300: note = Description of Artificial Sequence: Synthetic polynucleotide
  source 1..300: mol_type = other DNA
atggaaggaatcatgaacccctacacggctctgcccaccccacagcagctcctggccatcgagcagagtgtctacagctcagatcccttccgacagggtctcaccccaccccagatgcctggagaccacatgcacccttatggtgccgagccccttttccatgacctggatagcgacgacacctccctcagtaacctgggtgactgtttcctagcaacctcagaagctgggcctctgcagtccagagtgggaaaccccattgaccatctgtactccatgcagaattcttacttcacatct

SEQ ID NO: 65 - DNA, 1419 bp, synthetic construct
  misc_feature 1..1419: note = Description of Artificial Sequence: Synthetic polynucleotide
  source 1..1419: mol_type = other DNA
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaaca

SEQ ID NO: 66 - DNA, 807 bp, synthetic construct
  misc_feature 1..807: note = Description of Artificial Sequence: Synthetic polynucleotide
  source 1..807: mol_type = other DNA
atggcccagcccctgtgcccgccgctctccgagtcctggatgctctctgcggcctggggcccaactcggcggccgccgccctccgacaaggactgcggccgctccctcgtctcgtccccagactcatggggcagcaccccagccgacagccccgtggcgagccccgcgcggccaggcaccctccgggacccccgcgccccctccgtaggtaggcgcggcgcgcgcagcagccgcctgggcagcgggcagaggcagagcgccagtgagcgggagaaactgcgcatgcgcacgctggcccgcgccctgcacgagctgcgccgctttctaccgccgtccgtggcgcccgcgggccagagcctgaccaagatcgagacgctgcgcctggctatccgctatatcggccacctgtcggccgtgctaggcctcagcgaggagagtctccagcgccggtgccggcagcgcggtgacgcggggtcccctcggggctgcccgctgtgccccgacgactgccccgcgcagatgcagacacggacgcaggctgaggggcaggggcaggggcgcgggctgggcctggtatccgccgtccgcgccggggcgtcctggggatccccgcctgcctgccccggagcccgagctgcacccgagccgcgcgacccgcctgcgctgttcgccgaggcggcgtgcccggaagggcaggcgatggagccaagcccaccgtccccgctccttccgggcgacgtgctggctctgttggagacctggatgcccctctcgcctctggagtggctgcctgaggagcccaagttg

SEQ ID NO: 67 - DNA, 1239 bp, synthetic construct
  misc_feature 1..1239: note = Description of Artificial Sequence: Synthetic polynucleotide
  source 1..1239: mol_type = other DNA
atgctggaaatgctagaatataatcactatcaggtgcagacccacctcgaaaaccccaccaagtaccacatacagcaagcccaacggcagcaggtaaagcagtacctttctaccactttagcaaataaacatgccaaccaagtcctgagcttgccatgtccaaaccagcctggcgatcatgtcatgccaccggtgccggggagcagcgcacccaacagccccatggctatgcttacgcttaactccaactgtgaaaaagagggattttataagtttgaagagcaaaacagggcagagagcgagtgcccaggcatgaacacacattcacgagcgtcctgtatgcagatggatgatgtaatcgatgacatcattagcctagaatcaagttataatgaggaaatcttgggcttgatggatcctgctttgcaaatggcaaatacgttgcctgtctcgggaaacttgattgatctttatggaaaccaaggtctgcccccaccaggcctcaccatcagcaactcctgtccagccaaccttcccaacataaaaagggagctcacagagtctgaagcaagagcactggccaaagagaggcagaaaaaggacaatcacaacctgattgaacgaagaagaagatttaacataaatgaccgcattaaagaactaggtactttgattcccaagtcaaatgatccagacatgcgctggaacaagggaaccatcttaaaagcatccgtggactatatccgaaagttgcaacgagaacagcaacgcgcaaaagaacttgaaaaccgacagaagaaactggagcacgccaaccggcatttgttgctcagaatacaggaacttgaaatgcaggctcgagctcatggactttcccttattccatccacgggtctctgctctccagatttggtgaatcggatcatcaagcaagaacccgttcttgagaactgcagccaagacctccttcagcatcatgcagacctaacctgtacaacaactctcgatctcacggatggcaccatcaccttcaacaacaacctcggaactgggactgaggccaaccaagcctatagtgtccccacaaaaatgggatccaaactggaagacatcctgatggacgacaccctttctcccgtcggtgtcactgatccactcctttcctcagtgtcccccggagcttccaaaacaagcagccggaggagcagtatgagcatggaagagacggagcacacttgt

SEQ ID NO: 68 - DNA, 1317 bp, synthetic construct
  misc_feature 1..1317: note = Description of Artificial Sequence: Synthetic polynucleotide
  source 1..1317: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 69 - DNA, 618 bp, synthetic construct
  misc_feature 1..618: note = Description of Artificial Sequence: Synthetic polynucleotide
  source 1..618: mol_type = other DNA
atggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcgggtaaggacctccccgagccatccaagagggggccaccccatgggtggccaaagctctgcccctgcctgaggtcaggcattggctcttctcaagctcttgggccatctccgcctctctttggc

SEQ ID NO: 70 - DNA, 1392 bp, synthetic construct
  misc_feature 1..1392: note = Description of Artificial Sequence: Synthetic polynucleotide
  source 1..1392: mol_type = other DNA
atgccgagttgttccacgtctacgatgccaggaatgatatgcaagaaccccgacttggagtttgactctttgcaaccatgcttttatccggatgaagacgacttttatttcggcggcccggacagcacccctcctggagaggacatctggaaaaaattcgaacttttgcctacacccccactcagtccctctcgaggatttgcggaacacagcagtgaaccgccgtcttgggtgacagagatgctcctcgagaacgaattgtggggaagccctgcggaggaagacgctttcgggctcggtggactcggaggtctcacgccgaacccagtcatactgcaggattgcatgtggtctggattctcagctcgggagaagctggaacgggcagtttctgagaaactccaacatggccggggccctccaacagcgggttctaccgcacagtcccctggtgctggagccgctagtcccgcggggagaggccatgggggcgcggcaggagcgggtagggccggcgctgcgttgcctgctgagcttgcgcaccccgccgctgaatgtgtagatcccgcggtagtgtttccgttccccgttaataagcgagaaccggcaccggtgccagccgctcctgcgtctgcacccgcggcaggtcctgctgtcgcctcaggagcaggtattgccgctcctgcaggggcaccaggagtagcccctccaaggcccggcggtaggcaaacctccggcggcgaccacaaagcactctcaacgagcggagaggatacactgtccgatagtgatgacgaggacgacgaagaggaggacgaggaggaggagatagatgttgtcacggtcgagaagcgaaggagttcttcaaatacaaaagcggtaacgacattcacgataacagtaagacctaagaacgcagccctcggtccagggcgggcccagtccagtgagcttatacttaagcgctgcctgccgattcaccagcagcataactacgcggcccctagtccctacgttgagagcgaggatgcccccccacaaaaaaaaataaagtctgaagcgtccccccgccccctgaaatccgtaatccccccaaaggcgaagtcactcagtcccaggaattcagattccgaggactccgaacggcggcggaatcataacatacttgagagacaacgacgcaatgacctgaggtcttcttttttgaccctccgagatcacgtccccgagctggttaagaatgagaaagctgcgaaggtagtcatactgaaaaaggccaccgagtatgtccatagtttgcaagctgaggagcaccagcttctccttgaaaaggagaaacttcaggcacgacaacagcaattgctgaaaaagattgagcatgcacgcacttgt

SEQ ID NO: 71 - DNA, 960 bp, synthetic construct
  misc_feature 1..960: note = Description of Artificial Sequence: Synthetic polynucleotide
  source 1..960: mol_type = other DNA
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctc

SEQ ID NO: 72 - DNA, 672 bp, synthetic construct
  misc_feature 1..672: note = Description of Artificial Sequence: Synthetic polynucleotide
  source 1..672: mol_type = other DNA
atggagctgtatgagacatccccctacttctaccaggaaccccgcttctatgatggggaaaactacctgcctgtccacctccagggcttcgaaccaccaggctacgagcggacggagctcaccctgagccccgaggccccagggccccttgaggacaaggggctggggacccccgagcactgtccaggccagtgcctgccgtgggcgtgtaaggtgtgtaagaggaagtcggtgtccgtggaccggcggcgggcggccacactgagggagaagcgcaggctcaagaaggtgaatgaggccttcgaggccctgaagagaagcaccctgctcaaccccaaccagcggctgcccaaggtggagatcctgcgcagtgccatccagtacatcgagcgcctccaggccctgctcagctccctcaaccaggaggagcgtgacctccgctaccggggcgggggcgggccccagccaggggtgcccagcgaatgcagctctcacagcgcctcctgcagtccagagtggggcagtgcactggagttcagcgccaacccaggggatcatctgctcacggctgaccctacagatgcccacaacctgcactccctcacctccatcgtggacagcatcacagtggaagatgtgtctgtggccttcccagatgaaaccatgcccaac

SEQ ID NO: 73 - DNA, 1068 bp, synthetic construct
  misc_feature 1..1068: note = Description of Artificial Sequence: Synthetic polynucleotide
  source 1..1068: mol_type = other DNA
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgat

SEQ ID NO: 74 - DNA, 711 bp, synthetic construct
  misc_feature 1..711: note = Description of Artificial Sequence: Synthetic polynucleotide
  source 1..711: mol_type = other DNA
atgccagcccgccttgagacctgcatctccgacctcgactgcgccagcagcagcggcagtgacctatccggcttcctcaccgacgaggaagactgtgccagactccaacaggcagcctccgcttcggggccgcccgcgccggcccgcaggggcgcgcccaatatctcccgggcgtctgaggttccaggggcacaggacgacgagcaggagaggcggcggcgccgcggccggacgcgggtccgctccgaggcgctgctgcactcgctgcgcaggagccggcgcgtcaaggccaacgatcgcgagcgcaaccgcatgcacaacttgaacgcggccctggacgcactgcgcagcgtgctgccctcgttccccgacgacaccaagctcaccaaaatcgagacgctgcgcttcgcctacaactacatctgggctctggccgagacactgcgcctggcggatcaagggctgcccggaggcggtgcccgggagcgcctcctgccgccgcagtgcgtcccctgcctgcccggtcccccaagccccgccagcgacgcggagtcctggggctcaggtgccgccgccgcctccccgctctctgaccccagtagcccagccgcctccgaagacttcacctaccgccccggcgaccctgttttctccttcccaagcctgcccaaagacttgctccacacaacgccctgtttcattccttaccac

SEQ ID NO: 75 - DNA, 642 bp, synthetic construct
  misc_feature 1..642: note = Description of Artificial Sequence: Synthetic polynucleotide
  source 1..642: mol_type = other DNA
atgacaccacaaccatctggtgctcccacagtccaggtgacgcgagagactgaaagatcattcccacgcgcgtccgaggatgaggtgacatgtccaactagcgcacccccctctcctacccggacccgcgggaattgtgctgaggccgaagagggaggatgcagaggagcaccaaggaaacttcgagcccgacggggtggaagaagccgccccaagtctgagctcgcccttagcaagcagcgccgcagtcggaggaaaaaggcaaacgaccgggaaaggaataggatgcataatcttaattctgctctggacgctctgcgaggcgtacttcctactttcccggatgacgcgaaattgaccaagatagagactctccggtttgcacataattacatctgggctcttacacaaacactgagaattgccgatcacagtctttacgctcttgagccacccgccccgcactgtggcgagctgggtagccccggcggctctcctggagactgggggtctttgtattctcctgtcagccaagcgggatctttgagtccggctgccagtctcgaagaaagacccggactccttggagcgactttttcagcatgtctgtcccctggctcattggctttctcagactttttg

SEQ ID NO: 76 - DNA, 741 bp, synthetic construct
  misc_feature 1..741: note = Description of Artificial Sequence: Synthetic polynucleotide
  source 1..741: mol_type = other DNA
atggccctgcctcccagcccgctggccatggaatatgtcaatgactttgacttgatgaagtttgaggtaaagcgggaaccctctgagggccgacctggcccacctacagcctcactgggatccacaccttacagctcagtgcctccttcacccaccttcagtgaaccaggcatggtaggggcaaccgagggtacacgaccaggtttggaggagctgtactggcttgctaccctgcagcagcagcttggggctggggaggcattgggactgagtcctgaagaggccatggagctactgcaaggtcagggcccagtccctgttgatggaccccatggttactacccagggagcccagaggagacaggagcccagcacgttcagttggcagagcggttttccgacgcggcgcttgtctcgatgtctgtgcgagaactaaaccggcagctgcggggatgcgggagagacgaggctctacgactgaagcagaggcgtcgaacgctgaagaaccgtggctatgcgcaagcatgtcgttccaagaggctgcaacagaggcgaggtcttgaggccgagcgcgcccgtcttgcagcccagctagatgcgctacgagctgaagtagcacgtttggcaagagagcgagatctctacaaggctcgctgtgaccggctaacctcgagtggccccgggtccggggatccctcccaccttttcctctgcccaactttcttgtacaaagttgtcccc

SEQ ID NO: 77 - DNA, 1395 bp, synthetic construct
  misc_feature 1..1395: note = Description of Artificial Sequence: Synthetic polynucleotide
  source 1..1395: mol_type = other DNA
atgaacgcgcagctgaccatggaagcgatcggcgagctgcacggggtgagccatgagccggtgcccgcccctgccgacctgctgggcggcagcccccacgcgcgcagctccgtggcgcaccgcggcagccacctgccccccgcgcacccgcgctccatgggcatggcgtccctgctggacggcggcagcggcggcggagattaccaccaccaccaccgggcccctgagcacagcctggccggccccctgcatcccaccatgaccatggcctgcgagactcccccaggtatgagcatgcccaccacctacaccaccttgacccctctgcagccgctgcctcccatctccacagtctcggacaagttcccccaccatcaccaccaccaccatcaccaccaccacccgcaccaccaccagcgcctggcgggcaacgtgagcggtagcttcacgctcatgcgggatgagcgcgggctggcctccatgaataacctctataccccctaccacaaggacgtggccggcatgggccagagcctctcgcccctctccagctccggtctgggcagcatccacaactcccagcaagggctcccccactatgcccacccgggggccgccatgcccaccgacaagatgctcacccccaacggcttcgaagcccaccacccggccatgctcggccgccacggggagcagcacctcacgcccacctcggccggcatggtgcccatcaacggccttcctccgcaccatccccacgcccacctgaacgcccagggccacgggcaactcctgggcacagcccgggagcccaacccttcggtgaccggcgcgcaggtcagcaatggaagtaattcagggcagatggaagagatcaataccaaagaggtggcgcagcgtatcaccaccgagctcaagcgctacagcatcccacaggccatcttcgcgcagagggtgctctgccgctcccaggggaccctctcggacctgctgcgcaaccccaaaccctggagcaaactcaaatccggccgggagaccttccggaggatgtggaagtggctgcaggagccggagttccagcgcatgtccgcgctccgcttagcagcatgcaaaaggaaagaacaagaacatgggaaggatagaggcaacacacccaaaaagcccaggttggtcttcacagatgtccagcgtcgaactctacatgcaatattcaaggaaaataagcgtccatccaaagaattgcaaatcaccatttcccagcagctggggttggagctgagcactgtcagcaacttcttcatgaacgcaagaaggaggagtctggacaagtggcaggacgagggcagctccaattcaggcaactcatcttcttcatcaagcacttgtaccaaagca

SEQ ID NO: 78 - DNA, 891 bp, synthetic construct
  misc_feature 1..891: note = Description of Artificial Sequence: Synthetic polynucleotide
  source 1..891: mol_type = other DNA
atgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttg

SEQ ID NO: 79 - DNA, 1554 bp, synthetic construct
  misc_feature 1..1554: note = Description of Artificial Sequence: Synthetic polynucleotide
  source 1..1554: mol_type = other DNA
atggcggcccttcccggcacggtaccgagaatgatgcggccggctccggggcagaactacccccgcacgggattccctttggaagtgtccaccccgcttggccaaggccgggtcaatcagctgggaggggtcttcatcaatgggcgacccctgcctaaccacatccgccacaagatagtggagatggcccaccatggcatccggccctgtgtcatctcccgacagctgcgtgtctcccacggctgcgtctccaagattctttgccgctaccaggagaccgggtccatccggcctggggccatcggcggcagcaagcccagacaggtggcgactccggatgtagagaaaaagattgaggagtacaagagggaaaacccaggcatgttcagctgggagatccgggacaggctgctgaaggatgggcactgtgaccgaagcactgtgccctcagtgagttcgattagccgcgtgctcagaatcaagttcgggaagaaagaggaggaggatgaagcggacaagaaggaggacgacggcgaaaagaaggccaaacacagcatcgacggcatcctgggcgacaaagggaaccggctggacgagggctcggatgtggagtcggaacctgacctcccactgaagcgcaagcagcgacgcagtcggaccacattcacggccgagcagctggaggagctggagaaggcctttgagaggacccactacccagacatatacacccgcgaggagctggcgcagaggaccaagctgacagaggcgcgtgtgcaggtctggttcagtaaccgccgcgcccgttggcgtaagcaggcaggagccaaccagctggcggcgttcaaccaccttctgccaggaggcttcccgcccaccggcatgcccacgctgcccccctaccagctgccggactccacctaccccaccaccaccatctcccaagatgggggcagcactgtgcaccggcctcagcccctgccaccgtccaccatgcaccagggcgggctggctgcagcggctgcagccgccgacaccagctctgcctacggagcccgccacagcttctccagctactctgacagcttcatgaatccggcggcgccctccaaccacatgaacccggtcagcaacggcctgtctcctcaggtgatgagcatcttgggcaaccccagtgcggtgcccccgcagccacaggctgacttctccatctccccgctgcatggcggcctggactcggccacctccatctcagccagctgcagccagcgggccgactccatcaagccaggagacagcctgcccacctcccaggcctactgcccacccacctacagcaccaccggctacagcgtggaccccgtggccggctatcagtacggccagtacggccagagtgagtgcctggtgccctgggcgtcccccgtccccattccttctcccacccccagggcctcctgcttgtttatggagagctacaaggtggtgtcagggtggggaatgtccatttcacagatggaaaaattgaagtccagccagatggaacagttcacc

SEQ ID NO: 80 - DNA, 903 bp, synthetic construct
  misc_feature 1..903: note = Description of Artificial Sequence: Synthetic polynucleotide
  source 1..903: mol_type = other DNA
atgagttgccaagcttttacttcggctgatacctttatacctctgaattctgacgcctctgcaactctgcctctgataatgcatcacagtgctgccgagtgtctaccagtctccaaccatgccaccaatgtgatgtctacagcaacaggacttcattattctgttccttcctgtcattatggaaaccagccatcaacctatggagtgatggcaggtagtttaaccccttgtctttataaatttcctgaccacaccttgagtcatggatttcctcctatacaccagcctcttctggcagaggaccccacagctgctgatttcaagcaggaactcaggcggaaaagtaaattggtggaagagccaatagacatggattctccagaaatcagagaacttgaaaagtttgccaatgaatttaaagtgagacgaattaaattaggatacacccagacaaatgttggggaggccctggcagctgtgcatggctctgaattcagtcaaacaacaatctgccgatttgaaaatctgcagctcagctttaaaaatgcatgcaaactgaaagcaatattatccaaatggctggaggaagctgagcaagtaggagctttgtacaatgaaaaagtgggagcaaatgaaaggaaaagaaaacgaagaacaactataagcattgctgctaaagatgctctggagagacactttggagaacagaataaaccttcttctcaagagatcatgaggatggctgaagaactgaatctggagaaagaagtagtaagagtttggttttgcaaccggaggcagagagaaaaacgggtgaaaacaagtctgaatcagagtttattttctatttctaaggaacatcttgagtgcagatcaggcctcatgggcccagctttcttgtac

SEQ ID NO: 81 - DNA, 1080 bp, synthetic construct
  misc_feature 1..1080: note = Description of Artificial Sequence: Synthetic polynucleotide
  source 1..1080: mol_type = other DNA
atggcgggacacctggcttcagattttgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaac

SEQ ID NO: 82 - DNA, 1440 bp, synthetic construct
  misc_feature 1..1440: note = Description of Artificial Sequence: Synthetic polynucleotide
  source 1..1440: mol_type = other DNA
atggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctac

SEQ ID NO: 83 - DNA, 852 bp, synthetic construct
  misc_feature 1..852: note = Description of Artificial Sequence: Synthetic polynucleotide
  source 1..852: mol_type = other DNA
atgtcgatgctgccgtcgtttggctttacgcaggagcaagtggcgtgcgtgtgcgaggttctgcagcaaggcggaaacctggagcgcctgggcaggttcctgtggtcactgcccgcctgcgaccacctgcacaagaacgagagcgtactcaaggccaaggcggtggtcgccttccaccgcggcaacttccgtgagctctacaagatcctggagagccaccagttctcgcctcacaaccaccccaaactgcagcaactgtggctgaaggcgcattacgtggaggccgagaagctgtgcggccgacccctgggcgccgtgggcaaatatcgggtgcgccgaaaatttccactgccgcgcaccatctgggacggcgaggagaccagctactgcttcaaggagaagtcgaggggtgtcctgcgggagtggtacgcgcacaatccctacccatcgccgcgtgagaagcgggagctggccgaggccaccggcctcaccaccacccaggtcagcaactggtttaagaaccggaggcaaagagaccgggccgcggaggccaaggaaagggagaacaccgaaaacaataactcctcctccaacaagcagaaccaactctctcctctggaagggggcaagccgctcatgtccagctcagaagaggaattctcacctccccaaagtccagaccagaactcggtccttctgctgcagggcaatatgggccacgccaggagctcaaactattctctcccgggcttaacagcctcgcagcccagtcacggcctgcagacccaccagcatcagctccaagactctctgctcggccccctcacctccagtctggtggacttggggtcc

SEQ ID NO: 84 - DNA, 873 bp, synthetic construct
  misc_feature 1..873: note = Description of Artificial Sequence: Synthetic polynucleotide
  source 1..873: mol_type = other DNA
atgtccatgctgcccaccttcggcttcacgcaggagcaagtggcgtgcgtgtgcgaggtgctgcagcagggcggcaacatcgagcggctgggccgcttcctgtggtcgctgcccgcctgcgagcaccttcacaagaatgaaagcgtgctcaaggccaaggccgtggtggccttccaccgcggcaacttccgcgagctctacaagatcctggagagccaccagttctcgccgcacaaccacgccaagctgcagcagctgtggctcaaggcacactacatcgaggcggagaagctgcgcggccgacccctgggcgccgtgggcaaataccgcgtgcgccgcaaattcccgctgccgcgctccatctgggacggcgaggagaccagctactgcttcaaggaaaagagtcgcagcgtgctgcgcgagtggtacgcgcacaacccctacccttcaccccgcgagaagcgtgagctgacggaggccacgggcctcaccaccacacaggtcagcaactggttcaagaaccggcggcagcgcgaccgggcggccgaggccaaggaaagggagaacaacgagaactccaattctaacagccacaacccgctgaatggcagcggcaagtcggtgttaggcagctcggaggatgagaagactccatcggggacgccagaccactcatcatccagccccgcactgctcctcagcccgccgccccctgggctgccgtccctgcacagcctgggccaccctccgggccccagcgcagtgccagtgccggtgccaggcggaggtggagcggacccactgcaacaccaccatggcctgcaggactccatcctcaaccccatgtcagccaacctcgtggacctgggctcc

SEQ ID NO: 85 - DNA, 804 bp, synthetic construct
  misc_feature 1..804: note = Description of Artificial Sequence: Synthetic polynucleotide
  source 1..804: mol_type = other DNA
atgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacac

SEQ ID NO: 86 - DNA, 1398 bp, synthetic construct
  misc_feature 1..1398: note = Description of Artificial Sequence: Synthetic polynucleotide
  source 1..1398: mol_type = other DNA
atggcggaggagcaggacctatcggaggtggagctgagccccgtgggctcggaggagccccgctgcctgtccccggggagcgcgccctcgctagggcccgacggcggcggcggcggatcgggcctgcgagccagcccggggccaggcgagctgggcaaggtcaagaaggagcagcaggacggcgaggcggacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatgggaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccc

SEQ ID NO: 87 - DNA, 951 bp, synthetic construct
  misc_feature 1..951: note = Description of Artificial Sequence: Synthetic polynucleotide
  source 1..951: mol_type = other DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 88 - DNA, 1338 bp, synthetic construct
  misc_feature 1..1338: note = Description of Artificial Sequence: Synthetic polynucleotide
  source 1..1338: mol_type = other DNA
atgcgacctgttcgagagaactcatcaggtgcgagaagcccgcgggttcctgctgatttggcgcggagcattttgataagcctacccttcccgccggactcgctggcccacaggcccccaagctccgctccgacggagtcccagggccttttcaccgtggccgctccagccccgggagcgccttctcctcccgccacgctggcgcaccttcttcccgccccggcaatgtacagccttctggagactgaactcaagaaccccgtagggacacccacacaagcggcgggcaccggcggccccgcagccccgggaggcgcaggcaagagtagtgcgaacgcagccggcggcgcgaactcgggcggcggcagcagcggtggtgcgagcggaggtggcgggggtacagaccaggaccgtgtgaaacggcccatgaacgccttcatggtatggtcccgcgggcagcggcgcaaaatggccctggagaaccccaagatgcacaattctgagatcagcaagcgcttgggcgccgactggaaactgctgaccgacgccgagaagcgaccattcatcgacgaggccaagcgacttcgcgccgtgcacatgaaggagtatccggactacaagtaccgaccgcgccgcaagaccaagacgctgctcaagaaagataagtactccctgcccagcggcctcctgcctcccggtgccgcggccgccgccgccgctgccgcggccgcagccgctgccgccagcagtccggtgggcgtgggccagcgcctggacacgtacacgcacgtgaacggctgggccaacggcgcgtactcgctggtgcaggagcagctgggctacgcgcagcccccgagcatgagcagcccgccgccgccgcccgcgctgccgccgatgcaccgctacgacatggccggcctgcagtacagcccaatgatgccgcccggcgctcagagctacatgaacgtcgctgccgcggccgccgccgcctcgggctacgggggcatggcgccctcagccacagcagccgcggccgccgcctacgggcagcagcccgccaccgccgcggccgcagctgcggccgcagccgccatgagcctgggccccatgggctcggtagtgaagtctgagcccagctcgccgccgcccgccatcgcatcgcactctcagcgcgcgtgcctcggcgacctgcgcgacatgatcagcatgtacctgccacccggcggggacgcggccgacgccgcctctccgctgcccggcggtcgcctgcacggcgtgcaccagcactaccagggcgccgggactgcagtcaacggaacggtgccgctgacccacatc

SEQ ID NO: 89 - DNA, 813 bp, synthetic construct
  misc_feature 1..813: note = Description of Artificial Sequence: Synthetic polynucleotide
  source 1..813: mol_type = other DNA
atgttacaggcgtgcaaaatggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccac

SEQ ID NO: 90 - DNA, 789 bp, synthetic construct
  misc_feature 1..789: note = Description of Artificial Sequence: Synthetic polynucleotide
  source 1..789: mol_type = other DNA
atgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccccgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggccttg

SEQ ID NO: 91 - DNA, 744 bp, synthetic construct
  misc_feature 1..744: note = Description of Artificial Sequence: Synthetic polynucleotide
  source 1..744: mol_type = other DNA
atgacgtgtgttgaacaagacaagctgggtcaagcatttgaagatgcttttgaggttctgaggcaacattcaactggagatcttcagtactcgccagattacagaaattacctggctttaatcaaccatcgtcctcatgtcaaaggaaattccagctgctatggagtgttgcctacagaggagcctgtctataattggagaacggtaattaacagtgctgcggacttctattttgaaggaaatattcatcaatctctgcagaacataactgaaaaccagctggtacaacccactcttctccagcaaaaggggggaaaaggcaggaagaagctccgactgtttgaataccttcacgaatccctgtataatccggagatggcatcttgtattcagtgggtagataaaaccaaaggcatctttcagtttgtatcaaaaaacaaagaaaaacttgccgagctttgggggaaaagaaaaggcaacaggaagaccatgacttaccagaaaatggccagggcactcagaaattacggaagaagtggggaaattaccaaaatccggaggaagctgacttaccagttcagtgaggccattctccaaagactctctccatcctatttcctggggaaagagatcttctattcacagtgtgttcaacctgatcaagaatatctcagtttaaataactggaatgcaaattataattatacatatgccaattaccatgagctaaatcaccatgattgc

SEQ ID NO: 92 - DNA, 612 bp, synthetic construct
  misc_feature 1..612: note = Description of Artificial Sequence: Synthetic polynucleotide
  source 1..612: mol_type = other DNA
atgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctg

SEQ ID NO: 93 - DNA, 1557 bp, synthetic construct
  misc_feature 1..1557: note = Description of Artificial Sequence: Synthetic polynucleotide
  source 1..1557: mol_type = other DNA
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagcttg

SEQ ID NO: 94 - DNA, 1350 bp, synthetic construct
  misc_feature 1..1350: note = Description of Artificial Sequence: Synthetic polynucleotide
  source 1..1350: mol_type = other DNA
atgttgtggaaaataaccgataatgtcaagtacgaagaggactgcgaggatcgccacgacgggagcagcaatgggaatccgcgggtcccccacctctcctccgccgggcagcacctctacagccccgcgccacccctctcccacactggagtcgccgaatatcagccgccaccctactttccccctccctaccagcagctggcctactcccagtcggccgacccctactcgcatctgggggaagcgtacgccgccgccatcaaccccctgcaccagccggcgcccacaggcagccagcagcaggcctggcccggccgccagagccaggagggagcggggctgccctcgcaccacgggcgcccggccggcctactgccccacctctccgggctggaggcgggcgcggtgagcgcccgcagggatgcctaccgccgctccgacctgctgctgccccacgcacacgccctggatgccgcgggcctggccgagaacctggggctccacgacatgcctcaccagatggacgaggtgcagaatgtcgacgaccagcacctgttgctgcacgatcagacagtcattcgcaaaggtcccatttccatgaccaagaaccctctgaacctcccctgtcagaaggagctggtgggggccgtaatgaaccccactgaggtcttctgctcagtccctggaagattgtcgctcctcagctctacgtctaaatacaaagtgacagtggctgaagtacagaggcgactgtccccacctgaatgcttaaatgcctcgttactgggaggtgttctcagaagagccaaatcgaaaaatggaggccggtccttgcgggagaagttggacaagattgggttgaatcttccggccgggaggcggaaagccgctcatgtgactctcctgacatccttagtagaaggtgaagctgttcatttggctagggactttgcctatgtctgtgaagccgaatttcctagtaaaccagtggcagaatatttaaccagacctcatcttggaggacgaaatgagatggcagctaggaagaacatgctattggcggcccagcaactgtgtaaagaattcacagaacttctcagccaagaccggacaccccatgggaccagcaggctcgccccagtcttggagacgaacatacagaactgcttgtctcatttcagcctgattacccacgggtttggcagccaggccatctgtgccgcggtgtctgccctgcagaactacatcaaagaagccctgattgtcatagacaaatcctacatgaaccctggagaccagagtccagctgattctaacaaaaccctggagaaaatggagaaacacaggaaa

SEQ ID NO: 95 - DNA, 28 bp, synthetic construct
  misc_feature 1..28: note = Description of Artificial Sequence: Synthetic primer
  source 1..28: mol_type = other DNA
agaccacgcctctgtcatgtaccaaatc

SEQ ID NO: 96 - DNA, 27 bp, synthetic construct
  misc_feature 1..27: note = Description of Artificial Sequence: Synthetic primer
  source 1..27: mol_type = other DNA
ggtcagcagcatcgtggtcaacataac

SEQ ID NO: 97 - DNA, 25 bp, synthetic construct
  misc_feature 1..25: note = Description of Artificial Sequence: Synthetic primer
  source 1..25: mol_type = other DNA
tctccgtggtcctgaagcagacata

SEQ ID NO: 98 - DNA, 28 bp, synthetic construct
  misc_feature 1..28: note = Description of Artificial Sequence: Synthetic primer
  source 1..28: mol_type = other DNA
agccatgtggtctctctggttgtgtatg

SEQ ID NO: 99 - DNA, 21 bp, synthetic construct
  misc_feature 1..21: note = Description of Artificial Sequence: Synthetic primer
  source 1..21: mol_type = other DNA
tttgtgggcctgaagaaaact

SEQ ID NO: 100 - DNA, 22 bp, synthetic construct
  misc_feature 1..22: note = Description of Artificial Sequence: Synthetic primer
  source 1..22: mol_type = other DNA
cttgaatcccgaatggaaaggg

SEQ ID NO: 101 - DNA, 21 bp, synthetic construct
  misc_feature 1..21: note = Description of Artificial Sequence: Synthetic primer
  source 1..21: mol_type = other DNA
tacagcatgtcctactcgcag

SEQ ID NO: 102 - DNA, 22 bp, synthetic construct
  misc_feature 1..22: note = Description of Artificial Sequence: Synthetic primer
  source 1..22: mol_type = other DNA
gagtccattgctgttggaaccg

SEQ ID NO: 103 - DNA, 21 bp, synthetic construct
  misc_feature 1..21: note = Description of Artificial Sequence: Synthetic primer
  source 1..21: mol_type = other DNA
cagcggaaaccccaacagtta

SEQ ID NO: 104 - DNA, 21 bp, synthetic construct
  misc_feature 1..21: note = Description of Artificial Sequence: Synthetic primer
  source 1..21: mol_type = other DNA
gacctccacagagaagtcgag

SEQ ID NO: 105 - DNA, 21 bp, synthetic construct
  misc_feature 1..21: note = Description of Artificial Sequence: Synthetic primer
  source 1..21: mol_type = other DNA
agtccactgagtaccggagac

SEQ ID NO: 106 - DNA, 20 bp, synthetic construct
  misc_feature 1..20: note = Description of Artificial Sequence: Synthetic primer
  source 1..20: mol_type = other DNA
cgagagctacacgttcacgg

SEQ ID NO: 107 - DNA, 21 bp, synthetic construct
  misc_feature 1..21: note = Description of Artificial Sequence: Synthetic primer
  source 1..21: mol_type = other DNA
agccaaccttaactgaggagt

SEQ ID NO: 108 - DNA, 21 bp, synthetic construct
  misc_feature 1..21: note = Description of Artificial Sequence: Synthetic primer
  source 1..21: mol_type = other DNA
tgatcctgactgcgatgagag

SEQ ID NO: 109 - DNA, 20 bp, synthetic construct
  misc_feature 1..20: note = Description of Artificial Sequence: Synthetic primer
  source 1..20: mol_type = other DNA
ggcaacgtggccttttctac

SEQ ID NO: 110 - DNA, 21 bp, synthetic construct
  misc_feature 1..21: note = Description of Artificial Sequence: Synthetic primer
  source 1..21: mol_type = other DNA
gaagtttcgcagacctgacat

SEQ ID NO: 111 - DNA, 21 bp, synthetic construct
  misc_feature 1..21: note = Description of Artificial Sequence: Synthetic primer
  source 1..21: mol_type = other DNA
atcgctctcctgctaacagtc

SEQ ID NO: 112 - DNA, 21 bp, synthetic construct
  misc_feature 1..21: note = Description of Artificial Sequence: Synthetic primer
  source 1..21: mol_type = other DNA
ctgagacccgagcagagtttg

SEQ ID NO: 113 - DNA, 25 bp, synthetic construct
  misc_feature 1..25: note = Description of Artificial Sequence: Synthetic primer
  source 1..25: mol_type = other DNA
cacgatctcatacctggcctgcttc

SEQ ID NO: 114 - DNA, 26 bp, synthetic construct
  misc_feature 1..26: note = Description of Artificial Sequence: Synthetic primer
  source 1..26: mol_type = other DNA
tggagcaggacaggttcagtctttca

SEQ ID NO: 115 - DNA, 23 bp, synthetic construct
  misc_feature 1..23: note = Description of Artificial Sequence: Synthetic primer
  source 1..23: mol_type = other DNA
aggttgctgctggtgaggtcatt

SEQ ID NO: 116 - DNA, 26 bp, synthetic construct
  misc_feature 1..26: note = Description of Artificial Sequence: Synthetic primer
  source 1..26: mol_type = other DNA
gtttgagtggtgccgtactggtagga

SEQ ID NO: 117 - DNA, 21 bp, synthetic construct
  misc_feature 1..21: note = Description of Artificial Sequence: Synthetic primer
  source 1..21: mol_type = other DNA
agggctgtcctgaataagcag

SEQ ID NO: 118 - DNA, 23 bp, synthetic construct
  misc_feature 1..23: note = Description of Artificial Sequence: Synthetic primer
  source 1..23: mol_type = other DNA
gtgtatatcccagggtgatcctc

SEQ ID NO: 119 - DNA, 21 bp, synthetic construct
  misc_feature 1..21: note = Description of Artificial Sequence: Synthetic primer
  source 1..21: mol_type = other DNA
gaggaagaggtaaccacaggg

SEQ ID NO: 120 - DNA, 22 bp, synthetic construct
  misc_feature 1..22: note = Description of Artificial Sequence: Synthetic primer
  source 1..22: mol_type = other DNA
atgtccctcttgtcgccaacct

SEQ ID NO: 121 - DNA, 21 bp, synthetic construct
  misc_feature 1..21: note = Description of Artificial Sequence: Synthetic primer
  source 1..21: mol_type = other DNA
gagggtcagtagaacatgcgt

SEQ ID NO: 122 - DNA, 21 bp, synthetic construct
  misc_feature 1..21: note = Description of Artificial Sequence: Synthetic primer
  source 1..21: mol_type = other DNA
tgcctttttcttagggcagag

SEQ ID NO: 123 - DNA, 21 bp, synthetic construct
  misc_feature 1..21: note = Description of Artificial Sequence: Synthetic primer
  source 1..21: mol_type = other DNA
catttcacgcatctggcgttc

SEQ ID NO: 124 - DNA, 21 bp, synthetic construct
  misc_feature 1..21: note = Description of Artificial Sequence: Synthetic primer
  source 1..21: mol_type = other DNA
gggtgtcgagggaaaaatagg

SEQ ID NO: 125 - DNA, 21 bp, synthetic construct
  misc_feature 1..21: note = Description of Artificial Sequence: Synthetic primer
  source 1..21: mol_type = other DNA
ggcaagttgattggagggatg

SEQ ID NO: 126 - DNA, 21 bp, synthetic construct
  misc_feature 1..21: note = Description of Artificial Sequence: Synthetic primer
  source 1..21: mol_type = other DNA
cttgtctgttcttctgacccc

SEQ ID NO: 127 - DNA, 21 bp, synthetic construct
  misc_feature 1..21: note = Description of Artificial Sequence: Synthetic primer
  source 1..21: mol_type = other DNA
agtggcagttacccattcctg

SEQ ID NO: 128 - DNA, 22 bp, synthetic construct
  misc_feature 1..22: note = Description of Artificial Sequence: Synthetic primer
  source 1..22: mol_type = other DNA
gtatgcaccattcaactcctcg

SEQ ID NO: 129 - DNA, 21 bp, synthetic construct
  misc_feature 1..21: note = Description of Artificial Sequence: Synthetic primer
  source 1..21: mol_type = other DNA
ctcgtactggatgggtgaact

SEQ ID NO: 130 - DNA, 21 bp, synthetic construct
  misc_feature 1..21: note = Description of Artificial Sequence: Synthetic primer
  source 1..21: mol_type = other DNA
tgaatctcgacgttctcctcc

(19) United States
(12) Patent Application Publication
(10) Pub. No.: US 2024/0327823 A1
(43) Pub. Date: Oct. 3, 2024

(54) METHODS FOR SCREENING GENETIC PERTURBATIONS

(71) Applicant: The Regents of the University of California, Oakland, CA (US)

(72) Inventors: Prashant Mali, La Jolla, CA (US); Udit Parekh, La Jolla, CA (US); Yan Wu, La Jolla, CA (US); Kun Zhang, La Jolla, CA (US)

(21) Appl. No.: 18/416,749

(22) Filed: Jan. 18, 2024

Related U.S. Application Data
(62) Division of application No. 17/028,836, filed on Sep. 22, 2020, now Pat. No. 11,912,986.
(60) Provisional application No. 62/904,614, filed on Sep. 23, 2019.

(51) Int. Cl. C12N 15/10; A61K 35/44; C12N 5/071; C12N 15/86

(52) U.S. Cl. CPC C12N 15/1065; A61K 35/44; C12N 5/069; C12N 15/86; C12N 2506/45; C12N 2740/15043; C12N 2740/15052

(57) ABSTRACT

Understanding the complex effects of genetic perturbations on cellular state and fitness in human pluripotent stem cells (hPSCs) has been challenging using traditional pooled screening techniques which typically rely on unidimensional phenotypic readouts. Here, Applicants use barcoded open reading frame (ORF) overexpression libraries with a coupled single-cell RNA sequencing (scRNA-seq) and fitness screening approach, a technique we call SEUSS (ScalablE fUnctional Screening by Sequencing), to establish a comprehensive assaying platform. Using this system, Applicants perturbed hPSCs with a library of developmentally critical transcription factors (TFs), and assayed the impact of TF overexpression on fitness and transcriptomic cell state across multiple media conditions. Applicants further leveraged the versatility of the ORF library approach to systematically assay mutant gene libraries and also whole gene families. From the transcriptomic responses, Applicants built genetic co-perturbation networks to identify key altered gene modules. Strikingly, we found that KLF4 and SNAI2 have opposing effects on the pluripotency gene module, highlighting the power of this method to characterize the effects of genetic perturbations. From the fitness responses, Applicants identified ETV2 as a driver of reprogramming towards an endothelial-like state.

### CROSS-REFERENCE TO RELATED APPLICATION

This application is a divisional of U.S. patent application Ser. No. 17/028,836, filed Sep. 22, 2020, which claims priority to 35 U.S.C. § 119 (e) of U.S. Provisional Application Ser. No. 62/904,614, filed Sep. 23, 2019, the contents of each of which are hereby incorporated by reference their entirety.

This invention was made with government support under HG009285 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 14, 2020, is named 114198-0152_SL.txt and is 155,507 bytes in size.

### BACKGROUND

Cellular reprogramming by the overexpression of transcription factors (TF), has widely impacted biological research, from the direct conversion of adult somatic cells to the induction of pluripotent stem cells, and the differentiation of hPSCs. To date, the choice of TFs that drive such reprogramming has been through a combination of the knowledge of their role in development and cellular transformation, and systematic trial-and-error. These challenges highlight the need for the development of a scalable screening method to assess the effects of TF overexpression. Such a screening method would have broad applicability in advancing a fundamental understanding of reprogramming, and as a means for the discovery of novel reprogramming factors. This disclosure addresses this need and provides related advantages as well.

### SUMMARY

Described herein is a comprehensive high-throughput platform to determine an optimal method to drive the differentiation of pluripotent cells to specific somatic lineages. In some aspects, the platform utilizes a novel open reading frame (ORF) gene overexpression vector library of developmentally critical transcription factors. The platform builds genetic co-perturbation networks to identified key altered gene modules and identifies key reprogramming/differentiation drivers from transcriptomic responses. The platform enabled identification of the key role of (previously not recognized) transcription factor ETV2 in reprogramming towards an endothelial state.

Thus, in one aspect, provided herein are isolated nucleic acids comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF. In some embodiments, the TF ORF encodes a developmentally critical TF.

In another aspect, provided herein is a TF screening library comprising, consisting of, or consisting essentially of at least one isolated nucleic acid comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF. In some embodiments, the TF ORF encodes a developmentally critical TF, optionally selected from the TFs listed in Table 1.

In some embodiments, the TF screening library comprises, consists of, or consists essentially of at least 10, at least 20, at least 30, at least 40, at least 50, at least 60, at least 70, at least 80, at least 90, or at least 100 nucleic acids or vectors, wherein each nucleic acid or vector comprises, consists of, or consists essentially of a distinct nucleic acid encoding a TF ORF.

In some embodiments, the TF screening library further comprises, consists of, or consists essentially of a nucleic acid encoding a selectable marker. In some embodiments, the TF screening library further comprises, consists of, or consists essentially of a nucleic acid encoding an expression control element. In some embodiments, the expression control element is a promoter or a long terminal repeat (LTR). In some embodiments, the TF screening library further comprises, consists of, or consists essentially of a nucleic acid encoding a translation elongation factor, optionally wherein the translation elongation factor is Ef1a.

In some embodiments, the vector is a retroviral vector, optionally a lentiviral vector.

In another aspect, provided herein is a viral packaging system comprising, consisting of, or consisting essentially of at least one isolated nucleic acid comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF; or aTF screening library; and a packaging plasmid.

In another aspect, provided herein is a method for producing a viral particle, the method comprising, consisting of, or consisting essentially of transfecting a packaging cell line with a viral packaging system comprising, consisting of, or consisting essentially of at least one isolated nucleic acid comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF; or aTF screening library; and a packaging plasmid under conditions suitable to package the vector or the TF screening library into a viral particle. In another aspect, also provided herein is a viral particle produced by this method, and optionally a carrier. In another aspect, also provided herein is an isolated cell comprising a nucleic acid, vector, or particle as described herein, and optionally a carrier.

In another aspect, provided herein is a kit comprising, consisting of, or consisting essentially of at least one of (a) a nucleic acid or vector according to any of the embodiments described herein; and/or (b) a TF screening library according to any of the embodiments described herein; and/or (c) a viral packaging system according to any of the embodiments described herein; and/or (d) a viral particle according to any of the embodiments described herein; and/or (e) an isolated cell according to any of the embodiments described herein, and optionally instructions for use.

In another aspect, provided herein is a method of performing a high throughput gene activation screen, the method comprising, consisting of, or consisting essentially of: (a) transducing a target cell with the viral particle according to any of the embodiments described herein; and (b) performing scRNA-seq on the transduced target cell to identify the nucleic acid barcode. In some embodiments, the method further comprises or consists of determining a fitness effect in the transduced target cell. In some embodiments, the method further comprises or consists of identifying a co-perturbation network. In some embodiments, the method further comprises or consists of identifying a functional gene module. In some embodiments, the target cell is a stem cell. In some embodiments, the stem cell is an embryonic stem cell (ESC) or an induced pluripotent stem cell (iPSC). In some embodiments, the target cell is a mammalian cell, optionally wherein the mammalian cell is an equine, bovine, canine, murine, porcine, feline, or human cell. In a particular embodiment, the target cell is a human cell.

In other aspects, also provided herein is a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell. In some embodiments, ectopic expression of ETV2 is induced by transducing the stem cell with a vector comprising a nucleic acid encoding ETV2 and a nucleic acid encoding an expression control element. In some embodiments, the stem cell is an ESC or an iPSC. In some embodiments, the stem cell is a mammalian cell, optionally wherein the mammalian cell is an equine, bovine, canine, murine, porcine, feline, or human cell. In some embodiments, the stem cell is a human cell. In some embodiments, the stem cell has been genetically modified. In some embodiments, the method further comprises or consists of genetically modifying the stem cell or the endothelial cell.

In further aspect, also provided herein is an endothelial cell produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, and optionally a carrier. In some embodiments, the endothelial cell expresses at least one of CDH5, PECAM1, or VWF.

In another aspect, also provided herein is a population of endothelial cells produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, and optionally a carrier.

In some aspects, provided herein is a composition comprising, consisting of, or consisting essentially of an endothelial cell produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, or a population of endothelial cells produced according to a method described herein, and one or more of: a pharmaceutically acceptable carrier, a cryopreservative or a preservative. In some embodiments, the carrier is a pharmaceutically acceptable carrier. In some embodiments, the cryopreservative is suitable for long term storage of the composition at a temperature ranging from −200° C. to 0° C., from −80° C. to 0° C., from −20° C. to 0° C., or from 0° C. to 10° C.

In some aspects, provided herein is a method of treating a subject in need thereof, the method comprising, consisting of, or consisting essentially of administering an endothelial cell produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, or a population of endothelial cells produced according to a method described herein, or a composition comprising, consisting of, or consisting essentially of the endothelial cell or population and a carrier to the subject. In some embodiments of the method, an effective amount of the endothelial cell, population, or composition is administered to the subject. In some embodiments, the endothelial cell or population is allogenic or autologous to the subject being treated.

In some embodiments of the method, the subject has a wound, a corneal disease or condition, a myocardial infarction, or a vascular disease or condition. In some embodiments, the subject has a corneal disease or condition. In some embodiments, the administration is local or systemic. In some embodiments, the endothelial cell, population, or composition is administered to the subject's eye.

In some embodiments of the method, the subject is a mammal and the mammal is an equine, bovine, canine, murine, porcine, feline, or human. In some embodiments, the mammal is a human. In some embodiments, the endothelial cells are autologous or allogeneic to the subject being treated.

### BRIEF DESCRIPTION OF THE FIGURES

FIGS. 1A-1F: SEUSS workflow and identification of significant TFs from fitness and scRNA-seq analysis. (FIG. 1A) Schematic of experimental and analytical framework for evaluation of effects of transcription factor (TF) overexpression in hPSCs: Individual TFs are cloned into the barcoded ORF overexpression vector, pooled and packaged into lentiviral libraries for transduction of hPSCs. Transduced cells are harvested at a fixed time point to be assayed as single cells using droplet based scRNA-seq to evaluate transcriptomic changes. Cells are genotyped by amplifying the overexpression transcript from scRNA-seq cDNA prior to fragmentation and library construction, and identifying the overexpressed TF barcode for each cell. The cell count for each genotype is used to estimate fitness. Gene expression matrices from scRNA-seq are used to obtain differential gene expression and clustering signatures which in turn are used for evaluation of cell state reprogramming and gene regulatory network analysis. (FIG. 1B) Fitness effect of TFs: log fold change of individual TFs, calculated as cell counts normalized against plasmid library read counts. (FIG. 1C) t-SNE projection (left panel), and cluster enrichment of significant TFs in clusters (right panel) from screens in pluripotent stem cell medium. (FIG. 1D) t-SNE projection (left panel), and cluster enrichment of significant TFs in clusters (right panel) from screens in unilineage (endothelial) growth medium. (FIG. 1E) t-SNE projection (left panel), and enrichment of significant TFs in clusters (right panel) from screens in multilineage differentiation medium. (FIG. 1F) Number of differentially expressed genes for TFs across different growth media. The TFs in (FIG. 1C), (FIG. 1D), (FIG. 1E) and (FIG. 1F) were chosen as significant with the following criteria: cluster enrichment with a false discovery rate (FDR) of less than 10⁻⁶ and a cluster enrichment profile different from control (mCherry) with a FDR less than 10⁻⁶, or if the TF drove differential expression of more than 100 genes.

FIGS. 2A-2G: Effect of TF overexpression on gene-to-gene co-perturbation network (FIG. 2A) Schematic for gene-gene co-perturbation network analysis: A SNN network is built from the linear model coefficients and the network is then segmented into gene modules. Genes have a highly weighted edge between them if they respond similarly to TF overexpression. (FIG. 2B) Gene module network: Node size indicates the number of genes in the module; Edge size indicates distance between modules. (FIG. 2C) Effect of TF overexpression on gene modules: (FIG. 2D) Schematic of functional domains of c-MYC: MYC Box I (MBI) and MYC Box II (II) which are essential for transactivation of target genes are housed in the amino-terminal domain (NTD); the basic (b) helix-loop-helix (HLH) leucine zipper (LZ) motif, which is required for heterodimerization with the MAX protein is housed in the carboxy-terminal domain (CTD); the nuclear localization signal domain (NLS) is located in the central region of the protein. (FIG. 2E) Effect of MYC mutant overexpression on gene modules. (FIG. 2F) Schematic of KLF gene family protein structure grouped by common structural and functional features (FIG. 2G) Effect of KLF family overexpression on gene modules. For heatmaps in (FIG. 2C), (FIG. 2E), (FIG. 2F), effect size was calculated as the average of the linear model coefficients for a given TF perturbation across all genes within a module.

FIGS. 3A-3H: Elucidating effects of KLF4, SNAI2 and ETV2 (FIG. 3A) Effect of KLF4 and SNAI2 on a subnetwork of the pluripotent state module, encompassing key pluripotency regulators. Node size indicates the effect size; blue nodes are downregulated, red nodes are upregulated. (FIG. 3B) PC plot of performing PCA on 200 genes from the Hallmark Epithelial Mesenchymal Transition geneset from MSigDB⁴². PCI corresponds to an EMT-like signature. (FIG. 3C) Effect of KLF4 and SNAI2 on selected epithelial and mesenchymal markers, including key Cadherin genes. (FIG. 3D) Correlation between fitness estimate from scRNA-seq genotype counts and bulk fitness estimate from gDNA in hPSC medium. (FIG. 3E) Morphology change for cells transduced with either ETV2 or mCherry in EGM. (FIG. 3F) Immunofluorescence micrograph of CDH5 labelled day 6 ETV2- or mCherry-transduced cells. (FIG. 3G) qRT-PCR analysis of signature endothelial genes CDH5, PECAM1, VWF and KDR, at day 6 post-transduction. Data were normalized to GAPDH and expressed relative to control cells in pluripotent stem cell medium. (FIG. 3H) Tube formation assay for day 6 ETV2- or mCherry-transduced cells

FIG. 4: Schematic of cloning strategy for synthesis of barcoded ORF vectors. The construction involved two steps: (i) insertion of a pool of barcodes into the backbone after digestion with HpaI, (ii) individually substituting mCherry with TFs after digestion with BamHI.

FIGS. 5A-5C: Fitness analysis from genomic DNA and correlation with fitness from scRNA-seq genotyped cell counts (FIG. 5A) Log fold-change of TF read counts amplified from genomic DNA vs plasmid library control (FIG. 5B) Log fold change of TF counts vs plasmid library control for genomic DNA reads vs cell counts fitness for: (FIG. 5B) Unilineage medium (endothelial growth medium) (FIG. 5C) Multilineage medium.

FIGS. 6A-6D: Differential gene expression analysis of significant TFs (FIG. 6A) Heatmap of differentially expressed genes for significant TFs in hPSC medium. (FIG. 6B) Heatmap of differentially expressed genes for significant TFs in endothelial growth medium. (FIG. 6C) Heatmap of differentially expressed genes for significant TFs in multilineage medium (FIG. 6D) Heatmap showing signed log p-values of enrichment for differentially expressed homologous genes in mESCs upon overexpression of TFs²⁵. ASCL1, CDX2, KLF4, MYOD1, and OTX2 display a high degree of overlap with overexpression of their homologs in mESCs.

FIGS. 7A-7F: Correlation between aggregated samples. For all plots, correlation was between the coefficients of significant hits, with a hit being defined as a gene-TF pair with the following significance criteria: (FDR<0.05, |coef|>0.025). (FIGS. 7A-7E) Correlation between significant hits in the combined hPSC dataset with hits in each individual dataset. (FIG. 7F) Correlation of hits between the two multilineage datasets.

FIGS. 8A-8C: Correlation between fitness and transcriptomic effects. (FIG. 8A) Correlation of the number of differentially expressed genes for each TF vs the fitness effect (log-FC) for hPSC medium (FIG. 8B) Correlation of the number of differentially expressed genes for each TF vs the fitness effect (log-FC) for endothelial medium (FIG. 8C) Correlation of the number of differentially expressed genes for each TF vs the fitness effect (log-FC) for multilineage medium.

FIGS. 9A-9D: Confirmatory assays for effects of KLF4 and SNAI2 on key genes in the pluripotency network and involved in EMT (FIG. 9A) qRT-PCR analysis of signature pluripotency network genes SOX2, POU5F1, NANOG, DNMT3B, DPPA4 and SALL2 at day 5 post-transduction in in pluripotent stem cell medium. (FIG. 9B) qRT-PCR analysis of signature cadherins during EMT: CDH1 and CDH2 at day 5 post-transduction in pluripotent stem cell medium. (FIG. 9C) qRT-PCR analysis of signature epithelial marker genes during EMT: EPCAM, LAMC1 and SPP1 at day 5 post-transduction in pluripotent stem cell medium. (FIG. 9D) qRT-PCR analysis of signature mesenchymal marker genes during EMT: TPM2, THY1 and VIM at day 5 post-transduction in pluripotent stem cell medium. Data for all assays were normalized to GAPDH and expressed relative to control cells.

FIGS. 10A-10B: Correlation of KLF4 and MYC effects across samples. (FIG. 10A) Correlation of KLF4 effects in the KLF family screen with KLF4 effects in the hPSC screen. (FIG. 10B) Correlation of MYC effects in the MYC mutants screen with KLF4 effects in the hPSC screen.

### DETAILED DESCRIPTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

A “viral vector” is defined as a recombinantly produced virus or viral particle that comprises a nucleic acid to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5 (7): 823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure include vectors based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to this disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “an expression control element” as used herein, intends a polynucleotide that is operatively linked to a target polynucleotide to be transcribed, and facilitates the expression of the target polynucleotide. A promoter is an example of an expression control element.

The term “promoter” refers to a nucleic acid sequence (e.g., a region of genomic DNA) that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Non-tissue specific promoters include but are not limited to human cytomegalovirus (CMV), CMV enhancer/chicken β-actin (CBA) promoter, Rous sarcoma virus (RSV), simian virus 40 (SV40) and mammalian elongation factor 1α (EF1α), are non-specific promoters and are commonly used in gene therapy vectors. Promoters can also be tissue specific. A tissue specific promoter allows for the production of a protein in a certain population of cells that have the appropriate transcriptional factors to activate the promoter.

A “target cell” as used herein, shall intend a cell containing the genome into which polynucleotides that are operatively linked to an expression control element are to be integrated. Cells that are infected with a lentivirus or susceptible to lentiviral infection are non-limiting examples of target cells.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and is meant to encompass both cultured and engineered cells or tissues.

As used herein, “stem cell” defines a cell with the ability to divide for indefinite periods in culture and give rise to specialized cells. At this time and for convenience, stem cells are categorized as somatic (adult), embryonic or induced pluripotent stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

The term “culturing” refers to the in vitro propagation of cells or organisms on or in synthetic culture conditions such as culture media of various kinds. In some aspects, the medium is changed daily. It is understood that the descendants of a cell grown in culture may not be completely identical (i.e., morphologically, genetically, or phenotypically) to the parent cell. By “expanded” is meant any proliferation, growth, or division of cells. Disclosed herein are culture methods that support differentiation by in inclusion of nutrients and effector molecules necessary to promote or support the differentiation of stem cells into differentiated cells.

“Differentiation” describes the process whereby an unspecialized cell acquires the features of a specialized cell such as a heart, liver, pancreas, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell and may also include maturation or development of the cell. As used herein, “a cell that differentiates into pancreatic beta cell” defines any cell that can become a committed pancreatic cells that produces insulin. Non-limiting examples of cells that are capable of differentiating into endothelial cells include embryonic stem cells, pluripotent stem cells, induced pluripotent stem cells (iPSCs), mesenchymal stem cell, hematopoietic stem cells, and adipose stem cells.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, produced by inducing expression of one or more stem cell specific genes.

A “composition” is intended to encompass a combination of active agent and another “carrier,” e.g., compound or composition, inert (for example, a detectable agent or label) or active, such as an adjuvant, diluent, binder, stabilizer, buffers, salts, lipophilic solvents, preservative, adjuvant or the like. Compositions may include stabilizers and preservatives. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. For examples of carriers, stabilizers and adjuvants, see Martin (1975) Remington's Pharm. Sci., 15th Ed. (Mack Publ. Co., Easton). Carriers also include biocompatible scaffolds, pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A population of cells intends a collection of more than one cell that is identical (clonal) or non-identical in phenotype and/or genotype.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by assaying for expression of a pre-selected cell surface marker or other marker.

An “effective amount” is an amount sufficient to effect beneficial or desired results. In the context of a therapeutic cell, population, or composition, the term “effective amount” as used herein refers to the amount to alleviate at least one or more symptom of a disease, disorder, or condition (e.g., corneal condition), and relates to a sufficient amount of the cell, population, or composition to provide the desired effect (e.g., repair of the cornea). An effective amount as used herein would also include an amount sufficient to delay the development of a disease, disorder, or condition symptom, alter the course of disease, disorder, or condition symptom (for example but not limited to, slow the progression of corneal degradation), or reverse a symptom of a disease, disorder, or condition. Thus, it is not possible to specify the exact “effective amount.” However, for any given case, an appropriate “effective amount” can be determined by one of ordinary skill in the art using only routine experimentation.

An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. The dosage can be determined by a physician and adjusted, as necessary, to suit observed effects of the treatment. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication ex vivo, in vitro or in vivo. Consistent with this definition, as used herein, the term “therapeutically effective amount” is an amount sufficient to achieve the result of the method.

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

An “enriched population” of cells intends a substantially homogenous population of cells having certain defined characteristics. The cells are greater than 60%, or alternatively greater than 65%, or alternatively greater than 70%, or alternatively greater than 75%, or alternatively greater than 80%, or alternatively greater than 85%, or alternatively greater than 90%, or alternatively greater than 95%, or alternatively greater than 98% identical in the defined characteristics. In one aspect, the substantially homogenous population of cells express markers that correlate with pluripotent cell identity such as expression of stem-cell specific genes like OCT4 and NANOG. In another aspect, the substantially homogenous population of cells express markers that are correlated with definitive endoderm cell identity such SOX17, CXCR4, FOXA2, and GATA4. In another aspect, the substantially homogenous population of cells express markers that are correlated with posterior foregut cell identity such as HNF1B, HNF4A while suppressing expression of HHEX, HOXA3, CDX2, OCT4, and NANOG. In another aspect, the substantially homogenous population of cells express markers that are correlated with pancreatic progenitor cell identity such as PDX1 (pancreatic duodenal homeobox gene 1). In another aspect, the substantially homogenous population of cells express markers that are correlated with endocrine pancreas cell identity such as NKX6.1, NEURO-DI, and NGN3. In yet another aspect, the substantially homogenous population of cells express markers that are correlated with islet precursor cell identity such as INS. This population may further be identified by its ability to secrete C-peptide.

A “gene” refers to a polynucleotide containing at least one open reading frame that is capable of encoding a particular RNA, polypeptide, or protein after being transcribed and/or translated. The term “express” refers to the production of a gene product. As used herein, “expression” refers to the process by which polynucleotides are transcribed into RNA and/or the process by which the transcribed RNA such as mRNA is subsequently being translated into peptides, polypeptides, or proteins. If the polynucleotide is derived from genomic DNA, expression may include splicing of the mRNA in a eukaryotic cell. A “gene product” or alternatively a “gene expression product” refers to the amino acid (e.g., peptide or polypeptide) or functional RNA (e.g. a tRNA, miRNA, rRNA, or shRNA) generated when a gene is transcribed and translated.

The term “treating” (or “treatment”) of a pancreatic or immune disorder or condition refers to ameliorating the effects of, or delaying, halting or reversing the progress of, or delaying or preventing the onset of, a pancreatic or immune condition such as diabetes, pre-diabetes, juvenile onset (Type I) diabetes mellitus, including pediatric insulin-dependent diabetes mellitus (IDDM), and adult onset diabetes mellitus (Type II diabetes). Treatment includes preventing the disease or condition (i.e., causing the clinical symptoms of the disease not to develop in a patient that may be predisposed to the disease but does not yet experience or display symptoms of the disease), inhibiting the disease or condition (i.e., arresting or reducing the development of the disease or its clinical symptoms), or relieving the disease or condition (i.e., causing regression of the disease or its clinical symptoms).

A mammalian stem cell, as used herein, intends a stem cell having an origin from a mammal. Non-limiting examples include, e.g., a murine, a canine, an equine, a simian and a human. An animal stem cell intends a stem cell having an origin from an animal, e.g., a mammalian stem cell.

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the methods and compositions disclosed herein are also useful for veterinary treatment of companion mammals, exotic animals and domesticated animals, including mammals, rodents, and the like which is susceptible to diabetes or other immune or pancreatic diseases or conditions. In one embodiment, the mammals include horses, dogs, and cats. In another embodiment of the present disclosure, the human is an adolescent or infant under the age of eighteen years.

An immature stem cell, as compared to a mature stem cell, intends a phenotype wherein the cell expresses or fails to express one or more markers of a mature phenotype. Examples of such are known in the art, e.g., telomerase length or the expression of actin for mature cardiomyocytes derived or differentiated from a less mature phenotype such as an embryonic stem cell. An immature beta cell intends a pancreatic cell that has insulin secretory granules but lacks GSIS. In contrast, mature beta cells typically are positive for GSIS and have low lactate dehydrogenase (LDH).

### Descriptive Embodiments

Understanding the complex effects of genetic perturbations on cellular state and fitness in human pluripotent stem cells (hPSCs) has been challenging using traditional pooled screening techniques which typically rely on unidimensional phenotypic readouts. Here, Applicants use barcoded open reading frame (ORF) overexpression libraries with a coupled single-cell RNA sequencing (scRNA-seq) and fitness screening approach, a technique Applicants call SEUSS (ScalablE fUnctional Screening by Sequencing), to establish a comprehensive assaying platform. Using this system, Applicants perturbed hPSCs with a library of developmentally critical transcription factors (TFs), and assayed the impact of TF overexpression on fitness and transcriptomic cell state across multiple media conditions. Applicants further leveraged the versatility of the ORF library approach to systematically assay mutant gene libraries and also whole gene families. From the transcriptomic responses, Applicants built genetic co-perturbation networks to identify key altered gene modules. Strikingly, Applicants found that KLF4 and SNAI2 have opposing effects on the pluripotency gene module, highlighting the power of Applicants' method to characterize the effects of genetic perturbations. From the fitness responses, Applicants identified ETV2 as a driver of reprogramming towards an endothelial-like state.

### Isolated Nucleic Acids and Transcription Factor Screening Libraries

This disclosure provides isolated polynucleotides or nucleic acids comprising, consisting of, or consisting essentially of (a) a polynucleotide or nucleic acid encoding a transcription factor (TF) open reading frame (ORF); (b) a nucleic acid barcode, and (c) an optional vector comprising (a) and (b); wherein the nucleic acid barcode is located 3′ to the TF ORF.

Transcription factors are proteins that bind (directly or indirectly through recruitment factors) to enhancer or promoter regions of DNA (e.g. a genome) and interact to activate, repress, or maintain the current level of transcription of a particular gene or genetic locus. Many transcription factors can bind to specific DNA sequences. Non-limiting examples of TFs can be found at TFCat (Genome Biol. 2009; 10 (3): R29).

An ORF refers to the part of a gene or polynucleotide that has the potential to be transcribed and/or translated. ORFs span intron/exon regions, which in some embodiments can be spliced together after transcription of the ORF to yield a final mRNA for protein translation. Thus, ORFs include both introns and exons, when applicable. In some embodiments, an ORF is a continuous stretch of codons that contain a start codon and a stop codon. In some embodiments, the transcription termination site is located after the ORF, beyond the translation stop codon.

In some embodiments, the TF ORF encodes a developmentally critical TF. As used herein, “developmentally critical” refers to a transcription factor that regulates development and/or differentiation by modulating transcription. Regulation may include, for example, suppression of one or more specific developmental or differentiation gene expression programs, activation of one or more specific developmental or differentiation gene expression programs, and/or maintenance of a specific level of activation or suppression of a specific developmental or differentiation program. For example, a developmentally critical transcription factor may function upstream of a lineage-specific gene network and direct a stem or progenitor cell to differentiate into that specific cell lineage. Examples of developmentally critical TFs include but are not limited to ASCL1, ASCL3, ASCL4, ASCL5, ATF7, CDX2, CRX, ERG, ESRRG, ETV2, FLI1, FOXA1, FOXA2, FOXA3, FOXP1, GATA1, GATA2, GATA4, GATA6, GLI1, HAND2, HNF1A, HNF1B, HNF4A, HOXA1, HOXA10, HOXA11, HOXB6, KLF4, LHX3, LMXIA, MEF2C, MESP1, MITF, MYC, MYCL, MYCN, MYOD1, MYOG, NEUROD1, NEUROG1, NEUROG3, NRL, ONECUT1, OTX2, PAX7, POU1F1, POU5F1, RUNX, SIX1, SIX2, SNAI2, SOX10, SOX2, SOX3, SPI1, SPIB, SPIC, SRY, TBX5, and TFAP2C.

In some embodiments, the vector is a retroviral vector, optionally a lentiviral vector.

This disclosure provides a vector comprising, or alternatively consisting essentially of, or yet further consisting of a viral backbone. In one aspect, the viral backbone contains essential nucleic acids or sequences for integration into a target cell's genome. In one aspect, the essential nucleic acids necessary for integration of the genome of the target cell include at the 5′ and 3′ ends the minimal LTR regions required for integration of the vector.

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such, include without limitation, equine infectious anaemia virus (EIAV), simian immunodeficiency virus (SIV), feline immunodeficiency virus (FIV), and human immunodeficiency virus (HIV). Alternatively, it is contemplated that other retrovirus can be used as a basis for a vector backbone such murine leukemia virus (MLV). It will be evident that a viral vector need not be confined to the components of a particular virus. The viral vector may comprise components derived from two or more different viruses, and may also comprise synthetic components. Vector components can be manipulated to obtain desired characteristics, such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,07,993; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

In another aspect, provided herein is a TF screening library comprising, consisting of, or consisting essentially of at least one isolated nucleic acid comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF. In some embodiments, the TF ORF encodes a developmentally critical TF, optionally selected from the TFs listed in Table 1.

In some embodiments, the TF screening library comprises, consists of, or consists essentially of at least 10, at least 20, at least 30, at least 40, at least 50, at least 60, at least 70, at least 80, at least 90, or at least 100 nucleic acids or vectors, wherein each nucleic acid or vector comprises, consists of, or consists essentially of a distinct nucleic acid encoding a TF ORF.

In some embodiments, the TF screening library further comprises, consists of, or consists essentially of a nucleic acid encoding a selectable marker (e.g., hygromycin). In some embodiments, the TF screening library further comprises, consists of, or consists essentially of a nucleic acid encoding an expression control element. In some embodiments, the expression control element is a promoter or a long terminal repeat (LTR). In some embodiments, the TF screening library further comprises, consists of, or consists essentially of a nucleic acid encoding a translation elongation factor, optionally wherein the translation elongation factor is Ef1a.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

In another aspect, provided herein is a viral packaging system comprising, consisting of, or consisting essentially of at least one isolated nucleic acid comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF; or aTF screening library; and a packaging plasmid.

In another aspect, provided herein is a method for producing a viral particle, the method comprising, consisting of, or consisting essentially of transfecting a packaging cell line with a viral packaging system comprising, consisting of, or consisting essentially of at least one isolated nucleic acid comprising, consisting of, or consisting essentially of (a) a nucleic acid encoding a transcription factor (TF) open reading frame (ORF); (b) a nucleic acid barcode, and (c) an optional vector comprising (a) and (b); wherein the nucleic acid barcode is located 3′ to the TF ORF; or aTF screening library; and a packaging plasmid under conditions suitable to package the vector or the TF screening library into a viral particle. In another aspect, also provided herein is a viral particle produced by this method, and optionally a carrier. In another aspect, also provided herein is an isolated cell comprising a nucleic acid, vector, or particle as described herein, and optionally a carrier.

Retroviral vectors for use in the methods and compositions described herein include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

This invention also provides the suitable packaging cell line. In one aspect, the packaging cell line is the HEK-293 cell line. Other suitable cell lines are known in the art, for example, described in the patent literature within U.S. Pat. Nos. 7,070,994; 6,995,919; 6,475,786; 6,372,502; 6,365,150 and 5,591,624, each incorporated herein by reference.

Yet further provided is an isolated cell or population of cells, comprising, or alternatively consisting essentially of, or yet further consisting of, a retroviral particle of this invention, which in one aspect, is a viral particle. In one aspect, the isolated host cell is a packaging cell line.

### Kits

In another aspect, provided herein is a kit comprising, consisting of, or consisting essentially of at least one of (a) a nucleic acid or vector according to any of the embodiments described herein; and/or (b) a TF screening library according to any of the embodiments described herein; and/or (c) a viral packaging system according to any of the embodiments described herein; and/or (d) a viral particle according to any of the embodiments described herein; and/or (e) an isolated cell according to any of the embodiments described herein, and optionally instructions for use.

### High Throughput Gene Activation Screens

In another aspect, provided herein is a method of performing a high throughput gene activation screen, the method comprising, consisting of, or consisting essentially of: (a) transducing a target cell with the viral particle according to any of the embodiments described herein; and (b) performing single cell RNA sequencing (scRNA-seq) on the transduced target cell to identify the nucleic acid barcode.

In some embodiments, scRNA-seq methods comprise the following steps: isolation of single cell and RNA, reverse transcription (RT), optional amplification, library generation, and sequencing. Several scRNA-seq protocols appropriate for use with the disclosed methods have been published: Tang et al. (Nat Methods. 6 (5): 377-82) STRT (Islam, S. et al. (2011). Genome Res. 21 (7): 1160-7), SMART-seq (Ramsköld, D. et al. (2012). Nat. Biotechnol. 30 (8): 777-82) CEL-seq (Hashimshony, T. et al. (2012) Cell Rep. 2 (3): 666-73), and Quartz-seq (Sasagawa, Y. et al. (2013) Genome Biol. 14 (4): R31).

In some embodiments, the method further comprises or consists of determining a fitness effect in the transduced target cell. Fitness effects include but are not limited to effects on cell proliferation, effects on cell viability, effects on rate of senescence, effects on apoptosis, effects on DNA repair mechanisms, effects on genome stability, effects on gene transcription, and effects on stress response. In some embodiments, fitness effects are calculated from genomic DNA or mRNA reads,

In some embodiments, the method further comprises or consists of identifying a co-perturbation network. In some embodiments, the method further comprises or consists of identifying a functional gene module. In some embodiments, the target cell is a stem cell. In some embodiments, the stem cell is an embryonic stem cell (ESC) or an induced pluripotent stem cell (iPSC). In some embodiments, the target cell is a mammalian cell, optionally wherein the mammalian cell is an equine, bovine, canine, murine, porcine, feline, or human cell. In a particular embodiment, the target cell is a human cell.

### Endothelial Differentiation Methods and Compositions

Also provided herein is a method driving or directing differentiation of a stem cell into an endothelial cell, the method comprising, consisting of, or consisting essentially of inducing ectopic expression of ETV2 (Ets variant 2, Entrez gene: 2116) in a stem cell under conditions suitable to support differentiation of the stem cell into an endothelial cell.

In some embodiments, ectopic expression of ETV2 is induced by transducing the stem cell with a vector (e.g., AAV) comprising a nucleic acid encoding ETV2 and a nucleic acid encoding an expression control element. In other embodiments, the vector encodes an open reading frame of ETV2. In other embodiments, the vector encodes a cDNA of ETV2 (RefSeq: NM_001300974; NM_001304549; NM_014209). A non-limiting example of the sequence of an ETV2 cDNA is provided:

| (SEQ ID NO: 1) | |
|---|---|
| 1 | ttcctgttgc agataagccc agcttagccc agctgacccc agaccctctc ccctcactcc |
| 61 | ccccatgtcg caggatcgag accctgaggc agacagcccg ttcaccaagc cccccgcccc |
| 121 | gcccccatca ccccgtaaac ttctcccagc ctccgccctg ccctcaccca gcccgctgtt |
| 181 | ccccaagcct cgctccaagc ccacgccacc cctgcagcag ggcagcccca gaggccagca |
| 241 | cctatccccg aggctggggt cgaggctcgg ccccgcccct gcctctgcaa cttgagcctg |
| 301 | gctgcgaccc ctgctctgac gtctcggaaa attccccctt gcccaggccc ttgggggagg |
| 361 | gggtgcatgg tatgaaatgg ggctgagacc cccggctggg ggcagaggaa cccgccagag |
| 421 | aaggagccaa attaggcttc tgtttccctg atctggcact ccaaggggac acgccgacag |
| 481 | cgacagcaga gacatgctgg aaaggtacaa gctcatccct ggcaagcttc ccacagctgg |
| 541 | actggggctc cgcgttactg cacccagaag ttccatgggg ggcggagccc gactctcagg |
| 601 | ctcttccgtg gtccggggac tggacagaca tggcgtgcac agcctgggac tcttggagcg |
| 661 | gcgcctcgca gaccctgggc cccgcccctc tcggcccggg ccccatcccc gccgccggct |
| 721 | ccgaaggcgc cgcgggccag aactgcgtcc ccgtggcggg agaggccacc tcgtggtcgc |
| 781 | gcgcccaggc cgccgggagc aacaccagct gggactgttc tgtggggccc gacggcgata |
| 841 | cctactgggg cagtggcctg ggcggggagc cgcgcacgga ctgtaccatt tcgtggggcg |
| 901 | ggcccgcggg cccggactgt accacctcct ggaacccggg gctgcatgcg ggtggcacca |
| 961 | cctctttgaa gcggtaccag agctcagctc tcaccgtttg ctccgaaccg agcccgcagt |
| 1021 | cggaccgtgc cagtttggct cgatgcccca aaactaacca ccgaggtccc attcagctgt |
| 1081 | ggcagttcct cctggagctg ctccacgacg gggcgcgtag cagctgcatc cgttggactg |
| 1141 | gcaacagccg cgagttccag ctgtgcgacc ccaaagaggt ggctcggctg tggggcgagc |
| 1201 | gcaagagaaa gccgggcatg aattacgaga agctgagccg gggccttcgc tactactatc |
| 1261 | gccgcgacat cgtgcgcaag agcggggggc gaaagtacac gtaccgcttc gggggccgcg |
| 1321 | tgcccagcct agcctatccg gactgtgcgg gaggcggacg gggagcagag acacaataaa |
| 1381 | aattcccggt caaacctcaa aaaaaaaaaa aaa |

In some embodiments, the stem cell is an ESC or an iPSC. In some embodiments, the stem cell is a mammalian cell, optionally wherein the mammalian cell is an equine, bovine, canine, murine, porcine, feline, or human cell. In some embodiments, the stem cell is a human cell. In some embodiments, the stem cell has been genetically modified. In some embodiments, the method further comprises or consists of genetically modifying the stem cell or the endothelial cell.

In further aspect, also provided herein is an endothelial cell produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, and optionally a carrier. In some embodiments, the endothelial cell expresses at least one of CDH5 (VE-Cadherin, Entrez gene: 1003; RefSeq: NM_001114117, NM_00179, PECAM1 (Platelet endothelial cell adhesion molecule, Entrez gene: 5175; RefSeq: NM_000442), or VWF (Von Willebrand Factor, Entrez gene: 7450, RefSeq: NM_000552).

In another aspect, also provided herein is a population of endothelial cells produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, and optionally a carrier.

In some aspects, provided herein is a composition comprising, consisting of, or consisting essentially of an endothelial cell produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, or a population of endothelial cells produced according to a method described herein, and one or more of: a pharmaceutically acceptable carrier, a cryopreservative or a preservative. In some embodiments, the carrier is a pharmaceutically acceptable carrier. In some embodiments, the cryopreservative is suitable for long term storage of the composition at a temperature ranging from −200° C. to 0° C., from −80° C. to 0° C., from −20° C. to 0° C., or from 0° C. to 10° C.

### Methods of Treatment

In some aspects, provided herein is a method of treating a subject in need thereof, the method comprising, consisting of, or consisting essentially of administering an endothelial cell produced by a method driving differentiation of a stem cell into an endothelial cell, the method comprising, consisting of, or consisting essentially of inducing ectopic expression of ETV2 in a stem cell under conditions suitable to support differentiation of the stem cell into an endothelial cell, or a population of endothelial cells produced according to a method described herein, or a composition comprising, consisting of, or consisting essentially of the endothelial cell or population and a carrier to the subject. In some embodiments of the method, an effective amount of the endothelial cell, population, or composition is administered to the subject. In some embodiments, the endothelial cell or population is allogenic or autologous to the subject being treated. In one aspect, the treatment excludes prevention.

In some embodiments of the method, the subject has a wound, a corneal disease or condition, a myocardial infarction, or a vascular disease or condition. In some embodiments, the subject has a corneal disease or condition. In some embodiments, the administration is local or systemic. In some embodiments, the endothelial cell, population, or composition is administered to the subject's eye.

An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. The dosage can be determined by a physician and adjusted, as necessary, to suit observed effects of the treatment. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to achieve the result of the method.

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

In some embodiments of the method, the subject is a mammal and the mammal is an equine, bovine, canine, murine, porcine, feline, or human. In some embodiments, the mammal is a human. In some embodiments, the endothelial cells are autologous or allogeneic to the subject being treated.

Having been generally described herein, the follow examples are provided to further illustrate this invention.

### Example 1

Recently, screens combining genetic perturbations with scRNA-seq readouts have emerged as promising alternatives to traditional screens, enabling high-throughput, high-content screening by profiling the transcriptomes of tens of thousands of individual cells simultaneously. Unlike array-based methods scRNA-seq screens are scalable, while unlike traditional pooled screening techniques, they enable direct readout of cell state changes. In addition, they also enable the evaluation of heterogeneous cellular response to perturbations. While several groups have demonstrated CRISPR-Cas9 based knock-out and knock-down scRNA-seq screens, to Applicants' knowledge, gene activation screens have yet to be demonstrated.

Here, Applicants use barcoded ORF overexpression libraries with a coupled scRNA-seq and fitness screen, a technique Applicants call SEUSS, to systematically overexpress TFs and assay both, the transcriptomic and fitness effects on hPSCs. Applicants chose open-reading frame (ORF) constructs for several reasons, namely that ORF constructs yield strong, stable expression of the gene of interest, enable the ability to express a targeted isoform of the gene, and allow for the ability to express engineered or mutant forms of the gene, aspects otherwise not accessible through endogenous gene activation. Applicants screened a pooled library of TFs that are either developmentally critical, specific to key lineages, or are pioneer factors capable of binding closed chromatin (Table 1). From the transcriptomic readouts, Applicants built a gene-gene co-perturbation network, segmented the network genes into functional gene modules, and used these gene modules to also elucidate the impact of TF overexpression on the pluripotent cell state. Notably, Applicants also leveraged the versatility of the ORF library approach and SEUSS to systematically assay mutant gene libraries (MYC) and whole gene families (KLF). Finally, Applicants also leveraged the complementary fitness information via SEUSS to ascertain that ETV2 is a novel reprogramming factor for hPSCs, whose overexpression yields rapid differentiation towards the endothelial lineage.

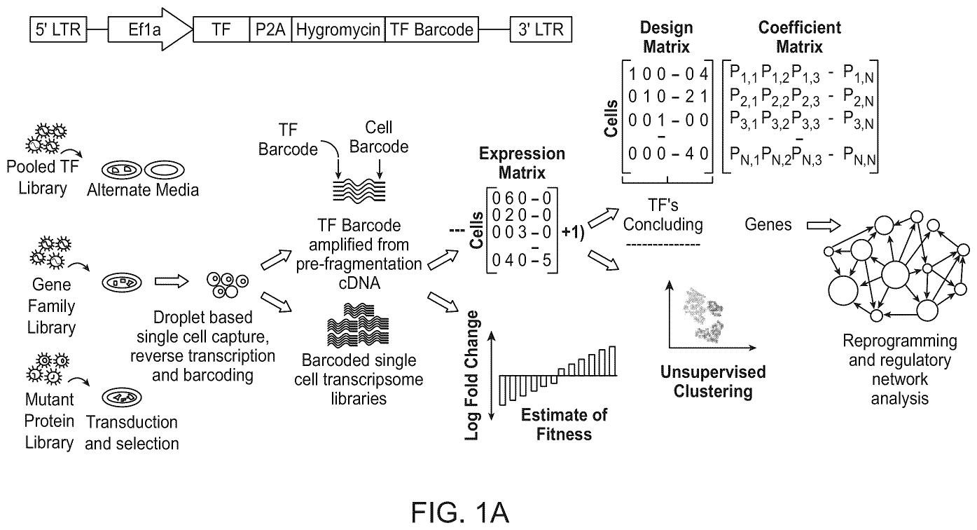

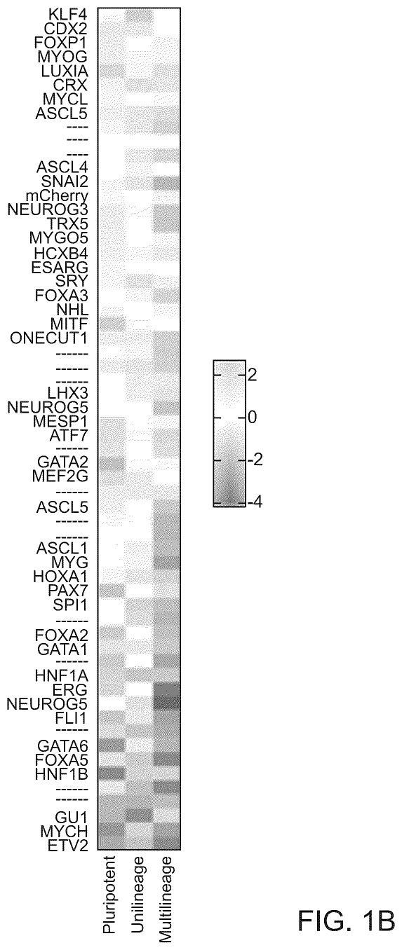

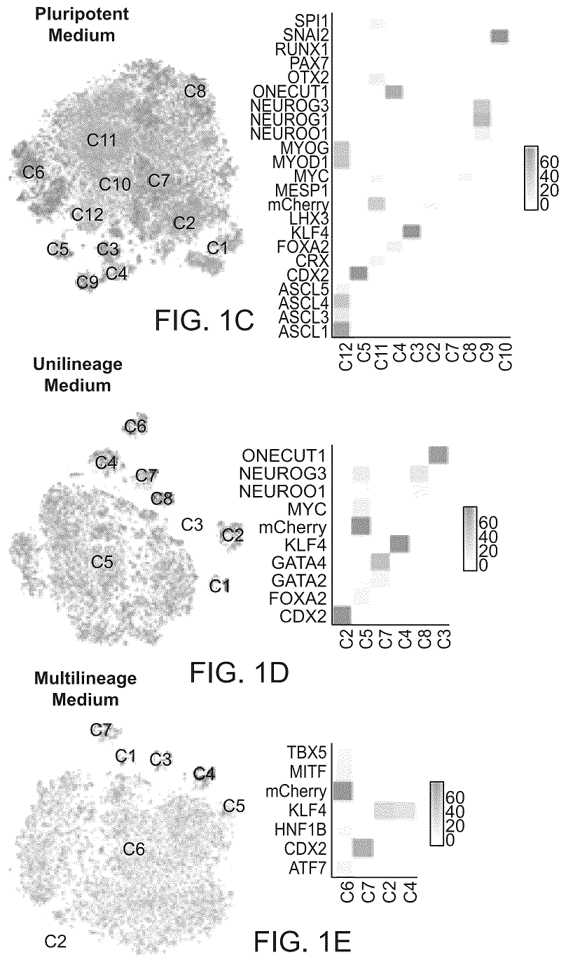

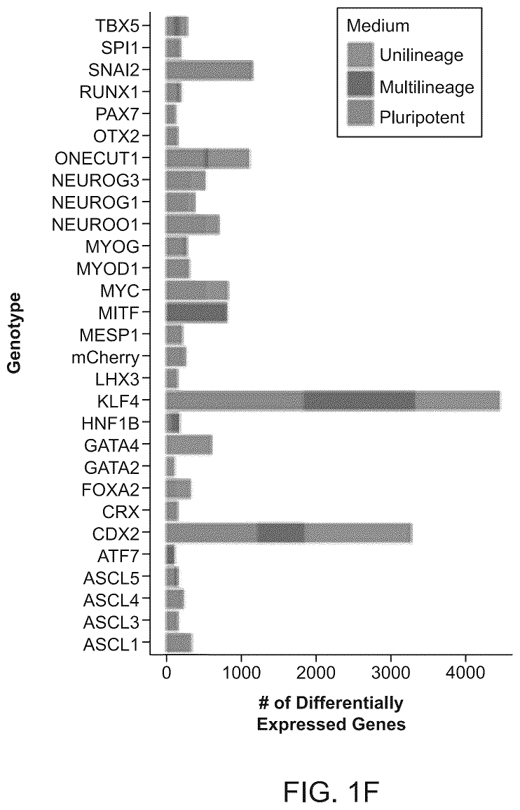

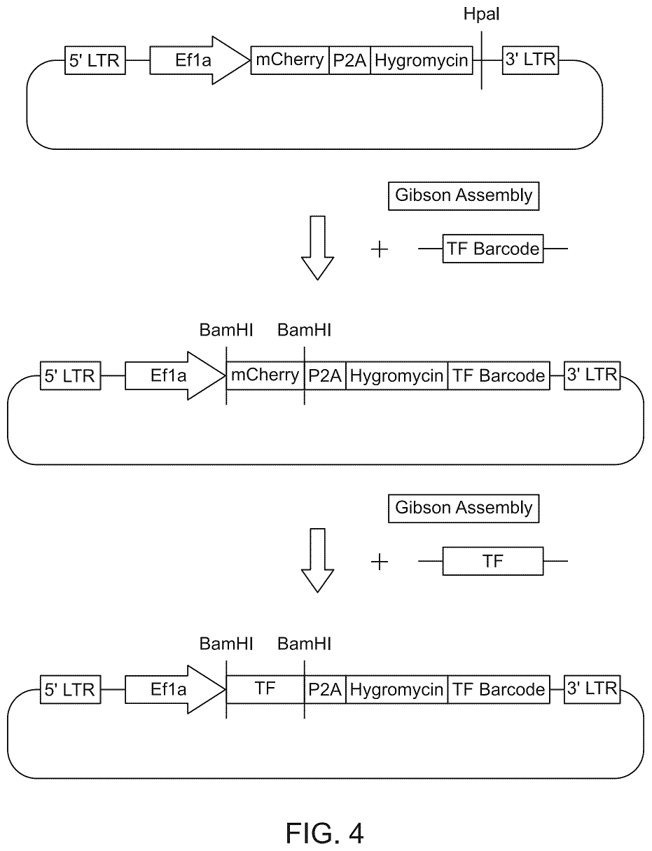

Applicants designed Applicants' ORF overexpression vector such that each TF was paired with a unique 20 bp barcode sequence located downstream of the 3′ end of a hygromycin resistance transgene (FIG. 1A, FIG. 4), and 200 bp upstream of the lentiviral 3′-long terminal repeat (LTR) region. This yields a polyadenylated transcript bearing the barcode proximal to the 3′ end, thereby facilitating efficient capture and detection in scRNA-seq. To construct the ORF library, transcription factors were amplified out of a multi-tissue human cDNA pool or directly synthesized as double-stranded DNA fragments, and individually cloned into the backbone vector (FIG. 4). The final library consisted of 61 developmentally critical or pioneer TFs (Table 1). Applicants chose this library size to ensure that within a single scRNA-seq run of up to 10,000 cells, each perturbation was represented by at least 50-100 cells. However, SEUSS can be scaled up to include all known TFs.

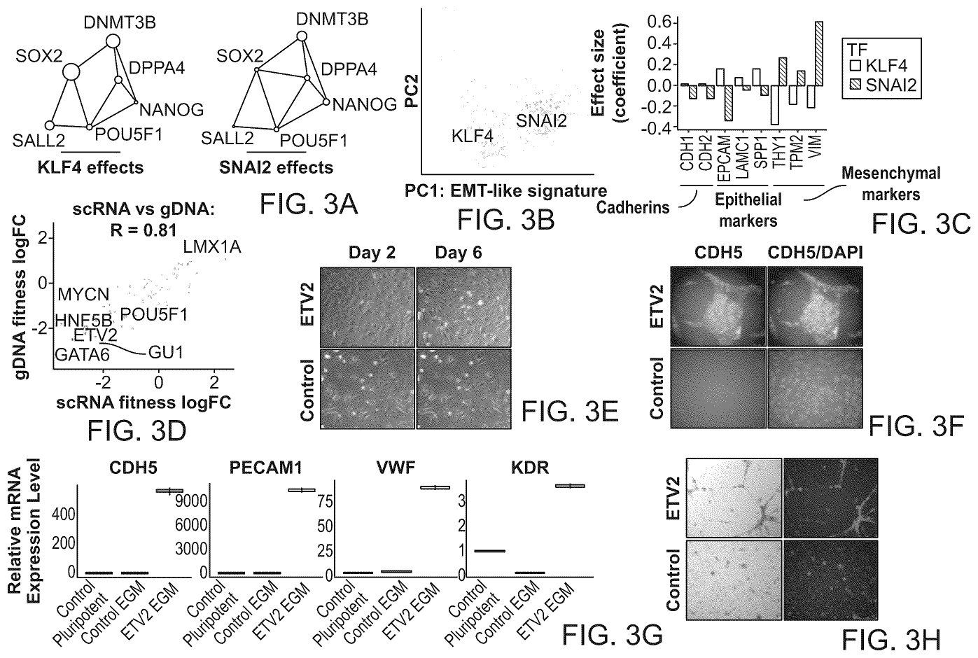

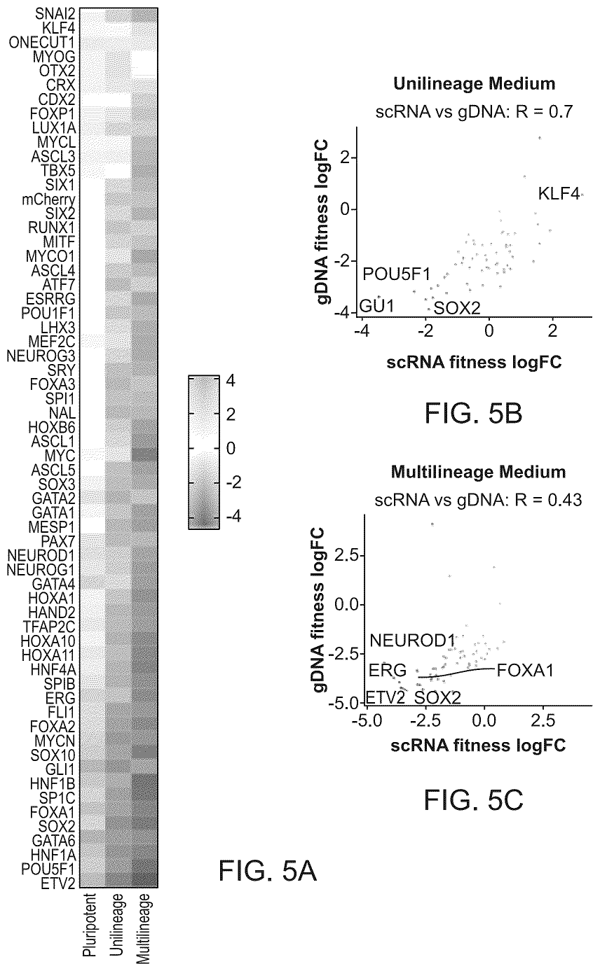

Applicants conducted the overexpression screens by transducing lentiviral ORF libraries into human embryonic stem cells (hESCs), maintaining them under antibiotic selection for 5 days after transduction, for screens in hPSC medium, and 6 days after transduction, for screens in unlineage (endothelial) and multilineage (high serum) medium, and then performing scRNA-seq on the transduced and selected cells. TF barcodes were recovered and associated with scRNA-seq cell barcodes by targeted amplification from the unfragmented cDNA, allowing genotyping of each cell for downstream analysis (FIG. 1A). Genotyped cell counts, although an under-sampling of the bulk population, also allowed Applicants to obtain an estimate of fitness, which was strongly correlated with bulk fitness obtained from genomic DNA (FIG. 1A, FIG. 3D, FIGS. 5A-5C).

To analyze the effect of the TF perturbations, Applicants used the Seurat computational pipeline to cluster the cells from the scRNA-seq expression matrix (FIG. 1C, FIG. 1D, FIG. 1E). In parallel, a linear model was used to identify genes whose expression levels are appreciably changed by the perturbation. To select TFs for downstream analysis, Applicants calculated over-enrichment of TFs in clusters using Fisher's exact test (FIG. 1C, FIG. 1D, FIG. 1E). Subsequently, Applicants focused Applicants' analysis on TFs that were either significantly enriched for at least one cluster (FDR <10⁻⁶), or had at least 100 significant differentially expressed genes. For TEs that had significant over-enrichment in a cluster, Applicants repeated the linear regression analysis, only including cells that fell into enriched clusters (FIG. 1F).

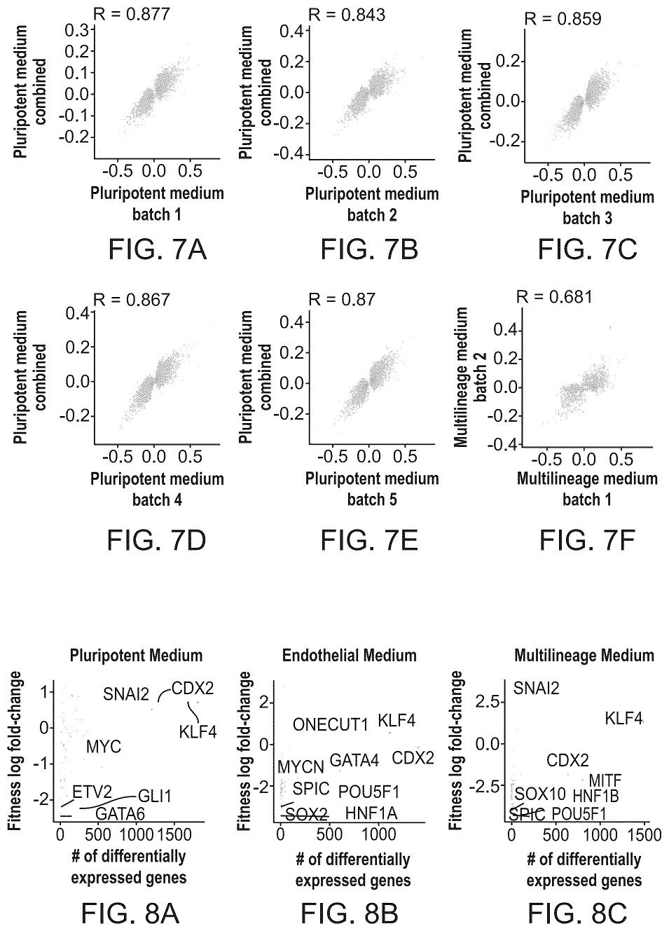

This framework was used to conduct screens in hPSC medium, aggregating 12,873 cells across five samples. Applicants found that these independent experiments were well correlated with the combined dataset (Pearson R >0.84), implying overall reproducibility and the absence of strong batch effects (FIGS. 7A-7E). To study the interplay of ORF overexpression with growth media conditions, Applicants also conducted screens in a unilineage medium, specifically endothelial growth medium, on 5,646 cells and in a multilineage (ML) differentiation medium, specifically a high serum growth medium, on 3476 cells (Table 3). Two samples were aggregated for analysis in the ML medium, again showing good correlation (FIG. 7F; Pearson R=0.68).

From Applicants' screen in hPSC medium, Applicants found that transcriptomic changes do not necessarily correlate with changes in fitness (FIG. 5), thus Applicants' coupled screening method enables a more comprehensive profiling of impacts on both fitness and cell state. Among the most significantly depleted TFs, was the haemato-endothelial master regulator ETV2, (FIG. 3D, FIG. 5), which guided Applicants' choice of EGM for a unilineage medium screen.

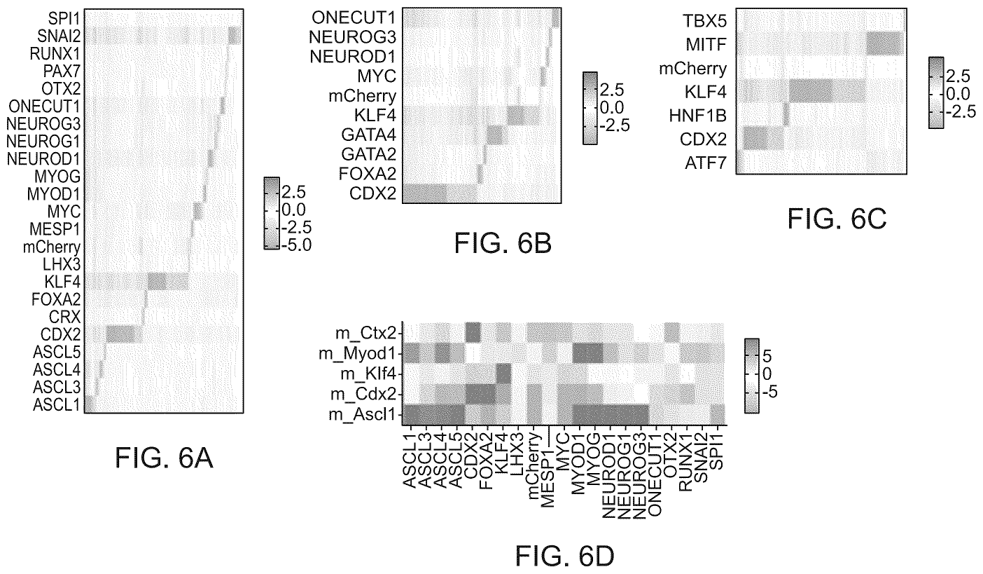

Applicants find that certain TFs show consistent effects across all media conditions (CDX2, KLF4), while some TFs have medium-specific effects. For instance, SNAI2 effects were specific to hPSC medium, MITF to ML medium, and GATA4 to EGM (FIG. 1F). To benchmark Applicants' results, Applicants compared expression profiles for significant TFs in hPSC medium with a previously reported bulk RNA-seq screen of TF perturbations in mESCs. For TFs present in both datasets, Applicants found a strong overlap, suggesting the effectiveness of Applicants' screen for studying perturbations (FIG. 6D).

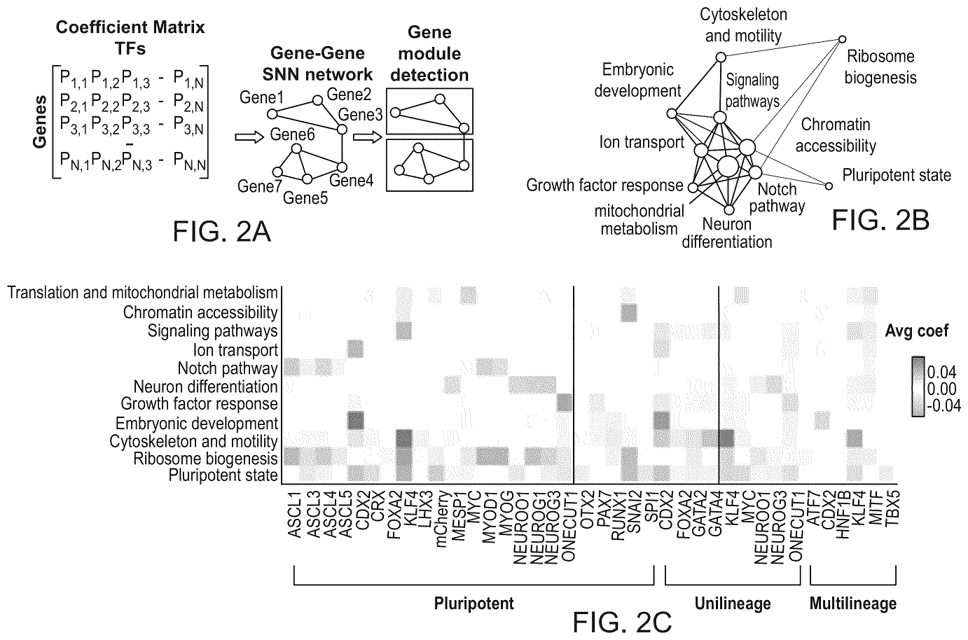

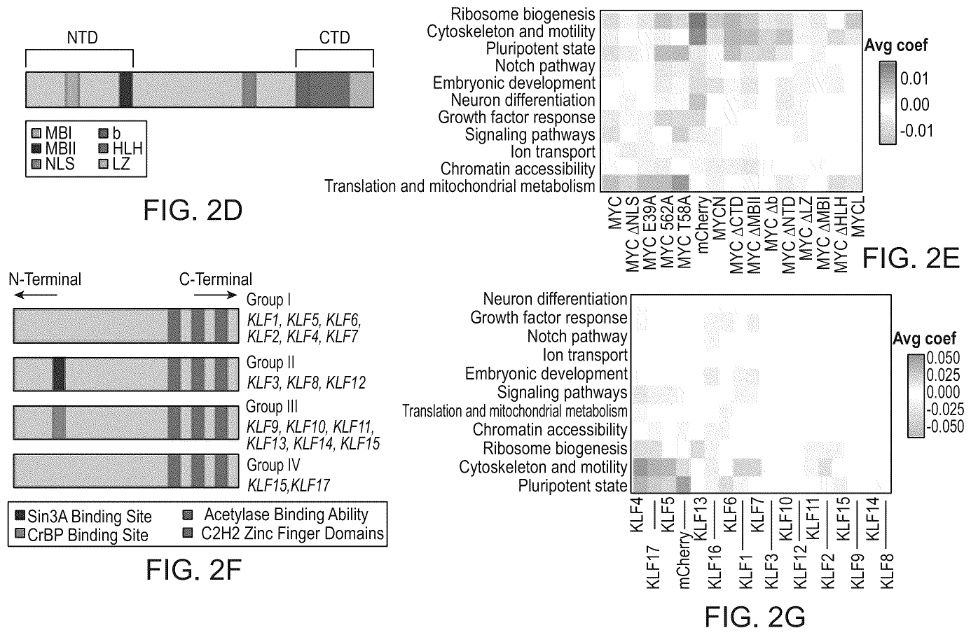

To interpret the effects of the significant TFs, Applicants used the regression coefficients of the linear model to build a weighted gene-to-gene co-perturbation network, where genes with a highly weighted edge between them respond to TF perturbations in a similar manner (FIG. 2A). Using this network, Applicants identified 11 altered gene modules via a modularity optimization graph clustering algorithm. Many of these gene modules showed a strong enrichment for Gene Ontology (GO) terms, and gene module identity was assigned using GO enrichment paired with manual inspection of genes in each module. In this network, Applicants found that the pluripotency gene module and the chromatin accessibility module are highly interconnected, reflecting the relationship between those two biological processes (FIG. 2B), and suggesting that this network may serve as a resource to understand the cascading effects of genetic perturbations (FIG. 2B, Table 5).

Applicants next calculated the effect of each significant TF on the gene modules (FIG. 2C). Applicants found that the annotated neural specifiers NEUROD1, NEUROG1, and NEUROG3, which show similar cluster enrichment and differential expression patterns, upregulate the neuron differentiation module, consistent with their known effects. ASCL1 and MYOD1, which also show similarity in clustering and expression patterns, upregulate the Notch pathway module (FIG. 2C). This similarity between ASCL1 and MYOD1 may be due to a myogenic program initiated by ASCL1. Notably, for the TFs with consistent effects across medium conditions, Applicants find that both CDX2 and KLF4 strongly downregulate the pluripotency gene module, while CDX2 also upregulates the embryonic development gene module, potentially reflecting its role in trophectoderm development, and KLF4 tends to upregulate the cytoskeleton and motility gene modules.

Next, since in Applicants' screens MYC was found to drive significant transcriptomic changes in hPSC medium in its wild type form (FIG. 1F), Applicants chose to focus on it in demonstrating the ability of Applicants' platform to also systematically screen mutant forms of proteins. Specifically, Applicants constructed a library of mutant MYC proteins, where functional domains were systematically deleted (FIG. 2D), or mutations at known hotspots were incorporated (Glu-39, Thr-58 and Ser-62). Screening this library in pluripotent stem cell medium, Applicants found that while some variants, such as known hotspot mutations, as well as deletion of the nuclear localization signal (NLS) sequence maintain an effect similar to the wild type MYC, a majority of the other mutant forms show a greater overlap with the control mCherry-transduced cells, suggesting the essential requirement of the mapped domains for function of MYC in hPSCs (FIG. 2E).

### MYC Mutants Library:

| | | SEQ | |
|---|---|---|---|
| | | ID | |
| GENE | SEQUENCE | NO: | MUTATION |
| MYC | ATGCCCCTCAACGTTAGCTTCACCAACAGGAACTATGACC | 2 | Deletion of MYC |
| ΔMBI | TCGACTACGACTCGGTGCAGCCGTATTTCTACTGCGACGA | | Box I |
| | GGAGGAGAACTTCTACCAGCAGCAGCAGCAGAGCGAGCT | | |
| | GCAGCCCCCGGCGGGATCAGGTAGCGGTAGCCGCCGCTC | | |
| | CGGGCTCTGCTCGCCCTCCTACGTTGCGGTCACACCCTTCT | | |
| | CCCTTCGGGGAGACAACGACGGCGGTGGCGGGAGCTTCT | | |
| | CCACGGCCGACCAGCTGGAGATGGTGACCGAGCTGCTGG | | |
| | GAGGAGACATGGTGAACCAGAGTTTCATCTGCGACCCGG | | |
| | ACGACGAGACCTTCATCAAAAACATCATCATCCAGGACTG | | |
| | TATGTGGAGCGGCTTCTCGGCCGCCGCCAAGCTCGTCTCA | | |
| | GAGAAGCTGGCCTCCTACCAGGCTGCGCGCAAAGACAGC | | |
| | GGCAGCCCGAACCCCGCCCGCGGCCACAGCGTCTGCTCCA | | |
| | CCTCCAGCTTGTACCTGCAGGATCTGAGCGCCGCCGCCTC | | |
| | AGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCAAGAGGCGAAC | | |
| | ACACAACGTCTTGGAGCGCCAGAGGAGGAACGAGCTAAA | | |
| | ACGGAGCTTTTTTGCCCTGCGTGACCAGATCCCGGAGTTG | | |
| | GAAAACAATGAAAAGGCCCCCAAGGTAGTTATCCTTAAA | | |
| | AAAGCCACAGCATACATCCTGTCCGTCCAAGCAGAGGAG | | |
| | CAAAAGCTCATTTCTGAAGAGGACTTGTTGCGGAAACGAC | | |
| | GAGAACAGTTGAAACACAAACTTGAACAGCTACGGAACT | | |
| | CTTGTGCG | | |
| c-MYC | ATGCCCCTCAACGTTAGCTTCACCAACAGGAACTATGACC | 3 | Deletion of MYC |
| ΔMBII | TCGACTACGACTCGGTGCAGCCGTATTTCTACTGCGACGA | | Box II |
| | GGAGGAGAACTTCTACCAGCAGCAGCAGCAGAGCGAGCT | | |
| | GCAGCCCCCGGCGCCCAGCGAGGATATCTGGAAGAAATT | | |
| | CGAGCTGCTGCCCACCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCGGATCAGGTAGC | | |
| | GGTCTCGTCTCAGAGAAGCTGGCCTCCTACCAGGCTGCGC | | |
| | GCAAAGACAGCGGCAGCCCGAACCCCGCCCGCGGCCACA | | |
| | GCGTCTGCTCCACCTCCAGCTTGTACCTGCAGGATCTGAG | | |
| | CGCCGCCGCCTCAGAGTGCATCGACCCCTCGGTGGTCTTC | | |
| | CCCTACCCTCTCAACGACAGCAGCTCGCCCAAGTCCTGCG | | |
| | CCTCGCAAGACTCCAGCGCCTTCTCTCCGTCCTCGGATTCT | | |
| | CTGCTCTCCTCGACGGAGTCCTCCCCGCAGGGCAGCCCCG | | |
| | AGCCCCTGGTGCTCCATGAGGAGACACCGCCCACCACCAG | | |
| | CAGCGACTCTGAGGAGGAACAAGAAGATGAGGAAGAAAT | | |
| | CGATGTTGTTTCTGTGGAAAAGAGGCAGGCTCCTGGCAAA | | |
| | AGGTCAGAGTCTGGATCACCTTCTGCTGGAGGCCACAGCA | | |
| | AACCTCCTCACAGCCCACTGGTCCTCAAGAGGTGCCACGT | | |
| | CTCCACACATCAGCACAACTACGCAGCGCCTCCCTCCACT | | |
| | CGGAAGGACTATCCTGCTGCCAAGAGGGTCAAGTTGGAC | | |
| | AGTGTCAGAGTCCTGAGACAGATCAGCAACAACCGAAAA | | |
| | TGCACCAGCCCCAGGTCCTCGGACACCGAGGAGAATGTC | | |
| | AAGAGGCGAACACACAACGTCTTGGAGCGCCAGAGGAGG | | |
| | AACGAGCTAAAACGGAGCTTTTTTGCCCTGCGTGACCAGA | | |
| | TCCCGGAGTTGGAAAACAATGAAAAGGCCCCCAAGGTAG | | |
| | TTATCCTTAAAAAAGCCACAGCATACATCCTGTCCGTCCA | | |
| | AGCAGAGGAGCAAAAGCTCATTTCTGAAGAGGACTTGTT | | |
| | GCGGAAACGACGAGAACAGTTGAAACACAAACTTGAACA | | |
| | GCTACGGAACTCTTGTGCG | | |
| MYC | ATGCCCCTCAACGTTAGCTTCACCAACAGGAACTATGACC | 4 | Deletion of nuclear |
| ΔNLS | TCGACTACGACTCGGTGCAGCCGTATTTCTACTGCGACGA | | localization signal |
| | GGAGGAGAACTTCTACCAGCAGCAGCAGCAGAGCGAGCT | | sequence |
| | GCAGCCCCCGGCGCCCAGCGAGGATATCTGGAAGAAATT | | |
| | CGAGCTGCTGCCCACCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATGGATCAGGTAGCGGTAGTGTCAGAGTCCTGAGACAGA | | |
| | TCAGCAACAACCGAAAATGCACCAGCCCCAGGTCCTCGG | | |
| | ACACCGAGGAGAATGTCAAGAGGCGAACACACAACGTCT | | |
| | TGGAGCGCCAGAGGAGGAACGAGCTAAAACGGAGCTTTT | | |
| | TTGCCCTGCGTGACCAGATCCCGGAGTTGGAAAACAATGA | | |
| | AAAGGCCCCCAAGGTAGTTATCCTTAAAAAAGCCACAGC | | |
| | ATACATCCTGTCCGTCCAAGCAGAGGAGCAAAAGCTCATT | | |
| | TCTGAAGAGGACTTGTTGCGGAAACGACGAGAACAGTTG | | |
| | AAACACAAACTTGAACAGCTACGGAACTCTTGTGCG | | |
| MYC | ATGCCCCTCAACGTTAGCTTCACCAACAGGAACTATGACC | 5 | Deletion of basic |
| Δb | TCGACTACGACTCGGTGCAGCCGTATTTCTACTGCGACGA | | motif |
| | GGAGGAGAACTTCTACCAGCAGCAGCAGCAGAGCGAGCT | | |
| | GCAGCCCCCGGCGCCCAGCGAGGATATCTGGAAGAAATT | | |
| | CGAGCTGCTGCCCACCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCGGATCAGGTAG | | |
| | CGGTGAGCTAAAACGGAGCTTTTTTGCCCTGCGTGACCAG | | |
| | ATCCCGGAGTTGGAAAACAATGAAAAGGCCCCCAAGGTA | | |
| | GTTATCCTTAAAAAAGCCACAGCATACATCCTGTCCGTCC | | |
| | AAGCAGAGGAGCAAAAGCTCATTTCTGAAGAGGACTTGT | | |
| | TGCGGAAACGACGAGAACAGTTGAAACACAAACTTGAAC | | |
| | AGCTACGGAACTCTTGTGCG | | |
| MYC | ATGCCCCTCAACGTTAGCTTCACCAACAGGAACTATGACC | 6 | Deletion of helix- |
| ΔHLH | TCGACTACGACTCGGTGCAGCCGTATTTCTACTGCGACGA | | loop-helix motif |
| | GGAGGAGAACTTCTACCAGCAGCAGCAGCAGAGCGAGCT | | |
| | GCAGCCCCCGGCGCCCAGCGAGGATATCTGGAAGAAATT | | |
| | CGAGCTGCTGCCCACCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCAAGAGGCGAAC | | |
| | ACACAACGTCTTGGAGCGCCAGAGGAGGAACGGATCAGG | | |
| | TAGCGGTCAAAAGCTCATTTCTGAAGAGGACTTGTTGCGG | | |
| | AAACGACGAGAACAGTTGAAACACAAACTTGAACAGCTA | | |
| | CGGAACTCTTGTGCG | | |
| MYC | ATGCCCCTCAACGTTAGCTTCACCAACAGGAACTATGACC | 7 | Deletion of leucine |
| ΔLZ | TCGACTACGACTCGGTGCAGCCGTATTTCTACTGCGACGA | | zipper motif |
| | GGAGGAGAACTTCTACCAGCAGCAGCAGCAGAGCGAGCT | | |
| | GCAGCCCCCGGCGCCCAGCGAGGATATCTGGAAGAAATT | | |
| | CGAGCTGCTGCCCACCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCAAGAGGCGAAC | | |
| | ACACAACGTCTTGGAGCGCCAGAGGAGGAACGAGCTAAA | | |
| | ACGGAGCTTTTTTGCCCTGCGTGACCAGATCCCGGAGTTG | | |
| | GAAAACAATGAAAAGGCCCCCAAGGTAGTTATCCTTAAA | | |
| | AAAGCCACAGCATACATCCTGTCCGTCCAAGCAGAGGAG | | |
| MYC | ATGGGATCAGGTAGCGGTCTCGTCTCAGAGAAGCTGGCCT | 8 | Deletion of amino- |
| ΔNTD | CCTACCAGGCTGCGCGCAAAGACAGCGGCAGCCCGAACC | | terminal domain: |
| | CCGCCCGCGGCCACAGCGTCTGCTCCACCTCCAGCTTGTA | | Housing MYC Box I |
| | CCTGCAGGATCTGAGCGCCGCCGCCTCAGAGTGCATCGAC | | and II |
| | CCCTCGGTGGTCTTCCCCTACCCTCTCAACGACAGCAGCT | | |
| | CGCCCAAGTCCTGCGCCTCGCAAGACTCCAGCGCCTTCTC | | |
| | TCCGTCCTCGGATTCTCTGCTCTCCTCGACGGAGTCCTCCC | | |
| | CGCAGGGCAGCCCCGAGCCCCTGGTGCTCCATGAGGAGA | | |
| | CACCGCCCACCACCAGCAGCGACTCTGAGGAGGAACAAG | | |
| | AAGATGAGGAAGAAATCGATGTTGTTTCTGTGGAAAAGA | | |
| | GGCAGGCTCCTGGCAAAAGGTCAGAGTCTGGATCACCTTC | | |
| | TGCTGGAGGCCACAGCAAACCTCCTCACAGCCCACTGGTC | | |
| | CTCAAGAGGTGCCACGTCTCCACACATCAGCACAACTACG | | |
| | CAGCGCCTCCCTCCACTCGGAAGGACTATCCTGCTGCCAA | | |
| | GAGGGTCAAGTTGGACAGTGTCAGAGTCCTGAGACAGAT | | |
| | CAGCAACAACCGAAAATGCACCAGCCCCAGGTCCTCGGA | | |
| | CACCGAGGAGAATGTCAAGAGGCGAACACACAACGTCTT | | |
| | GGAGCGCCAGAGGAGGAACGAGCTAAAACGGAGCTTTTT | | |
| | TGCCCTGCGTGACCAGATCCCGGAGTTGGAAAACAATGA | | |
| | AAAGGCCCCCAAGGTAGTTATCCTTAAAAAAGCCACAGC | | |
| | ATACATCCTGTCCGTCCAAGCAGAGGAGCAAAAGCTCATT | | |
| | TCTGAAGAGGACTTGTTGCGGAAACGACGAGAACAGTTG | | |
| | AAACACAAACTTGAACAGCTACGGAACTCTTGTGCG | | |
| MYC | ATGCCCCTCAACGTTAGCTTCACCAACAGGAACTATGACC | 9 | Deletion of carboxy- |
| ΔCTD | TCGACTACGACTCGGTGCAGCCGTATTTCTACTGCGACGA | | terminal domain: |
| | GGAGGAGAACTTCTACCAGCAGCAGCAGCAGAGCGAGCT | | Housing basic helix- |
| | GCAGCCCCCGGCGCCCAGCGAGGATATCTGGAAGAAATT | | loop-helix leucine |
| | CGAGCTGCTGCCCACCCCGCCCCTGTCCCCTAGCCGCCGC | | zipper motif, |
| | TCCGGGCTCTGCTCGCCCTCCTACGTTGCGGTCACACCCTT | | governing |
| | CTCCCTTCGGGGAGACAACGACGGCGGTGGCGGGAGCTT | | heterodimerization |
| | CTCCACGGCCGACCAGCTGGAGATGGTGACCGAGCTGCTG | | with MAX protein |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTC | | |
| MYC | ATGCCCCTCAACGTTAGCTTCACCAACAGGAACTATGACC | 10 | Point mutation |
| Glu39Ala | TCGACTACGACTCGGTGCAGCCGTATTTCTACTGCGACGA | | changing Glutamic |
| | GGAGGAGAACTTCTACCAGCAGCAGCAGCAGAGCGcGCT | | Acid to Alanine at |
| | GCAGCCCCCGGCGCCCAGCGAGGATATCTGGAAGAAATT | | amino acid 39 |
| | CGAGCTGCTGCCCACCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCAAGAGGCGAAC | | |
| | ACACAACGTCTTGGAGCGCCAGAGGAGGAACGAGCTAAA | | |
| | ACGGAGCTTTTTTGCCCTGCGTGACCAGATCCCGGAGTTG | | |
| | GAAAACAATGAAAAGGCCCCCAAGGTAGTTATCCTTAAA | | |
| | AAAGCCACAGCATACATCCTGTCCGTCCAAGCAGAGGAG | | |
| | CAAAAGCTCATTTCTGAAGAGGACTTGTTGCGGAAACGAC | | |
| | GAGAACAGTTGAAACACAAACTTGAACAGCTACGGAACT | | |
| | CTTGTGCG | | |
| MYC | ATGCCCCTCAACGTTAGCTTCACCAACAGGAACTATGACC | 11 | Point mutation |
| Thr58Ala | TCGACTACGACTCGGTGCAGCCGTATTTCTACTGCGACGA | | changing Threonine |
| | GGAGGAGAACTTCTACCAGCAGCAGCAGCAGAGCGAGCT | | to Alanine at amino |
| | GCAGCCCCCGGCGCCCAGCGAGGATATCTGGAAGAAATT | | acid 58 |
| | CGAGCTGCTGCCCGCCCCGCCCCTGTCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCAAGAGGCGAAC | | |
| | ACACAACGTCTTGGAGCGCCAGAGGAGGAACGAGCTAAA | | |
| | ACGGAGCTTTTTTGCCCTGCGTGACCAGATCCCGGAGTTG | | |
| | GAAAACAATGAAAAGGCCCCCAAGGTAGTTATCCTTAAA | | |
| | AAAGCCACAGCATACATCCTGTCCGTCCAAGCAGAGGAG | | |
| | CAAAAGCTCATTTCTGAAGAGGACTTGTTGCGGAAACGAC | | |
| | GAGAACAGTTGAAACACAAACTTGAACAGCTACGGAACT | | |
| | CTTGTGCG | | |
| MYC | ATGCCCCTCAACGTTAGCTTCACCAACAGGAACTATGACC | 12 | Point mutation |
| Ser62Ala | TCGACTACGACTCGGTGCAGCCGTATTTCTACTGCGACGA | | changing Serine to |
| | GGAGGAGAACTTCTACCAGCAGCAGCAGCAGAGCGAGCT | | Alanine at amino acid |
| | GCAGCCCCCGGCGCCCAGCGAGGATATCTGGAAGAAATT | | 58 |
| | CGAGCTGCTGCCCACCCCGCCCCTGGCCCCTAGCCGCCGC | | |
| | TCCGGGCTCTGCTCGCCCTCCTACGTTGCGGTCACACCCTT | | |
| | CTCCCTTCGGGGAGACAACGACGGCGGTGGCGGGAGCTT | | |
| | CTCCACGGCCGACCAGCTGGAGATGGTGACCGAGCTGCTG | | |
| | GGAGGAGACATGGTGAACCAGAGTTTCATCTGCGACCCG | | |
| | GACGACGAGACCTTCATCAAAAACATCATCATCCAGGACT | | |
| | GTATGTGGAGCGGCTTCTCGGCCGCCGCCAAGCTCGTCTC | | |
| | AGAGAAGCTGGCCTCCTACCAGGCTGCGCGCAAAGACAG | | |
| | CGGCAGCCCGAACCCCGCCCGCGGCCACAGCGTCTGCTCC | | |
| | ACCTCCAGCTTGTACCTGCAGGATCTGAGCGCCGCCGCCT | | |
| | CAGAGTGCATCGACCCCTCGGTGGTCTTCCCCTACCCTCTC | | |
| | AACGACAGCAGCTCGCCCAAGTCCTGCGCCTCGCAAGACT | | |
| | CCAGCGCCTTCTCTCCGTCCTCGGATTCTCTGCTCTCCTCG | | |
| | ACGGAGTCCTCCCCGCAGGGCAGCCCCGAGCCCCTGGTGC | | |
| | TCCATGAGGAGACACCGCCCACCACCAGCAGCGACTCTG | | |
| | AGGAGGAACAAGAAGATGAGGAAGAAATCGATGTTGTTT | | |
| | CTGTGGAAAAGAGGCAGGCTCCTGGCAAAAGGTCAGAGT | | |
| | CTGGATCACCTTCTGCTGGAGGCCACAGCAAACCTCCTCA | | |
| | CAGCCCACTGGTCCTCAAGAGGTGCCACGTCTCCACACAT | | |
| | CAGCACAACTACGCAGCGCCTCCCTCCACTCGGAAGGACT | | |
| | ATCCTGCTGCCAAGAGGGTCAAGTTGGACAGTGTCAGAGT | | |
| | CCTGAGACAGATCAGCAACAACCGAAAATGCACCAGCCC | | |
| | CAGGTCCTCGGACACCGAGGAGAATGTCAAGAGGCGAAC | | |
| | ACACAACGTCTTGGAGCGCCAGAGGAGGAACGAGCTAAA | | |
| | ACGGAGCTTTTTTGCCCTGCGTGACCAGATCCCGGAGTTG | | |
| | GAAAACAATGAAAAGGCCCCCAAGGTAGTTATCCTTAAA | | |
| | AAAGCCACAGCATACATCCTGTCCGTCCAAGCAGAGGAG | | |
| | CAAAAGCTCATTTCTGAAGAGGACTTGTTGCGGAAACGAC | | |
| | GAGAACAGTTGAAACACAAACTTGAACAGCTACGGAACT | | |
| | CTTGTGCG | | |

Additionally, the consistent and strong effects of KLF4 overexpression motivated the investigation of the full KLF zinc finger transcription factor family (FIG. 2F) as a demonstration of the utility of Applicants' technique in studying patterns of perturbation effects across gene families. A screen including all 17 members of the KLF family was conducted in pluripotent stem cell medium. Gene module analysis showed that KLF5 and KLF17 also have similar effects as KLF4 (FIG. 2G), which may reflect their similar role in promoting or maintaining epithelial cell states. On the other hand, unlike most of the KLF family, KLF13 and KLF16 fail to activate the cytoskeleton and motility module (FIG. 2G).

### KLF Family Library

| | | SEQ ID |
|---|---|---|
| GENE | SEQUENCE | NO: |
| KLF1 | ATGGCGACTGCGGAGACAGCACTTCCATCAATCTCAACACTCACTGCACTG | 13 |
| | GGGCCATTTCCAGATACCCAGGACGATTTCCTTAAGTGGTGGCGGTCCGAA | |
| | GAGGCTCAAGACATGGGACCTGGTCCGCCGGATCCCACCGAACCTCCTCTG | |
| | CATGTCAAAAGTGAAGATCAGCCTGGCGAGGAAGAGGATGACGAAAGGG | |
| | GTGCCGACGCCACTTGGGACTTGGATCTTCTCCTTACCAATTTCTCTGGTCC | |
| | GGAACCTGGCGGGGCACCACAGACGTGCGCTCTCGCTCCCTCAGAAGCGA | |
| | GCGGGGCTCAGTACCCACCCCCTCCCGAAACTCTGGGAGCCTATGCTGGGG | |
| | GTCCTGGACTGGTGGCTGGGTTGCTTGGTAGTGAGGACCATTCTGGCTGGG | |
| | TACGCCCCGCTTTGAGGGCCCGCGCTCCGGACGCCTTTGTGGGACCGGCGC | |
| | TCGCTCCTGCACCGGCTCCGGAACCAAAAGCCCTCGCGCTGCAGCCCGTGT | |
| | ACCCCGGACCCGGAGCCGGATCCTCAGGGGGATACTTCCCACGGACCGGA | |
| | CTCAGCGTTCCAGCGGCTTCCGGGGCGCCATACGGATTGTTGAGCGGCTAC | |
| | CCGGCTATGTATCCCGCTCCCCAGTACCAAGGACACTTCCAATTGTTCCGG | |
| | GGTCTTCAAGGGCCTGCGCCCGGGCCTGCTACCAGTCCCAGTTTCCTCAGT | |
| | TGTCTGGGACCGGGAACTGTTGGCACTGGACTTGGCGGGACTGCAGAGGA | |
| | CCCAGGCGTTATAGCAGAGACAGCGCCAAGTAAAAGGGGCCGACGAAGCT | |
| | GGGCCAGGAAACGCCAAGCTGCGCACACTTGTGCCCATCCAGGTTGCGGT | |
| | AAATCCTACACGAAGAGCAGTCATCTTAAAGCACATCTTCGCACACACAC | |
| | GGGCGAGAAGCCCTACGCCTGTACTTGGGAAGGTTGCGGCTGGAGATTCG | |
| | CTAGATCTGACGAGCTCACCCGGCATTATCGAAAACACACTGGCCAGCGA | |
| | CCGTTCCGGTGCCAACTCTGCCCAAGGGCGTTCAGTCGCTCAGATCATCTG | |
| | GCTTTGCATATGAAGCGACACCTT | |
| KLF2 | ATGGCCCTTAGTGAACCCATTCTTCCCAGCTTTTCCACGTTCGCGTCTCCTT | 14 |
| | GCCGAGAGAGAGGCCTTCAGGAAAGGTGGCCGAGGGCTGAACCCGAGTCT | |
| | GGAGGTACGGATGATGATCTTAACAGTGTGCTCGATTTCATACTCTCAATG | |
| | GGACTGGACGGGCTGGGAGCGGAGGCAGCTCCTGAACCACCACCACCCCC | |
| | TCCGCCCCCAGCGTTTTACTACCCGGAGCCAGGTGCGCCGCCGCCATATTC | |
| | AGCCCCGGCGGGTGGCTTGGTGTCCGAGCTCCTCCGGCCTGAATTGGATGC | |
| | CCCGCTCGGCCCGGCGCTGCATGGTAGATTTCTGCTCGCGCCTCCGGGTCG | |
| | ACTCGTTAAGGCTGAACCTCCTGAGGCTGATGGTGGAGGTGGCTACGGAT | |
| | GTGCCCCCGGGCTTACCCGAGGACCGAGAGGTCTTAAGCGGGAAGGGGCA | |
| | CCTGGCCCGGCTGCAAGCTGTATGCGGGGGCCCGGTGGGAGGCCTCCCCC | |
| | GCCCCCTGATACACCCCCCCTTAGTCCAGATGGACCAGCTCGACTTCCCGC | |
| | ACCTGGCCCCAGAGCGAGTTTCCCCCCTCCATTTGGAGGACCGGGGTTTGG | |
| | CGCCCCAGGTCCTGGACTTCACTACGCCCCTCCTGCCCCCCCAGCTTTTGGT | |
| | CTTTTCGACGATGCTGCTGCTGCCGCAGCAGCCTTGGGCCTTGCGCCGCCC | |
| | GCAGCCAGGGGACTGCTCACGCCACCGGCAAGCCCCCTGGAGCTCCTTGA | |
| | AGCCAAGCCGAAGCGAGGACGCAGATCATGGCCGCGCAAGCGGACAGCT | |
| | ACGCATACCTGCTCATATGCGGGCTGCGGAAAAACCTACACAAAGAGTTC | |
| | ACACCTTAAAGCGCACCTTCGCACACACACAGGCGAGAAACCATATCATT | |
| | GTAACTGGGACGGATGTGGATGGAAATTTGCTCGGTCTGATGAGCTTACGA | |
| | GACATTATCGAAAGCATACCGGACATCGGCCCTTTCAATGCCATCTTTGTG | |
| | ACAGAGCTTTTTCCCGGTCTGACCACCTCGCTCTGCACATGAAGAGGCACA | |
| | TG | |
| KLF3 | ATGCTCATGTTTGACCCAGTTCCTGTCAAGCAAGAGGCCATGGACCCTGTC | 15 |
| | TCAGTGTCATACCCATCTAATTACATGGAATCCATGAAGCCTAACAAGTAT | |
| | GGGGTCATCTACTCCACACCATTGCCTGAGAAGTTCTTTCAGACCCCAGAA | |
| | GGTCTGTCGCACGGAATACAGATGGAGCCAGTGGACCTCACGGTGAACAA | |
| | GCGGAGTTCACCCCCTTCGGCTGGGAATTCGCCCTCCTCTCTGAAGTTCCC | |
| | GTCCTCACACCGGAGAGCCTCGCCTGGGTTGAGCATGCCTTCTTCCAGCCC | |
| | ACCGATAAAAAAATACTCACCCCCTTCTCCAGGCGTGCAGCCCTTCGGCGT | |
| | GCCGCTGTCCATGCCACCAGTGATGGCAGCTGCCCTCTCGCGGCATGGAAT | |
| | ACGGAGCCCGGGGATCCTGCCCGTCATCCAGCCGGTGGTGGTGCAGCCCG | |
| | TCCCCTTTATGTACACAAGTCACCTCCAGCAGCCTCTCATGGTCTCCTTATC | |
| | GGAGGAGATGGAAAATTCCAGTAGTAGCATGCAAGTACCTGTAATTGAAT | |
| | CATATGAGAAGCCTATATCACAGAAAAAAATTAAAATAGAACCTGGGATC | |
| | GAACCACAGAGGACAGATTATTATCCTGAAGAAATGTCACCCCCCTTAATG | |
| | AACTCAGTGTCCCCCCCGCAAGCATTGTTGCAAGAGAATCACCCTTCGGTC | |
| | ATCGTGCAGCCTGGGAAGAGACCTTTACCTGTGGAATCCCCGGATACTCAA | |
| | AGGAAGCGGAGGATACACAGATGTGATTATGATGGATGCAACAAAGTGTA | |
| | CACTAAAAGCTCCCACTTGAAAGCACACAGAAGAACACACACAGGAGAAA | |
| | AACCCTACAAATGTACATGGGAAGGGTGCACATGGAAGTTTGCTCGGTCT | |
| | GATGAACTAACAAGACATTTCCGAAAACATACTGGAATCAAACCTTTCCA | |
| | GTGCCCGGACTGTGACCGCAGCTTCTCCCGTTCTGACCATCTTGCCCTCCAT | |
| | AGGAAACGCCACATGCTAGTC | |
| KLF5 | ATGGCTACAAGGGTGCTGAGCATGAGCGCCCGCCTGGGACCCGTGCCCCA | 16 |
| | GCCGCCGGCGCCGCAGGACGAGCCGGTGTTCGCGCAGCTCAAGCCGGTGC | |
| | TGGGCGCCGCGAATCCGGCCCGCGACGCGGCGCTCTTCCCCGGCGAGGAG | |
| | CTGAAGCACGCGCACCACCGCCCGCAGGCGCAGCCCGCGCCCGCGCAGGC | |
| | CCCGCAGCCGGCCCAGCCGCCCGCCACCGGCCCGCGGCTGCCTCCAGAGG | |
| | ACCTGGTCCAGACAAGATGTGAAATGGAGAAGTATCTGACACCTCAGCTT | |
| | CCTCCAGTTCCTATAATTCCAGAGCATAAAAAGTATAGACGAGACAGTGCC | |
| | TCAGTCGTAGACCAGTTCTTCACTGACACTGAAGGGTTACCTTACAGTATC | |
| | AACATGAACGTCTTCCTCCCTGACATCACTCACCTGAGAACTGGCCTCTAC | |
| | AAATCCCAGAGACCGTGCGTAACACACATCAAGACAGAACCTGTTGCCAT | |
| | TTTCAGCCACCAGAGTGAAACGACTGCCCCTCCTCCGGCCCCGACCCAGGC | |
| | CCTCCCTGAGTTCACCAGTATATTCAGCTCACACCAGACCGCAGCTCCAGA | |
| | GGTGAACAATATTTTCATCAAACAAGAACTTCCTACACCAGATCTTCATCT | |
| | TTCTGTCCCTACCCAGCAGGGCCACCTGTACCAGCTACTGAATACACCGGA | |
| | TCTAGATATGCCCAGTTCTACAAATCAGACAGCAGCAATGGACACTCTTAA | |
| | TGTTTCTATGTCAGCTGCCATGGCAGGCCTTAACACACACACCTCTGCTGTT | |
| | CCGCAGACTGCAGTGAAACAATTCCAGGGCATGCCCCCTTGCACATACAC | |
| | AATGCCAAGTCAGTTTCTTCCACAACAGGCCACTTACTTTCCCCCGTCACC | |
| | ACCAAGCTCAGAGCCTGGAAGTCCAGATAGACAAGCAGAGATGCTCCAGA | |
| | ATTTAACCCCACCTCCATCCTATGCTGCTACAATTGCTTCTAAACTGGCAAT | |
| | TCACAATCCAAATTTACCCACCACCCTGCCAGTTAACTCACAAAACATCCA | |
| | ACCTGTCAGATACAATAGAAGGAGTAACCCCGATTTGGAGAAACGACGCA | |
| | TCCACTACTGCGATTACCCTGGTTGCACAAAAGTTTATACCAAGTCTTCTC | |
| | ATTTAAAAGCTCACCTGAGGACTCACACTGGTGAAAAGCCATACAAGTGT | |
| | ACCTGGGAAGGCTGCGACTGGAGGTTCGCGCGATCGGATGAGCTGACCCG | |
| | CCACTACCGGAAGCACACAGGCGCCAAGCCCTTCCAGTGCGGGGTGTGCA | |
| | ACCGCAGCTTCTCGCGCTCTGACCACCTGGCCCTGCATATGAAGAGGCACC | |
| | AGAAC | |
| KLF6 | ATGGACGTGCTCCCCATGTGCAGCATCTTCCAGGAGCTCCAGATCGTGCAC | 17 |
| | GAGACCGGCTACTTCTCGGCGCTGCCGTCTCTGGAGGAGTACTGGCAACAG | |
| | ACCTGCCTAGAGCTGGAACGTTACCTCCAGAGCGAGCCCTGCTATGTTTCA | |
| | GCCTCAGAAATCAAATTTGACAGCCAGGAAGATCTGTGGACCAAAATCAT | |
| | TCTGGCTCGGGAGAAAAAGGAGGAATCCGAACTGAAGATATCTTCCAGTC | |
| | CTCCAGAGGACACTCTCATCAGCCCGAGCTTTTGTTACAACTTAGAGACCA | |
| | ACAGCCTGAACTCAGATGTCAGCAGCGAATCCTCTGACAGCTCCGAGGAA | |
| | CTTTCTCCCACGGCCAAGTTTACCTCCGACCCCATTGGCGAAGTTTTGGTCA | |
| | GCTCGGGAAAATTGAGCTCCTCTGTCACCTCCACGCCTCCATCTTCTCCGG | |
| | AACTGAGCAGGGAACCTTCTCAACTGTGGGGTTGCGTGCCCGGGGAGCTG | |
| | CCCTCGCCAGGGAAGGTGCGCAGCGGGACTTCGGGGAAGCCAGGTGACAA | |
| | GGGAAATGGCGATGCCTCCCCCGACGGCAGGAGGAGGGTGCACCGGTGCC | |
| | ACTTTAACGGCTGCAGGAAAGTTTACACCAAAAGCTCCCACTTGAAAGCA | |
| | CACCAGCGGACGCACACAGGAGAAAAGCCTTACAGATGCTCATGGGAAGG | |
| | GTGTGAGTGGCGTTTTGCAAGAAGTGATGAGTTAACCAGGCACTTCCGAA | |
| | AGCACACCGGGGCCAAGCCTTTTAAATGCTCCCACTGTGACAGGTGTTTTT | |
| | CCAGGTCTGACCACCTGGCCCTGCACATGAAGAGGCACCTC | |
| KLF7 | ATGGACGTGTTGGCTAGTTATAGTATATTCCAGGAGCTACAACTTGTCCAC | 18 |
| | GACACCGGCTACTTCTCAGCTTTACCATCCCTGGAGGAGACCTGGCAGCAG | |
| | ACATGCCTTGAATTGGAACGCTACCTACAGACGGAGCCCCGGAGGATCTC | |
| | AGAGACCTTTGGTGAGGACTTGGACTGTTTCCTCCACGCTTCCCCTCCCCC | |
| | GTGCATTGAGGAAAGCTTCCGTCGCTTAGACCCCCTGCTGCTCCCCGTGGA | |
| | AGCGGCCATCTGTGAGAAGAGCTCGGCAGTGGACATCTTGCTCTCTCGGGA | |
| | CAAGTTGCTATCTGAGACCTGCCTCAGCCTCCAGCCGGCCAGCTCTTCTCT | |
| | AGACAGCTACACAGCCGTCAACCAGGCCCAGCTCAACGCAGTGACCTCAT | |
| | TAACGCCCCCATCGTCCCCTGAGCTCAGCCGCCATCTGGTCAAAACCTCAC | |
| | AAACTCTCTCTGCCGTGGATGGCACGGTGACGTTGAAACTGGTGGCCAAG | |
| | AAGGCTGCTCTCAGCTCCGTAAAGGTGGGAGGGGTCGCAACAGCTGCAGC | |
| | AGCCGTGACGGCTGCGGGGGCCGTTAAGAGTGGACAGAGCGACAGTGACC | |
| | AAGGAGGGCTAGGGGCTGAAGCATGTCCCGAAAACAAGAAGAGGGTTCA | |
| | CCGCTGTCAGTTTAACGGGTGCCGGAAAGTTTATACAAAAAGCTCCCACTT | |
| | AAAGGCCCACCAGAGGACTCACACAGGTGAGAAGCCTTATAAGTGCTCAT | |
| | GGGAGGGATGTGAGTGGCGTTTTGCACGAAGCGATGAGCTCACGAGGCAC | |
| | TACAGGAAACACACAGGTGCAAAGCCCTTCAAATGCAACCACTGCGACAG | |
| | GTGTTTTTCCAGGTCTGACCATCTTGCCCTCCACATGAAGAGACATATC | |
| KLF8 | ATGGTCGATATGGATAAACTCATAAACAACTTGGAGGTCCAACTTAATTCA | 19 |
| | GAAGGTGGCTCAATGCAGGTATTCAAGCAGGTCACTGCTTCTGTTCGGAAC | |
| | AGAGATCCCCCTGAGATAGAATACAGAAGTAATATGACTTCTCCAACACTC | |
| | CTGGATGCCAACCCCATGGAGAACCCAGCACTGTTTAATGACATCAAGATT | |
| | GAGCCCCCAGAAGAACTTTTGGCTAGTGATTTCAGCCTGCCCCAAGTGGAA | |
| | CCAGTTGACCTCTCCTTTCACAAGCCCAAGGCTCCTCTCCAGCCTGCTAGC | |
| | ATGCTACAAGCTCCAATACGTCCCCCCAAGCCACAGTCTTCTCCCCAGACC | |
| | CTTGTGGTGTCCACGTCAACATCTGACATGAGCACTTCAGCAAACATTCCT | |
| | ACTGTTCTGACCCCAGGCTCTGTCCTGACCTCCTCTCAGAGCACTGGTAGC | |
| | CAGCAGATCTTACATGTCATTCACACTATCCCCTCAGTCAGTCTGCCAAAT | |
| | AAGATGGGTGGCCTGAAGACCATCCCAGTGGTAGTGCAGTCTCTGCCCATG | |
| | GTGTATACTACTTTGCCTGCAGATGGGGGCCCTGCAGCCATTACAGTCCCA | |
| | CTCATTGGAGGAGATGGTAAAAATGCTGGATCAGTGAAAGTTGACCCCAC | |
| | CTCCATGTCTCCACTGGAAATTCCAAGTGACAGTGAGGAGAGTACAATTGA | |
| | GAGTGGATCCTCAGCCTTGCAGAGTCTGCAGGGACTACAGCAAGAACCAG | |
| | CAGCAATGGCCCAAATGCAGGGAGAAGAGTCGCTTGACTTGAAGAGAAGA | |
| | CGGATTCACCAATGTGACTTTGCAGGATGCAGCAAAGTGTACACCAAAAG | |
| | CTCTCACCTGAAAGCTCACCGCAGAATCCATACAGGAGAGAAGCCTTATA | |
| | AATGCACCTGGGATGGCTGCTCCTGGAAATTTGCTCGCTCAGATGAGCTCA | |
| | CTCGCCATTTCCGCAAGCACACAGGCATCAAGCCTTTTCGGTGCACAGACT | |
| | GCAACCGCAGCTTTTCTCGTTCTGACCACCTGTCCCTGCATCGCCGTCGCCA | |
| | TGACACCATG | |
| KLF9 | ATGTCCGCGGCCGCCTACATGGACTTCGTGGCTGCCCAGTGTCTGGTTTCC | 20 |
| | ATTTCGAACCGCGCTGCGGTGCCGGAGCATGGGGTCGCTCCGGACGCCGA | |
| | GCGGCTGCGACTACCTGAGCGCGAGGTGACCAAGGAGCACGGTGACCCGG | |
| | GGGACACCTGGAAGGATTACTGCACACTGGTCACCATCGCCAAGAGCTTG | |
| | TTGGACCTGAACAAGTACCGACCCATCCAGACCCCCTCCGTGTGCAGCGAC | |
| | AGTCTGGAAAGTCCAGATGAGGATATGGGATCCGACAGCGACGTGACCAC | |
| | CGAATCTGGGTCGAGTCCTTCCCACAGCCCGGAGGAGAGACAGGATCCTG | |
| | GCAGCGCGCCCAGCCCGCTCTCCCTCCTCCATCCTGGAGTGGCTGCGAAGG | |
| | GGAAACACGCCTCCGAAAAGAGGCACAAGTGCCCCTACAGTGGCTGTGGG | |
| | AAAGTCTATGGAAAATCCTCCCATCTCAAAGCCCATTACAGAGTGCATACA | |
| | GGTGAACGGCCCTTTCCCTGCACGTGGCCAGACTGCCTTAAAAAGTTCTCC | |
| | CGCTCAGACGAGCTGACCCGCCACTACCGGACCCACACTGGGGAAAAGCA | |
| | GTTCCGCTGTCCGCTGTGTGAGAAGCGCTTCATGAGGAGTGACCACCTCAC | |
| | AAAGCACGCCCGGCGGCACACCGAGTTCCACCCCAGCATGATCAAGCGAT | |
| | CGAAAAAGGCGCTGGCCAACGCTTTG | |
| KLF10 | ATGCTCAACTTCGGTGCCTCTCTCCAGCAGACTGCGGAGGAAAGAATGGA | 21 |
| | AATGATTTCTGAAAGGCCAAAAGAGAGTATGTATTCCTGGAACAAAACTG | |
| | CAGAGAAAAGTGATTTTGAAGCTGTAGAAGCACTTATGTCAATGAGCTGC | |
| | AGTTGGAAGTCTGATTTTAAGAAATACGTTGAAAACAGACCTGTTACACCA | |
| | GTATCTGATTTGTCAGAGGAAGAGAATCTGCTTCCGGGAACACCTGATTTT | |
| | CATACAATCCCAGCATTTTGTTTGACTCCACCTTACAGTCCTTCTGACTTTG | |
| | AACCCTCTCAAGTGTCAAATCTGATGGCACCAGCGCCATCTACTGTACACT | |
| | TCAAGTCACTCTCAGATACTGCCAAACCTCACATTGCCGCACCTTTCAAAG | |
| | AGGAAGAAAAGAGCCCAGTATCTGCCCCCAAACTCCCCAAAGCTCAGGCA | |
| | ACAAGTGTGATTCGTCATACAGCTGATGCCCAGCTATGTAACCACCAGACC | |
| | TGCCCAATGAAAGCAGCCAGCATCCTCAACTATCAGAACAATTCTTTTAGA | |
| | AGAAGAACCCACCTAAATGTTGAGGCTGCAAGAAAGAACATACCATGTGC | |
| | CGCTGTGTCACCAAACAGATCCAAATGTGAGAGAAACACAGTGGCAGATG | |
| | TTGATGAGAAAGCAAGTGCTGCACTTTATGACTTTTCTGTGCCTTCCTCAG | |
| | AGACGGTCATCTGCAGGTCTCAGCCAGCCCCTGTGTCCCCACAACAGAAGT | |
| | CAGTGTTGGTCTCTCCACCTGCAGTATCTGCAGGGGGAGTGCCACCTATGC | |
| | CGGTCATCTGCCAGATGGTTCCCCTTCCTGCCAACAACCCTGTTGTGACAA | |
| | CAGTCGTTCCCAGCACTCCTCCCAGCCAGCCACCAGCCGTTTGCCCCCCTG | |
| | TTGTGTTCATGGGCACACAAGTCCCCAAAGGCGCTGTCATGTTTGTGGTAC | |
| | CCCAGCCCGTTGTGCAGAGTTCAAAGCCTCCGGTGGTGAGCCCGAATGGC | |
| | ACCAGACTCTCTCCCATTGCCCCTGCTCCTGGGTTTTCCCCTTCAGCAGCAA | |
| | AAGTCACTCCTCAGATTGATTCATCAAGGATAAGGAGTCACATCTGTAGCC | |
| | ACCCAGGATGTGGCAAGACATACTTTAAAAGTTCCCATCTGAAGGCCCAC | |
| | ACGAGGACGCACACAGGAGAAAAGCCTTTCAGCTGTAGCTGGAAAGGTTG | |
| | TGAAAGGAGGTTTGCCCGTTCTGATGAACTGTCCAGACACAGGCGAACCC | |
| | ACACGGGTGAGAAGAAATTTGCGTGCCCCATGTGTGACCGGCGGTTCATG | |
| | AGGAGTGACCATTTGACCAAGCATGCCCGGCGCCATCTATCAGCCAAGAA | |
| | GCTACCAAACTGGCAGATGGAAGTGAGCAAGCTAAATGACATTGCTCTAC | |
| | CTCCAACCCCTGCTCCCACACAG | |
| KLF11 | ATGCATACTCCTGATTTCGCTGGACCTGACGACGCCCGAGCCGTGGACATT | 22 |
| | ATGGACATTTGTGAATCTATACTCGAAAGAAAGAGACATGATTCAGAGCG | |
| | AAGTACATGCTCTATCCTCGAGCAAACAGACATGGAGGCGGTAGAAGCTC | |
| | TGGTGTGCATGTCCAGTTGGGGTCAGAGATCCCAGAAGGGGGACTTGCTTA | |
| | GAATCCGACCGCTTACTCCAGTTTCCGATAGCGGCGACGTAACAACTACTG | |
| | TTCATATGGACGCAGCCACGCCTGAGCTGCCCAAAGACTTTCACAGCCTCT | |
| | CAACTCTTTGCATCACTCCACCACAGTCCCCCGATCTTGTCGAACCATCAA | |
| | CCCGGACCCCTGTTAGCCCGCAAGTTACAGATTCAAAGGCGTGTACCGCGA | |
| | CCGATGTTCTGCAGAGTTCAGCGGTTGTAGCGCGGGCATTGAGCGGAGGG | |
| | GCTGAACGAGGTCTGTTGGGTCTTGAACCCGTACCGAGTTCTCCTTGTAGA | |
| | GCCAAGGGTACTAGTGTTATTCGGCATACCGGCGAGAGTCCGGCAGCTTGT | |
| | TTCCCCACCATACAAACCCCAGACTGTCGCCTTAGTGATTCCCGGGAAGGG | |
| | GAGGAACAGCTGTTGGGCCACTTCGAGACACTTCAAGATACACACTTGAC | |
| | AGATAGCTTGCTGTCCACCAACCTGGTGTCATGTCAACCTTGTTTGCACAA | |
| | GTCCGGGGGTCTCCTTCTGACTGACAAAGGTCAACAAGCGGGATGGCCTG | |
| | GCGCTGTCCAAACATGCAGTCCTAAAAACTACGAAAATGATTTGCCTAGG | |
| | AAAACCACGCCGCTTATCAGTGTGAGTGTTCCCGCTCCACCTGTCCTGTGC | |
| | CAGATGATCCCTGTAACCGGGCAATCATCTATGTTGCCTGCGTTCTTGAAG | |
| | CCCCCCCCACAACTGTCCGTTGGTACTGTTCGCCCGATCCTTGCGCAAGCA | |
| | GCGCCCGCCCCGCAACCCGTGTTCGTGGGGCCCGCTGTCCCGCAGGGTGCA | |
| | GTCATGTTGGTTCTTCCCCAGGGGGCCCTCCCGCCACCAGCTCCGTGTGCA | |
| | GCGAATGTCATGGCTGCCGGAAACACGAAATTGTTGCCCCTTGCACCCGCT | |
| | CCAGTTTTCATAACGAGCTCACAGAATTGTGTGCCACAAGTCGACTTCTCA | |
| | CGAAGACGGAACTATGTGTGCTCTTTCCCAGGTTGCAGAAAAACATATTTC | |
| | AAATCCTCTCATCTGAAAGCACATCTTCGGACCCATACAGGAGAGAAGCCT | |
| | TTTAATTGTAGCTGGGATGGCTGTGATAAAAAATTCGCAAGAAGTGATGA | |
| | GCTCAGTCGACATCGCAGGACGCATACCGGGGAAAAAAAATTCGTTTGTC | |
| | CAGTTTGTGACAGAAGATTTATGAGGTCCGACCATCTCACCAAGCACGCGC | |
| | GACGCCACATGACTACAAAGAAAATTCCTGGCTGGCAAGCCGAGGTGGGA | |
| | AAACTCAACCGAATCGCTTCCGCTGAATCCCCCGGCAGCCCGCTGGTAAGT | |
| | ATGCCTGCCAGTGCC | |
| KLF12 | ATGAACATTCACATGAAGCGCAAGACGATAAAGAACATCAATACATTCGA | 23 |
| | GAACCGAATGTTGATGTTGGATGGCATGCCCGCTGTACGGGTAAAAACCG | |
| | AGCTCCTGGAGTCTGAACAAGGATCCCCAAACGTCCACAACTACCCGGAT | |
| | ATGGAGGCAGTGCCGCTCTTGCTCAACAATGTGAAGGGAGAGCCGCCTGA | |
| | GGACTCTCTCTCCGTAGATCATTTCCAGACACAGACTGAGCCCGTAGATCT | |
| | TTCAATTAACAAAGCCAGAACATCTCCTACTGCGGTAAGTTCTTCTCCCGT | |
| | AAGTATGACAGCAAGTGCATCTAGTCCAAGTTCTACGAGCACTAGCAGTTC | |
| | TTCATCTAGTAGACTTGCTAGTTCACCAACGGTGATCACAAGTGTTTCTAG | |
| | CGCCAGCAGCAGCTCAACGGTACTGACTCCCGGTCCACTCGTGGCAAGCG | |
| | CTAGTGGCGTGGGTGGCCAACAATTTCTCCATATTATTCACCCCGTGCCTC | |
| | CGTCTAGTCCGATGAATCTCCAGAGCAACAAGCTTAGTCACGTACATAGGA | |
| | TCCCCGTCGTCGTCCAGTCAGTTCCCGTCGTCTACACAGCTGTGCGATCCCC | |
| | TGGGAATGTCAATAATACTATAGTTGTTCCTTTGCTTGAGGATGGTAGGGG | |
| | CCATGGGAAAGCACAGATGGACCCCCGCGGCTTGTCACCGAGACAGTCTA | |
| | AATCCGATAGTGACGACGATGATTTGCCTAACGTAACACTGGACTCTGTGA | |
| | ACGAGACCGGGAGTACCGCTCTGTCAATCGCTAGGGCCGTACAGGAGGTC | |
| | CACCCAAGCCCTGTGTCACGAGTCCGAGGTAACAGGATGAATAATCAGAA | |
| | ATTTCCCTGTAGCATCAGCCCATTTTCTATAGAGTCCACTCGGAGACAGCG | |
| | ACGAAGTGAATCACCCGACTCCAGAAAAAGGAGGATACATCGCTGTGACT | |
| | TTGAGGGCTGTAACAAGGTCTACACAAAAAGTTCACACCTCAAGGCGCAT | |
| | CGACGGACGCATACTGGGGAAAAACCGTACAAATGCACCTGGGAGGGATG | |
| | CACGTGGAAATTTGCACGCTCTGACGAGTTGACACGCCACTATCGAAAGC | |
| | ATACGGGCGTAAAGCCGTTTAAATGCGCTGATTGCGACAGGAGTTTTAGCC | |
| | GCTCTGATCACCTTGCTCTTCACCGGAGGCGACACATGCTTGTT | |
| KLF13 | ATGGCTGCGGCTGCATATGTGGATCATTTTGCGGCTGAGTGCCTGGTGTCA | 24 |
| | ATGTCTAGTAGAGCGGTGGTACACGGTCCCAGAGAAGGCCCAGAATCACG | |
| | CCCAGAGGGCGCCGCCGTCGCTGCAACACCGACGCTGCCTCGGGTCGAGG | |
| | AGCGCCGCGACGGGAAGGACAGTGCGTCACTTTTCGTAGTAGCGAGAATA | |
| | TTGGCAGATCTGAATCAACAGGCTCCAGCACCTGCGCCCGCTGAACGCCG | |
| | GGAGGGCGCCGCTGCCAGAAAGGCCAGAACACCATGCCGCTTGCCGCCAC | |
| | CTGCGCCAGAACCCACAAGTCCAGGTGCCGAAGGTGCGGCGGCTGCCCCT | |
| | CCTTCACCGGCCTGGTCTGAACCAGAACCAGAGGCAGGTCTTGAACCTGA | |
| | GCGCGAACCCGGCCCTGCAGGCTCTGGGGAACCTGGCCTGAGGCAGCGGG | |
| | TGAGGCGCGGCCGGAGCAGGGCCGACCTGGAATCACCGCAAAGGAAACAT | |
| | AAATGCCATTATGCTGGTTGCGAAAAGGTTTATGGAAAGTCATCCCACCTG | |
| | AAAGCACACCTCCGCACTCACACGGGTGAGCGACCTTTTGCGTGTTCCTGG | |
| | CAAGACTGCAATAAAAAGTTTGCTAGATCTGATGAACTTGCACGGCATTAT | |
| | CGAACTCATACCGGTGAAAAGAAGTTCTCATGCCCTATATGTGAGAAACG | |
| | GTTCATGCGCTCTGACCACTTGACGAAACATGCAAGACGACATGCTAATTT | |
| | TCATCCGGGGATGTTGCAGAGACGGGGAGGGGGAAGTAGGACTGGAAGTC | |
| | TCTCCGACTATTCCCGATCCGACGCTTCCTCACCAACGATTAGCCCCGCAA | |
| | GCAGTCCC | |
| KLF14 | ATGTCAGCCGCAGTCGCATGCCTTGATTACTTCGCGGCCGAGTGTCTTGTTT | 25 |
| | CCATGTCAGCGGGGGCTGTCGTTCACAGAAGACCACCAGACCCGGAGGGA | |
| | GCGGGAGGGGCAGCTGGATCTGAAGTCGGCGCGGCTCCACCTGAATCAGC | |
| | GCTTCCCGGCCCTGGTCCTCCAGGTCCCGCTAGCGTGCCCCAACTCCCACA | |
| | AGTGCCTGCTCCGAGTCCTGGAGCGGGCGGAGCAGCCCCGCATCTCCTTGC | |
| | AGCATCAGTGTGGGCCGATCTTCGCGGAAGCTCCGGGGAGGGCTCCTGGG | |
| | AAAACAGCGGAGAGGCCCCGCGAGCTTCAAGCGGCTTTTCCGATCCAATC | |
| | CCTTGCAGTGTTCAAACCCCATGCTCCGAGCTCGCGCCCGCGTCCGGAGCT | |
| | GCGGCAGTGTGCGCACCTGAAAGCTCATCCGATGCGCCGGCCGTTCCATCT | |
| | GCGCCAGCTGCTCCCGGTGCACCCGCAGCATCTGGCGGCTTTAGTGGTGGA | |
| | GCTCTTGGGGCGGGTCCCGCCCCTGCGGCGGATCAAGCTCCTCGCAGGCGC | |
| | AGTGTTACGCCCGCAGCAAAACGGCATCAATGCCCCTTTCCTGGTTGTACA | |
| | AAAGCATACTATAAGTCATCCCATCTCAAGAGTCACCAGAGGACGCATAC | |
| | AGGTGAGAGACCTTTTAGCTGTGACTGGCTCGATTGCGACAAGAAATTTAC | |
| | GCGGAGCGACGAACTTGCGCGGCACTACCGCACTCACACTGGAGAAAAGA | |
| | GGTTCTCTTGTCCCCTGTGTCCCAAGCAGTTCTCACGCAGTGATCACTTGAC | |
| | AAAACATGCTAGGAGACATCCAACATACCATCCCGACATGATAGAGTATC | |
| | GAGGTAGGCGACGCACACCTAGAATTGATCCTCCGCTGACTAGTGAAGTC | |
| | GAGTCAAGTGCCAGTGGAAGCGGACCGGGTCCCGCGCCCTCATTTACAAC | |
| | CTGTCTT | |
| KLF15 | ATGGTGGACCACTTACTTCCAGTGGACGAGAACTTCTCGTCGCCAAAATGC | 26 |
| | CCAGTTGGGTATCTGGGTGATAGGCTGGTTGGCCGGCGGGCATATCACATG | |
| | CTGCCCTCACCCGTCTCTGAAGATGACAGCGATGCCTCCAGCCCCTGCTCC | |
| | TGTTCCAGTCCCGACTCTCAAGCCCTCTGCTCCTGCTATGGTGGAGGCCTG | |
| | GGCACCGAGAGCCAGGACAGCATCTTGGACTTCCTATTGTCCCAGGCCACG | |
| | CTGGGCAGTGGCGGGGGCAGCGGCAGTAGCATTGGGGCCAGCAGTGGCCC | |
| | CGTGGCCTGGGGGCCCTGGCGAAGGGCAGCGGCCCCTGTGAAGGGGGAGC | |
| | ATTTCTGCTTGCCCGAGTTTCCTTTGGGTGATCCTGATGACGTCCCACGGCC | |
| | CTTCCAGCCTACCCTGGAGGAGATTGAAGAGTTTCTGGAGGAGAACATGG | |
| | AGCCTGGAGTCAAGGAGGTCCCTGAGGGCAACAGCAAGGACTTGGATGCC | |
| | TGCAGCCAGCTCTCAGCTGGGCCACACAAGAGCCACCTCCATCCTGGGTCC | |
| | AGCGGGAGAGAGCGCTGTTCCCCTCCACCAGGTGGTGCCAGTGCAGGAGG | |
| | TGCCCAGGGCCCAGGTGGGGGCCCCACGCCTGATGGCCCCATCCCAGTGTT | |
| | GCTGCAGATCCAGCCCGTGCCTGTGAAGCAGGAATCGGGCACAGGGCCTG | |
| | CCTCCCCTGGGCAAGCCCCAGAGAATGTCAAGGTTGCCCAGCTCCTGGTCA | |
| | ACATCCAGGGGCAGACCTTCGCACTCGTGCCCCAGGTGGTACCCTCCTCCA | |
| | ACTTGAACCTGCCCTCCAAGTTTGTGCGCATTGCCCCTGTGCCCATTGCCGC | |
| | CAAGCCTGTTGGATCGGGACCCCTGGGGCCTGGCCCTGCCGGTCTCCTCAT | |
| | GGGCCAGAAGTTCCCCAAGAACCCAGCCGCAGAACTCATCAAAATGCACA | |
| | AATGTACTTTCCCTGGCTGCAGCAAGATGTACACCAAAAGCAGCCACCTCA | |
| | AGGCCCACCTGCGCCGGCACACGGGTGAGAAGCCCTTCGCCTGCACCTGG | |
| | CCAGGCTGCGGCTGGAGGTTCTCGCGCTCTGACGAGCTGTCGCGGCACAG | |
| | GCGCTCGCACTCAGGTGTGAAGCCGTACCAGTGTCCTGTGTGCGAGAAGA | |
| | AGTTCGCGCGGAGCGACCACCTCTCCAAGCACATCAAGGTGCACCGCTTCC | |
| | CGCGGAGCAGCCGCTCCGTGCGCTCCGTGAAC | |
| KLF16 | ATGTCAGCCGCGGTCGCGTGCGTGGATTATTTTGCAGCAGATGTGCTGATG | 27 |
| | GCAATTTCATCCGGTGCAGTAGTTCATCGCGGAAGACCAGGTCCTGAGGGT | |
| | GCGGGGCCTGCGGCCGGGTTGGATGTTCGCGCCGCGCGCAGGGAAGCCGC | |
| | TTCTCCCGGAACACCTGGCCCTCCTCCTCCTCCGCCGGCGGCATCAGGCCC | |
| | GGGTCCTGGTGCAGCTGCGGCTCCTCACCTGTTGGCAGCCTCCATACTGGC | |
| | TGACCTGCGAGGGGGGCCAGGCGCTGCACCTGGTGGCGCGAGTCCAGCAA | |
| | GTTCCAGCTCCGCGGCGTCCTCCCCGAGTAGTGGGCGAGCTCCGGGCGCGG | |
| | CACCTTCTGCTGCCGCTAAATCACACCGATGCCCTTTCCCAGACTGCGCGA | |
| | AGGCGTATTATAAGTCCAGTCATTTGAAATCACACTTGAGGACACATACCG | |
| | GCGAGAGACCTTTTGCGTGCGACTGGCAGGGTTGTGATAAGAAATTTGCG | |
| | AGAAGCGACGAACTGGCCCGCCATCACCGCACCCACACAGGGGAAAAAA | |
| | GATTCTCATGCCCACTCTGTTCTAAGCGCTTCACGCGAAGCGACCATCTTG | |
| | CAAAGCACGCTAGGAGACACCCTGGGTTCCACCCCGACCTCTTGCGACGA | |
| | CCTGGCGCCCGGTCTACTAGCCCGTCTGACTCATTGCCGTGCTCTCTCGCA | |
| | GGGTCCCCTGCTCCGAGCCCCGCACCGTCCCCAGCTCCTGCCGGGCTT | |
| KLF17 | ATGTACGGCCGACCGCAGGCTGAGATGGAACAGGAGGCTGGGGAGCTGAG | 28 |
| | CCGGTGGCAGGCGGCGCACCAGGCTGCCCAGGATAACGAGAACTCAGCGC | |
| | CCATCTTGAACATGTCTTCATCTTCTGGAAGCTCTGGAGTGCACACCTCTTG | |
| | GAACCAAGGCCTACCAAGCATTCAGCACTTTCCTCACAGCGCAGAGATGCT | |
| | GGGGTCCCCTTTGGTGTCTGTTGAGGCGCCGGGGCAGAATGTGAATGAAG | |
| | GGGGGCCACAGTTCAGTATGCCACTGCCTGAGCGTGGTATGAGCTACTGCC | |
| | CCCAAGCGACTCTCACTCCTTCCCGGATGATTTACTGTCAGAGAATGTCTC | |
| | CCCCTCAGCAAGAGATGACGATTTTCAGTGGGCCCCAACTAATGCCCGTAG | |
| | GAGAGCCCAATATTCCAAGGGTAGCCAGGCCCTTCGGTGGGAATCTAAGG | |
| | ATGCCCCCCAATGGGCTGCCAGTCTCGGCTTCCACTGGAATCCCAATAATG | |
| | TCCCACACTGGGAACCCTCCAGTGCCTTACCCTGGCCTCTCGACAGTACCT | |
| | TCTGACGAAACATTGTTGGGCCCGACTGTGCCTTCCACTGAGGCCCAGGCA | |
| | GTGCTCCCCTCCATGGCTCAGATGTTGCCCCCGCAAGATGCCCATGACCTT | |
| | GGGATGCCCCCAGCTGAGTCCCAGTCATTGCTGGTTTTAGGATCTCAGGAC | |
| | TCTCTTGTCAGTCAGCCAGACTCTCAAGAAGGCCCATTTCTACCAGAGCAG | |
| | CCCGGACCTGCTCCACAGACAGTAGAGAAGAACTCCAGGCCTCAGGAAGG | |
| | GACTGGTAGAAGGGGCTCCTCAGAGGCAAGGCCTTACTGCTGCAACTACG | |
| | AGAACTGCGGAAAAGCTTATACCAAACGCTCCCACCTCGTGAGCCACCAG | |
| | CGCAAGCACACAGGTGAGAGGCCATATTCTTGCAACTGGGAAAGTTGTTC | |
| | ATGGTCTTTCTTCCGTTCTGATGAGCTTAGACGACATATGCGGGTACACAC | |
| | CAGATATCGACCATATAAATGTGATCAGTGCAGCCGGGAGTTCATGAGGT | |
| | CTGACCATCTCAAGCAACACCAGAAGACTCATCGGCCGGGACCCTCAGAC | |
| | CCACAGGCCAACAACAACAATGGAGAGCAGGACAGTCCTCCTGCTGCTGG | |
| | TCCT | |

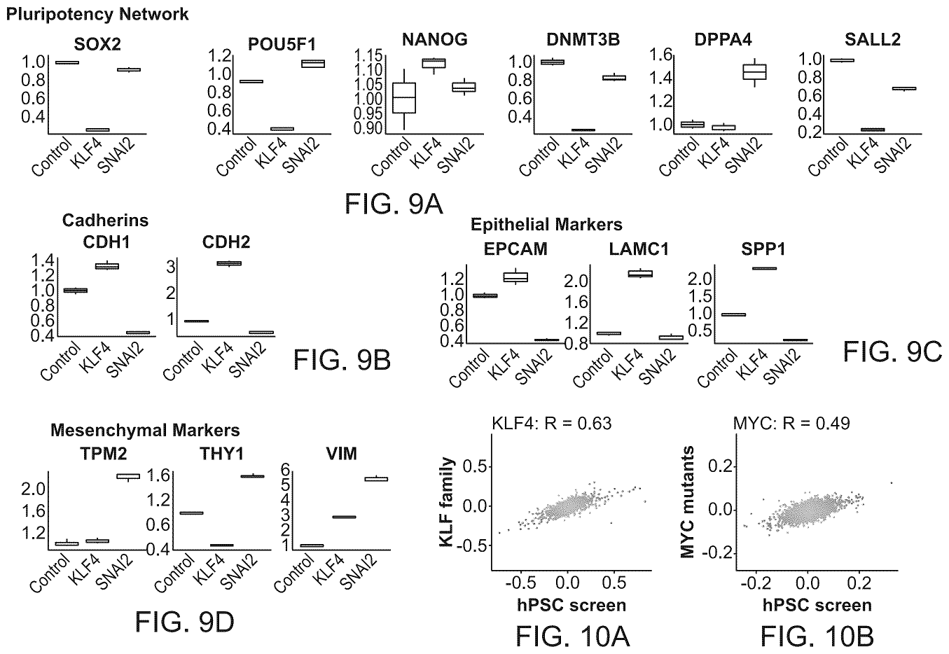

To further demonstrate the applicability of the network analysis to uncover novel phenomena, Applicants focused on two TFs, SNAI2 and KLF4, which seemed to have opposite effects on the pluripotency module. Since KLF4 and SNAI2 are known to play critical and opposing roles in epithelial-mesenchymal transition (EMT) Applicants assessed whether they cause changes along an EMT-like axis in hPSCs as well. A PCA analysis using 200 genes from a consensus EMT geneset from MSigDB demonstrated a distinct stratification of KLF4-transduced cells towards an epithelial-like state and SNAI2-transduced cells towards a mesenchymal-like state. The scRNA-seq data also demonstrates expression level changes in signature genes consistent with EMT (FIG. 3C), which Applicants confirmed with qRT-PCR (FIG. 9).

Finally, Applicants chose to focus on ETV2, which has the greatest average fitness loss across all medium conditions (FIG. 1B), as an exemplary case for investigation of a TF showing markedly reduced fitness in all medium conditions. Applicants hypothesized that the reduced fitness could be due to a proliferation disadvantage if ETV2-transduced cells are undergoing massive reprogramming without division. Focused experiments revealed that while ETV2-transduced cells undergo extensive cell death in pluripotent medium, there is a morphology change, indicative of an endothelial phenotype, in endothelial medium (FIG. 3E). Confirmatory qRT-PCR assays demonstrated a strong upregulation of the key endothelial markers CDH5, PECAM1 and VWF (FIG. 3F). Immunofluorescence revealed a distinct distribution of CDH5, with greater localization at cell-cell junctions (FIG. 3G), consistent with known results. In addition, functional testing confirmed tube formation (FIG. 3H), suggesting that a single TF, ETV2, may be able to drive reprogramming from a pluripotent to an endothelial-like state.

To Applicants' knowledge, this is the first demonstration of a high-throughput gene over-expression screening approach that can simultaneously assay both fitness and transcriptome-wide effects. Applicants' use of ORF overexpression drove strong phenotypic effects, allowing Applicants to capture subtle transcriptomic signals. Additionally, Applicants demonstrated the versatility of the SEUSS screening platform, by assaying mutant forms of a single TF, and assaying all the TFs in a gene family to uncover patterns and differences. Applicants note that the effects of gene overexpression are context dependent. In Applicants' assays, since hPSCs were transduced with pooled libraries, transcriptomic changes driven by cell-cell interactions could increase variability, even supporting the survival of certain cells or disrupting the pluripotent state of control cells. Applicants also assume, in aggregating multiple batches from independent experiments, that each batch is relatively similar. Additionally, while Applicants believe the gene co-perturbation network is a valuable resource, it is dependent on the set of perturbations and conditions used in the experiment.

Taken together, SEUSS has broad applicability to study the effects of overexpression in diverse cell types and contexts; it may be extended to novel applications such as high-throughput screening of large-scale protein mutagenesis, and is amenable to scale-up. In combination with other methods of genetic and epigenetic perturbation it may allow Applicants to generate a comprehensive understanding of the pluripotent and differentiation landscape.

### Example 1 Methods

### Cell Culture

H1 hESC cell line was maintained under feeder-free conditions in mTeSR1 medium (Stem Cell Technologies). Prior to passaging, tissue-culture plates were coated with growth factor-reduced Matrigel (Corning) diluted in DMEM/F-12 medium (Thermo Fisher Scientific) and incubated for 30 minutes at 37° C., 5% CO₂. Cells were dissociated and passaged using the dissociation reagent Versene (Thermo Fisher Scientific).

### Library Preparation

A lentiviral backbone plasmid was constructed containing the EF1α promoter, mCherry transgene flanked by BamHI restriction sites, followed by a P2A peptide and hygromycin resistance enzyme gene immediately downstream. Each transcription factor in the library was individually inserted in place of the mCherry transgene. Since the ectopically expressed transcription factor would lack a poly-adenylation tail due to the presence of the 2A peptide immediately downstream of it, the transcript will not be captured during single-cell transcriptome sequencing which relies on binding the poly-adenylation tail of mRNA. Thus, a barcode sequence was introduced to allow for identification of the ectopically expressed transcription factor. The backbone was digested with HpaI, and a pool of 20 bp long barcodes with flanking sequences compatible with the HpaI site, was inserted immediately downstream of the hygromycin resistance gene by Gibson assembly. The vector was constructed such that the barcodes were located only 200 bp upstream of the 3′-LTR region. This design enabled the barcodes to be transcribed near the poly-adenylation tail of the transcripts and a high fraction of barcodes to be captured during sample processing for scRNA-seq.

To create the transcription factor library, individual transcription factors were PCR amplified out of a human cDNA pool (Promega Corporation) or obtained as synthesized double-stranded DNA fragments (gBlocks, IDT Inc) with flanking sequences compatible with the BamHI restriction sites. MYC mutants were obtained as gBlocks with a 6-amino acid GSGSGS linker (SEQ ID NO: 29) substituted in place of deleted domains (Table 1). The lentiviral backbone was digested with BamHI HF (New England Biolabs) at 37° C. for 3 hours in a reaction consisting of: lentiviral backbone, 4 μg, CutSmart buffer, 5 μl, BamHI, 0.625 μl, H₂0 up to 50 μl. After digestion, the vector was purified using a QIAquick PCR Purification Kit (Qiagen). Each transcription factor vector was then individually assembled via Gibson assembly. The Gibson assembly reactions were set up as follows: 100 ng digested lentiviral backbone, 3:10 molar ratio of transcription factor insert, 2× Gibson assembly master mix (New England Biolabs), H₂0 up to 20 μl. After incubation at 50° C. for 1 h, the product was transformed into One Shot Stb13 chemically competent Escherichia coli (Invitrogen). A fraction (150 μL) of cultures was spread on carbenicillin (50 μg/ml) LB plates and incubated overnight at 37° C. Individual colonies were picked, introduced into 5 ml of carbenicillin (50 μg/ml) LB medium and incubated overnight in a shaker at 37° C. The plasmid DNA was then extracted with a QIAprep Spin Miniprep Kit (Qiagen), and Sanger sequenced to verify correct assembly of the vector and to extract barcode sequences.

To assemble the library, individual transcription factor vectors were pooled together in an equal mass ratio along with a control vector containing the mCherry transgene which constituted 10% of the final pool.

### Viral Production

HEK 293T cells were maintained in high glucose DMEM supplemented with 10% fetal bovine serum (FBS). In order to produce lentivirus particles, cells were seeded in a 15 cm dish 1 day prior to transfection, such that they were 60-70% confluent at the time of transfection. For each 15 cm dish 36 μl of Lipofectamine 2000 (Life Technologies) was added to 1.5 ml of Opti-MEM (Life Technologies). Separately 3 μg of pMD2.G (Addgene no. 12259), 12 μg of pCMV delta R8.2 (Addgene no. 12263) and 9 μg of an individual vector or pooled vector library was added to 1.5 ml of Opti-MEM. After 5 minutes of incubation at room temperature, the Lipofectamine 2000 and DNA solutions were mixed and incubated at room temperature for 30 minutes. During the incubation period, medium in each 15 cm dish was replaced with 25 ml of fresh, pre-warmed medium. After the incubation period, the mixture was added dropwise to each dish of HEK 293T cells. Supernatant containing the viral particles was harvested after 48 and 72 hours, filtered with 0.45 μm filters (Steriflip, Millipore), and further concentrated using Amicon Ultra-15 centrifugal ultrafilters with a 100,000 NMWL cutoff (Millipore) to a final volume of 600-800 μl, divided into aliquots and frozen at −80° C.

### Viral Transduction

For viral transduction, on day-1, H1 cells were dissociated to a single cell suspension using Accutase (Innovative Cell Technologies) and seeded into Matrigel-coated plates in mTeSR containing ROCK inhibitor, Y-27632 (10 μM, Sigma-Aldrich). For transduction with the TF library, cells were seeded into 10 cm dishes at a density of 6×10⁶ cells for screens conducted in mTeSR or 4.5×10⁶ cells for screens conducted in endothelial growth medium (EGM) or multilineage (ML) medium (DMEM+20% FBS.) For transduction with individual transcription factors cells were seeded at a density of 4×10⁵ cells per well of a 12 well plate for experiments conducted in mTeSR or 3×10⁵ cells per well for experiments conducted in the alternate media.

On day 0, medium was replaced with fresh mTeSR to allow cells to recover for 6-8 hours. Recovered cells were then transduced with lentivirus added to fresh mTeSR containing polybrene (5 μg/ml, Millipore). On day 1, medium was replaced with the appropriate fresh medium: mTeSR, endothelial growth medium or high glucose DMEM+20% FBS. Hygromycin (Thermo Fisher Scientific) selection was started from day 2 onward at a selection dose of 50 μg/ml, medium containing hygromycin was replaced daily.

### Single Cell Library Preparation

For screens conducted in mTeSR cells were harvested 5 days after transduction while for alternate media, EGM or ML, cells were harvested 6 days after transduction with the TF library. Cells were dissociated to single cell suspensions using Accutase (Innovative Cell Technologies). For samples sorted with magnetically assisted cell sorting (MACS), cells were labelled with anti-TRA-1-60 antibodies or with dead cell removal microbeads and sorted as per manufacturer's instructions (Miltenyi Biotec). Samples were then resuspended in 1×PBS with 0.04% BSA at a concentration between 600-2000 per μl. Samples were loaded on the 10× Chromium system and processed as per manufacturer's instructions (10× Genomics). Unused cells were centrifuged at 300 rcf for 5 minutes and stored as pellets at −80° C. until extraction of genomic DNA.

Single cell libraries were prepared as per the manufacturer's instructions using the Single Cell 3′ Reagent Kit v2 (10× Genomics). Prior to fragmentation, a fraction of the sample post-cDNA amplification was used to amplify the transcripts containing both the TF barcode and cell barcode.

### Barcode Amplification

Barcodes were amplified from cDNA generated by the single cell system as well as from genomic DNA from cells not used for single cell sequencing. Barcodes were amplified from both types of samples and prepared for deep sequencing through a two-step PCR process.

For amplification of barcodes from cDNA, the first step was performed as three separate 50 μl reactions for each sample. 2 μl of the cDNA was input per reaction with Kapa Hifi Hotstart ReadyMix (Kapa Biosystems). The PCR primers used were, Nexterai7_TF_Barcode_F: GTCTCGTGGGCTCGGAGATGTGTATAAGAGACAGAGAACTATTTCCTGGCTGTTACG CG (SEQ ID NO: 30) and NEBNext Universal PCR Primer for Illumina (New England Biolabs). The thermocycling parameters were 95° C. for 3 min; 26-28 cycles of 98° C. for 20 s; 65° C. for 15 s; and 72° C. for 30 s; and a final extension of 72° C. for 5 min. The numbers of cycles were tested to ensure that they fell within the linear phase of amplification. Amplicons (˜500 bp) of 3 reactions for each sample were pooled, size-selected and purified with Agencourt AMPure XP beads at a 0.8 ratio. The second step of PCR was performed with two separate 50 μl reactions with 50 ng of first step purified PCR product per reaction. Nextera XT Index primers were used to attach Illumina adapters and indices to the samples. The thermocycling parameters were: 95° C. for 3 min; 6-8 cycles of (98° C. for 20 s; 65° C. for 15 s; 72° C. for 30 s); and 72° C. for 5 min. The amplicons from these two reactions for each sample were pooled, size-selected and purified with Agencourt AMPure XP beads at a 0.8 ratio. The purified second-step PCR library was quantified by Qubit dsDNA HS assay (Thermo Fisher Scientific) and used for downstream sequencing on an Illumina HiSeq platform.

For amplification of barcodes from genomic DNA, genomic DNA was extracted from stored cell pellets with a DNeasy Blood and Tissue Kit (Qiagen). The first step PCR was performed as three separate 50 μl reactions for each sample. 2 μg of genomic DNA was input per reaction with Kapa Hifi Hotstart ReadyMix. The PCR primers used were, NGS_TF-Barcode_F: ACACTCTTTCCCTACACGACGCTCTTCCGATCTAGAACTATTTCCTGGCTGTTACGCG (SEQ ID NO: 31) and NGS_TF-Barcode_R: GACTGGAGTTCAGACGTGTGCTCTTCCGATCTTGTCTTCGTTGGGAGTGAATTAGC (SEQ ID NO: 32). The thermocycling parameters were: 95° C. for 3 min; 26-28 cycles of 98° C. for 20 s; 55° C. for 15 s; and 72° C. for 30 s; and a final extension of 72° C. for 5 min. The numbers of cycles were tested to ensure that they fell within the linear phase of amplification. Amplicons (200 bp) of 3 reactions for each sample were pooled, size-selected with Agencourt AMPure XP beads (Beckman Coulter, Inc.) at a ratio of 0.8, and the supernatant from this was further size-selected and purified at a ratio of 1.6. The second step of PCR was performed as two separate 50 μl reactions with 50 ng of first step purified PCR product per reaction. Next Multiplex Oligos for Illumina (New England Biolabs) Index primers were used to attach Illumina adapters and indices to the samples. The thermocycling parameters were: 95° C. for 3 min; 6 cycles of (98° C. for 20 s; 65° C. for 20 s; 72° C. for 30 s); and 72° C. for 2 min. The amplicons from these two reactions for each sample were pooled, size-selected with Agencourt AMPure XP beads at a ratio of 0.8, and the supernatant from this was further size-selected and purified at a ratio of 1.6. The purified second-step PCR library was quantified by Qubit dsDNA HS assay (Thermo Fisher Scientific) and used for downstream sequencing on an Illumina MiSeq platform.

### Single Cell RNA-Seq Processing and Genotype Deconvolution

Using the 10× genomics CellRanger pipeline [citation], Applicants aligned Fastq files to hg38, counted UMIs to generate counts matrices, and aggregated samples across 10× runs with cellranger aggr. All cellranger commands were run using default settings.

To assign one or more transcription factor genotypes to each cell, Applicants aligned the plasmid barcode reads to hg38 using BWA, and then labeled each read with its corresponding cell and UMI tags. To remove potential chimeric reads, Applicants used a two-step filtering process. First, Applicants only kept UMIs that made up at least 0.5% of the total amount of reads for each cell. Applicants then counted the number of UMIs and reads for each plasmid barcode within each cell, and only assigned that cell any barcode that contained at least 10% of the cell's read and UMI counts. Barcodes were mapped to transcription factors within one edit distance of the expected barcode. The code for assigning genotypes to each cell can be found on github at: github.com/yanwu2014/genotyping-matrices

### Clustering and Cluster Enrichment

Clustering was performed on the aggregated counts matrices using the Seurat pipeline. Applicants first filtered the counts matrix for genes that are expressed in at least 2% of cells, and cells that express at least 500 genes. Applicants then normalized the counts matrix, found overdispersed genes, and used a negative binomial linear model to regress away library depth, batch effects, and mitochondrial gene fraction. Applicants performed PCA on the overdispersed genes, keeping the first 20 principal components. Applicants then used the PCs to generate a K Nearest Neighbors graph, with K=30, used the KNN graph to calculate a shared nearest neighbors graph, and used a modularity optimization algorithm on the SNN graph to find clusters. Clusters were recursively merged until all clusters could be distinguished from every other cluster with an out of the box error (oobe) of less than 5% using a random forest classifier trained on the top 15 genes by loading magnitude for the first 20 PCs. Applicants used tSNE on the first 20 PCs to visualize the results.

Cluster enrichment was performed using Fisher's exact test, testing each genotype for over-enrichment in each cluster. The p-value from the Fisher test for each genotype and cluster combination was corrected using the Benjamini-Hochberg method.

### Differential Expression, Identification of Significant Genotypes, and Genotype Trimming

Applicants used a modified version of the MIMOSCA linear model to analyze the differentially expressed genes for each genotype. In this model, Applicants used the R glmnet package with the multigaussian family, with alpha (the lasso vs ridge parameter) set to 0.5. Lambda (the coefficient magnitude regularization parameter) was set using 5-fold cross validation.

In order to account for unperturbed cells, Applicants “trimmed” the cells in each transcription factor genotype to only include cells that belonged to a cluster that the genotype was enriched for. Specifically, Applicants first obtained a set of transcription factor genotypes with strong cluster enrichment, such that each significantly enriched genotype was enriched for a cluster with an FDR >1e-6, and whose cluster enrichment profile was different from the control mCherry profile with an adjusted chi-squared p-value of less than 1e-6. For each significantly enriched genotype, Applicants only kept cells that were part of a cluster that the genotype was enriched for at FDR <0.01 level. Each genotype can be enriched for more than one cluster. After trimming the significantly enriched genotypes, Applicants repeated the differential expression.

TFs were chosen as significant for downstream analysis if they were enriched for one or more clusters as described, or if the TF drove statistically significant differential expression of greater than 100 genes.

### Gene Co-Perturbation Network and Module Detection

Applicants took the genes by genotypes coefficients matrix from the regression analysis with trimmed genotypes and used it to calculate the Euclidean distance between genes, using the significant genotypes as features. Applicants then built a k-nearest neighbors graph from the Euclidean distances between genes, with k=30. From this kNN graph, Applicants calculated the fraction of shared nearest neighbors (SNN) for each pair of genes to build and SNN graph. For example, if two genes share 23/30 neighbors, Applicants create an edge between them in the SNN graph with a weight of 23/30=0.767.

To identify gene modules, Applicants used the Louvain modularity optimization algorithm. For each gene module, Applicants identified enriched Gene Ontology terms using Fisher's exact test (Table 5). Applicants also ranked genes in each gene module by the number of enriched Gene Ontology terms the gene is part of, to identify the most biologically significant genes in each module (Table 5). Gene module identities were assigned based on manual inspection of enriched GO terms and the genes within each module. The effect of each genotype on a gene module was calculated by taking the average of the regression coefficients for the genotype and the genes within the module.

### Dataset Correlation

To compare how the combined hPSC medium dataset correlated with the five individual datasets, Applicants correlated the regression coefficients of the combined dataset with the coefficients for each individual dataset, subsetting for coefficients that were statistically significant in either the individual dataset, or the combined dataset. Each coefficient represents the effect of a single TF on a single gene. The two datasets for the multilineage lineage screens were correlated in the same manner.

### Fitness Effect Analysis

To calculate fitness effects from genomic DNA reads, Applicants first used MagECK to align reads to genotype barcodes and count the number of reads for each genotype in each sample, resulting in a genotypes by samples read counts matrix. Applicants normalized the read counts matrix by dividing each column by the sum of that column, and then calculated log fold-change by dividing each sample by the normalized plasmid library counts, and then taking a log 2 transform. For the stem cell media, Applicants averaged the log fold change across the non MACS sorted samples.

To calculate fitness effects from genotype counts identified from single cell RNA-seq, Applicants used a cell counts matrix instead of a read counts matrix, and repeated the above protocol.

### Epithelial Mesenchymal Transition Analysis

Applicants took 200 genes from the Hallmark Epithelial Mesenchymal Transition geneset from MSigDB and ran PCA on those genes with the stem cell medium dataset, visualizing the first two principal components. The first principal component was an EMT-like signature and Applicants used the gene loadings, along with literature research to identify a relevant panel of EMT related genes to display. All analysis code can be found at github.com/yanwu2014/SEUSS-Analysis.

RNA Extraction, and qRT-PCR

RNA was extracted from cells using the RNeasy Mini Kit (Qiagen) as per the manufacturer's instructions. The quality and concentration of the RNA samples was measured using a spectrophotometer (Nanodrop 2000, Thermo Fisher Scientific). cDNA was prepared using the Protoscript II First Strand cDNA synthesis kit (New England Biolabs) in a 20 μl reaction and diluted up to 1:5 with nuclease-free water. qRT-PCR reactions were setup as: 2 μl cDNA, 400 nM of each primer, 2× Kapa SYBR Fast Master Mix (Kapa Biosystems), H₂O up to 20 μl. qRT-PCR was performed using a CFX Connect Real Time PCR Detection System (Bio-Rad) with the thermocycling parameters: 95° C. for 3 min; 95° C. for 3 s; 60° C. for 20 s, for 40 cycles. All experiments were performed in triplicate and results were normalized against a housekeeping gene, GAPDH. Relative mRNA expression levels, compared with GAPDH, were determined by the comparative cycle threshold (ΔΔC_{T}) method. Primers used for qRT-PCR are listed in Table 6.

### Immunofluorescence

Cells were fixed with 4% (wt/vol) paraformaldehyde in PBS at room temperature for 30 minutes. Cells were then incubated with a blocking buffer: 5% donkey serum, 0.2% Triton X-100 in PBS for 1 hour at room temperature followed by incubation with primary antibodies diluted in the blocking buffer at 4° C. overnight. Primary antibodies used were: VE-Cadherin (D87F2, Cell Signaling Technology; 1:400). Secondary antibodies used were: DyLight 488 labelled donkey anti-rabbit IgG (ab96891, Abcam; 1:250).

After overnight incubation with primary antibodies, cells were labelled with secondary antibodies diluted in 1% BSA in PBS for 1 hour at 37° C. Nuclear staining was done by incubating cells with DAPI for 5 minutes at room temperature. All imaging was conducted on a Leica DMi8 inverted microscope equipped with an Andor Zyla sCMOS camera and a Lumencor Spectra X multi-wavelength fluorescence light source.

### Endothelial Tube Formation Assay

A mCherry expressing H1 cell line was created by transducing H1 cells with a lentivirus containing the EF1α promoter driving expression of the mCherry transgene, internal ribosome entry site (IRES) and a puromycin resistance gene. Cells were then maintained under constant puromycin selection at a dose of 0.75 μg/ml. mCherry labelled H1 cells were transduced with either ETV2 lentivirus or control mCherry lentivirus, hygromycin selection was started on day 2 and cells were used for tube formation assay on day 6.

Growth-factor reduced Matrigel (Corning) was thawed on ice and 250 μl was deposited cold per well of a 24-well plate. The deposited Matrigel was incubated for 60 minutes at 37° C., 5% CO₂, to allow for complete gelation and the ETV2-transduced or control cells were then seeded on it at a density of 3.2×10⁵ cells per well in a volume of 500 μl EGM. Imaging was conducted 24 hours after deposition of the cells.

### Example 2

### Corneal Endothelial Stem Cell Transplant

Skin fibroblasts are isolated from a patient with a corneal eye disease. iPSCs are generated from the fibroblasts using techniques known in the art. Briefly, the isolated fibroblasts are reprogrammed by forced expression of one or more pluripotency genes selected from: OCT3/4, SOX1, SOX2, SOX15, SOX18, KLF1, KLF2, KLF4, KLF5, n-MYC, c-MYC, L-MYC, NANOG, LIN28, and GLIS1.

Next, the iPSCs are directed to differentiate into endothelial cells by introducing expression of ETV2. Expression is introduced by infecting the cells with an AAV virus encoding ETV2. After the cells differentiate into endothelial cells, they are expanded ex vivo and harvested.

The cells are administered to the patient by transplant to the cornea following removal of the diseased corneal tissue. After corneal transplant with the endothelial cells, repair of the cornea is identified by achieving full or partial restoration of corneal function in the patient.

TABLE 1

| | | SEQ ID | | |
|---|---|---|---|---|
| GENE | SEQUENCE | NO: | ROLE | REFERENCES |
| mCherry | ATGGTGAGCAAGGGCGAGGAGGAT | 33 | Non-functional | |
| Control | AACATGGCCATCATCAAGGAGTTC | | control vector | |
| | ATGCGCTTCAAGGTGCACATGGAG | | | |
| | GGCTCCGTGAACGGCCACGAGTTC | | | |
| | GAGATCGAGGGCGAGGGCGAGGGC | | | |
| | CGCCCCTACGAGGGCACCCAGACC | | | |
| | GCCAAGCTGAAGGTGACCAAGGGT | | | |
| | GGCCCCCTGCCCTTCGCCTGGGACA | | | |
| | TCCTGTCCCCTCAGTTCATGTACGG | | | |
| | CTCCAAGGCCTACGTGAAGCACCC | | | |
| | CGCCGACATCCCCGACTACTTGAAG | | | |
| | CTGTCCTTCCCCGAGGGCTTCAAGT | | | |
| | GGGAGCGCGTGATGAACTTCGAGG | | | |
| | ACGGCGGCGTGGTGACCGTGACCC | | | |
| | AGGACTCCTCCCTGCAGGACGGCG | | | |
| | AGTTCATCTACAAGGTGAAGCTGC | | | |
| | GCGGCACCAACTTCCCCTCCGACGG | | | |
| | CCCCGTAATGCAGAAGAAGACCAT | | | |
| | GGGCTGGGAGGCCTCCTCCGAGCG | | | |
| | GATGTACCCCGAGGACGGCGCCCT | | | |
| | GAAGGGCGAGATCAAGCAGAGGCT | | | |
| | GAAGCTGAAGGACGGCGGCCACTA | | | |
| | CGACGCTGAGGTCAAGACCACCTA | | | |
| | CAAGGCCAAGAAGCCCGTGCAGCT | | | |
| | GCCCGGCGCCTACAACGTCAACAT | | | |
| | CAAGTTGGACATCACCTCCCACAAC | | | |
| | GAGGACTACACCATCGTGGAACAG | | | |
| | TACGAACGCGCCGAGGGCCGCCAC | | | |
| | TCCACCGGCGGCATGGACGAGCTG | | | |
| | TACAAG | | | |
| ASCL1 | ATGGAGTCTTCTGCTAAAATGGAGT | 34 | Involved in | Wilkinson, G. |
| | CCGGAGGCGCGGGACAACAACCAC | | neuronal | et al. |
| | AACCGCAACCACAACAACCCTTCCT | | specification | Proneural |
| | GCCGCCGGCCGCATGTTTTTTCGCG | | and | genes in |
| | ACCGCTGCTGCTGCTGCAGCGGCG | | differentiation. | neocortical |
| | GCGGCTGCTGCCGCCGCGCAATCC | | Demonstrated to | development. |
| | GCCCAACAGCAACAACAACAACAG | | drive neuronal | Neuroscience |
| | CAGCAGCAGCAACAAGCGCCTCAA | | differentiation | 253, 256-273 |
| | CTTCGACCCGCTGCAGACGGGCAG | | from hPSCs | (2013). |
| | CCCTCAGGGGGAGGGCACAAGAGC | | | Chanda, S. et |
| | GCTCCGAAGCAGGTTAAAAGGCAG | | | al. Generation |
| | AGGAGCAGTAGTCCCGAACTGATG | | | of induced |
| | CGATGTAAGAGGCGCCTCAATTTTA | | | neuronal cells |
| | GCGGTTTTGGTTACTCTTTGCCCCA | | | by the single |
| | GCAGCAGCCGGCTGCCGTAGCTCG | | | reprogramming |
| | CCGAAATGAGCGGGAAAGGAACCG | | | factor |
| | CGTTAAACTTGTGAATCTCGGTTTC | | | ASCL1. Stem |
| | GCGACACTTCGAGAGCACGTACCA | | | cell reports 3, |
| | AATGGGGCAGCTAACAAGAAAATG | | | 282-96 |
| | AGTAAAGTTGAGACACTGCGGTCT | | | (2014). |
| | GCAGTGGAGTATATTAGAGCTCTTC | | | |
| | AACAATTGCTTGACGAGCACGATG | | | |
| | CCGTATCAGCCGCATTTCAAGCCGG | | | |
| | GGTGCTGTCCCCAACAATATCTCCG | | | |
| | AACTACAGCAATGATCTTAATAGC | | | |
| | ATGGCGGGAAGTCCCGTTTCCTCCT | | | |
| | ACTCCTCTGATGAGGGCAGCTACG | | | |
| | ACCCTCTCAGTCCCGAGGAGCAAG | | | |
| | AGCTTCTTGACTTCACTAACTGGTT | | | |
| | C | | | |
| ASCL3 | ATGATGGACAACAGAGGCAACTCT | 35 | Involved in | Bullard, T. et |
| | AGTCTACCTGACAAACTTCCTATCT | | salivary gland | al. Ascl3 |
| | TCCCTGATTCTGCCCGCTTGCCACT | | cell | expression |
| | TACCAGGTCCTTCTATCTGGAGCCC | | development | marks a |
| | ATGGTCACTTTCCACGTGCACCCAG | | | progenitor |
| | AGGCCCCGGTGTCATCTCCTTACTC | | | population of |
| | TGAGGAGCTGCCACGGCTGCCTTTT | | | both acinar |
| | CCCAGCGACTCTCTTATCCTGGGAA | | | and ductal |
| | ATTACAGTGAACCCTGCCCCTTCTC | | | cells in mouse |
| | TTTCCCGATGCCTTATCCAAATTAC | | | salivary |
| | AGAGGGTGCGAGTACTCCTACGGG | | | glands. Dev. |
| | CCAGCCTTCACCCGGAAAAGGAAT | | | Biol. 320, 72- |
| | GAGCGGGAAAGGCAGCGGGTGAAA | | | 78(2008) |
| | TGTGTCAATGAAGGCTACGCCCAG | | | |
| | CTCCGACATCATCTGCCAGAGGAGT | | | |
| | ATTTGGAGAAGCGACTCAGCAAAG | | | |
| | TGGAAACCCTCAGAGCTGCGATCA | | | |
| | AGTACATTAACTACCTGCAGTCTCT | | | |
| | TCTGTACCCTGATAAAGCTGAGACA | | | |
| | AAGAATAACCCTGGAAAAGTTTCC | | | |
| | TCCATGATAGCAACCACCAGCCAC | | | |
| | CATGCTGACCCTATGTTCAGAATTG | | | |
| | TTTGCCCAACTTTCTTGTACAAAGT | | | |
| | TGTCCCC | | | |
| ASCL4 | ATGGAGACGCGTAAACCGGCGGAA | 36 | Involved in | Jonsson, M. et |
| | CGGCTGGCCTTGCCATACTCGCTGC | | development of | al. Hash4, a |
| | GCACCGCGCCCCTGGGCGTTCCGG | | skin | novel human |
| | GGACCCTGCCCGGACTCCCGCGGA | | | achaete-scute |
| | GGGACCCCCTCAGGGTCGCCCTGC | | | homologue |
| | GTCTGGACGCCGCGTGCTGGGAGT | | | found in fetal |
| | GGGCGCGCAGCGGCTGCGCACGGG | | | skin. |
| | GATGGCAGTACTTGCCCGTGCCGCT | | | Genomics 84, |
| | GGACAGCGCCTTCGAGCCCGCCTTC | | | 859-866 |
| | CTCCGCAAGCGCAACGAGCGCGAG | | | (2004) |
| | CGGCAGCGGGTGCGCTGCGTGAAC | | | |
| | GAGGGCTATGCGCGCCTCCGAGAC | | | |
| | CACCTGCCCCGGGAGCTGGCAGAC | | | |
| | AAGCGCCTCAGCAAAGTGGAGACG | | | |
| | CTCCGCGCTGCCATCGACTACATCA | | | |
| | AGCACCTGCAGGAGCTGCTGGAGC | | | |
| | GCCAGGCCTGGGGGCTCGAGGGCG | | | |
| | CGGCCGGCGCCGTCCCCCAGCGCA | | | |
| | GGGCGGAATGCAACAGCGACGGGG | | | |
| | AGTCCAAGGCCTCTTCGGCGCCTTC | | | |
| | GCCCAGCAGCGAGCCCGAGGAGGG | | | |
| | GGGCAGC | | | |
| ASCL5 | ATGCCGATGGGGGCAGCAGAAAGA | 37 | Paralog of | Wang, C. et |
| | GGTGCTGGGCCCCAATCATCTGCAG | | ASCL4 | al. Systematic |
| | CACCATGGGCTGGTTCAGAAAAGG | | | analysis of the |
| | CGGCAAAGAGAGGGCCATCAAAAA | | | achaete-scute |
| | GCTGGTACCCAAGAGCTGCTGCATC | | | complex-like |
| | TGATGTCACGTGCCCGACTGGTGGT | | | gene signature |
| | GATGGAGCTGACCCAAAACCTGGA | | | in clinical |
| | CCTTTTGGAGGTGGTTTAGCTTTAG | | | cancer |
| | GGCCTGCGCCCAGAGGAACAATGA | | | patients. |
| | ATAATAATTTCTGCAGGGCCCTTGT | | | Molecular and |
| | TGACAGAAGGCCTTTAGGACCCCCT | | | Clinical |
| | TCATGTATGCAATTAGGTGTAATGC | | | Oncology 6, |
| | CACCGCCAAGACAAGCGCCCCTCC | | | (Spandidos |
| | CGCCGGCTGAACCCCTTGGAAATGT | | | Publications, |
| | ACCTTTCCTCCTATACCCTGGCCCA | | | 2017). |
| | GCTGAACCACCATATTATGATGCAT | | | |
| | ATGCTGGTGTTTTCCCATATGTGCC | | | |
| | TTTCCCTGGTGCTTTTGGTGTATAT | | | |
| | GAATACCCTTTTGAGCCGGCTTTTA | | | |
| | TCCAAAAGAGGAATGAAAGAGAGA | | | |
| | GACAGAGAGTGAAGTGTGTGAATG | | | |
| | AAGGATACGCCAGATTGAGAGGCC | | | |
| | ATTTGCCTGGTGCCCTGGCAGAAAA | | | |
| | GAGATTATCAAAAGTTGAAACCCT | | | |
| | GAGGGCGGCAATCAGATATATAAA | | | |
| | ATACCTCCAAGAACTCCTTTCATCA | | | |
| | GCACCTGATGGATCGACACCACCG | | | |
| | GCTTCAAGAGGTTTACCTGGAACTG | | | |
| | GACCATGCCCTGCACCGCCTGCTAC | | | |
| | ACCAAGGCCAGACAGACCTGGAGA | | | |
| | TGGAGAAGCAAGAGCACCTTCTTC | | | |
| | CCTTGTCCCTGAATCTTCTGAATCA | | | |
| | TCATGTTTTTCGCCTTCCCCTTTTTT | | | |
| | AGAAAGTGAAGAATCCTGGCA | | | |
| ATF7 | ATGGGAGACGACAGACCGTTTGTG | 38 | Involved in | Peters, C. S. |
| | TGCAATGCCCCGGGCTGTGGACAG | | early cell | et al. ATF-7, |
| | AGATTTACAAACGAGGACCACCTG | | signaling, binds | a novel bZIP |
| | GCAGTTCATAAACACAAGCATGAG | | cAMP response | protein, |
| | ATGACATTGAAATTTGGCCCAGCCC | | element | interacts with |
| | GAACTGACTCAGTCATCATTGCAGA | | | the PRL-1 |
| | TCAAACGCCTACTCCAACTAGATTC | | | protein- |
| | CTGAAGAACTGTGAGGAGGTGGGA | | | tyrosine |
| | CTCTTCAATGAACTAGCTAGCTCCT | | | phosphatase. |
| | TTGAACATGAATTCAAGAAAGCTG | | | J. Biol. Chem. |
| | CAGATGAGGATGAGAAAAAGGCAA | | | 276, 13718- |
| | GAAGCAGGACTGTTGCCAAAAAAC | | | 26 (2001). |
| | TGGTGGCTGCTGCTGGGCCCCTTGA | | | Hamard, P.-J. |
| | CATGTCTCTGCCTTCCACACCAGAC | | | et al. A |
| | ATCAAAATCAAAGAAGAAGAGCCA | | | functional |
| | GTGGAGGTAGACTCATCCCCACCTG | | | interaction |
| | ATAGCCCTGCCTCTAGTCCCTGTTC | | | between |
| | CCCACCACTGAAGGAGAAGGAGGT | | | ATF7 and |
| | TACCCCAAAGCCTGTTCTGATCTCT | | | TAF12 that is |
| | ACCCCCACACCCACCATTGTACGTC | | | modulated by |
| | CTGGCTCCCTGCCTCTCCACTTGGG | | | TAF4. |
| | CTATGATCCACTTCATCCAACCCTT | | | Oncogene 24, |
| | CCCTCCCCAACCTCTGTCATCACAC | | | 3472-3483 |
| | AGGCTCCACCATCCAACAGGCAAA | | | (2005). |
| | TGGGGTCTCCCACTGGCTCCCTCCC | | | |
| | TCTTGTCATGCATCTTGCTAATGGA | | | |
| | CAGACCATGCCTGTGTTGCCAGGGC | | | |
| | CTCCAGTACAGATGCCGTCTGTTAT | | | |
| | ATCGCTGGCCAGACCTGTGTCCATG | | | |
| | GTGCCCAACATTCCTGGTATCCCTG | | | |
| | GCCCACCAGTTAACAGTAGTGGCTC | | | |
| | CATTTCTCCCTCTGGCCACCCTATA | | | |
| | CCATCAGAAGCCAAGATGAGACTG | | | |
| | AAAGCCACCCTAACTCACCAAGTCT | | | |
| | CCTCAATCAATGGTGGTTGTGGAAT | | | |
| | GGTGGTGGGTACTGCCAGCACCAT | | | |
| | GGTGACAGCCCGCCCAGAGCAGAG | | | |
| | CCAGATTCTCATCCAGCACCCTGAT | | | |
| | GCCCCATCCCCTGCCCAGCCACAG | | | |
| | GTCTCACCAGCTCAGCCCACCCCTA | | | |
| | GTACTGGGGGGCGACGGCGGCGCA | | | |
| | CAGTAGATGAAGATCCAGATGAGC | | | |
| | GACGGCAGCGCTTTCTGGAGCGCA | | | |
| | ACCGGGCTGCAGCCTCCCGCTGCCG | | | |
| | CCAAAAGCGAAAGCTGTGGGTGTC | | | |
| | CTCCCTAGAGAAGAAGGCCGAAGA | | | |
| | ACTCACTTCTCAGAACATTCAGCTG | | | |
| | AGTAATGAAGTCACATTACTACGC | | | |
| | AATGAGGTGGCCCAGTTGAAACAG | | | |
| | CTACTGTTAGCTCATAAAGACTGCC | | | |
| | CAGTCACTGCACTACAGAAAAAGA | | | |
| | CTCAAGGCTATTTAGAAAGCCCCA | | | |
| | AGGAAAGCTCAGAGCCAACGGGTT | | | |
| | CTCCAGCCCCTGTGATTCAGCACAG | | | |
| | CTCAGCAACAGCCCCTAGCAATGG | | | |
| | CCTCAGTGTTCGCTCTGCAGCTGAA | | | |
| | GCTGTGGCCACCTCGGTCCTCACTC | | | |
| | AGATGGCCAGCCAAAGGACAGAAC | | | |
| | TGAGCATGCCGATACAATCGCATGT | | | |
| | AATCATGACCCCACAGTCCCAGTCT | | | |
| | GCGGGCAGA | | | |
| CDX2 | ATGTACGTGAGCTACCTCCTGGACA | 39 | Involved in | Strumpf, D. et |
| | AGGACGTGAGCATGTACCCTAGCT | | trophectoderm | al. Cdx2 is |
| | CCGTGCGCCACTCTGGCGGCCTCAA | | specification | required for |
| | CCTGGCGCCGCAGAACTTCGTCAGC | | and | correct cell |
| | CCCCCGCAGTACCCGGACTACGGC | | differentiation | fate |
| | GGTTACCACGTGGCGGCCGCAGCT | | | specification |
| | GCAGCGGCAGCGAACTTGGACAGC | | | and |
| | GCGCAGTCCCCGGGGCCATCCTGG | | | differentiation |
| | CCGGCAGCGTATGGCGCCCCACTCC | | | of |
| | GGGAGGACTGGAATGGCTACGCGC | | | trophectoderm |
| | CCGGAGGCGCCGCGGCCGCCGCCA | | | in the |
| | ACGCCGTGGCTCACGGCCTCAACG | | | mouse |
| | GTGGCTCCCCGGCCGCAGCCATGG | | | blastocyst. |
| | GCTACAGCAGCCCCGCAGACTACC | | | Development |
| | ATCCGCACCACCACCCGCATCACC | | | 132, 2093- |
| | ACCCGCACCACCCGGCCGCCGCGC | | | 102 (2005). |
| | CTTCCTGCGCTTCTGGGCTGCTGCA | | | |
| | AACGCTCAACCCCGGCCCTCCTGGG | | | |
| | CCCGCCGCCACCGCTGCCGCCGAG | | | |
| | CAGCTGTCTCCCGGCGGCCAGCGG | | | |
| | CGGAACCTGTGCGAGTGGATGCGG | | | |
| | AAGCCGGCGCAGCAGTCCCTCGGC | | | |
| | AGCCAAGTGAAAACCAGGACGAAA | | | |
| | GACAAATATCGAGTGGTGTACACG | | | |
| | GACCACCAGCGGCTGGAGCTGGAG | | | |
| | AAGGAGTTTCACTACAGTCGCTACA | | | |
| | TCACCATCCGGAGGAAAGCCGAGC | | | |
| | TAGCCGCCACGCTGGGGCTCTCTGA | | | |
| | GAGGCAGGTTAAAATCTGGTTTCA | | | |
| | GAACCGCAGAGCAAAGGAGAGGA | | | |
| | AAATCAACAAGAAGAAGTTGCAGC | | | |
| | AGCAACAGCAGCAGCAGCCACCAC | | | |
| | AGCCGCCTCCGCCGCCACCACAGC | | | |
| | CTCCCCAGCCTCAGCCAGGTCCTCT | | | |
| | GAGAAGTGTCCCAGAGCCCTTGAG | | | |
| | TCCGGTGTCTTCCCTGCAAGCCTCA | | | |
| | GTGTCTGGCTCTGTCCCTGGGGTTC | | | |
| | TGGGGCCAACTGGGGGGGTGCTAA | | | |
| | ACCCCACCGTCACCCAG | | | |
| CRX | ATGATGGCGTATATGAACCCGGGG | 40 | Involved in | Furukawa, T., |
| | CCCCACTATTCTGTCAACGCCTTGG | | photoreceptor | Morrow, E. |
| | CCCTAAGTGGCCCCAGTGTGGATCT | | differentiation | M. & Cepko, |
| | GATGCACCAGGCTGTGCCCTACCCA | | | C. L. Crx, a |
| | AGCGCCCCCAGGAAGCAGCGGCGG | | | novel otx-like |
| | GAGCGCACCACCTTCACCCGGAGC | | | homeobox |
| | CAACTGGAGGAGCTGGAGGCACTG | | | gene, shows |
| | TTTGCCAAGACCCAGTACCCAGAC | | | photoreceptor- |
| | GTCTATGCCCGTGAGGAGGTGGCTC | | | specific |
| | TGAAGATCAATCTGCCTGAGTCCAG | | | expression |
| | GGTTCAGGTTTGGTTCAAGAACCGG | | | and regulates |
| | AGGGCTAAATGCAGGCAGCAGCGA | | | photoreceptor |
| | CAGCAGCAGAAACAGCAGCAGCAG | | | differentiation. |
| | CCCCCAGGGGGCCAGGCCAAGGCC | | | Cell 91, |
| | CGGCCTGCCAAGAGGAAGGCGGGC | | | 531-541 |
| | ACGTCCCCAAGACCCTCCACAGAT | | | (1997). |
| | GTGTGTCCAGACCCTCTGGGCATCT | | | |
| | CAGATTCCTACAGTCCCCCTCTGCC | | | |
| | CGGCCCCTCAGGCTCCCCAACCAC | | | |
| | GGCAGTGGCCACTGTGTCCATCTGG | | | |
| | AGCCCAGCCTCAGAGTCCCCTTTGC | | | |
| | CTGAGGCGCAGCGGGCTGGGCTGG | | | |
| | TGGCCTCAGGGCCGTCTCTGACCTC | | | |
| | CGCCCCCTATGCCATGACCTACGCC | | | |
| | CCGGCCTCCGCTTTCTGCTCTTCCC | | | |
| | CCTCCGCCTATGGGTCTCCGAGCTC | | | |
| | CTATTTCAGCGGCCTAGACCCCTAC | | | |
| | CTTTCTCCCATGGTGCCCCAGCTAG | | | |
| | GGGGCCCGGCTCTTAGCCCCCTCTC | | | |
| | TGGCCCCTCCGTGGGACCTTCCCTG | | | |
| | GCCCAGTCCCCCACCTCCCTATCAG | | | |
| | GCCAGAGCTATGGCGCCTACAGCC | | | |
| | CCGTGGATAGCTTGGAATTCAAGG | | | |
| | ACCCCACGGGCACCTGGAAATTCA | | | |
| | CCTACAATCCCATGGACCCTCTGGA | | | |
| | CTACAAGGATCAGAGTGCCTGGAA | | | |
| | GTTTCAGATCTTG | | | |
| ERG | ATGGCCAGCACTATTAAGGAAGCC | 41 | Involved in | Mclaughlin, |
| | TTATCAGTTGTGAGTGAGGACCAGT | | endothelial cell | F. et al. |
| | CGTTGTTTGAGTGTGCCTACGGAAC | | specification | Combined |
| | GCCACACCTGGCTAAGACAGAGAT | | and | genomic and |
| | GACCGCGTCCTCCTCCAGCGACTAT | | differentiation | antisense |
| | GGACAGACTTCCAAGATGAGCCCA | | | analysis |
| | CGCGTCCCTCAGCAGGATTGGCTGT | | | reveals that |
| | CTCAACCCCCAGCCAGGGTCACCAT | | | the |
| | CAAAATGGAATGTAACCCTAGCCA | | | transcription |
| | GGTGAATGGCTCAAGGAACTCTCCT | | | factor Erg is |
| | GATGAATGCAGTGTGGCCAAAGGC | | | implicated in |
| | GGGAAGATGGTGGGCAGCCCAGAC | | | endothelial |
| | ACCGTTGGGATGAACTACGGCAGC | | | cell |
| | TACATGGAGGAGAAGCACATGCCA | | | differentiation. |
| | CCCCCAAACATGACCACGAACGAG | | | Blood 98, |
| | CGCAGAGTTATCGTGCCAGCAGAT | | | 3332-3339 |
| | CCTACGCTATGGAGTACAGACCAT | | | (2001). |
| | GTGCGGCAGTGGCTGGAGTGGGCG | | | |
| | GTGAAAGAATATGGCCTTCCAGAC | | | |
| | GTCAACATCTTGTTATTCCAGAACA | | | |
| | TCGATGGGAAGGAACTGTGCAAGA | | | |
| | TGACCAAGGACGACTTCCAGAGGC | | | |
| | TCACCCCCAGCTACAATGCCGACAT | | | |
| | CCTTCTCTCACATCTCCACTACCTC | | | |
| | AGAGAGACTCCTCTTCCACATTTGA | | | |
| | CTTCAGATGATGTTGATAAAGCCTT | | | |
| | ACAAAACTCTCCACGGTTAATGCAT | | | |
| | GCTAGAAACACAGGGGGTGCAGCT | | | |
| | TTTATTTTCCCAAATACTTCAGTAT | | | |
| | ATCCTGAAGCTACGCAAAGAATTA | | | |
| | CAACTAGGCCAGATTTACCATATGA | | | |
| | GCCCCCCAGGAGATCAGCCTGGAC | | | |
| | CGGTCACGGCCACCCCACGCCCCA | | | |
| | GTCGAAAGCTGCTCAACCATCTCCT | | | |
| | TCCACAGTGCCCAAAACTGAAGAC | | | |
| | CAGCGTCCTCAGTTAGATCCTTATC | | | |
| | AGATTCTTGGACCAACAAGTAGCC | | | |
| | GCCTTGCAAATCCAGGCAGTGGCC | | | |
| | AGATCCAGCTTTGGCAGTTCCTCCT | | | |
| | GGAGCTCCTGTCGGACAGCTCCAA | | | |
| | CTCCAGCTGCATCACCTGGGAAGG | | | |
| | CACCAACGGGGAGTTCAAGATGAC | | | |
| | GGATCCCGACGAGGTGGCCCGGCG | | | |
| | CTGGGGAGAGCGGAAGAGCAAACC | | | |
| | CAACATGAACTACGATAAGCTCAG | | | |
| | CCGCGCCCTCCGTTACTACTATGAC | | | |
| | AAGAACATCATGACCAAGGTCCAT | | | |
| | GGGAAGCGCTACGCCTACAAGTTC | | | |
| | GACTTCCACGGGATCGCCCAGGCC | | | |
| | CTCCAGCCCCACCCCCCGGAGTCAT | | | |
| | CTCTGTACAAGTACCCCTCAGACCT | | | |
| | CCCGTACATGGGCTCCTATCACGCC | | | |
| | CACCCACAGAAGATGAACTTTGTG | | | |
| | GCGCCCCACCCTCCAGCCCTCCCCG | | | |
| | TGACATCTTCCAGTTTTTTTGCTGCC | | | |
| | CCAAACCCATACTGGAATTCACCA | | | |
| | ACTGGGGGTATATACCCCAACACT | | | |
| | AGGCTCCCCACCAGCCATATGCCTT | | | |
| | CTCATCTGGGCACTTACTAC | | | |
| ESRRG | ATGTCAAACAAAGATCGACACATT | 42 | Involved in | Alaynick, W. |
| | GATTCCAGCTGTTCGTCCTTCATCA | | cardiac | A. et al. ERRγ |
| | AGACGGAACCTTCCAGCCCAGCCT | | development | Directs and |
| | CCCTGACGGACAGCGTCAACCACC | | | Maintains the |
| | ACAGCCCTGGTGGCTCTTCAGACGC | | | Transition |
| | CAGTGGGAGCTACAGTTCAACCAT | | | to Oxidative |
| | GAATGGCCATCAGAACGGACTTGA | | | Metabolism in |
| | CTCGCCACCTCTCTACCCTTCTGCT | | | the Postnatal |
| | CCTATCCTGGGAGGTAGTGGGCCTG | | | Heart. Cell |
| | TCAGGAAACTGTATGATGACTGCTC | | | Metab. 6, 13- |
| | CAGCACCATTGTTGAAGATCCCCAG | | | 24 (2007). |
| | ACCAAGTGTGAATACATGCTCAACT | | | |
| | CGATGCCCAAGAGACTGTGTTTAGT | | | |
| | GTGTGGTGACATCGCTTCTGGGTAC | | | |
| | CACTATGGGGTAGCATCATGTGAA | | | |
| | GCCTGCAAGGCATTCTTCAAGAGG | | | |
| | ACAATTCAAGGCAATATAGAATAC | | | |
| | AGCTGCCCTGCCACGAATGAATGT | | | |
| | GAAATCACAAAGCGCAGACGTAAA | | | |
| | TCCTGCCAGGCTTGCCGCTTCATGA | | | |
| | AGTGTTTAAAAGTGGGCATGCTGA | | | |
| | AAGAAGGGGTGCGTCTTGACAGAG | | | |
| | TACGTGGAGGTCGGCAGAAGTACA | | | |
| | AGCGCAGGATAGATGCGGAGAACA | | | |
| | GCCCATACCTGAACCCTCAGCTGGT | | | |
| | TCAGCCAGCCAAAAAGCCATTGCT | | | |
| | CTGGTCTGATCCTGCAGATAACAAG | | | |
| | ATTGTCTCACATTTGTTGGTGGCTG | | | |
| | AACCGGAGAAGATCTATGCCATGC | | | |
| | CTGACCCTACTGTCCCCGACAGTGA | | | |
| | CATCAAAGCCCTCACTACACTGTGT | | | |
| | GACTTGGCCGACCGAGAGTTGGTG | | | |
| | GTTATCATTGGATGGGCGAAGCAT | | | |
| | ATTCCAGGCTTCTCCACGCTGTCCC | | | |
| | TGGCGGACCAGATGAGCCTTCTGC | | | |
| | AGAGTGCTTGGATGGAAATTTTGAT | | | |
| | CCTTGGTGTCGTATACCGGTCTCTT | | | |
| | TCGTTTGAGGATGAACTTGTCTATG | | | |
| | CAGACGATTATATAATGGACGAAG | | | |
| | ACCAGTCCAAATTAGCAGGCCTTCT | | | |
| | TGATCTAAATAATGCTATCCTGCAG | | | |
| | CTGGTAAAGAAATACAAGAGCATG | | | |
| | AAGCTGGAAAAAGAAGAATTTGTC | | | |
| | ACCCTCAAAGCTATAGCTCTTGCTA | | | |
| | ATTCAGACTCCATGCACATAGAAG | | | |
| | ATGTTGAAGCCGTTCAGAAGCTTCA | | | |
| | GGATGTCTTACATGAAGCGCTGCA | | | |
| | GGATTATGAAGCTGGCCAGCACAT | | | |
| | GGAAGACCCTCGTCGAGCTGGCAA | | | |
| | GATGCTGATGACACTGCCACTCCTG | | | |
| | AGGCAGACCTCTACCAAGGCCGTG | | | |
| | CAGCATTTCTACAACATCAAACTAG | | | |
| | AAGGCAAAGTCCCAATGCACAAAC | | | |
| | TTTTTTTGGAAATGTTGGAGGCCAA | | | |
| | GGTC | | | |
| ETV2 | ATGGATCTTTGGAACTGGGATGAA | 43 | Involved in | Lee, D. et al. |
| | GCTTCCCCTCAAGAAGTTCCCCCCG | | haemato- | ER71 acts |
| | GAAATAAACTCGCGGGGCTTGGAA | | endothelial | downstream |
| | GACTCCCTCGCCTTCCGCAACGCGT | | specification | of BMP, |
| | CTGGGGCGGATGCCCTGGTGGAGC | | and | Notch, and |
| | CTCAGCGGACCCAAACCCTTTGTCT | | differentiation, | Wnt signaling |
| | CCAGCGGAGGGGGCAAAGTTGGGT | | and in | in blood and |
| | TTCTGCTTCCCGGATCTTGCTTTGC | | vasculogenesis | vessel |
| | AAGGCGATACTCCAACGGCGACGG | | | progenitor |
| | CAGAGACCTGTTGGAAAGGCACCA | | | specification. |
| | GTAGCTCCCTGGCCAGCTTTCCGCA | | | Cell Stem |
| | GCTCGATTGGGGGTCAGCCCTTCTC | | | Cell 2, 49- |
| | CATCCCGAAGTTCCCTGGGGGGCG | | | 507 (2008). |
| | GAACCCGACTCCCAAGCCCTTCCCT | | | |
| | GGAGTGGTGATTGGACAGATATGG | | | |
| | CATGCACAGCCTGGGACAGTTGGT | | | |
| | CCGGGGCGTCACAGACATTGGGAC | | | |
| | CAGCCCCACTTGGACCGGGGCCTAT | | | |
| | CCCCGCAGCAGGAAGCGAAGGAGC | | | |
| | TGCTGGTCAGAACTGTGTGCCCGTG | | | |
| | GCTGGTGAGGCTACCAGTTGGTCCA | | | |
| | GGGCCCAGGCAGCAGGCAGTAACA | | | |
| | CCAGCTGGGATTGCTCAGTGGGGC | | | |
| | CTGACGGGGATACTTATTGGGGCTC | | | |
| | TGGTCTTGGTGGAGAACCGAGAAC | | | |
| | GGACTGTACGATAAGTTGGGGCGG | | | |
| | TCCAGCTGGGCCTGATTGTACTACG | | | |
| | TCATGGAATCCTGGCTTGCACGCCG | | | |
| | GCGGCACGACAAGCCTTAAGAGAT | | | |
| | ATCAAAGTTCAGCCCTTACAGTTTG | | | |
| | CTCAGAACCTTCCCCGCAAAGTGAC | | | |
| | CGAGCGTCACTGGCGCGATGTCCTA | | | |
| | AAACTAATCATCGAGGGCCGATCC | | | |
| | AGTTGTGGCAGTTTTTGCTTGAACT | | | |
| | CCTTCACGATGGCGCGAGGAGCAG | | | |
| | TTGCATCAGATGGACCGGTAACAG | | | |
| | CAGGGAGTTCCAATTGTGTGACCCC | | | |
| | AAGGAAGTGGCTCGACTGTGGGGT | | | |
| | GAGCGCAAACGGAAGCCTGGTATG | | | |
| | AATTACGAAAAGTTGAGTAGGGGT | | | |
| | TTGCGATATTACTATAGGCGCGACA | | | |
| | TCGTTCGAAAGTCCGGTGGTCGAA | | | |
| | AGTACACATACAGATTCGGCGGTC | | | |
| | GCGTACCATCTCTTGCATACCCTGA | | | |
| | TTGCGCAGGCGGGGGTAGGGGTGC | | | |
| | GGAAACACAA | | | |
| FLI1 | ATGGACGGGACTATTAAGGAGGCT | 44 | Involved in | Liu, F. et al. |
| | CTGTCGGTGGTGAGCGACGACCAG | | haemato- | Fli1 Acts at |
| | TCCCTCTTTGACTCAGCGTACGGAG | | endothelial | the Top of the |
| | CGGCAGCCCATCTCCCCAAGGCCG | | specification | Transcriptional |
| | ACATGACTGCCTCGGGGAGTCCTG | | and | Network |
| | ACTACGGGCAGCCCCACAAGATCA | | differentiation | Driving Blood |
| | ACCCCCTCCCACCACAGCAGGAGT | | | and |
| | GGATCAATCAGCCAGTGAGGGTCA | | | Endothelial |
| | ACGTCAAGCGGGAGTATGACCACA | | | Development. |
| | TGAATGGATCCAGGGAGTCTCCGG | | | Curr. Biol. 18, |
| | TGGACTGCAGCGTTAGCAAATGCA | | | 1234-1240 |
| | GCAAGCTGGTGGGCGGAGGCGAGT | | | (2008). |
| | CCAACCCCATGAACTACAACAGCT | | | |
| | ATATGGACGAGAAGAATGGCCCCC | | | |
| | CTCCTCCCAACATGACCACCAACGA | | | |
| | GAGGAGAGTCATCGTCCCCGCAGA | | | |
| | CCCCACACTGTGGACACAGGAGCA | | | |
| | TGTGAGGCAATGGCTGGAGTGGGC | | | |
| | CATAAAGGAGTACAGCTTGATGGA | | | |
| | GATCGACACATCCTTTTTCCAGAAC | | | |
| | ATGGATGGCAAGGAACTGTGTAAA | | | |
| | ATGAACAAGGAGGACTTCCTCCGC | | | |
| | GCCACCACCCTCTACAACACGGAA | | | |
| | GTGCTGTTGTCACACCTCAGTTACC | | | |
| | TCAGGGAAAGTTCACTGCTGGCCTA | | | |
| | TAATACAACCTCCCACACCGACCA | | | |
| | ATCCTCACGATTGAGTGTCAAAGA | | | |
| | AGACCCTTCTTATGACTCAGTCAGA | | | |
| | AGAGGAGCTTGGGGCAATAACATG | | | |
| | AATTCTGGCCTCAACAAAAGTCCTC | | | |
| | CCCTTGGAGGGGCACAAACGATCA | | | |
| | GTAAGAATACAGAGCAACGGCCCC | | | |
| | AGCCAGATCCGTATCAGATCCTGG | | | |
| | GCCCGACCAGCAGTCGCCTAGCCA | | | |
| | ACCCTGGAAGCGGGCAGATCCAGC | | | |
| | TGTGGCAATTCCTCCTGGAGCTGCT | | | |
| | CTCCGACAGCGCCAACGCCAGCTG | | | |
| | TATCACCTGGGAGGGGACCAACGG | | | |
| | GGAGTTCAAAATGACGGACCCCGA | | | |
| | TGAGGTGGCCAGGCGCTGGGGCGA | | | |
| | GCGGAAAAGCAAGCCCAACATGAA | | | |
| | TTACGACAAGCTGAGCCGGGCCCT | | | |
| | CCGTTATTACTATGATAAAAACATT | | | |
| | ATGACCAAAGTGCACGGCAAAAGA | | | |
| | TATGCTTACAAATTTGACTTCCACG | | | |
| | GCATTGCCCAGGCTCTGCAGCCACA | | | |
| | TCCGACCGAGTCGTCCATGTACAAG | | | |
| | TACCCTTCTGACATCTCCTACATGC | | | |
| | CTTCCTACCATGCCCACCAGCAGAA | | | |
| | GGTGAACTTTGTCCCTCCCCATCCA | | | |
| | TCCTCCATGCCTGTCACTTCCTCCA | | | |
| | GCTTCTTTGGAGCCGCATCACAATA | | | |
| | CTGGACCTCCCCCACGGGGGGAAT | | | |
| | CTACCCCAACCCCAACGTCCCCCGC | | | |
| | CATCCTAACACCCACGTGCCTTCAC | | | |
| | ACTTAGGCAGCTACTAC | | | |
| FOXA1 | ATGTTGGGCACCGTGAGATGGAG | 45 | Involved in | Friedman, J. |
| | GGGCATGAGACAAGCGACTGGAAT | | branching | R. et al. The |
| | TCCTACTACGCGGATACCCAAGAA | | morphogenesis, | Foxa family |
| | GCGTATTCTTCAGTTCCCGTAAGCA | | development of | of |
| | ATATGAACTCCGGATTGGGGAGCA | | lung, liver, | transcription |
| | TGAATAGTATGAACACGTATATGA | | prostate, and | factors in |
| | CAATGAATACGATGACCACCAGCG | | pancreas | development |
| | GCAACATGACACCGGCCTCCTTTAA | | | and |
| | TATGTCATATGCGAACCCTGGTCTT | | | metabolism. |
| | GGCGCTGGCCTCTCACCAGGTGCG | | | Cell. Mol. |
| | GTCGCTGGAATGCCCGGGGGGAGC | | | Life Sci. 63, |
| | GCCGGAGCGATGAACTCCATGACC | | | 2317-2328 |
| | GCTGCGGGCGTGACGGCCATGGGT | | | (2006). |
| | ACGGCCCTTGTCACCCAGTGGAATG | | | |
| | GGCGCTGGCCTCTCACCAGGTGCG | | | |
| | GTCGCTGGAATGCCCGGGGGGAGC | | | |
| | GCCGGAGCGATGAACTCCATGACC | | | |
| | GCTGCGGGCGTGACGGCCATGGGT | | | |
| | ACGGCCCTGTCACCCAGTGGAATG | | | |
| | GGAGCTATGGGGGCCCAGCAAGCC | | | |
| | GCCTCAATGAATGGATTGGGGCCCT | | | |
| | ATGCCGCGGCGATGAATCCCTGCAT | | | |
| | GTCCCCTATGGCTTATGCCCCCAGC | | | |
| | AATTTGGGTCGCAGTAGAGCCGGC | | | |
| | GGTGGTGGCGATGCCAAAACCTTC | | | |
| | AAGCGAAGTTATCCTCATGCGAAG | | | |
| | CCTCCTTATTCATATATATCCTTGAT | | | |
| | TACGATGGCGATACAGCAGGCCCC | | | |
| | GTCTAAGATGCTGACTCTGAGTGAG | | | |
| | ATATACCAGTGGATCATGGACCTTT | | | |
| | TTCCTTACTACCGGCAAAACCAACA | | | |
| | GAGATGGCAAAACTCAATACGCCA | | | |
| | TAGCCTTTCCTTCAATGATTGCTTT | | | |
| | GTCAAAGTCGCTCGGAGCCCTGAC | | | |
| | AAGCCCGGTAAAGGGTCCTATTGG | | | |
| | ACCCTTCATCCAGATAGCGGCAATA | | | |
| | TGTTCGAGAATGGTTGTTATCTTAG | | | |
| | ACGGCAGAAACGATTCAAATGTGA | | | |
| | GAAACAGCCAGGTGCCGGCGGTGG | | | |
| | TGGCGGCAGCGGTTCAGGCGGAAG | | | |
| | TGGTGCCAAGGGTGGGCCTGAGTC | | | |
| | TAGAAAAGACCCCAGCGGAGCAAG | | | |
| | CAATCCAAGCGCGGACTCTCCCCTG | | | |
| | CACCGCGGTGTTCATGGTAAGACA | | | |
| | GGTCAGCTTGAGGGGGCGCCTGCT | | | |
| | CCAGGCCCGGCTGCGTCACCGCAA | | | |
| | ACACTGGACCATAGTGGAGCTACA | | | |
| | GCGACCGGAGGTGCTTCAGAACTC | | | |
| | AAGACGCCTGCGTCCTCCACTGCGC | | | |
| | CTCCGATCTCCAGTGGTCCCGGTGC | | | |
| | ACTTGCCTCTGTTCCTGCATCTCAT | | | |
| | CCAGCACACGGACTCGCGCCGCAC | | | |
| | GAGTCCCAGCTCCATTTGAAAGGG | | | |
| | GACCCACACTACAGCTTTAACCACC | | | |
| | CATTCTCTATTAACAATTTGATGTC | | | |
| | ATCCTCAGAACAGCAGCATAAACT | | | |
| | CGACTTCAAAGCCTATGAACAGGC | | | |
| | CCTGCAGTATTCTCCATATGGCTCT | | | |
| | ACACTTCCTGCTTCTCTTCCATTGG | | | |
| | GGTCTGCAAGTGTGACAACGCGCT | | | |
| | CCCCAATCGAGCCAAGTGCCCTCG | | | |
| | AGCCTGCTTATTATCAAGGAGTATA | | | |
| | TTCCCGACCAGTTTTGAATACAAGT | | | |
| FOXA2 | ATGCTGGGAGCGGTGAAGATGGAA | 46 | Involved in | Friedman, J. |
| | GGGCACGAGCCGTCCGACTGGAGC | | branching | R. et al. The |
| | AGCTACTATGCAGAGCCCGAGGGC | | morphogenesis, | Foxa family |
| | TACTCCTCCGTGAGCAACATGAACG | | development of | of |
| | CCGGCCTGGGGATGAACGGCATGA | | notochord, lung, | transcription |
| | ACACGTACATGAGCATGTCGGCGG | | liver, prostate, | factors in |
| | CCGCCATGGGCAGCGGCTCGGGCA | | and pancreas. | development |
| | ACATGAGCGCGGGCTCCATGAACA | | | and |
| | TGTCGTCGTACGTGGGCGCTGGCAT | | | metabolism. |
| | GAGCCCGTCCCTGGCGGGGATGTC | | | Cell. Mol. |
| | CCCCGGCGCGGGCGCCATGGCGGG | | | Life Sci. 63, |
| | CATGGGCGGCTCGGCCGGGGCGGC | | | 2317-2328 |
| | TGGCGTGGCGGGCATGGGGCCGCA | | | (2006). |
| | CTTGAGTCCCAGCCTGAGCCCGCTC | | | |
| | GGGGGGCAGGCGGCCGGGGCCATG | | | |
| | GGCGGCCTGGCCCCCTACGCCAAC | | | |
| | ATGAACTCCATGAGCCCCATGTACG | | | |
| | GGCAGGCGGGCCTGAGCCGCGCCC | | | |
| | GCGACCCCAAGACCTACAGGCGCA | | | |
| | GCTACACGCACGCAAAGCCGCCCT | | | |
| | ACTCGTACATCTCGCTCATCACCAT | | | |
| | GGCCATCCAGCAGAGCCCCAACAA | | | |
| | GATGCTGACGCTGAGCGAGATCTA | | | |
| | CCAGTGGATCATGGACCTCTTCCCC | | | |
| | TTCTACCGGCAGAACCAGCAGCGC | | | |
| | TGGCAGAACTCCATCCGCCACTCGC | | | |
| | TCTCCTTCAACGACTGTTTCCTGAA | | | |
| | GGTGCCCCGCTCGCCCGACAAGCC | | | |
| | CGGCAAGGGCTCCTTCTGGACCCTG | | | |
| | CACCCTGACTCGGGCAACATGTTCG | | | |
| | AGAACGGCTGCTACCTGCGCCGCC | | | |
| | AGAAGCGCTTCAAGTGCGAGAAGC | | | |
| | AGCTGGCGCTGAAGGAGGCCGCAG | | | |
| | GCGCCGCCGGCAGCGGCAAGAAGG | | | |
| | CGGCCGCCGGGGCCCAGGCCTCAC | | | |
| | AGGCTCAACTCGGGGAGGCCGCCG | | | |
| | GGCCGGCCTCCGAGACTCCGGCGG | | | |
| | GCACCGAGTCGCCTCACTCGAGCG | | | |
| | CCTCCCCGTGCCAGGAGCACAAGC | | | |
| | GAGGGGGCCTGGGAGAGCTGAAGG | | | |
| | GGACGCCGGCTGCGGCGCTGAGCC | | | |
| | CCCCAGAGCCGGCGCCCTCTCCCG | | | |
| | GGCAGCAGCAGCAGGCCGCGGCCC | | | |
| | ACCTGCTGGGCCCGCCCCACCACCC | | | |
| | GGGCCTGCCGCCTGAGGCCCACCT | | | |
| | GAAGCCGGAACACCACTACGCCTT | | | |
| | CAACCACCCGTTCTCCATCAACAAC | | | |
| | CTCATGTCCTCGGAGCAGCAGCACC | | | |
| | ACCACAGCCACCACCACCACCAGC | | | |
| | CCCACAAAATGGACCTCAAGGCCT | | | |
| | ACGAACAGGTGATGCACTACCCCG | | | |
| | GCTACGGTTCCCCCATGCCTGGCAG | | | |
| | CTTGGCCATGGGCCCGGTCACGAA | | | |
| | CAAAACGGGCCTGGACGCCTCGCC | | | |
| | CCTGGCCGCAGATACCTCCTACTAC | | | |
| | CAGGGGGTGTACTCCCGGCCCATTA | | | |
| | TGAACTCCTCTTTG | | | |
| FOXA3 | ATGCTGGGCTCAGTGAAGATGGAG | 47 | Involved in cell | Friedman, J. |
| | GCCCATGACCTGGCCGAGTGGAGC | | glucose | R. et al. The |
| | TACTACCCGGAGGCGGGCGAGGTC | | homeostasis | Foxa family |
| | TACTCGCCGGTGACCCCAGTGCCCA | | | of |
| | CCATGGCCCCCCTCAACTCCTACAT | | | transcription |
| | GACCCTGAATCCTCTAAGCTCTCCC | | | factors in |
| | TATCCCCCTGGGGGGCTCCCTGCCT | | | development |
| | CCCCACTGCCCTCAGGACCCCTGGC | | | and |
| | ACCCCCAGCACCTGCAGCCCCCCTG | | | metabolism. |
| | GGGCCCACTTTCCCAGGCCTGGGTG | | | Cell. Mol. |
| | TCAGCGGTGGCAGCAGCAGCTCCG | | | Life Sci. 63, |
| | GGTACGGGGCCCCGGGTCCTGGGC | | | 2317-2328 |
| | TGGTGCACGGGAAGGAGATGCCGA | | | (2006). |
| | AGGGGTATCGGCGGCCCCTGGCAC | | | |
| | ACGCCAAGCCACCGTATTCCTATAT | | | |
| | CTCACTCATCACCATGGCCATCCAG | | | |
| | CAGGCGCCGGGCAAGATGCTGACC | | | |
| | TTGAGTGAAATCTACCAGTGGATCA | | | |
| | TGGACCTCTTCCCTTACTACCGGGA | | | |
| | GAATCAGCAGCGCTGGCAGAACTC | | | |
| | CATTCGCCACTCGCTGTCTTTCAAC | | | |
| | GACTGCTTCGTCAAGGTGGCGCGTT | | | |
| | CCCCAGACAAGCCTGGCAAGGGCT | | | |
| | CCTACTGGGCCCTACACCCCAGCTC | | | |
| | AGGGAACATGTTTGAGAATGGCTG | | | |
| | CTACCTGCGCCGCCAGAAACGCTTC | | | |
| | AAGCTGGAGGAGAAGGTGAAAAAA | | | |
| | GGGGGCAGCGGGGCTGCCACCACC | | | |
| | ACCAGGAACGGGACAGGGTCTGCT | | | |
| | GCCTCGACCACCACCCCCGCGGCC | | | |
| | ACAGTCACCTCCCCGCCCCAGCCCC | | | |
| | CGCCTCCAGCCCCTGAGCCTGAGGC | | | |
| | CCAGGGCGGGGAAGATGTGGGGGC | | | |
| | TCTGGACTGTGGCTCACCCGCTTCC | | | |
| | TCCACACCCTATTTCACTGGCCTGG | | | |
| | AGCTCCCAGGGGAGCTGAAGCTGG | | | |
| | ACGCGCCCTACAACTTCAACCACCC | | | |
| | TTTCTCCATCAACAACCTAATGTCA | | | |
| | GAACAGACACCAGCACCTCCCAAA | | | |
| | CTGGACGTGGGGTTTGGGGGCTAC | | | |
| | GGGGCTGAAGGTGGGGAGCCTGGA | | | |
| | GTCTACTACCAGGGCCTCTATTCCC | | | |
| | GCTCTTTGCTTAATGCATCC | | | |
| FOXP1 | ATGATGCAAGAATCTGGGACTGAG | 48 | Involved in | Hu, H. et al. |
| | ACAAAAAGTAACGGTTCAGCCATC | | development of | Foxp1 is an |
| | CAGAATGGGTCGGGCGGCAGCAAC | | haematopoetic | essential |
| | CACTTACTAGAGTGCGGCGGTCTTC | | cells, lung and | transcriptional |
| | GGGAGGGGCGGTCCAACGGAGAGA | | oesophagus, and | regulator of B |
| | CGCCGGCCGTGGACATCGGGGCAG | | neuronal | cell |
| | CTGACCTCGCCCACGCCCAGCAGC | | development | development. |
| | AGCAGCAACAGTGGCATCTCATAA | | | Nat. |
| | ACCATCAGCCCTCTAGGAGTCCCAG | | | Immunol. 7, |
| | CAGTTGGCTTAAGAGACTAATTTCA | | | 819-826 |
| | AGCCCTTGGGAGTTGGAAGTCCTGC | | | (2006). |
| | AGGTCCCCTTGTGGGGAGCAGTTGC | | | Shu, W. et al. |
| | TGAGACGAAGATGAGTGGACCTGT | | | Foxp2 and |
| | GTGTCAGCCTAACCCTTCCCCATTT | | | Foxp1 |
| | | | | cooperatively |
| | | | | regulate lung |
| | | | | and |
| | | | | esophagus |
| | | | | development. |
| | | | | Development |
| | | | | 134, 1991- |
| | | | | 2000 (2007). |
| | | | | Bacon, C. et |
| | | | | al. Brain- |
| | | | | specific |
| | | | | Foxp1 |
| | | | | deletion |
| | | | | impairs |
| | | | | neuronal |
| | | | | development |
| | | | | and causes |
| | | | | autistic-like |
| | | | | behaviour. |
| | | | | Mol. |
| | | | | Psychiatry 20, |
| | | | | 632-639 |
| | | | | (2015). |
| GATA1 | ATGGAGTTCCCTGGCCTGGGGTCCC | 49 | Involved in | Fujiwara, Y., |
| | TGGGGACCTCAGAGCCCCTCCCCCA | | erythroid | Browne, C. |
| | GTTTGTGGATCCTGCTCTGGTGTCC | | development | P., Cunniff, |
| | TCCACACCAGAATCAGGGGTTTTCT | | | K., Goff, S. |
| | TCCCCTCTGGGCCTGAGGGCTTGGA | | | C. & Orkin, |
| | TGCAGCAGCTTCCTCCACTGCCCCG | | | S. H. Arrested |
| | AGCACAGCCACCGCTGCAGCTGCG | | | development |
| | GCACTGGCCTACTACAGGGACGCT | | | of embryonic |
| | GAGGCCTACAGACACTCCCCAGTCT | | | red cell |
| | TTCAGGTGTACCCATTGCTCAACTG | | | precursors in |
| | TATGGAGGGGATCCCAGGGGGCTC | | | mouse |
| | ACCATATGCCGGCTGGGCCTACGG | | | embryos |
| | CAAGACGGGGCTCTACCCTGCCTCA | | | lacking |
| | ACTGTGTGTCCCACCCGCGAGGACT | | | transcription |
| | CTCCTCCCCAGGCCGTGGAAGATCT | | | factor GATA- |
| | GGATGGAAAAGGCAGCACCAGCTT | | | 1. PNAS 93, |
| | CCTGGAGACTTTGAAGACAGAGCG | | | 12355-12358 |
| | GCTGAGCCCAGACCTCCTGACCCTG | | | (1996). |
| | GGACCTGCACTGCCTTCATCACTCC | | | |
| | CTGTCCCCAATAGTGCTTATGGGGG | | | |
| | CCCTGACTTTTCCAGTACCTTCTTTT | | | |
| | CTCCCACCGGGAGCCCCCTCAATTC | | | |
| | AGCAGCCTATTCCTCTCCCAAGCTT | | | |
| | CGTGGAACTCTCCCCCTGCCTCCCT | | | |
| | GTGAGGCCAGGGAGTGTGTGAACT | | | |
| | GCGGAGCAACAGCCACTCCACTGT | | | |
| | GGCGGAGGGACAGGACAGGCCACT | | | |
| | ACCTATGCAACGCCTGCGGCCTCTA | | | |
| | TCACAAGATGAATGGGCAGAACAG | | | |
| | GCCCCTCATCCGGCCCAAGAAGCG | | | |
| | CCTGATTGTCAGTAAACGGGCAGG | | | |
| | TACTCAGTGCACCAACTGCCAGAC | | | |
| | GACCACCACGACACTGTGGCGGAG | | | |
| | AAATGCCAGTGGGGATCCCGTGTG | | | |
| | CAATGCCTGCGGCCTCTACTACAAG | | | |
| | CTACACCACCAGCACTACTGTGGTG | | | |
| | GCTCCGCTCAGCTCATGAGGGCAC | | | |
| | AGAGCATGGCCTCCAGAGGAGGGG | | | |
| | TGGTGTCCTTCTCCTCTTGTAGCCA | | | |
| | GAATTCTGGACAACCCAAGTCTCTG | | | |
| | GGCCCCAGGCACCCCCTGGCT | | | |
| GATA2 | ATGGAGGTGGCGCCGGAGCAGCCG | 50 | Involved in | Pimanda, J. E. |
| | CGCTGGATGGCGCACCCGGCCGTG | | haematopoetic | et al. Gata2, |
| | CTGAATGCGCAGCACCCCGACTCA | | development | Fli1, and Scl |
| | CACCACCCGGGCCTGGCGCACAAC | | | form a |
| | TACATGGAACCCGCGCAGCTGCTG | | | recursively |
| | CCTCCAGACGAGGTGGACGTCTTCT | | | wired gene- |
| | TCAATCACCTCGACTCGCAGGGCA | | | regulatory |
| | ACCCCTACTATGCCAACCCCGCTCA | | | circuit during |
| | CGCGCGGGCGCGCGTCTCCTACAG | | | early |
| | CCCCGCGCACGCCCGCCTGACCGG | | | hematopoietic |
| | AGGCCAGATGTGCCGCCCACACTT | | | development. |
| | GTTGCACAGCCCGGGTTTGCCCTGG | | | Proc. Natl. |
| | CTGGACGGGGGCAAAGCAGCCCTC | | | Acad. Sci. U. |
| | TCTGCCGCTGCGGCCCACCACCACA | | | S. A. 104, |
| | ACCCCTGGACCGTGAGCCCCTTCTC | | | 17692-7 |
| | CAAGACGCCACTGCACCCCTCAGCT | | | (2007). |
| | GCTGGAGGCCCTGGAGGCCCACTC | | | Lugus, J. J. et |
| | TCTGTGTACCCAGGGGCTGGGGGT | | | al. GATA2 |
| | GGGAGCGGGGGAGGCAGCGGGAG | | | functions at |
| | CTCAGTGGCCTCCCTCACCCCTACA | | | multiple steps |
| | GCAACCCACTCTGGCTCCCACCTTT | | | in |
| | TCGGCTTCCCACCCACGCCACCCAA | | | hemangioblast |
| | AGAAGTGTCTCCTGACCCTAGCACC | | | development |
| | ACGGGGGCTGCGTCTCCAGCCTCAT | | | and |
| | CTTCCGCGGGGGGTAGTGCAGCCC | | | differentiation. |
| | GAGGAGAGGACAAGGACGGCGTCA | | | Development |
| | AGTACCAGGTGTCACTGACGGAGA | | | 134,393-405 |
| | GCATGAAGATGGAAAGTGGCAGTC | | | (2007). |
| | CCCTGCGCCCAGGCCTAGCTACTAT | | | |
| | GGGCACCCAGCCTGCTACACACCA | | | |
| | CCCCATCCCCACCTACCCCTCCTAT | | | |
| | GTGCCGGCGGCTGCCCACGACTAC | | | |
| | AGCAGCGGACTCTTCCACCCCGGA | | | |
| | GGCTTCCTGGGGGGACCGGCCTCC | | | |
| | AGCTTCACCCCTAAGCAGCGCAGC | | | |
| | AAGGCTCGTTCCTGTTCAGAAGGCC | | | |
| | GGGAGTGTGTCAACTGTGGGGCCA | | | |
| | CAGCCACCCCTCTCTGGCGGCGGG | | | |
| | ACGGCACCGGCCACTACCTGTGCA | | | |
| | ATGCCTGTGGCCTCTACCACAAGAT | | | |
| | GAATGGGCAGAACCGACCACTCAT | | | |
| | CAAGCCCAAGCGAAGACTGTCGGC | | | |
| | CGCCAGAAGAGCCGGCACCTGTTG | | | |
| | TGCAAATTGTCAGACGACAACCAC | | | |
| | CACCTTATGGCGCCGAAACGCCAA | | | |
| | CGGGGACCCTGTCTGCAACGCCTGT | | | |
| | GGCCTCTACTACAAGCTGCACAATG | | | |
| | TTAACAGGCCACTGACCATGAAGA | | | |
| | AGGAAGGGATCCAGACTCGGAACC | | | |
| | GGAAGATGTCCAACAAGTCCAAGA | | | |
| | AGAGCAAGAAAGGGGCGGAGTGCT | | | |
| | TCGAGGAGCTGTCAAAGTGCATGC | | | |
| | AGGAGAAGTCATCCCCCTTCAGTGC | | | |
| | AGCTGCCCTGGCTGGACACATGGC | | | |
| | ACCTGTGGGCCACCTCCCGCCCTTC | | | |
| | AGCCACTCCGGACACATCCTGCCCA | | | |
| | CTCCGACGCCCATCCACCCCTCCTC | | | |
| | CAGCCTCTCCTTCGGCCACCCCCAC | | | |
| | CCGTCCAGCATGGTGACCGCCATG | | | |
| | GGC | | | |
| GATA4 | ATGTACCAGAGCCTGGCTATGGCTG | 51 | Involved in | Xin, M. et al. |
| | CTAATCATGGACCTCCCCCTGGAGC | | cardiovascular | A threshold of |
| | CTATGAAGCCGGAGGACCTGGCGC | | development | GATA4 and |
| | TTTTATGCATGGAGCTGGCGCCGCT | | | GATA6 |
| | TCTTCTCCCGTGTATGTGCCTACAC | | | expression is |
| | CTAGAGTGCCCAGCAGCGTGCTGG | | | required for |
| | GCCTTTCTTATCTTCAGGGAGGAGG | | | cardiovascular |
| | AGCAGGATCTGCTTCTGGCGGAGCT | | | development. |
| | TCAGGCGGATCTTCTGGAGGCGCTG | | | Proc. Natl. |
| | CTTCAGGTGCTGGACCTGGAACTCA | | | Acad. Sci. U. |
| | ACAGGGATCTCCTGGATGGTCACA | | | S. A. 103, |
| | GGCAGGAGCTGATGGAGCCGCTTA | | | 11189-94 |
| | TACCCCTCCTCCTGTGAGCCCCAGG | | | (2006). |
| | TTTAGCTTTCCTGGCACAACAGGCT | | | Rivera- |
| | CTTTAGCTGCCGCTGCTGCTGCAGC | | | Feliciano, J. |
| | CGCAGCTAGAGAAGCAGCTGCATA | | | et al. |
| | TTCTAGTGGCGGAGGAGCTGCTGG | | | Development |
| | AGCCGGCTTAGCTGGAAGAGAGCA | | | of heart |
| | GTACGGAAGAGCCGGATTTGCCGG | | | valves |
| | AAGCTATAGCAGCCCTTACCCTGCC | | | requires |
| | TATATGGCCGATGTTGGCGCATCTT | | | Gata4 |
| | GGGCAGCCGCCGCAGCAGCTTCTG | | | expression in |
| | CAGGACCTTTTGACTCACCTGTGCT | | | endothelial- |
| | TCACTCTCTGCCTGGCAGAGCTAAT | | | derived cells. |
| | CCTGCCGCCAGACATCCCAACCTGG | | | Development |
| | ACATGTTCGACGACTTCAGCGAGG | | | 133, 3607-18 |
| | GCAGAGAATGCGTGAACTGCGGAG | | | (2006). |
| | CCATGAGCACCCCCCTTTGGAGAA | | | |
| | GAGACGGCACCGGCCACTACCTTT | | | |
| | GCAATGCCTGTGGCCTGTACCACAA | | | |
| | GATGAACGGCATCAACAGACCCCT | | | |
| | GATCAAGCCCCAGAGAAGACTGAG | | | |
| | CGCTAGCAGAAGAGTGGGCCTGTC | | | |
| | CTGCGCCAATTGCCAGACCACAAC | | | |
| | CACCACACTGTGGAGGAGAAATGC | | | |
| | CGAGGGCGAGCCTGTGTGTAACGC | | | |
| | CTGTGGACTGTACATGAAGCTGCAC | | | |
| | GGCGTGCCCAGACCTCTGGCCATG | | | |
| | AGAAAGGAGGGCATCCAGACCAGA | | | |
| | AAGAGAAAGCCCAAGAACCTGAAC | | | |
| | AAGAGCAAGACCCCCGCTGCTCCTT | | | |
| | CTGGAAGCGAGAGCCTGCCTCCAG | | | |
| | CCTCTGGAGCCAGCAGCAATAGCT | | | |
| | CTAACGCCACCACATCTTCTTCTGA | | | |
| | GGAGATGAGGCCCATCAAAACCGA | | | |
| | GCCAGGCCTGAGCAGCCACTACGG | | | |
| | CCACAGCTCTAGCGTGAGCCAGAC | | | |
| | TTTTAGCGTGTCTGCCATGTCAGGC | | | |
| | CACGGACCTAGCATTCACCCTGTGC | | | |
| | TGAGCGCCCTGAAGTTGAGCCCAC | | | |
| | AGGGCTATGCTTCTCCTGTGTCTCA | | | |
| | GAGCCCTCAGACCTCCAGCAAGCA | | | |
| | GGACTCCTGGAATTCTCTGGTGCTG | | | |
| | GCCGACAGCCACGGCGATATCATC | | | |
| | ACCGCC | | | |
| GATA6 | ATGGCCCTGACCGACGGCGGATGG | 52 | Involved in | Xin, M. et al. |
| | TGTCTCCCTAAAAGATTCGGCGCCG | | cardiac, lung, | A threshold of |
| | CTGGCGCTGATGCTTCTGACAGCAG | | endoderm and | GATA4 and |
| | AGCCTTCCCCGCTAGGGAACCCAG | | extraembryonic | GATA6 |
| | CACACCACCTAGCCCCATCAGCAG | | development | expression is |
| | CTCAAGCTCTAGCTGTAGCAGAGG | | | required for |
| | CGGAGAGAGAGGACCTGGAGGCGC | | | cardiovascular |
| | TTCTAACTGCGGCACACCTCAGCTG | | | development. |
| | GATACAGAAGCCGCCGCCGGACCA | | | Proc. Natl. |
| | CCAGCCAGATCTCTTTTACTTAGCA | | | Acad. Sci. U. |
| | GCTACGCCAGCCACCCTTTTGGCGC | | | S. A. 103, |
| | TCCTCATGGACCCTCTGCTCCTGGT | | | 11189-94 |
| | GTGGCCGGACCTGGCGGAAACCTG | | | (2006). |
| | AGCTCTTGGGAGGACCTTCTGCTGT | | | Morrisey, E. |
| | TTACCGACCTGGACCAGGCTGCCAC | | | E. et al. |
| | CGCTAGCAAGCTTCTGTGGAGCAG | | | GATA6 |
| | CAGGGGCGCTAAGCTGAGCCCTTTT | | | regulates |
| | GCCCCTGAGCAGCCCGAGGAGATG | | | HNF4 and is |
| | TACCAGACCCTGGCTGCTTTAAGCT | | | required for |
| | CTCAGGGACCTGCCGCTTATGACGG | | | differentiation |
| | AGCCCCTGGTGGATTTGTTCACTCA | | | of visceral |
| | GCGGCAGCAGCCGCAGCTGCTGCA | | | endoderm in |
| | GCCGCTGCCAGCTCACCTGTGTATG | | | the mouse |
| | TGCCTACCACAAGAGTGGGCAGCA | | | embryo. |
| | TGTTACCTGGACTTCCTTACCATCT | | | Genes Dev. |
| | GCAGGGCAGCGGAAGCGGCCCTGC | | | 12, 3579- |
| | TAACCATGCCGGAGGAGCTGGAGC | | | 3590 (1998). |
| | TCACCCCGGATGGCCTCAGGCTTCT | | | Koutsourakis, |
| | GCAGATTCTCCTCCTTATGGATCTG | | | M.; |
| | GAGGAGGAGCAGCTGGAGGGGGA | | | Langeveld, |
| | GCTGCAGGACCAGGTGGAGCCGGA | | | A.; Patient, |
| | AGCGCAGCAGCACATGTGTCTGCC | | | R.; |
| | AGATTTCCCTATAGCCCTAGCCCTC | | | Beddington, |
| | CTATGGCCAATGGCGCTGCTAGAG | | | R.; Grosveld, |
| | AACCCGGAGGATATGCTGCGGCAG | | | F. The |
| | GCTCTGGCGGCGCTGGCGGAGTTTC | | | transcription |
| | TGGAGGTGGATCTTCACTGGCCGCT | | | factor |
| | ATGGGAGGAAGAGAGCCTCAGTAC | | | GATA6 is |
| | TCTTCTCTGAGCGCCGCTAGACCAC | | | essential for |
| | TGAACGGCACCTATCATCACCACCA | | | early |
| | CCATCACCATCATCATCACCCCAGC | | | extraembryonic |
| | CCTTACTCCCCTTATGTGGGAGCCC | | | development. |
| | CCCTTACACCCGCTTGGCCTGCCGG | | | Development |
| | CCCTTTCGAGACACCTGTGCTGCAC | | | 126, 723-732 |
| | AGCCTTCAGTCTAGAGCTGGCGCAC | | | (1999). |
| | CTTTACCAGTGCCTAGAGGCCCCTC | | | Zhang, Y. et |
| | TGCCGACTTGCTGGAGGATCTGAGC | | | al. A Gata6- |
| | GAGAGCAGAGAGTGCGTGAACTGT | | | Wnt pathway |
| | GGCAGCATCCAGACACCCCTGTGG | | | required for |
| | AGAAGAGACGGCACCGGCCACTAC | | | epithelial |
| | CTGTGCAACGCTTGCGGCCTGTACA | | | stem cell |
| | GCAAGATGAATGGGCTGAGCAGAC | | | development |
| | CCCTGATCAAGCCCCAGAAGAGGG | | | and airway |
| | TGCCCAGCAGCAGACGGCTGGGAC | | | regeneration. |
| | TGAGCTGCGCCAACTGTCATACCAC | | | Nat. Genet. |
| | AACAACCACACTGTGGCGGAGAAA | | | 40, 862-870 |
| | CGCCGAGGGCGAGCCCGTGTGTAA | | | (2008). |
| | CGCCTGCGGCCTTTACATGAAGCTG | | | |
| | CACGGCGTGCCCAGACCTCTGGCC | | | |
| | ATGAAGAAGGAGGGAATCCAGACC | | | |
| | AGAAAGAGAAAGCCCAAGAACATC | | | |
| | AACAAGAGCAAGACCTGCAGCGGC | | | |
| | AACAGCAACAACAGCATCCCCATG | | | |
| | ACCCCCACCAGCACATCTAGCAAC | | | |
| | AGCGACGACTGTAGCAAGAACACA | | | |
| | TCACCTACCACCCAGCCCACAGCTA | | | |
| | GCGGAGCCGGCGCCCCCGTGATGA | | | |
| | CAGGCGCCGGAGAGTCCACAAATC | | | |
| | CCGAGAATAGCGAACTGAAGTACT | | | |
| | CTGGACAGGACGGACTGTATATCG | | | |
| | GCGTGAGCCTGGCTTCTCCCGCCGA | | | |
| | GGTGACCAGCTCTGTCAGACCTGAC | | | |
| | TCTTGGTGTGCCCTCGCCCTGGCC | | | |
| GLI1 | ATGTTCAACTCGATGACCCCACCAC | 53 | Involved in | Lee, J. et al. |
| | CAATCAGTAGCTATGGCGAGCCCT | | neural stem cell | Gli1 is a |
| | GCTGTCTCCGGCCCCTCCCCAGTCA | | proliferation | target of |
| | GGGGGCCCCCAGTGTGGGGACAGA | | and neural tube | Sonic |
| | AGGACTGTCTGGCCCGCCCTTCTGC | | development | hedgehog that |
| | CACCAAGCTAACCTCATGTCCGGCC | | | induces |
| | CCCACAGTTATGGGCCAGCCAGAG | | | ventral neural |
| | AGACCAACAGCTGCACCGAGGGCC | | | tube |
| | CACTCTTTTCTTCTCCCCGGAGTGC | | | development. |
| | AGTCAAGTTGACCAAGAAGCGGGC | | | Development |
| | ACTGTCCATCTCACCTCTGTCGGAT | | | 124, 2537- |
| | GCCAGCCTGGACCTGCAGACGGTT | | | 2552 (1997). |
| | ATCCGCACCTCACCCAGCTCCCTCG | | | Palma, V. et |
| | TAGCTTTCATCAACTCGCGATGCAC | | | al. Sonic |
| | ATCTCCAGGAGGCTCCTACGGTCAT | | | hedgehog |
| | CTCTCCATTGGCACCATGAGCCCAT | | | controls stem |
| | CTCTGGGATTCCCAGCCCAGATGAA | | | cell behavior |
| | TCACCAAAAAGGGCCCTCGCCTTCC | | | in the |
| | TTTGGGGTCCAGCCTTGTGGTCCCC | | | postnatal and |
| | ATGACTCTGCCCGGGGTGGGATGA | | | adult brain. |
| | TCCCACATCCTCAGTCCCGGGGACC | | | Development |
| | CTTCCCAACTTGCCAGCTGAAGTCT | | | 132, 335-44 |
| | GAGCTGGACATGCTGGTTGGCAAG | | | (2005). |
| | TGCCGGGAGGAACCCTTGGAAGGT | | | |
| | GATATGTCCAGCCCCAACTCCACAG | | | |
| | GCATACAGGATCCCCTGTTGGGGAT | | | |
| | GCTGGATGGGCGGGAGGACCTCGA | | | |
| | GAGAGAGGAGAAGCGTGAGCCTGA | | | |
| | ATCTGTGTATGAAACTGACTGCCGT | | | |
| | TGGGATGGCTGCAGCCAGGAATTT | | | |
| | GACTCCCAAGAGCAGCTGGTGCAC | | | |
| | CACATCAACAGCGAGCACATCCAC | | | |
| | GGGGAGCGGAAGGAGTTCGTGTGC | | | |
| | CACTGGGGGGGCTGCTCCAGGGAG | | | |
| | CTGAGGCCCTTCAAAGCCCAGTAC | | | |
| | ATGCTGGTGGTTCACATGCGCAGAC | | | |
| | ACACTGGCGAGAAGCCACACAAGT | | | |
| | GCACGTTTGAAGGGTGCCGGAAGT | | | |
| | CATACTCACGCCTCGAAAACCTGA | | | |
| | AGACGCACCTGCGGTCACACACGG | | | |
| | GTGAGAAGCCATACATGTGTGAGC | | | |
| | ACGAGGGCTGCAGTAAAGCCTTCA | | | |
| | GCAATGCCAGTGACCGAGCCAAGC | | | |
| | ACCAGAATCGGACCCATTCCAATG | | | |
| | AGAAGCCGTATGTATGTAAGCTCCC | | | |
| | TGGCTGCACCAAACGCTATACAGA | | | |
| | TCCTAGCTCGCTGCGAAAACATGTC | | | |
| | AAGACAGTGCATGGTCCTGACGCC | | | |
| | CATGTGACCAAACGGCACCGTGGG | | | |
| | GATGGCCCCCTGCCTCGGGCACCAT | | | |
| | CCATTTCTACAGTGGAGCCCAAGA | | | |
| | GGGAGCGGGAAGGAGGTCCCATCA | | | |
| | GGGAGGAAAGCAGACTGACTGTGC | | | |
| | CAGAGGGTGCCATGAAGCCACAGC | | | |
| | CAAGCCCTGGGGCCCAGTCATCCTG | | | |
| | CAGCAGTGACCACTCCCCGGCAGG | | | |
| | GAGTGCAGCCAATACAGACAGTGG | | | |
| | TGTGGAAATGACTGGCAATGCAGG | | | |
| | GGGCAGCACTGAAGACCTCTCCAG | | | |
| | CTTGGACGAGGGACCTTGCATTGCT | | | |
| | GGCACTGGTCTGTCCACTCTTCGCC | | | |
| | GCCTTGAGAACCTCAGGCTGGACC | | | |
| | AGCTACATCAACTCCGGCCAATAG | | | |
| | GGACCCGGGGTCTCAAACTGCCCA | | | |
| | GCTTGTCCCACACCGGTACCACTGT | | | |
| | GTCCCGCCGCGTGGGCCCCCCAGTC | | | |
| | TCTCTTGAACGCCGCAGCAGCAGCT | | | |
| | CCAGCAGCATCAGCTCTGCCTATAC | | | |
| | TGTCAGCCGCCGCTCCTCCCTGGCC | | | |
| | TCTCCTTTCCCCCCTGGCTCCCCAC | | | |
| | CAGAGAATGGAGCATCCTCCCTGC | | | |
| | CTGGCCTTATGCCTGCCCAGCACTA | | | |
| | CCTGCTTCGGGCAAGATATGCTTCA | | | |
| | GCCAGAGGGGGTGGTACTTCGCCC | | | |
| | ACTGCAGCATCCAGCCTGGATCGG | | | |
| | ATAGGTGGTCTTCCCATGCCTCCTT | | | |
| | GGAGAAGCCGAGCCGAGTATCCAG | | | |
| | GATACAACCCCAATGCAGGGGTCA | | | |
| | CCCGGAGGGCCAGTGACCCAGCCC | | | |
| | AGGCTGCTGACCGTCCTGCTCCAGC | | | |
| | TAGAGTCCAGAGGTTCAAGAGCCT | | | |
| | GGGCTGTGTCCATACCCCACCCACT | | | |
| | GTGGCAGGGGGAGGACAGAACTTT | | | |
| | GATCCTTACCTCCCAACCTCTGTCT | | | |
| | ACTCACCACAGCCCCCCAGCATCA | | | |
| | CTGAGAATGCTGCCATGGATGCTA | | | |
| | GAGGGCTACAGGAAGAGCCAGAAG | | | |
| | TTGGGACCTCCATGGTGGGCAGTG | | | |
| | GTCTGAACCCCTATATGGACTTCCC | | | |
| | ACCTACTGATACTCTGGGATATGGG | | | |
| | GGACCTGAAGGGGCAGCAGCTGAG | | | |
| | CCTTATGGAGCGAGGGGTCCAGGC | | | |
| | TCTCTGCCTCTTGGGCCTGGTCCAC | | | |
| | CCACCAACTATGGCCCCAACCCCTG | | | |
| | TCCCCAGCAGGCCTCATATCCTGAC | | | |
| | CCCACCCAAGAAACATGGGGTGAG | | | |
| | TTCCCTTCCCACTCTGGGCTGTACC | | | |
| | CAGGCCCCAAGGCTCTAGGTGGAA | | | |
| | CCTACAGCCAGTGTCCTCGACTTGA | | | |
| | ACATTATGGACAAGTGCAAGTCAA | | | |
| | GCCAGAACAGGGGTGCCCAGTGGG | | | |
| | GTCTGACTCCACAGGACTGGCACCC | | | |
| | TGCCTCAATGCCCACCCCAGTGAGG | | | |
| | GGCCCCCACATCCACAGCCTCTCTT | | | |
| | TTCCCATTACCCCCAGCCCTCTCCT | | | |
| | CCCCAATATCTCCAGTCAGGCCCCT | | | |
| | ATACCCAGCCACCCCCTGATTATCT | | | |
| | TCCTTCAGAACCCAGGCCTTGCCTG | | | |
| | GACTTTGATTCCCCCACCCATTCCA | | | |
| | CAGGGCAGCTCAAGGCTCAGCTTG | | | |
| | TGTGTAATTATGTTCAATCTCAACA | | | |
| | GGAGCTACTGTGGGAGGGTGGGGG | | | |
| | CAGGGAAGATGCCCCCGCCCAGGA | | | |
| | ACCTTCCTACCAGAGTCCCAAGTTT | | | |
| | CTGGGGGGTTCCCAGGTTAGCCCA | | | |
| | AGCCGTGCTAAAGCTCCAGTGAAC | | | |
| | ACATATGGACCTGGCTTTGGACCCA | | | |
| | ACTTGCCCAATCACAAGTCAGGTTC | | | |
| | CTATCCCACCCCTTCACCATGCCAT | | | |
| | GAAAATTTTGTAGTGGGGGCAAAT | | | |
| | AGGGCTTCACATAGGGCAGCAGCA | | | |
| | CCACCTCGACTTCTGCCCCCATTGC | | | |
| | CCACTTGCTATGGGCCTCTCAAAGT | | | |
| | GGGAGGCACAAACCCCAGCTGTGG | | | |
| | TCATCCTGAGGTGGGCAGGCTAGG | | | |
| | AGGGGGTCCTGCCTTGTACCCTCCT | | | |
| | CCCGAAGGACAGGTATGTAACCCC | | | |
| | CTGGACTCTCTTGATCTTGACAACA | | | |
| | CTCAGCTGGACTTTGTGGCTATTCT | | | |
| | GGATGAGCCCCAGGGGCTGAGTCC | | | |
| | TCCTCCTTCCCATGATCAGCGGGGC | | | |
| | AGCTCTGGACATACCCCACCTCCCT | | | |
| | CTGGGCCCCCCAACATGGCTGTGG | | | |
| | GCAACATGAGTGTCTTACTGAGATC | | | |
| | CCTACCTGGGGAAACAGAATTCCTC | | | |
| | AACTCTAGTGCC | | | |
| HAND2 | ATGAGTCTGGTAGGTGGTTTTCCCC | 54 | Involved in | Srivastava, D. |
| | ACCACCCGGTGGTGCACCACGAGG | | cardiac | et al. |
| | GCTACCCGTTTGCCGCCGCCGCCGC | | development | Regulation of |
| | CGCCAGCCGCTGCAGCCATGAGGA | | | cardiac |
| | GAACCCCTACTTCCATGGCTGGCTC | | | mesodermal |
| | ATCGGCCACCCCGAGATGTCGCCCC | | | and neural |
| | CCGACTACAGCATGGCCCTGTCCTA | | | crest |
| | CAGCCCCGAGTATGCCAGCGGCAC | | | development |
| | CGCCAACCGCAAGGAGCGGCGCAG | | | by the bHLH |
| | GACTCAGAGCATCAACAGCGCCTT | | | transcription |
| | CGCCGAACTGCGCGAGTGCATCCC | | | factor, |
| | CAACGTACCCGCCGACACCAAACT | | | dHAND. Nat. |
| | CTCCAAAATCAAGACCCTGCGCCTG | | | Genet. 16, |
| | GCCACCAGCTACATCGCCTACCTCA | | | 154-160 |
| | TGGACCTGCTGGCCAAGGACGACC | | | (1997). |
| | AGAATGGCGAGGCGGAGGCCTTCA | | | |
| | AGGCAGAGATCAAGAAGACCGACG | | | |
| | TGAAAGAGGAGAAGAGGAAGAAG | | | |
| | GAGCTGAACGAAATCTTGAAAAGC | | | |
| | ACAGTGAGCAGCAACGACAAGAAA | | | |
| | ACCAAAGGCCGGACGGGCTGGCCG | | | |
| | CAGCACGTCTGGGCCCTGGAGCTC | | | |
| | AAGCAG | | | |
| HNF1A | ATGGTTTCTAAACTGAGCCAGCTGC | 55 | Involved in | D' Angelo, A. |
| | AGACGGAGCTCCTGGCGGCCCTGC | | liver, kidney, | et al. |
| | TGGAGTCAGGGCTGAGCAAAGAGG | | pancreatic and | Hepatocyte |
| | CACTGCTCCAGGCACTGGGTGAGC | | gut | nuclear factor |
| | CGGGGCCCTACCTCCTGGCTGGAG | | development | 1alpha and |
| | AAGGCCCCCTGGACAAGGGGGAGT | | | beta control |
| | CCTGCGGCGGCGGTCGAGGGGAGC | | | terminal |
| | TGGCTGAGCTGCCCAATGGGCTGG | | | differentiation |
| | GGGAGACTCGGGGCTCCGAGGACG | | | and cell fate |
| | AGACGGACGACGATGGGGAAGACT | | | commitment |
| | TCACGCCACCCATCCTCAAAGAGCT | | | in the gut |
| | GGAGAACCTCAGCCCTGAGGAGGC | | | epithelium. |
| | GGCCCACCAGAAAGCCGTGGTGGA | | | Development |
| | GACCCTTCTGCAGGAGGACCCGTG | | | 137,1573-82 |
| | GCGTGTGGCGAAGATGGTCAAGTC | | | (2010). |
| | CTACCTGCAGCAGCACAACATCCC | | | Servitj a, J.-M. |
| | ACAGCGGGAGGTGGTCGATACCAC | | | et al. |
| | TGGCCTCAACCAGTCCCACCTGTCC | | | Hnf1 alpha |
| | CAACACCTCAACAAGGGCACTCCC | | | (MODY3) |
| | ATGAAGACGCAGAAGCGGGCCGCC | | | controls |
| | CTGTACACCTGGTACGTCCGCAAGC | | | tissue-specific |
| | AGCGAGAGGTGGCGCAGCAGTTCA | | | transcriptional |
| | CCCATGCAGGGCAGGGAGGGCTGA | | | programs and |
| | TTGAAGAGCCCACAGGTGATGAGC | | | exerts |
| | TACCAACCAAGAAGGGGCGGAGGA | | | opposed |
| | ACCGTTTCAAGTGGGGCCCAGCATC | | | effects on cell |
| | CCAGCAGATCCTGTTCCAGGCCTAT | | | growth in |
| | GAGAGGCAGAAGAACCCTAGCAAG | | | pancreatic |
| | GAGGAGCGAGAGACGCTAGTGGAG | | | islets and |
| | GAGTGCAATAGGGCGGAATGCATC | | | liver. Mol. |
| | CAGAGAGGGGTGTCCCCATCACAG | | | Cell. Biol. 29, |
| | GCACAGGGGCTGGGCTCCAACCTC | | | 2945-59 |
| | GTCACGGAGGTGCGTGTCTACAACT | | | (2009). |
| | GGTTTGCCAACCGGCGCAAAGAAG | | | Si-Tayeb, K.; |
| | AAGCCTTCCGGCACAAGCTGGCCA | | | Lemaigre, F. |
| | TGGACACGTACAGCGGGCCCCCCC | | | P.; Duncan, S. |
| | CAGGGCCAGGCCCGGGACCTGCGC | | | A. |
| | TGCCCGCTCACAGCTCCCCTGGCCT | | | Organogenesis |
| | GCCTCCACCTGCCCTCTCCCCCAGT | | | and |
| | AAGGTCCACGGTGTGCGCTATGGA | | | Development |
| | CAGCCTGCGACCAGTGAGACTGCA | | | of the Liver. |
| | GAAGTACCCTCAAGCAGCGGCGGT | | | Dev. Cell 18, |
| | CCCTTAGTGACAGTGTCTACACCCC | | | 175-189 |
| | TCCACCAAGTGTCCCCCACGGGCCT | | | (2010). |
| | GGAGCCCAGCCACAGCCTGCTGAG | | | Martovetsky, |
| | TACAGAAGCCAAGCTGGTCTCAGC | | | G., Tee, J. B. |
| | AGCTGGGGGCCCCCTCCCCCCTGTC | | | & Nigam, S. |
| | AGCACCCTGACAGCACTGCACAGC | | | K. Hepatocyte |
| | TTGGAGCAGACATCCCCAGGCCTC | | | nuclear |
| | AACCAGCAGCCCCAGAACCTCATC | | | factors 4α and |
| | ATGGCCTCACTTCCTGGGGTCATGA | | | 1α regulate |
| | CCATCGGGCCTGGTGAGCCTGCCTC | | | kidney |
| | CCTGGGTCCTACGTTCACCAACACA | | | developmental |
| | GGTGCCTCCACCCTGGTCATCGGCC | | | expression |
| | TGGCCTCCACGCAGGCACAGAGTG | | | of drug- |
| | TGCCGGTCATCAACAGCATGGGCA | | | metabolizing |
| | GCAGCCTGACCACCCTGCAGCCCGT | | | enzymes and |
| | CCAGTTCTCCCAGCCGCTGCACCCC | | | drug |
| | TCCTACCAGCAGCCGCTCATGCCAC | | | transporters. |
| | CTGTGCAGAGCCATGTGACCCAGA | | | Mol. |
| | GCCCCTTCATGGCCACCATGGCTCA | | | Pharmacol. |
| | GCTGCAGAGCCCCCACGCCCTCTAC | | | 84,808-23 |
| | AGCCACAAGCCCGAGGTGGCCCAG | | | (2013). |
| | TACACCCACACAGGCCTGCTCCCGC | | | |
| | AGACTATGCTCATCACCGACACCAC | | | |
| | CAACCTGAGCGCCCTGGCCAGCCTC | | | |
| | ACGCCCACCAAGCAGGTCTTCACCT | | | |
| | CAGACACTGAGGCCTCCAGTGAGT | | | |
| | CCGGGCTTCACACGCCGGCATCTCA | | | |
| | GGCCACCACCCTCCACGTCCCCAGC | | | |
| | CAGGACCCTGCCGGCATCCAGCAC | | | |
| | CTGCAGCCGGCCCACCGGCTCAGC | | | |
| | GCCAGCCCCACAGTGTCCTCCAGCA | | | |
| | GCCTGGTGCTGTACCAGAGCTCAG | | | |
| | ACTCCAGCAATGGCCAGAGCCACC | | | |
| | TGCTGCCATCCAACCACAGCGTCAT | | | |
| | CGAGACCTTCATCTCCACCCAGATG | | | |
| | GCCTCTTCCTCCCAGTTG | | | |
| HNF1B | ATGGTTAGCAAACTGACATCCCTCC | 56 | Involved in | D' Angelo, A. |
| | AGCAGGAACTTCTTTCTGCCCTCCT | | liver, kidney, | et al. |
| | CTCCAGTGGGGTAACCAAAGAGGT | | pancreatic and | Hepatocyte |
| | ACTGGTCCAGGCTTTGGAGGAGTTG | | gut | nuclear factor |
| | CTCCCCTCACCGAATTTTGGTGTAA | | development | 1alpha and |
| | AGTTGGAGACTCTCCCCCTCTCCCC | | | beta control |
| | TGGTTCTGGAGCAGAGCCGGATAC | | | terminal |
| | TAAACCGGTATTTCATACGCTTACA | | | differentiation |
| | AACGGACACGCAAAGGGTCGGCTT | | | and cell fate |
| | TCAGGTGACGAAGGGTCTGAGGAC | | | commitment |
| | GGCGATGATTATGACACCCCGCCC | | | in the gut |
| | ATCCTCAAAGAACTGCAGGCCCTTA | | | epithelium. |
| | ATACAGAGGAAGCGGCGGAGCAGC | | | Development |
| | GAGCTGAAGTTGACAGAATGCTCT | | | 137,1573-82 |
| | CAGAAGATCCGTGGAGAGCTGCGA | | | (2010). |
| | AAATGATTAAGGGATATATGCAGC | | | Si-Tayeb, K.; |
| | AACATAACATTCCCCAGAGAGAGG | | | Lemaigre, F. |
| | TAGTTGATGTTACCGGCCTTAACCA | | | P.; Duncan, S. |
| | GAGCCACCTGTCTCAGCATCTCAAT | | | A. |
| | AAGGGTACTCCTATGAAAACACAG | | | Organogenesis |
| | AAGCGAGCGGCCCTTTACACATGG | | | and |
| | TACGTGCGGAAGCAACGAGAAATT | | | Development |
| | CTCCGACAGTTCAATCAGACAGTAC | | | of the Liver. |
| | AATCTTCAGGGAACATGACGGATA | | | Dev. Cell 18, |
| | AAAGCTCACAGGATCAGCTCTTGTT | | | 175-189 |
| | TCTCTTCCCCGAGTTCAGCCAACAG | | | (2010). |
| | TCCCACGGTCCAGGTCAATCTGATG | | | Clissold, R. |
| | ATGCTTGCAGTGAACCTACAAACA | | | L., Hamilton, |
| | AAAAAATGAGGAGGAACAGGTTTA | | | A. J., |
| | AATGGGGACCGGCCTCTCAGCAGA | | | Hattersley, A. |
| | TACTGTACCAAGCGTACGATCGGC | | | T., Ellard, S. |
| | AGAAAAACCCAAGCAAAGAGGAGC | | | & Bingham, |
| | GCGAGGCATTGGTCGAGGAGTGTA | | | C. HNF1B- |
| | ATCGGGCCGAGTGCTTGCAACGGG | | | associated |
| | GTGTAAGTCCTAGCAAAGCCCATG | | | renal and |
| | GTCTCGGCTCAAACTTGGTCACGGA | | | extra-renal |
| | GGTGAGGGTATATAATTGGTTTGCC | | | disease-an |
| | AACAGGCGGAAGGAGGAAGCATTC | | | expanding |
| | CGGCAAAAGCTGGCGATGGATGCC | | | clinical |
| | TACTCAAGCAACCAGACACATAGC | | | spectrum. |
| | CTCAACCCTCTGTTGTCACACGGGT | | | Nat. Rev. |
| | CCCCTCATCACCAACCTTCTTCCTC | | | Nephrol. 11, |
| | TCCACCCAACAAACTTTCTGGTGTC | | | 102-112 |
| | CGATATTCCCAGCAGGGGAACAAC | | | (2014). |
| | GAGATAACATCTTCCTCTACTATAA | | | De Vas, M. |
| | GTCATCACGGAAATTCTGCAATGGT | | | G. et al. |
| | AACGTCACAGAGTGTGTTGCAACA | | | Hnf1b |
| | GGTATCACCCGCGTCTCTTGATCCA | | | controls |
| | GGCCACAATCTGTTGAGCCCTGACG | | | pancreas |
| | GAAAGATGATCTCTGTTTCTGGTGG | | | morphogenesis |
| | CGGACTCCCGCCGGTCTCCACACTT | | | and the |
| | ACCAACATACATAGTCTCAGTCATC | | | generation of |
| | ATAATCCTCAGCAGAGCCAAAACC | | | Ngn3+ |
| | TGATTATGACTCCTCTTAGCGGAGT | | | endocrine |
| | GATGGCTATTGCGCAATCTTTGAAC | | | progenitors. |
| | ACCTCACAAGCACAATCTGTACCCG | | | Development |
| | TCATAAACAGCGTAGCGGGCTCATT | | | 142,871-82 |
| | GGCGGCGCTCCAACCAGTGCAGTT | | | (2015). |
| | CTCCCAGCAGCTCCATTCACCCCAT | | | E1-Khairi, R. |
| | CAACAGCCTCTGATGCAGCAGAGC | | | & Vallier, L. |
| | CCTGGTAGTCACATGGCTCAACAGC | | | The role of |
| | CGTTCATGGCAGCTGTCACTCAGCT | | | hepatocyte |
| | CCAGAACTCCCATATGTATGCCCAC | | | nuclear factor |
| | AAGCAAGAACCACCACAATACAGT | | | 1β in disease |
| | CACACATCAAGATTCCCCAGTGCTA | | | and |
| | TGGTTGTTACTGACACATCCTCTAT | | | development. |
| | CTCAACTCTGACGAACATGTCCAGT | | | Diabetes, |
| | AGTAAACAATGTCCTCTGCAAGCAT | | | Obes. Metab. |
| | GG | | | 18,23-32 |
| | | | | (2016). |
| HNF4A | ATGCGACTCTCCAAAACCCTCGTCG | 57 | Involved in | Si-Tayeb, K.; |
| | ACATGGACATGGCCGACTACAGTG | | liver, kidney, | Lemaigre, F. |
| | CTGCACTGGACCCAGCCTACACCAC | | pancreatic and | P.; Duncan, S. |
| | CCTGGAATTTGAGAATGTGCAGGT | | gut | A. |
| | GTTGACGATGGGCAATGACACGTC | | development | Organogenesis |
| | CCCATCAGAAGGCACCAACCTCAA | | | and |
| | CGCGCCCAACAGCCTGGGTGTCAG | | | Development |
| | CGCCCTGTGTGCCATCTGCGGGGAC | | | of the Liver. |
| | CGGGCCACGGGCAAACACTACGGT | | | Dev. Cell 18, |
| | GCCTCGAGCTGTGACGGCTGCAAG | | | 175-189 |
| | GGCTTCTTCCGGAGGAGCGTGCGG | | | (2010). |
| | AAGAACCACATGTACTCCTGCAGA | | | Martovetsky, |
| | TTTAGCCGGCAGTGCGTGGTGGAC | | | G., Tee, J. B. |
| | AAAGACAAGAGGAACCAGTGCCGC | | | & Nigam, S. |
| | TACTGCAGGCTCAAGAAATGCTTCC | | | K. Hepatocyte |
| | GGGCTGGCATGAAGAAGGAAGCCG | | | nuclear |
| | TCCAGAATGAGCGGGACCGGATCA | | | factors 4α and |
| | GCACTCGAAGGTCAAGCTATGAGG | | | 1α regulate |
| | ACAGCAGCCTGCCCTCCATCAATGC | | | kidney |
| | GCTCCTGCAGGCGGAGGTCCTGTCC | | | developmental |
| | CGACAGATCACCTCCCCCGTCTCCG | | | expression |
| | GGATCAACGGCGACATTCGGGCGA | | | of drug- |
| | AGAAGATTGCCAGCATCGCAGATG | | | metabolizing |
| | TGTGTGAGTCCATGAAGGAGCAGC | | | enzymes and |
| | TGCTGGTTCTCGTTGAGTGGGCCAA | | | drug |
| | GTACATCCCAGCTTTCTGCGAGCTC | | | transporters. |
| | CCCCTGGACGACCAGGTGGCCCTG | | | Mol. |
| | CTCAGAGCCCATGCTGGCGAGCAC | | | Pharmacol. |
| | CTGCTGCTCGGAGCCACCAAGAGA | | | 84,808-23 |
| | TCCATGGTGTTCAAGGACGTGCTGC | | | (2013). |
| | TCCTAGGCAATGACTACATTGTCCC | | | Maestro, M. |
| | TCGGCACTGCCCGGAGCTGGCGGA | | | A. et al. |
| | GATGAGCCGGGTGTCCATACGCAT | | | Distinct roles |
| | CCTTGACGAGCTGGTGCTGCCCTTC | | | of HNF1b eta, |
| | CAGGAGCTGCAGATCGATGACAAT | | | HNF1alpha, |
| | GAGTATGCCTACCTCAAAGCCATCA | | | and |
| | TCTTCTTTGACCCAGATGCCAAGGG | | | HNF4alpha in |
| | GCTGAGCGATCCAGGGAAGATCAA | | | regulating |
| | GCGGCTGCGTTCCCAGGTGCAGGT | | | pancreas |
| | GAGCTTGGAGGACTACATCAACGA | | | development, |
| | CCGCCAGTATGACTCGCGTGGCCGC | | | beta-cell |
| | TTTGGAGAGCTGCTGCTGCTGCTGC | | | function and |
| | CCACCTTGCAGAGCATCACCTGGCA | | | growth. |
| | GATGATCGAGCAGATCCAGTTCATC | | | Endocr. Dev. |
| | AAGCTCTTCGGCATGGCCAAGATTG | | | 12,33-45 |
| | ACAACCTGTTGCAGGAGATGCTGCT | | | (2007). |
| | GGGAGGTCCGTGCCAAGCCCAGGA | | | Garrison, W. |
| | GGGGCGGGGTTGGAGTGGGGACTC | | | D. et al. |
| | CCCAGGAGACAGGCCTCACACAGT | | | Hepatocyte |
| | GAGCTCACCCCTCAGCTCCTTGGCT | | | nuclear factor |
| | TCCCCACTGTGCCGCTTTGGGCAAG | | | 4alpha is |
| | TTGCT | | | essential for |
| | | | | embryonic |
| | | | | development |
| | | | | of the mouse |
| | | | | colon. |
| | | | | Gastroenterol |
| | | | | ogy 130, |
| | | | | 1207-20 |
| | | | | (2006). |
| HOXA1 | ATGGACAACGCGCGGATGAATTCC | 58 | Involved in | Tischfield, M. |
| | TTCCTCGAGTACCCAATTTTGTCTA | | neural and | A. et al. |
| | GTGGAGACAGTGGCACTTGCAGTG | | cardiovascular | Homozygous |
| | CCCGAGCCTATCCATCAGACCACA | | development | HOXA1 |
| | GAATTACAACATTCCAAAGCTGTGC | | | mutations |
| | GGTGTCAGCCAACAGTTGCGGCGG | | | disrupt human |
| | AGACGACCGCTTCCTGGTCGGAAG | | | brainstem, |
| | AGGGGTTCAAATTGGATCACCTCAC | | | inner ear, |
| | CATCACCATCACCACCACCATCACC | | | cardiovascular |
| | ACCCCCAACCGGCGACTTACCAAA | | | and |
| | CCAGCGGCAATTTGGGCGTGAGCT | | | cognitive |
| | ATAGCCATTCCTCATGTGGACCTTC | | | development. |
| | CTATGGGTCTCAGAATTTCTCCGCC | | | Nat. Genet. |
| | CCTTATAGCCCATACGCCCTGAACC | | | 37, 1035- |
| | AAGAGGCCGATGTATCAGGAGGCT | | | 1037 (2005). |
| | ATCCCCAGTGCGCGCCAGCGGTTTA | | | |
| | CTCAGGTAATCTTTCTAGCCCGATG | | | |
| | GTCCAGCACCACCATCACCATCAA | | | |
| | GGTTATGCCGGCGGTGCAGTCGGA | | | |
| | TCCCCACAATACATACACCATAGTT | | | |
| | ACGGCCAAGAGCACCAATCCCTGG | | | |
| | CCCTCGCTACATATAACAACTCACT | | | |
| | GTCTCCGCTTCATGCTTCCCACCAA | | | |
| | GAAGCTTGTCGGAGTCCCGCCTCAG | | | |
| | AAACTTCCTCTCCAGCTCAGACTTT | | | |
| | TGATTGGATGAAGGTCAAGCGGAA | | | |
| | TCCGCCTAAAACGGGCAAAGTAGG | | | |
| | TGAATATGGCTATTTGGGACAGCCT | | | |
| | AATGCTGTCCGCACCAATTTCACAA | | | |
| | CAAAACAGCTTACTGAACTCGAGA | | | |
| | AGGAATTTCATTTTAATAAGTATTT | | | |
| | GACTCGAGCGAGACGAGTCGAAAT | | | |
| | CGCCGCTAGTCTTCAACTTAACGAG | | | |
| | ACCCAGGTTAAGATATGGTTCCAG | | | |
| | AACAGAAGAATGAAACAAAAAAA | | | |
| | GCGGGAGAAGGAAGGACTCCTCCC | | | |
| | TATATCACCAGCCACACCCCCAGGT | | | |
| | AACGACGAGAAGGCGGAGGAATCT | | | |
| | TCAGAGAAGAGTTCCAGCTCCCCTT | | | |
| | GTGTTCCTTCTCCTGGTAGCTCAAC | | | |
| | CAGCGATACCCTCACGACGAGTCA | | | |
| | C | | | |
| HOXA10 | ATGTGTCAAGGCAATTCCAAAGGT | 59 | Involved | Buske, C. et |
| | GAAAACGCAGCCAACTGGCTCACG | | function in | al. |
| | GCAAAGAGTGGTCGGAAGAAGCGC | | fertility, | Overexpression |
| | TGCCCCTACACGAAGCACCAGACA | | embryo | of HOXA10 |
| | CTGGAGCTGGAGAAGGAGTTTCTG | | viability, and | perturbs |
| | TTCAATATGTACCTTACTCGAGAGC | | regulation of | human |
| | GGCGCCTAGAGATTAGCCGCAGCG | | hematopoetic | lympho- |
| | TCCACCTCACGGACAGACAAGTGA | | lineage | myelopoiesis in |
| | AAATCTGGTTTCAGAACCGCAGGA | | commitment | vitro and in |
| | TGAAACTGAAGAAAATGAATCGAG | | | vivo. Blood |
| | AAAACCGGATCCGGGAGCTCACAG | | | 97, 2286- |
| | CCAACTTTAATTTTTCC | | | 2292 (2001). |
| | | | | Satokata, I., |
| | | | | Benson, G. & |
| | | | | Maas, R. |
| | | | | Sexually |
| | | | | dimorphic |
| | | | | sterility |
| | | | | phenotypes in |
| | | | | Hoxa10- |
| | | | | deficient |
| | | | | mice. Nature |
| | | | | 374, 460-463 |
| | | | | (1995). |
| HOXA11 | ATGGATTTTGATGAGCGTGGTCCCT | 60 | Involved in | Patterson, L. |
| | GCTCCTCTAACATGTATTTGCCAAG | | kidney | T., Pembaur, |
| | TTGTACTTACTACGTCTCGGGTCCA | | development | M. & Potter, |
| | GATTTCTCCAGCCTCCCTTCTTTTCT | | | S. S. Hoxa11 |
| | GCCCCAGACCCCGTCTTCGCGCCCA | | | and Hoxd11 |
| | ATGACATACTCCTACTCCTCCAACC | | | regulate |
| | TGCCCCAGGTCCAACCCGTGCGCG | | | branching |
| | AAGTGACCTTCAGAGAGTACGCCA | | | morphogenesis |
| | TTGAGCCCGCCACTAAATGGCACCC | | | of the |
| | CCGCGGCAATCTGGCCCACTGCTAC | | | ureteric bud |
| | TCCGCGGAGGAGCTCGTGCACAGA | | | in the |
| | GACTGCCTGCAGGCGCCCAGCGCG | | | developing |
| | GCCGGCGTGCCTGGCGACGTGCTG | | | kidney. |
| | GCCAAGAGCTCGGCCAACGTCTAC | | | Development |
| | CACCACCCCACCCCCGCAGTCTCGT | | | 2153-2161 |
| | CCAATTTCTATAGCACCGTGGGCAG | | | (2001). |
| | GAACGGCGTCCTGCCACAGGCTTTC | | | |
| | GACCAGTTTTTCGAGACAGCCTACG | | | |
| | GCACCCCGGAAAACCTCGCCTCCTC | | | |
| | CGACTACCCCGGGGACAAGAGCGC | | | |
| | CGAGAAGGGGCCCCCGGCGGCCAC | | | |
| | GGCGACCTCCGCGGCGGCGGCGGC | | | |
| | GGCTGCAACGGGCGCGCCGGCAAC | | | |
| | TTCAAGTTCGGACAGCGGCGGCGG | | | |
| | CGGCGGCTGCCGGGAGATGGCGGC | | | |
| | GGCAGCAGAGGAGAAAGAGCGGC | | | |
| | GGCGGCGCCCCGAGAGCAGCAGCA | | | |
| | GCCCCGAGTCGTCTTCCGGCCACAC | | | |
| | TGAGGACAAGGCCGGCGGCTCCAG | | | |
| | TGGCCAACGCACCCGCAAAAAGCG | | | |
| | CTGCCCCTATACCAAGTACCAGATC | | | |
| | CGAGAGCTGGAACGGGAGTTCTTC | | | |
| | TTCAGCGTCTACATTAACAAAGAG | | | |
| | AAGCGCCTGCAACTGTCCCGCATGC | | | |
| | TCAACCTCACTGATCGTCAAGTCAA | | | |
| | AATCTGGTTTCAGAACAGGAGAAT | | | |
| | GAAGGAAAAAAAAATTAACAGAGA | | | |
| | CCGTTTACAGTACTACTCAGCAAAT | | | |
| | CCACTCCTCTTG | | | |
| HOXB6 | ATGAGTTCCTATTTCGTGAACTCCA | 61 | Involved in lung | 1. Patterson, |
| | CCTTCCCCGTCACTCTGGCCAGCGG | | and epidermal | L. T., |
| | GCAGGAGTCCTTCCTGGGCCAGCTA | | development | Pembaur, M. |
| | CCGCTCTATTCGTCGGGCTATGCGG | | | & Potter, S. S. |
| | ACCCGCTGAGACATTACCCCGCGCC | | | Hoxa11 and |
| | CTACGGGCCAGGGCCGGGCCAGGA | | | Hoxd11 |
| | CAAGGGCTTTGCCACTTCCTCCTAT | | | regulate |
| | TACCCGCCGGCGGGCGGTGGCTAC | | | branching |
| | GGCCGAGCGGCGCCCTGCGACTAC | | | morphogenesis |
| | GGGCCGGCGCCGGCCTTCTACCGC | | | of the |
| | GAGAAAGAGTCGGCCTGCGCACTC | | | ureteric bud |
| | TCCGGCGCCGACGAGCAGCCCCCG | | | in the |
| | TTCCACCCCGAGCCGCGGAAGTCG | | | developing |
| | GACTGCGCGCAGGACAAGAGCGTG | | | kidney. |
| | TTCGGCGAGACAGAAGAGCAGAAG | | | Development |
| | TGCTCCACTCCGGTCTACCCGTGGA | | | 2153-2161 |
| | TGCAGCGGATGAATTCGTGCAACA | | | (2001). |
| | GTTCCTCCTTTGGGCCCAGCGGCCG | | | Komuves, L. |
| | GCGAGGCCGCCAGACATACACACG | | | G. et al. |
| | TTACCAGACGCTGGAGCTGGAGAA | | | Changes in |
| | GGAGTTTCACTACAATCGCTACCTG | | | HOXB6 |
| | ACGCGGCGGCGGCGCATCGAGATC | | | homeodomain |
| | GCGCACGCCCTGTGCCTGACGGAG | | | protein |
| | AGGCAGATCAAGATATGGTTCCAG | | | structure and |
| | AACCGACGCATGAAGTGGAAAAAG | | | localization |
| | GAGAGCAAACTGCTCAGCGCGTCT | | | during human |
| | CAGCTCAGTGCCGAGGAGGAGGAA | | | epidermal |
| | GAAAAACAGGCCGAG | | | development |
| | | | | and |
| | | | | differentiation. |
| | | | | Dev. Dyn. |
| | | | | 218, 636-647 |
| | | | | (2000). |
| | | | | Cardoso, W. |
| | | | | V., Mitsialis, |
| | | | | S. A., Brody, |
| | | | | J. S. & |
| | | | | Williams, M. |
| | | | | C. Retinoic |
| | | | | acid alters the |
| | | | | expression of |
| | | | | pattern- |
| | | | | related genes |
| | | | | in the |
| | | | | developing rat |
| | | | | lung. Dev. |
| | | | | Dyn. 207, 47- |
| | | | | 59 (1996). |
| KLF4 | ATGGCTGTCAGCGACGCGCTGCTCC | 62 | Involved in | Fuchs, E., |
| | CATCTTTCTCCACGTTCGCGTCTGG | | regulation of | Segre, J. A. & |
| | CCCGGCGGGAAGGGAGAAGACACT | | pluripotency | Bauer, C. |
| | GCGTCAAGCAGGTGCCCCGAATAA | | and | Klf4 is a |
| | CCGCTGGCGGGAGGAGCTCTCCCA | | development of | transcription |
| | CATGAAGCGACTTCCCCCAGTGCTT | | skin. | factor |
| | CCCGGCCGCCCCTATGACCTGGCGG | | Reprogramming | required for |
| | CGGCGACCGTGGCCACAGACCTGG | | factor for | establishing |
| | AGAGCGGCGGAGCCGGTGCGGCTT | | induction of | the barrier |
| | GCGGCGGTAGCAACCTGGCGCCCC | | pluripotency. | function of |
| | TACCTCGGAGAGAGACCGAGGAGT | | | the skin. Nat. |
| | TCAACGATCTCCTGGACCTGGACTT | | | Genet. 22, |
| | TATTCTCTCCAATTCGCTGACCCAT | | | 356-400 |
| | CCTCCGGAGTCAGTGGCCGCCACC | | | (1999). |
| | GTGTCCTCGTCAGCGTCAGCCTCCT | | | Jiang, J. et al. |
| | CTTCGTCGTCGCCGTCGAGCAGCGG | | | A core Klf |
| | CCCTGCCAGCGCGCCCTCCACCTGC | | | circuitry |
| | AGCTTCACCTATCCGATCCGGGCCG | | | regulates self- |
| | GGAACGACCCGGGCGTGGCGCCGG | | | renewal of |
| | GCGGCACGGGCGGAGGCCTCCTCT | | | embryonic |
| | ATGGCAGGGAGTCCGCTCCCCCTCC | | | stem cells. |
| | GACGGCTCCCTTCAACCTGGCGGAC | | | Nat. Cell |
| | ATCAACGACGTGAGCCCCTCGGGC | | | Biol. 10, 353- |
| | GGCTTCGTGGCCGAGCTCCTGCGGC | | | 360 (2008). |
| | CAGAATTGGACCCGGTGTACATTCC | | | Takahashi, K. |
| | GCCGCAGCAGCCGCAGCCGCCAGG | | | & Yamanaka, |
| | TGGCGGGCTGATGGGCAAGTTCGT | | | S. Induction |
| | GCTGAAGGCGTCGCTGAGCGCCCC | | | of pluripotent |
| | TGGCAGCGAGTACGGCAGCCCGTC | | | stem cells |
| | GGTCATCAGCGTCAGCAAAGGCAG | | | from mouse |
| | CCCTGACGGCAGCCACCCGGTGGT | | | embryonic |
| | GGTGGCGCCCTACAACGGCGGGCC | | | and adult |
| | GCCGCGCACGTGCCCCAAGATCAA | | | fibroblast |
| | GCAGGAGGCGGTCTCTTCGTGCACC | | | cultures by |
| | CACTTGGGCGCTGGACCCCCTCTCA | | | defined |
| | GCAATGGCCACCGGCCGGCTGCAC | | | factors. Cell |
| | ACGACTTCCCCCTGGGGCGGCAGCT | | | 126, 663-76 |
| | CCCCAGCAGGACTACCCCGACCCT | | | (2006). |
| | GGGTCTTGAGGAAGTGCTGAGCAG | | | Takahashi, K. |
| | CAGGGACTGTCACCCTGCCCTGCCG | | | et al. |
| | CTTCCTCCCGGCTTCCATCCCCACC | | | Induction of |
| | CGGGGCCCAATTACCCATCCTTCCT | | | pluripotent |
| | GCCCGATCAGATGCAGCCGCAAGT | | | stem cells |
| | CCCGCCGCTCCATTACCAAGAGCTC | | | from adult |
| | ATGCCACCCGGTTCCTGCATGCCAG | | | human |
| | AGGAGCCCAAGCCAAAGAGGGGAA | | | fibroblasts by |
| | GACGATCGTGGCCCCGGAAAAGGA | | | defined |
| | CCGCCACCCACACTTGTGATTACGC | | | factors. Cell |
| | GGGCTGCGGCAAAACCTACACAAA | | | 131, 861-72 |
| | GAGTTCCCATCTCAAGGCACACCTG | | | (2007). |
| | CGAACCCACACAGGTGAGAAACCT | | | Yu, J. et al. |
| | TACCACTGTGACTGGGACGGCTGTG | | | Induced |
| | GATGGAAATTCGCCCGCTCAGATG | | | Pluripotent |
| | AACTGACCAGGCACTACCGTAAAC | | | Stem Cell |
| | ACACGGGGCACCGCCCGTTCCAGT | | | Lines Derived |
| | GCCAAAAATGCGACCGAGCATTTT | | | from Human |
| | CCAGGTCGGACCACCTCGCCTTACA | | | Somatic |
| | CATGAAGAGGCATTTT | | | Cells. Science |
| | | | | (80-.). 318, |
| | | | | 1917-1920 |
| | | | | (2007). |
| LHX3 | ATGGAGGCGCGCGGGGAGCTGGGC | 63 | Involved in | Sheng, H. Z. |
| | CCGGCCCGGGAGTCGGCGGGAGGC | | pituitary gland | et al. |
| | GACCTGCTGCTAGCACTGCTGGCGC | | development | Multistep |
| | GGAGGGCGGACCTGCGCCGAGAGA | | | Control of |
| | TCCCGCTGTGCGCTGGCTGTGACCA | | | Pituitary |
| | GCACATCCTGGACCGCTTCATCCTC | | | Organogenesis. |
| | AAGGCTCTGGACCGCCACTGGCAC | | | Science |
| | AGCAAGTGTCTCAAGTGCAGCGAC | | | (80-. ). 278, |
| | TGCCACACGCCACTGGCCGAGCGC | | | 1809-1812 |
| | TGCTTCAGCCGAGGGGAGAGCGTT | | | (1997). |
| | TACTGCAAGGACGACTTTTTCAAGC | | | |
| | GCTTCGGGACCAAGTGCGCCGCGT | | | |
| | GCCAGCTGGGCATCCCGCCCACGC | | | |
| | AGGTGGTGCGCCGCGCCCAGGACT | | | |
| | TCGTGTACCACCTGCACTGCTTTGC | | | |
| | CTGCGTCGTGTGCAAGCGGCAGCT | | | |
| | GGCCACGGGCGACGAGTTCTACCT | | | |
| | CATGGAGGACAGCCGGCTCGTGTG | | | |
| | CAAGGCGGACTACGAAACCGCCAA | | | |
| | GCAGCGAGAGGCCGAGGCCACGGC | | | |
| | CAAGCGGCCGCGCACGACCATCAC | | | |
| | CGCCAAGCAGCTGGAGACGCTGAA | | | |
| | GAGCGCTTACAACACCTCGCCCAA | | | |
| | GCCGGCGCGCCACGTGCGCGAGCA | | | |
| | GCTCTCGTCCGAGACGGGCCTGGA | | | |
| | CATGCGCGTGGTGCAGGTTTGGTTC | | | |
| | CAGAACCGCCGGGCCAAGGAGAAG | | | |
| | AGGCTGAAGAAGGACGCCGGCCGG | | | |
| | CAGCGCTGGGGGCAGTATTTCCGC | | | |
| | AACATGAAGCGCTCCCGCGGCGGC | | | |
| | TCCAAGTCGGACAAGGACAGCGTT | | | |
| | CAGGAGGGGCAGGACAGCGACGCT | | | |
| | GAGGTCTCCTTCCCCGATGAGCCTT | | | |
| | CCTTGGCGGAAATGGGCCCGGCCA | | | |
| | ATGGCCTCTACGGGAGCTTGGGGG | | | |
| | AACCCACCCAGGCCTTGGGCCGGC | | | |
| | CCTCGGGAGCCCTGGGCAACTTCTC | | | |
| | CCTGGAGCATGGAGGCCTGGCAGG | | | |
| | CCCAGAGCAGTACCGAGAGCTGCG | | | |
| | TCCCGGCAGCCCCTACGGTGTCCCC | | | |
| | CCATCCCCCGCCGCCCCGCAGAGC | | | |
| | CTCCCTGGCCCCCAGCCCCTCCTCT | | | |
| | CCAGCCTGGTGTACCCAGACACCA | | | |
| | GCTTGGGCCTTGTGCCCTCGGGAGC | | | |
| | CCCCGGCGGGCCCCCACCCATGAG | | | |
| | GGTGCTGGCAGGGAACGGACCCAG | | | |
| | TTCTGACCTATCCACGGGGAGCAGC | | | |
| | GGGGGTTACCCCGACTTCCCTGCCA | | | |
| | GCCCCGCCTCCTGGCTGGATGAGGT | | | |
| | AGACCACGCTCAGTTCTCAGGCCTC | | | |
| | ATGGGCCCAGCTTTCTTGTAC | | | |
| LMX1A | ATGGAAGGAATCATGAACCCCTAC | 64 | Involved in | Lin, W. et al. |
| | ACGGCTCTGCCCACCCCACAGCAG | | neuronal | Foxa1 and |
| | CTCCTGGCCATCGAGCAGAGTGTCT | | development | Foxa2 |
| | ACAGCTCAGATCCCTTCCGACAGG | | | function both |
| | GTCTCACCCCACCCCAGATGCCTGG | | | upstream of |
| | AGACCACATGCACCCTTATGGTGCC | | | and |
| | GAGCCCCTTTTCCATGACCTGGATA | | | cooperatively |
| | GCGACGACACCTCCCTCAGTAACCT | | | with Lmx1a |
| | GGGTGACTGTTTCCTAGCAACCTCA | | | and Lmx1b in |
| | GAAGCTGGGCCTCTGCAGTCCAGA | | | a feedforward |
| | GTGGGAAACCCCATTGACCATCTGT | | | loop |
| | ACTCCATGCAGAATTCTTACTTCAC | | | promoting |
| | ATCT | | | meso- |
| | | | | diencephalic |
| | | | | dopaminergic |
| | | | | neuron |
| | | | | development. |
| | | | | Dev. Biol. |
| | | | | 333, 386-396 |
| | | | | (2009). |
| | | | | Qiaolin, D. et |
| | | | | al. Specific |
| | | | | and integrated |
| | | | | roles of |
| | | | | Lmx1a, |
| | | | | Lmx1b and |
| | | | | Phox2a in |
| | | | | ventral |
| | | | | midbrain |
| | | | | development. |
| | | | | Development |
| | | | | 138, 3399- |
| | | | | 3408 (2011). |
| MEF2C | ATGGGGAGAAAAAAGATTCAGATT | 65 | Involved in | Lin, Q. et al. |
| | ACGAGGATTATGGATGAACGTAAC | | cardiac | Control of |
| | AGACAGGTGACATTTACAAAGAGG | | development | mouse cardiac |
| | AAATTTGGGTTGATGAAGAAGGCT | | | morphogenesis |
| | TATGAGCTGAGCGTGCTGTGTGACT | | | and |
| | GTGAGATTGCGCTGATCATCTTCAA | | | myogenesis |
| | CAGCACCAACAAGCTGTTCCAGTAT | | | by |
| | GCCAGCACCGACATGGACAAAGTG | | | transcription |
| | CTTCTCAAGTACACGGAGTACAAC | | | factor |
| | GAGCCGCATGAGAGCCGGACAAAC | | | MEF2C. |
| | TCAGACATCGTGGAGACGTTGAGA | | | Science 276, |
| | AAGAAGGGCCTTAATGGCTGTGAC | | | 1404-7 |
| | AGCCCAGACCCCGATGCGGACGAT | | | (1997). |
| | TCCGTAGGTCACAGCCCTGAGTCTG | | | |
| | AGGACAAGTACAGGAAAATTAACG | | | |
| | AAGATATTGATCTAATGATCAGCA | | | |
| | GGCAAAGATTGTGTGCTGTTCCACC | | | |
| | TCCCAACTTCGAGATGCCAGTCTCC | | | |
| | ATCCCAGTGTCCAGCCACAACAGTT | | | |
| | TGGTGTACAGCAACCCTGTCAGCTC | | | |
| | ACTGGGAAACCCCAACCTATTGCC | | | |
| | ACTGGCTCACCCTTCTCTGCAGAGG | | | |
| | AATAGTATGTCTCCTGGTGTAACAC | | | |
| | ATCGACCTCCAAGTGCAGGTAACA | | | |
| | CAGGTGGTCTGATGGGTGGAGACC | | | |
| | TCACGTCTGGTGCAGGCACCAGTGC | | | |
| | AGGGAACGGGTATGGCAATCCCCG | | | |
| | AAACTCACCAGGTCTGCTGGTCTCA | | | |
| | CCTGGTAACTTGAACAAGAATATG | | | |
| | CAAGCAAAATCTCCTCCCCCAATGA | | | |
| | ATTTAGGAATGAATAACCGTAAAC | | | |
| | CAGATCTCCGAGTTCTTATTCCACC | | | |
| | AGGCAGCAAGAATACGATGCCATC | | | |
| | AGTGTCTGAGGATGTCGACCTGCTT | | | |
| | TTGAATCAAAGGATAAATAACTCC | | | |
| | CAGTCGGCTCAGTCATTGGCTACCC | | | |
| | CAGTGGTTTCCGTAGCAACTCCTAC | | | |
| | TTTACCAGGACAAGGAATGGGAGG | | | |
| | ATATCCATCAGCCATTTCAACAACA | | | |
| | TATGGTACCGAGTACTCTCTGAGTA | | | |
| | GTGCAGACCTGTCATCTCTGTCTGG | | | |
| | GTTTAACACCGCCAGCGCTCTTCAC | | | |
| | CTTGGTTCAGTAACTGGCTGGCAAC | | | |
| | AGCAACACCTACATAACATGCCAC | | | |
| | CATCTGCCCTCAGTCAGTTGGGAGC | | | |
| | TTGCACTAGCACTCATTTATCTCAG | | | |
| | AGTTCAAATCTCTCCCTGCCTTCTA | | | |
| | CTCAAAGCCTCAACATCAAGTCAG | | | |
| | AACCTGTTTCTCCTCCTAGAGACCG | | | |
| | TACCACCACCCCTTCGAGATACCCA | | | |
| | CAACACACGCGCCACGAGGCGGGG | | | |
| | AGATCTCCTGTTGACAGCTTGAGCA | | | |
| | GCTGTAGCAGTTCGTACGACGGGA | | | |
| | GCGACCGAGAGGATCACCGGAACG | | | |
| | AATTCCACTCCCCCATTGGACTCAC | | | |
| | CAGACCTTCGCCGGACGAAAGGGA | | | |
| | AAGTCCCTCAGTCAAGCGCATGCG | | | |
| | ACTTTCTGAAGGATGGGCAACA | | | |
| MESP1 | ATGGCCCAGCCCCTGTGCCCGCCGC | 66 | Involved in | Bondue, A. et |
| | TCTCCGAGTCCTGGATGCTCTCTGC | | cardiac | al. Mesp1 |
| | GGCCTGGGGCCCAACTCGGCGGCC | | development | Acts as a |
| | GCCGCCCTCCGACAAGGACTGCGG | | | Master |
| | CCGCTCCCTCGTCTCGTCCCCAGAC | | | Regulator of |
| | TCATGGGGCAGCACCCCAGCCGAC | | | Multipotent |
| | AGCCCCGTGGCGAGCCCCGCGCGG | | | Cardiovascular |
| | CCAGGCACCCTCCGGGACCCCCGC | | | Progenitor |
| | GCCCCCTCCGTAGGTAGGCGCGGC | | | Specification. |
| | GCGCGCAGCAGCCGCCTGGGCAGC | | | Cell Stem |
| | GGGCAGAGGCAGAGCGCCAGTGAG | | | Cell 3,69-84 |
| | CGGGAGAAACTGCGCATGCGCACG | | | (2008). |
| | CTGGCCCGCGCCCTGCACGAGCTGC | | | |
| | GCCGCTTTCTACCGCCGTCCGTGGC | | | |
| | GCCCGCGGGCCAGAGCCTGACCAA | | | |
| | GATCGAGACGCTGCGCCTGGCTATC | | | |
| | CGCTATATCGGCCACCTGTCGGCCG | | | |
| | TGCTAGGCCTCAGCGAGGAGAGTC | | | |
| | TCCAGCGCCGGTGCCGGCAGCGCG | | | |
| | GTGACGCGGGGTCCCCTCGGGGCT | | | |
| | GCCCGCTGTGCCCCGACGACTGCCC | | | |
| | CGCGCAGATGCAGACACGGACGCA | | | |
| | GGCTGAGGGGCAGGGGCAGGGGCG | | | |
| | CGGGCTGGGCCTGGTATCCGCCGTC | | | |
| | CGCGCCGGGGCGTCCTGGGGATCC | | | |
| | CCGCCTGCCTGCCCCGGAGCCCGA | | | |
| | GCTGCACCCGAGCCGCGCGACCCG | | | |
| | CCTGCGCTGTTCGCCGAGGCGGCGT | | | |
| | GCCCGGAAGGGCAGGCGATGGAGC | | | |
| | CAAGCCCACCGTCCCCGCTCCTTCC | | | |
| | GGGCGACGTGCTGGCTCTGTTGGA | | | |
| | GACCTGGATGCCCCTCTCGCCTCTG | | | |
| | GAGTGGCTGCCTGAGGAGCCCAAG | | | |
| | TTG | | | |
| MITF | ATGCTGGAAATGCTAGAATATAAT | 67 | Involved in | Widlund, H. |
| | CACTATCAGGTGCAGACCCACCTCG | | pigment cell | R. & Fisher, |
| | AAAACCCCACCAAGTACCACATAC | | and melanocyte | D. E. |
| | AGCAAGCCCAACGGCAGCAGGTAA | | differentiation | Microphthala |
| | AGCAGTACCTTTCTACCACTTTAGC | | | mia- |
| | AAATAAACATGCCAACCAAGTCCT | | | associated |
| | GAGCTTGCCATGTCCAAACCAGCCT | | | transcription |
| | GGCGATCATGTCATGCCACCGGTGC | | | factor: a |
| | CGGGGAGCAGCGCACCCAACAGCC | | | critical |
| | CCATGGCTATGCTTACGCTTAACTC | | | regulator of |
| | CAACTGTGAAAAAGAGGGATTTTA | | | pigment cell |
| | TAAGTTTGAAGAGCAAAACAGGGC | | | development |
| | AGAGAGCGAGTGCCCAGGCATGAA | | | and survival. |
| | CACACATTCACGAGCGTCCTGTATG | | | Oncogene 22, |
| | CAGATGGATGATGTAATCGATGAC | | | 3035-3041 |
| | ATCATTAGCCTAGAATCAAGTTATA | | | (2003). |
| | ATGAGGAAATCTTGGGCTTGATGG | | | |
| | ATCCTGCTTTGCAAATGGCAAATAC | | | |
| | GTTGCCTGTCTCGGGAAACTTGATT | | | |
| | GATCTTTATGGAAACCAAGGTCTGC | | | |
| | CCCCACCAGGCCTCACCATCAGCA | | | |
| | ACTCCTGTCCAGCCAACCTTCCCAA | | | |
| | CATAAAAAGGGAGCTCACAGAGTC | | | |
| | TGAAGCAAGAGCACTGGCCAAAGA | | | |
| | GAGGCAGAAAAAGGACAATCACAA | | | |
| | CCTGATTGAACGAAGAAGAAGATT | | | |
| | TAACATAAATGACCGCATTAAAGA | | | |
| | ACTAGGTACTTTGATTCCCAAGTCA | | | |
| | AATGATCCAGACATGCGCTGGAAC | | | |
| | AAGGGAACCATCTTAAAAGCATCC | | | |
| | GTGGACTATATCCGAAAGTTGCAA | | | |
| | CGAGAACAGCAACGCGCAAAAGAA | | | |
| | CTTGAAAACCGACAGAAGAAACTG | | | |
| | GAGCACGCCAACCGGCATTTGTTGC | | | |
| | TCAGAATACAGGAACTTGAAATGC | | | |
| | AGGCTCGAGCTCATGGACTTTCCCT | | | |
| | TATTCCATCCACGGGTCTCTGCTCT | | | |
| | CCAGATTTGGTGAATCGGATCATCA | | | |
| | AGCAAGAACCCGTTCTTGAGAACT | | | |
| | GCAGCCAAGACCTCCTTCAGCATCA | | | |
| | TGCAGACCTAACCTGTACAACAACT | | | |
| | CTCGATCTCACGGATGGCACCATCA | | | |
| | CCTTCAACAACAACCTCGGAACTG | | | |
| | GGACTGAGGCCAACCAAGCCTATA | | | |
| | GTGTCCCCACAAAAATGGGATCCA | | | |
| | AACTGGAAGACATCCTGATGGACG | | | |
| | ACACCCTTTCTCCCGTCGGTGTCAC | | | |
| | TGATCCACTCCTTTCCTCAGTGTCC | | | |
| | CCCGGAGCTTCCAAAACAAGCAGC | | | |
| | CGGAGGAGCAGTATGAGCATGGAA | | | |
| | GAGACGGAGCACACTTGT | | | |
| MYC | ATGCCCCTCAACGTTAGCTTCACCA | 68 | Involved in cell | Pelengaris, S., |
| | ACAGGAACTATGACCTCGACTACG | | proliferation, | Khan, M. & |
| | ACTCGGTGCAGCCGTATTTCTACTG | | differentiation | Evan, G. c- |
| | CGACGAGGAGGAGAACTTCTACCA | | and apoptosis. | MYC: more |
| | GCAGCAGCAGCAGAGCGAGCTGCA | | Reprogramming | than just a |
| | GCCCCCGGCGCCCAGCGAGGATAT | | factor for | matter of life |
| | CTGGAAGAAATTCGAGCTGCTGCC | | induction of | and death. |
| | CACCCCGCCCCTGTCCCCTAGCCGC | | pluripotency. | Nat. Rev. |
| | CGCTCCGGGCTCTGCTCGCCCTCCT | | | Cancer 2, |
| | ACGTTGCGGTCACACCCTTCTCCCT | | | 764-776 |
| | TCGGGGAGACAACGACGGCGGTGG | | | (2002). |
| | CGGGAGCTTCTCCACGGCCGACCA | | | Takahashi, K. |
| | GCTGGAGATGGTGACCGAGCTGCT | | | & Yamanaka, |
| | GGGAGGAGACATGGTGAACCAGAG | | | S. Induction |
| | TTTCATCTGCGACCCGGACGACGAG | | | of pluripotent |
| | ACCTTCATCAAAAACATCATCATCC | | | stem cells |
| | AGGACTGTATGTGGAGCGGCTTCTC | | | from mouse |
| | GGCCGCCGCCAAGCTCGTCTCAGA | | | embryonic |
| | GAAGCTGGCCTCCTACCAGGCTGC | | | and adult |
| | GCGCAAAGACAGCGGCAGCCCGAA | | | fibroblast |
| | CCCCGCCCGCGGCCACAGCGTCTG | | | cultures by |
| | CTCCACCTCCAGCTTGTACCTGCAG | | | defined |
| | GATCTGAGCGCCGCCGCCTCAGAG | | | factors. Cell |
| | TGCATCGACCCCTCGGTGGTCTTCC | | | 126,663-76 |
| | CCTACCCTCTCAACGACAGCAGCTC | | | (2006). |
| | GCCCAAGTCCTGCGCCTCGCAAGA | | | Takahashi, K. |
| | CTCCAGCGCCTTCTCTCCGTCCTCG | | | et al. |
| | GATTCTCTGCTCTCCTCGACGGAGT | | | Induction of |
| | CCTCCCCGCAGGGCAGCCCCGAGC | | | pluripotent |
| | CCCTGGTGCTCCATGAGGAGACAC | | | stem cells |
| | CGCCCACCACCAGCAGCGACTCTG | | | from adult |
| | AGGAGGAACAAGAAGATGAGGAA | | | human |
| | GAAATCGATGTTGTTTCTGTGGAAA | | | fibroblasts by |
| | AGAGGCAGGCTCCTGGCAAAAGGT | | | defined |
| | CAGAGTCTGGATCACCTTCTGCTGG | | | factors. Cell |
| | AGGCCACAGCAAACCTCCTCACAG | | | 131,861-72 |
| | CCCACTGGTCCTCAAGAGGTGCCAC | | | (2007). |
| | GTCTCCACACATCAGCACAACTACG | | | Yu, J. et al. |
| | CAGCGCCTCCCTCCACTCGGAAGG | | | Induced |
| | ACTATCCTGCTGCCAAGAGGGTCA | | | Pluripotent |
| | AGTTGGACAGTGTCAGAGTCCTGA | | | Stem Cell |
| | GACAGATCAGCAACAACCGAAAAT | | | Lines Derived |
| | GCACCAGCCCCAGGTCCTCGGACA | | | from Human |
| | CCGAGGAGAATGTCAAGAGGCGAA | | | Somatic |
| | CACACAACGTCTTGGAGCGCCAGA | | | Cells. Science |
| | GGAGGAACGAGCTAAAACGGAGCT | | | (80-. ). 318, |
| | TTTTTGCCCTGCGTGACCAGATCCC | | | 1917-1920 |
| | GGAGTTGGAAAACAATGAAAAGGC | | | (2007). |
| | CCCCAAGGTAGTTATCCTTAAAAAA | | | |
| | GCCACAGCATACATCCTGTCCGTCC | | | |
| | AAGCAGAGGAGCAAAAGCTCATTT | | | |
| | CTGAAGAGGACTTGTTGCGGAAAC | | | |
| | GACGAGAACAGTTGAAACACAAAC | | | |
| | TTGAACAGCTACGGAACTCTTGTGC | | | |
| | G | | | |
| MYCL | ATGGACTACGACTCGTACCAGCACT | 69 | Involved in cell | Hatton, K. S. |
| | ATTTCTACGACTATGACTGCGGGGA | | proliferation, | et al. |
| | GGATTTCTACCGCTCCACGGCGCCC | | differentiation | Expression |
| | AGCGAGGACATCTGGAAGAAATTC | | and apoptosis. | and activity of |
| | GAGCTGGTGCCATCGCCCCCCACGT | | | L-Myc in |
| | CGCCGCCCTGGGGCTTGGGTCCCGG | | | normal mouse |
| | CGCAGGGGACCCGGCCCCCGGGAT | | | development. |
| | TGGTCCCCCGGAGCCGTGGCCCGG | | | Mol. Cell. |
| | AGGGTGCACCGGAGACGAAGCGGA | | | Biol. 16, |
| | ATCCCGGGGCCACTCGAAAGGCTG | | | 1794-804 |
| | GGGCAGGAACTACGCCTCCATCAT | | | (1996). |
| | ACGCCGTGACTGCATGTGGAGCGG | | | |
| | CTTCTCGGCCCGGGAACGGCTGGA | | | |
| | GAGAGCTGTGAGCGACCGGCTCGC | | | |
| | TCCTGGCGCGCCCCGGGGGAACCC | | | |
| | GCCCAAGGCGTCCGCCGCCCCGGA | | | |
| | CTGCACTCCCAGCCTCGAAGCCGGC | | | |
| | AACCCGGCGCCCGCCGCCCCCTGTC | | | |
| | CGCTGGGCGAACCCAAGACCCAGG | | | |
| | CCTGCTCCGGGTCCGAGAGCCCAA | | | |
| | GCGACTCGGGTAAGGACCTCCCCG | | | |
| | AGCCATCCAAGAGGGGGCCACCCC | | | |
| | ATGGGTGGCCAAAGCTCTGCCCCTG | | | |
| | CCTGAGGTCAGGCATTGGCTCTTCT | | | |
| | CAAGCTCTTGGGCCATCTCCGCCTC | | | |
| | TCTTTGGC | | | |
| MYCN | ATGCCGAGTTGTTCCACGTCTACGA | 70 | Involved in cell | Malynn, B. A. |
| | TGCCAGGAATGATATGCAAGAACC | | proliferation | et al. N-myc |
| | CCGACTTGGAGTTTGACTCTTTGCA | | and | can |
| | ACCATGCTTTTATCCGGATGAAGAC | | differentiation | functionally |
| | GACTTTTATTTCGGCGGCCCGGACA | | | replace c-myc |
| | GCACCCCTCCTGGAGAGGACATCT | | | in murine |
| | GGAAAAAATTCGAACTTTTGCCTAC | | | development, |
| | ACCCCCACTCAGTCCCTCTCGAGGA | | | cellular |
| | TTTGCGGAACACAGCAGTGAACCG | | | growth, and |
| | CCGTCTTGGGTGACAGAGATGCTCC | | | differentiation. |
| | TCGAGAACGAATTGTGGGGAAGCC | | | Genes Dev. |
| | CTGCGGAGGAAGACGCTTTCGGGC | | | 14, 1390-9 |
| | TCGGTGGACTCGGAGGTCTCACGCC | | | (2000). |
| | GAACCCAGTCATACTGCAGGATTG | | | Sawai, S. et |
| | CATGTGGTCTGGATTCTCAGCTCGG | | | al. Defects of |
| | GAGAAGCTGGAACGGGCAGTTTCT | | | embryonic |
| | GAGAAACTCCAACATGGCCGGGGC | | | organogenesis |
| | CCTCCAACAGCGGGTTCTACCGCAC | | | resulting from |
| | AGTCCCCTGGTGCTGGAGCCGCTAG | | | targeted |
| | TCCCGCGGGGAGAGGCCATGGGGG | | | disruption of |
| | CGCGGCAGGAGCGGGTAGGGCCGG | | | the N-myc |
| | CGCTGCGTTGCCTGCTGAGCTTGCG | | | gene in the |
| | CACCCCGCCGCTGAATGTGTAGATC | | | mouse. |
| | CCGCGGTAGTGTTTCCGTTCCCCGT | | | Development |
| | TAATAAGCGAGAACCGGCACCGGT | | | 117, 1445- |
| | GCCAGCCGCTCCTGCGTCTGCACCC | | | 1455 (1993). |
| | GCGGCAGGTCCTGCTGTCGCCTCAG | | | Stanton, B. |
| | GAGCAGGTATTGCCGCTCCTGCAG | | | R., Perkins, |
| | GGGCACCAGGAGTAGCCCCTCCAA | | | A. S., |
| | GGCCCGGCGGTAGGCAAACCTCCG | | | Tessarollo, L., |
| | GCGGCGACCACAAAGCACTCTCAA | | | Sassoon, D. |
| | CGAGCGGAGAGGATACACTGTCCG | | | A. & Parada, |
| | ATAGTGATGACGAGGACGACGAAG | | | L. F. Loss of |
| | AGGAGGACGAGGAGGAGGAGATA | | | N-myc |
| | GATGTTGTCACGGTCGAGAAGCGA | | | function |
| | AGGAGTTCTTCAAATACAAAAGCG | | | results in |
| | GTAACGACATTCACGATAACAGTA | | | embryonic |
| | AGACCTAAGAACGCAGCCCTCGGT | | | lethality and |
| | CCAGGGCGGGCCCAGTCCAGTGAG | | | failure of the |
| | CTTATACTTAAGCGCTGCCTGCCGA | | | epithelial |
| | TTCACCAGCAGCATAACTACGCGG | | | component of |
| | CCCCTAGTCCCTACGTTGAGAGCGA | | | the embryo to |
| | GGATGCCCCCCCACAAAAAAAAAT | | | develop. |
| | AAAGTCTGAAGCGTCCCCCCGCCCC | | | Genes Dev. 6, |
| | CTGAAATCCGTAATCCCCCCAAAG | | | 2235-47 |
| | GCGAAGTCACTCAGTCCCAGGAAT | | | (1992). |
| | TCAGATTCCGAGGACTCCGAACGG | | | |
| | CGGCGGAATCATAACATACTTGAG | | | |
| | AGACAACGACGCAATGACCTGAGG | | | |
| | TCTTCTTTTTTGACCCTCCGAGATC | | | |
| | ACGTCCCCGAGCTGGTTAAGAATG | | | |
| | AGAAAGCTGCGAAGGTAGTCATAC | | | |
| | TGAAAAAGGCCACCGAGTATGTCC | | | |
| | ATAGTTTGCAAGCTGAGGAGCACC | | | |
| | AGCTTCTCCTTGAAAAGGAGAAAC | | | |
| | TTCAGGCACGACAACAGCAATTGC | | | |
| | TGAAAAAGATTGAGCATGCACGCA | | | |
| | CTTGT | | | |
| MYOD1 | ATGGAGCTACTGTCGCCACCGCTCC | 71 | Involved in | Tapscott, S. J. |
| | GCGACGTAGACCTGACGGCCCCCG | | skeletal muscle | The circuitry |
| | ACGGCTCTCTCTGCTCCTTTGCCAC | | specification | of a master |
| | AACGGACGACTTCTATGACGACCC | | and | switch: Myod |
| | GTGTTTCGACTCCCCGGACCTGCGC | | differentiation | and the |
| | TTCTTCGAAGACCTGGACCCGCGCC | | Demonstrated to | regulation of |
| | TGATGCACGTGGGCGCGCTCCTGA | | induce | skeletal |
| | AACCCGAAGAGCACTCGCACTTCC | | differentiation | muscle gene |
| | CCGCGGCGGTGCACCCGGCCCCGG | | of hPSCs to | transcription. |
| | GCGCACGTGAGGACGAGCATGTGC | | skeletal muscle | Development |
| | GCGCGCCCAGCGGGCACCACCAGG | | | 132, 2685- |
| | CGGGCCGCTGCCTACTGTGGGCCTG | | | 2695 (2005). |
| | CAAGGCGTGCAAGCGCAAGACCAC | | | Abujarour, R. |
| | CAACGCCGACCGCCGCAAGGCCGC | | | et al. |
| | CACCATGCGCGAGCGGCGCCGCCT | | | Myogenic |
| | GAGCAAAGTAAATGAGGCCTTTGA | | | differentiation |
| | GACACTCAAGCGCTGCACGTCGAG | | | of muscular |
| | CAATCCAAACCAGCGGTTGCCCAA | | | dystrophy- |
| | GGTGGAGATCCTGCGCAACGCCAT | | | specific |
| | CCGCTATATCGAGGGCCTGCAGGCT | | | induced |
| | CTGCTGCGCGACCAGGACGCCGCG | | | pluripotent |
| | CCCCCTGGCGCCGCAGCCGCCTTCT | | | stem cells for |
| | ATGCGCCGGGCCCGCTGCCCCCGG | | | use in drug |
| | GCCGCGGCGGCGAGCACTACAGCG | | | discovery. |
| | GCGACTCCGACGCGTCCAGCCCGC | | | Stem Cells |
| | GCTCCAACTGCTCCGACGGCATGAT | | | Transl. Med. |
| | GGACTACAGCGGCCCCCCGAGCGG | | | 3,149-60 |
| | CGCCCGGCGGCGGAACTGCTACGA | | | (2014). |
| | AGGCGCCTACTACAACGAGGCGCC | | | |
| | CAGCGAACCCAGGCCCGGGAAGAG | | | |
| | TGCGGCGGTGTCGAGCCTAGACTG | | | |
| | CCTGTCCAGCATCGTGGAGCGCATC | | | |
| | TCCACCGAGAGCCCTGCGGCGCCC | | | |
| | GCCCTCCTGCTGGCGGACGTGCCTT | | | |
| | CTGAGTCGCCTCCGCGCAGGCAAG | | | |
| | AGGCTGCCGCCCCCAGCGAGGGAG | | | |
| | AGAGCAGCGGCGACCCCACCCAGT | | | |
| | CACCGGACGCCGCCCCGCAGTGCC | | | |
| | CTGCGGGTGCGAACCCCAACCCGA | | | |
| | TATACCAGGTGCTC | | | |
| MYOG | ATGGAGCTGTATGAGACATCCCCCT | 72 | Involved in | Pownall, M. |
| | ACTTCTACCAGGAACCCCGCTTCTA | | skeletal muscle | E., |
| | TGATGGGGAAAACTACCTGCCTGTC | | specification | Gustafsson, |
| | CACCTCCAGGGCTTCGAACCACCA | | and | M. K. & |
| | GGCTACGAGCGGACGGAGCTCACC | | differentiation | Emerson, C. |
| | CTGAGCCCCGAGGCCCCAGGGCCC | | | P. Myogenic |
| | CTTGAGGACAAGGGGCTGGGGACC | | | Regulatory |
| | CCCGAGCACTGTCCAGGCCAGTGC | | | Factors and |
| | CTGCCGTGGGCGTGTAAGGTGTGTA | | | the |
| | AGAGGAAGTCGGTGTCCGTGGACC | | | Specification |
| | GGCGGCGGGCGGCCACACTGAGGG | | | of Muscle |
| | AGAAGCGCAGGCTCAAGAAGGTGA | | | Progenitors in |
| | ATGAGGCCTTCGAGGCCCTGAAGA | | | Vertebrate |
| | GAAGCACCCTGCTCAACCCCAACC | | | Embryos. |
| | AGCGGCTGCCCAAGGTGGAGATCC | | | Annu. Rev. |
| | TGCGCAGTGCCATCCAGTACATCGA | | | Cell Dev. |
| | GCGCCTCCAGGCCCTGCTCAGCTCC | | | Biol. 18,747- |
| | CTCAACCAGGAGGAGCGTGACCTC | | | 783 (2002). |
| | CGCTACCGGGGCGGGGGCGGGCCC | | | Shi, X. & |
| | CAGCCAGGGGTGCCCAGCGAATGC | | | Garry, D. J. |
| | AGCTCTCACAGCGCCTCCTGCAGTC | | | Muscle stem |
| | CAGAGTGGGGCAGTGCACTGGAGT | | | cells in |
| | TCAGCGCCAACCCAGGGGATCATC | | | development, |
| | TGCTCACGGCTGACCCTACAGATGC | | | regeneration, |
| | CCACAACCTGCACTCCCTCACCTCC | | | and disease. |
| | ATCGTGGACAGCATCACAGTGGAA | | | Genes Dev. |
| | GATGTGTCTGTGGCCTTCCCAGATG | | | 20,1692-708 |
| | AAACCATGCCCAAC | | | (2006). |
| NEURO | ATGACCAAATCGTACAGCGAGAGT | 73 | Involved in | Pataskar, A. |
| D1 | GGGCTGATGGGCGAGCCTCAGCCC | | neuronal | et al. |
| | CAAGGTCCTCCAAGCTGGACAGAC | | specification | NeuroD1 |
| | GAGTGTCTCAGTTCTCAGGACGAG | | and | reprograms |
| | GAGCACGAGGCAGACAAGAAGGA | | differentiation | chromatin and |
| | GGACGACCTCGAAGCCATGAACGC | | Demonstrated to | transcription |
| | AGAGGAGGACTCACTGAGGAACGG | | induce neuronal | factor |
| | GGGAGAGGAGGAGGACGAAGATG | | differentiation | landscapes to |
| | AGGACCTGGAAGAGGAGGAAGAA | | in hPSCs | induce the |
| | GAGGAAGAGGAGGATGACGATCAA | | | neuronal |
| | AAGCCCAAGAGACGCGGCCCCAAA | | | program. |
| | AAGAAGAAGATGACTAAGGCTCGC | | | EMBO J. 35, |
| | CTGGAGCGTTTTAAATTGAGACGCA | | | 24-45 (2016). |
| | TGAAGGCTAACGCCCGGGAGCGGA | | | Zhang, Y. et |
| | ACCGCATGCACGGACTGAACGCGG | | | al. Rapid |
| | CGCTAGACAACCTGCGCAAGGTGG | | | single-step |
| | TGCCTTGCTATTCTAAGACGCAGAA | | | induction of |
| | GCTGTCCAAAATCGAGACTCTGCGC | | | functional |
| | TTGGCCAAGAACTACATCTGGGCTC | | | neurons from |
| | TGTCGGAGATCCTGCGCTCAGGCA | | | human |
| | AAAGCCCAGACCTGGTCTCCTTCGT | | | pluripotent |
| | TCAGACGCTTTGCAAGGGCTTATCC | | | stem cells. |
| | CAACCCACCACCAACCTGGTTGCG | | | Neuron 78, |
| | GGCTGCCTGCAACTCAATCCTCGGA | | | 785-98 |
| | CTTTTCTGCCTGAGCAGAACCAGGA | | | (2013). |
| | CATGCCCCCCCACCTGCCGACGGCC | | | |
| | AGCGCTTCCTTCCCTGTACACCCCT | | | |
| | ACTCCTACCAGTCGCCTGGGCTGCC | | | |
| | CAGTCCGCCTTACGGTACCATGGAC | | | |
| | AGCTCCCATGTCTTCCACGTTAAGC | | | |
| | CTCCGCCGCACGCCTACAGCGCAG | | | |
| | CGCTGGAGCCCTTCTTTGAAAGCCC | | | |
| | TCTGACTGATTGCACCAGCCCTTCC | | | |
| | TTTGATGGACCCCTCAGCCCGCCGC | | | |
| | TCAGCATCAATGGCAACTTCTCTTT | | | |
| | CAAACACGAACCGTCCGCCGAGTT | | | |
| | TGAGAAAAATTATGCCTTTACCATG | | | |
| | CACTATCCTGCAGCGACACTGGCA | | | |
| | GGGGCCCAAAGCCACGGATCAATC | | | |
| | TTCTCAGGCACCGCTGCCCCTCGCT | | | |
| | GCGAGATCCCCATAGACAATATTAT | | | |
| | GTCCTTCGATAGCCATTCACATCAT | | | |
| | GAGCGAGTCATGAGTGCCCAGCTC | | | |
| | AATGCCATATTTCATGAT | | | |
| NEURO | ATGCCAGCCCGCCTTGAGACCTGCA | 74 | Involved in | Bertrand, N., |
| G1 | TCTCCGACCTCGACTGCGCCAGCAG | | neuronal | Castro, D. S. |
| | CAGCGGCAGTGACCTATCCGGCTTC | | specification | & Guillemot, |
| | CTCACCGACGAGGAAGACTGTGCC | | and | F. Proneural |
| | AGACTCCAACAGGCAGCCTCCGCTT | | differentiation | genes and the |
| | CGGGGCCGCCCGCGCCGGCCCGCA | | | specification |
| | GGGGCGCGCCCAATATCTCCCGGG | | | of neural cell |
| | CGTCTGAGGTTCCAGGGGCACAGG | | | types. Nat. |
| | ACGACGAGCAGGAGAGGCGGCGGC | | | Rev. |
| | GCCGCGGCCGGACGCGGGTCCGCT | | | Neurosci. 3, |
| | CCGAGGCGCTGCTGCACTCGCTGCG | | | 517-530 |
| | CAGGAGCCGGCGCGTCAAGGCCAA | | | (2002). |
| | CGATCGCGAGCGCAACCGCATGCA | | | |
| | CAACTTGAACGCGGCCCTGGACGC | | | |
| | ACTGCGCAGCGTGCTGCCCTCGTTC | | | |
| | CCCGACGACACCAAGCTCACCAAA | | | |
| | ATCGAGACGCTGCGCTTCGCCTACA | | | |
| | ACTACATCTGGGCTCTGGCCGAGAC | | | |
| | ACTGCGCCTGGCGGATCAAGGGCT | | | |
| | GCCCGGAGGCGGTGCCCGGGAGCG | | | |
| | CCTCCTGCCGCCGCAGTGCGTCCCC | | | |
| | TGCCTGCCCGGTCCCCCAAGCCCCG | | | |
| | CCAGCGACGCGGAGTCCTGGGGCT | | | |
| | CAGGTGCCGCCGCCGCCTCCCCGCT | | | |
| | CTCTGACCCCAGTAGCCCAGCCGCC | | | |
| | TCCGAAGACTTCACCTACCGCCCCG | | | |
| | GCGACCCTGTTTTCTCCTTCCCAAG | | | |
| | CCTGCCCAAAGACTTGCTCCACACA | | | |
| | ACGCCCTGTTTCATTCCTTACCAC | | | |
| NEURO | ATGACACCACAACCATCTGGTGCTC | 75 | Involved in | Bertrand, N., |
| G3 | CCACAGTCCAGGTGACGCGAGAGA | | pancreatic | Castro, D. S. |
| | CTGAAAGATCATTCCCACGCGCGTC | | development, | & Guillemot, |
| | CGAGGATGAGGTGACATGTCCAAC | | and neuronal | F. Proneural |
| | TAGCGCACCCCCCTCTCCTACCCGG | | specification | genes and the |
| | ACCCGCGGGAATTGTGCTGAGGCC | | and | specification |
| | GAAGAGGGAGGATGCAGAGGAGC | | differentiation | of neural cell |
| | ACCAAGGAAACTTCGAGCCCGACG | | | types. Nat. |
| | GGGTGGAAGAAGCCGCCCCAAGTC | | | Rev. |
| | TGAGCTCGCCCTTAGCAAGCAGCG | | | Neurosci. 3, |
| | CCGCAGTCGGAGGAAAAAGGCAAA | | | 517-530 |
| | CGACCGGGAAAGGAATAGGATGCA | | | (2002). |
| | TAATCTTAATTCTGCTCTGGACGCT | | | Arda, H. E. et |
| | CTGCGAGGCGTACTTCCTACTTTCC | | | al. Gene |
| | CGGATGACGCGAAATTGACCAAGA | | | Regulatory |
| | TAGAGACTCTCCGGTTTGCACATAA | | | Networks |
| | TTACATCTGGGCTCTTACACAAACA | | | Governing |
| | CTGAGAATTGCCGATCACAGTCTTT | | | Pancreas |
| | ACGCTCTTGAGCCACCCGCCCCGCA | | | Development. |
| | CTGTGGCGAGCTGGGTAGCCCCGG | | | Dev. Cell 25, |
| | CGGCTCTCCTGGAGACTGGGGGTCT | | | 5-13 (2013). |
| | TTGTATTCTCCTGTCAGCCAAGCGG | | | |
| | GATCTTTGAGTCCGGCTGCCAGTCT | | | |
| | CGAAGAAAGACCCGGACTCCTTGG | | | |
| | AGCGACTTTTTCAGCATGTCTGTCC | | | |
| | CCTGGCTCATTGGCTTTCTCAGACT | | | |
| | TTTTG | | | |
| NRL | ATGGCCCTGCCTCCCAGCCCGCTGG | 76 | Involved in | Mears, A. J. |
| | CCATGGAATATGTCAATGACTTTGA | | photoreceptor | et al. Nr1 is |
| | CTTGATGAAGTTTGAGGTAAAGCG | | development | required for |
| | GGAACCCTCTGAGGGCCGACCTGG | | | rod |
| | CCCACCTACAGCCTCACTGGGATCC | | | photoreceptor |
| | ACACCTTACAGCTCAGTGCCTCCTT | | | development. |
| | CACCCACCTTCAGTGAACCAGGCAT | | | Nat. Genet. |
| | GGTAGGGGCAACCGAGGGTACACG | | | 29, 447-452 |
| | ACCAGGTTTGGAGGAGCTGTACTG | | | (2001). |
| | GCTTGCTACCCTGCAGCAGCAGCTT | | | |
| | GGGGCTGGGGAGGCATTGGGACTG | | | |
| | AGTCCTGAAGAGGCCATGGAGCTA | | | |
| | CTGCAAGGTCAGGGCCCAGTCCCT | | | |
| | GTTGATGGACCCCATGGTTACTACC | | | |
| | CAGGGAGCCCAGAGGAGACAGGAG | | | |
| | CCCAGCACGTTCAGTTGGCAGAGC | | | |
| | GGTTTTCCGACGCGGCGCTTGTCTC | | | |
| | GATGTCTGTGCGAGAACTAAACCG | | | |
| | GCAGCTGCGGGGATGCGGGAGAGA | | | |
| | CGAGGCTCTACGACTGAAGCAGAG | | | |
| | GCGTCGAACGCTGAAGAACCGTGG | | | |
| | CTATGCGCAAGCATGTCGTTCCAAG | | | |
| | AGGCTGCAACAGAGGCGAGGTCTT | | | |
| | GAGGCCGAGCGCGCCCGTCTTGCA | | | |
| | GCCCAGCTAGATGCGCTACGAGCT | | | |
| | GAAGTAGCACGTTTGGCAAGAGAG | | | |
| | CGAGATCTCTACAAGGCTCGCTGTG | | | |
| | ACCGGCTAACCTCGAGTGGCCCCG | | | |
| | GGTCCGGGGATCCCTCCCACCTTTT | | | |
| | CCTCTGCCCAACTTTCTTGTACAAA | | | |
| | GTTGTCCCC | | | |
| ONECU | ATGAACGCGCAGCTGACCATGGAA | 77 | Involved in | Chakrabarti, |
| T1 | GCGATCGGCGAGCTGCACGGGGTG | | retinal, liver, | S. K., et al. |
| | AGCCATGAGCCGGTGCCCGCCCCT | | gallbladder and | Transcription |
| | GCCGACCTGCTGGGCGGCAGCCCC | | pancreatic | factors direct |
| | CACGCGCGCAGCTCCGTGGCGCAC | | development | the |
| | CGCGGCAGCCACCTGCCCCCCGCG | | | development |
| | CACCCGCGCTCCATGGGCATGGCGT | | | and function |
| | CCCTGCTGGACGGCGGCAGCGGCG | | | of pancreatic |
| | GCGGAGATTACCACCACCACCACC | | | β cells. |
| | GGGCCCCTGAGCACAGCCTGGCCG | | | Trends |
| | GCCCCCTGCATCCCACCATGACCAT | | | Endocrinol. |
| | GGCCTGCGAGACTCCCCCAGGTAT | | | Metab. 14, |
| | GAGCATGCCCACCACCTACACCAC | | | 78-84 (2003). |
| | CTTGACCCCTCTGCAGCCGCTGCCT | | | Clotman, F. et |
| | CCCATCTCCACAGTCTCGGACAAGT | | | al. The onecut |
| | TCCCCCACCATCACCACCACCACCA | | | transcription |
| | TCACCACCACCACCCGCACCACCA | | | factor HNF6 |
| | CCAGCGCCTGGCGGGCAACGTGAG | | | is required for |
| | CGGTAGCTTCACGCTCATGCGGGAT | | | normal |
| | GAGCGCGGGCTGGCCTCCATGAAT | | | development |
| | AACCTCTATACCCCCTACCACAAGG | | | of the biliary |
| | ACGTGGCCGGCATGGGCCAGAGCC | | | tract. |
| | TCTCGCCCCTCTCCAGCTCCGGTCT | | | Development |
| | GGGCAGCATCCACAACTCCCAGCA | | | 129,1819- |
| | AGGGCTCCCCCACTATGCCCACCCG | | | 1828 (2002). |
| | GGGGCCGCCATGCCCACCGACAAG | | | Sapkota, D. et |
| | ATGCTCACCCCCAACGGCTTCGAAG | | | al. Onecut1 |
| | CCCACCACCCGGCCATGCTCGGCC | | | and Onecut2 |
| | GCCACGGGGAGCAGCACCTCACGC | | | redundantly |
| | CCACCTCGGCCGGCATGGTGCCCAT | | | regulate early |
| | CAACGGCCTTCCTCCGCACCATCCC | | | retinal cell |
| | CACGCCCACCTGAACGCCCAGGGC | | | fates during |
| | CACGGGCAACTCCTGGGCACAGCC | | | development. |
| | CGGGAGCCCAACCCTTCGGTGACC | | | Proc. Natl. |
| | GGCGCGCAGGTCAGCAATGGAAGT | | | Acad. Sci. U. |
| | AATTCAGGGCAGATGGAAGAGATC | | | S. A. 111, |
| | AATACCAAAGAGGTGGCGCAGCGT | | | E4086-95 |
| | ATCACCACCGAGCTCAAGCGCTAC | | | (2014). |
| | AGCATCCCACAGGCCATCTTCGCGC | | | |
| | AGAGGGTGCTCTGCCGCTCCCAGG | | | |
| | GGACCCTCTCGGACCTGCTGCGCAA | | | |
| | CCCCAAACCCTGGAGCAAACTCAA | | | |
| | ATCCGGCCGGGAGACCTTCCGGAG | | | |
| | GATGTGGAAGTGGCTGCAGGAGCC | | | |
| | GGAGTTCCAGCGCATGTCCGCGCTC | | | |
| | CGCTTAGCAGCATGCAAAAGGAAA | | | |
| | GAACAAGAACATGGGAAGGATAGA | | | |
| | GGCAACACACCCAAAAAGCCCAGG | | | |
| | TTGGTCTTCACAGATGTCCAGCGTC | | | |
| | GAACTCTACATGCAATATTCAAGG | | | |
| | AAAATAAGCGTCCATCCAAAGAAT | | | |
| | TGCAAATCACCATTTCCCAGCAGCT | | | |
| | GGGGTTGGAGCTGAGCACTGTCAG | | | |
| | CAACTTCTTCATGAACGCAAGAAG | | | |
| | GAGGAGTCTGGACAAGTGGCAGGA | | | |
| | CGAGGGCAGCTCCAATTCAGGCAA | | | |
| | CTCATCTTCTTCATCAAGCACTTGT | | | |
| | ACCAAAGCA | | | |
| OTX2 | ATGATGTCTTATCTTAAGCAACCGC | 78 | Involved in | Rhinn, M. et |
| | CTTACGCAGTCAATGGGCTGAGTCT | | photoreceptor | al. Sequential |
| | GACCACTTCGGGTATGGACTTGCTG | | differentiation, | roles for Otx2 |
| | CACCCCTCCGTGGGCTACCCGGGGC | | pineal gland | in visceral |
| | CCTGGGCTTCTTGTCCCGCAGCCAC | | development | endoderm and |
| | CCCCCGGAAACAGCGCCGGGAGAG | | and induction | neuroectoderm |
| | GACGACGTTCACTCGGGCGCAGCT | | and | for |
| | AGATGTGCTGGAAGCACTGTTTGCC | | specification of | forebrain and |
| | AAGACCCGGTACCCAGACATCTTC | | forebrain and | midbrain |
| | ATGCGAGAGGAGGTGGCACTGAAA | | midbrain | induction and |
| | ATCAACTTGCCCGAGTCGAGGGTG | | | specification. |
| | CAGGTATGGTTTAAGAATCGAAGA | | | Development |
| | GCTAAGTGCCGCCAACAACAGCAA | | | 125, 845-856 |
| | CAACAGCAGAATGGAGGTCAAAAC | | | (1998). |
| | AAAGTGAGACCTGCCAAAAAGAAG | | | Nishida, A. et |
| | ACATCTCCAGCTCGGGAAGTGAGTT | | | al. Otx2 |
| | CAGAGAGTGGAACAAGTGGCCAAT | | | homeobox |
| | TCACTCCCCCCTCTAGCACCTCAGT | | | gene controls |
| | CCCGACCATTGCCAGCAGCAGTGCT | | | retinal |
| | CCTGTGTCTATCTGGAGCCCAGCTT | | | photoreceptor |
| | CCATCTCCCCACTGTCAGATCCCTT | | | cell fate and |
| | GTCCACCTCCTCTTCCTGCATGCAG | | | pineal gland |
| | AGGTCCTATCCCATGACCTATACTC | | | development. |
| | AGGCTTCAGGTTATAGTCAAGGAT | | | Nat. Neurosci. |
| | ATGCTGGCTCAACTTCCTACTTTGG | | | 6,1255-1263 |
| | GGGCATGGACTGTGGATCATATTTG | | | (2003). |
| | ACCCCTATGCATCACCAGCTTCCCG | | | |
| | GACCAGGGGCCACACTCAGTCCCA | | | |
| | TGGGTACCAATGCAGTCACCAGCC | | | |
| | ATCTCAATCAGTCCCCAGCTTCTCT | | | |
| | TTCCACCCAGGGATATGGAGCTTCA | | | |
| | AGCTTGGGTTTTAACTCAACCACTG | | | |
| | ATTGCTTGGATTATAAGGACCAAAC | | | |
| | TGCCTCCTGGAAGCTTAACTTCAAT | | | |
| | GCTGACTGCTTGGATTATAAAGATC | | | |
| | AGACATCCTCGTGGAAATTCCAGGT | | | |
| | TTTG | | | |
| PAX7 | ATGGCGGCCCTTCCCGGCACGGTAC | 79 | Involved in | Darabi, R. et |
| | CGAGAATGATGCGGCCGGCTCCGG | | specification | al. Human |
| | GGCAGAACTACCCCCGCACGGGAT | | and | ES- and iPS- |
| | TCCCTTTGGAAGTGTCCACCCCGCT | | differentiation | derived |
| | TGGCCAAGGCCGGGTCAATCAGCT | | of satellite | myogenic |
| | GGGAGGGGTCTTCATCAATGGGCG | | cells | progenitors |
| | ACCCCTGCCTAACCACATCCGCCAC | | Demonstrated to | restore |
| | AAGATAGTGGAGATGGCCCACCAT | | induce | DYSTROPHIN |
| | GGCATCCGGCCCTGTGTCATCTCCC | | myogenic | and |
| | GACAGCTGCGTGTCTCCCACGGCTG | | precursor | improve |
| | CGTCTCCAAGATTCTTTGCCGCTAC | | differentiation | contractility |
| | CAGGAGACCGGGTCCATCCGGCCT | | in hPSCs | upon |
| | GGGGCCATCGGCGGCAGCAAGCCC | | | transplantation |
| | AGACAGGTGGCGACTCCGGATGTA | | | in |
| | GAGAAAAAGATTGAGGAGTACAAG | | | dystrophic |
| | AGGGAAAACCCAGGCATGTTCAGC | | | mice. Cell |
| | TGGGAGATCCGGGACAGGCTGCTG | | | Stem Cell 10, |
| | AAGGATGGGCACTGTGACCGAAGC | | | 610-9 (2012). |
| | ACTGTGCCCTCAGTGAGTTCGATTA | | | Seale, P., et |
| | GCCGCGTGCTCAGAATCAAGTTCG | | | al. Pax7 Is |
| | GGAAGAAAGAGGAGGAGGATGAA | | | Required for |
| | GCGGACAAGAAGGAGGACGACGGC | | | the |
| | GAAAAGAAGGCCAAACACAGCATC | | | Specification |
| | GACGGCATCCTGGGCGACAAAGGG | | | of Myogenic |
| | AACCGGCTGGACGAGGGCTCGGAT | | | Satellite |
| | GTGGAGTCGGAACCTGACCTCCCA | | | Cells. Cell |
| | CTGAAGCGCAAGCAGCGACGCAGT | | | 102, 777-786 |
| | CGGACCACATTCACGGCCGAGCAG | | | (2000). |
| | CTGGAGGAGCTGGAGAAGGCCTTT | | | |
| | GAGAGGACCCACTACCCAGACATA | | | |
| | TACACCCGCGAGGAGCTGGCGCAG | | | |
| | AGGACCAAGCTGACAGAGGCGCGT | | | |
| | GTGCAGGTCTGGTTCAGTAACCGCC | | | |
| | GCGCCCGTTGGCGTAAGCAGGCAG | | | |
| | GAGCCAACCAGCTGGCGGCGTTCA | | | |
| | ACCACCTTCTGCCAGGAGGCTTCCC | | | |
| | GCCCACCGGCATGCCCACGCTGCC | | | |
| | CCCCTACCAGCTGCCGGACTCCACC | | | |
| | TACCCCACCACCACCATCTCCCAAG | | | |
| | ATGGGGGCAGCACTGTGCACCGGC | | | |
| | CTCAGCCCCTGCCACCGTCCACCAT | | | |
| | GCACCAGGGCGGGCTGGCTGCAGC | | | |
| | GGCTGCAGCCGCCGACACCAGCTC | | | |
| | TGCCTACGGAGCCCGCCACAGCTTC | | | |
| | TCCAGCTACTCTGACAGCTTCATGA | | | |
| | ATCCGGCGGCGCCCTCCAACCACAT | | | |
| | GAACCCGGTCAGCAACGGCCTGTC | | | |
| | TCCTCAGGTGATGAGCATCTTGGGC | | | |
| | AACCCCAGTGCGGTGCCCCCGCAG | | | |
| | CCACAGGCTGACTTCTCCATCTCCC | | | |
| | CGCTGCATGGCGGCCTGGACTCGG | | | |
| | CCACCTCCATCTCAGCCAGCTGCAG | | | |
| | CCAGCGGGCCGACTCCATCAAGCC | | | |
| | AGGAGACAGCCTGCCCACCTCCCA | | | |
| | GGCCTACTGCCCACCCACCTACAGC | | | |
| | ACCACCGGCTACAGCGTGGACCCC | | | |
| | GTGGCCGGCTATCAGTACGGCCAG | | | |
| | TACGGCCAGAGTGAGTGCCTGGTG | | | |
| | CCCTGGGCGTCCCCCGTCCCCATTC | | | |
| | CTTCTCCCACCCCCAGGGCCTCCTG | | | |
| | CTTGTTTATGGAGAGCTACAAGGTG | | | |
| | GTGTCAGGGTGGGGAATGTCCATTT | | | |
| | CACAGATGGAAAAATTGAAGTCCA | | | |
| | GCCAGATGGAACAGTTCACC | | | |
| POU1F1 | ATGAGTTGCCAAGCTTTTACTTCGG | 80 | Involved in | Turton, J. P. |
| | CTGATACCTTTATACCTCTGAATTC | | pituitary gland | G. et al. |
| | TGACGCCTCTGCAACTCTGCCTCTG | | development | Novel |
| | ATAATGCATCACAGTGCTGCCGAGT | | | Mutations |
| | GTCTACCAGTCTCCAACCATGCCAC | | | within the |
| | CAATGTGATGTCTACAGCAACAGG | | | POU1F1 |
| | ACTTCATTATTCTGTTCCTTCCTGTC | | | Gene |
| | ATTATGGAAACCAGCCATCAACCT | | | Associated |
| | ATGGAGTGATGGCAGGTAGTTTAA | | | with Variable |
| | CCCCTTGTCTTTATAAATTTCCTGA | | | Combined |
| | CCACACCTTGAGTCATGGATTTCCT | | | Pituitary |
| | CCTATACACCAGCCTCTTCTGGCAG | | | Hormone |
| | AGGACCCCACAGCTGCTGATTTCAA | | | Deficiency. J. |
| | GCAGGAACTCAGGCGGAAAAGTAA | | | Clin. |
| | ATTGGTGGAAGAGCCAATAGACAT | | | Endocrinol. |
| | GGATTCTCCAGAAATCAGAGAACT | | | Metab. 90, |
| | TGAAAAGTTTGCCAATGAATTTAAA | | | 4762-4770 |
| | GTGAGACGAATTAAATTAGGATAC | | | (2005). |
| | ACCCAGACAAATGTTGGGGAGGCC | | | |
| | CTGGCAGCTGTGCATGGCTCTGAAT | | | |
| | TCAGTCAAACAACAATCTGCCGATT | | | |
| | TGAAAATCTGCAGCTCAGCTTTAAA | | | |
| | AATGCATGCAAACTGAAAGCAATA | | | |
| | TTATCCAAATGGCTGGAGGAAGCT | | | |
| | GAGCAAGTAGGAGCTTTGTACAAT | | | |
| | GAAAAAGTGGGAGCAAATGAAAGG | | | |
| | AAAAGAAAACGAAGAACAACTATA | | | |
| | AGCATTGCTGCTAAAGATGCTCTGG | | | |
| | AGAGACACTTTGGAGAACAGAATA | | | |
| | AACCTTCTTCTCAAGAGATCATGAG | | | |
| | GATGGCTGAAGAACTGAATCTGGA | | | |
| | GAAAGAAGTAGTAAGAGTTTGGTT | | | |
| | TTGCAACCGGAGGCAGAGAGAAAA | | | |
| | ACGGGTGAAAACAAGTCTGAATCA | | | |
| | GAGTTTATTTTCTATTTCTAAGGAA | | | |
| | CATCTTGAGTGCAGATCAGGCCTCA | | | |
| | TGGGCCCAGCTTTCTTGTAC | | | |
| POU5F1 | ATGGCGGGACACCTGGCTTCAGATT | 81 | Involved in | Boyer, L. A., |
| | TTGCCTTCTCGCCCCCTCCAGGTGG | | regulation of | et al. Core |
| | TGGAGGTGATGGGCCAGGGGGGCC | | pluripotency | Transcriptional |
| | GGAGCCGGGCTGGGTTGATCCTCG | | and | Regulatory |
| | GACCTGGCTAAGCTTCCAAGGCCCT | | embryogenesis. | Circuitry in |
| | CCTGGAGGGCCAGGAATCGGGCCG | | Reprogramming | Human |
| | GGGGTTGGGCCAGGCTCTGAGGTG | | factor for | Embryonic |
| | TGGGGGATTCCCCCATGCCCCCCGC | | induction of | Stem Cells. |
| | CGTATGAGTTCTGTGGGGGGATGG | | pluripotency | Cell 122, |
| | CGTACTGTGGGCCCCAGGTTGGAGT | | | 947-956 |
| | GGGGCTAGTGCCCCAAGGCGGCTT | | | (2005). |
| | GGAGACCTCTCAGCCTGAGGGCGA | | | Takahashi, K. |
| | AGCAGGAGTCGGGGTGGAGAGCAA | | | & Yamanaka, |
| | CTCCGATGGGGCCTCCCCGGAGCCC | | | S. Induction |
| | TGCACCGTCACCCCTGGTGCCGTGA | | | of pluripotent |
| | AGCTGGAGAAGGAGAAGCTGGAGC | | | stem cells |
| | AAAACCCGGAGGAGTCCCAGGACA | | | from mouse |
| | TCAAAGCTCTGCAGAAAGAACTCG | | | embryonic |
| | AGCAATTTGCCAAGCTCCTGAAGC | | | and adult |
| | AGAAGAGGATCACCCTGGGATATA | | | fibroblast |
| | CACAGGCCGATGTGGGGCTCACCC | | | cultures by |
| | TGGGGGTTCTATTTGGGAAGGTATT | | | defined |
| | CAGCCAAACGACCATCTGCCGCTTT | | | factors. Cell |
| | GAGGCTCTGCAGCTTAGCTTCAAGA | | | 126,663-76 |
| | ACATGTGTAAGCTGCGGCCCTTGCT | | | (2006). |
| | GCAGAAGTGGGTGGAGGAAGCTGA | | | Takahashi, K. |
| | CAACAATGAAAATCTTCAGGAGAT | | | et al. |
| | ATGCAAAGCAGAAACCCTCGTGCA | | | Induction of |
| | GGCCCGAAAGAGAAAGCGAACCAG | | | pluripotent |
| | TATCGAGAACCGAGTGAGAGGCAA | | | stem cells |
| | CCTGGAGAATTTGTTCCTGCAGTGC | | | from adult |
| | CCGAAACCCACACTGCAGCAGATC | | | human |
| | AGCCACATCGCCCAGCAGCTTGGG | | | fibroblasts by |
| | CTCGAGAAGGATGTGGTCCGAGTG | | | defined |
| | TGGTTCTGTAACCGGCGCCAGAAG | | | factors. Cell |
| | GGCAAGCGATCAAGCAGCGACTAT | | | 131,861-72 |
| | GCACAACGAGAGGATTTTGAGGCT | | | (2007). |
| | GCTGGGTCTCCTTTCTCAGGGGGAC | | | Yu, J. et al. |
| | CAGTGTCCTTTCCTCTGGCCCCAGG | | | Induced |
| | GCCCCATTTTGGTACCCCAGGCTAT | | | Pluripotent |
| | GGGAGCCCTCACTTCACTGCACTGT | | | Stem Cell |
| | ACTCCTCGGTCCCTTTCCCTGAGGG | | | Lines Derived |
| | GGAAGCCTTTCCCCCTGTCTCTGTC | | | from Human |
| | ACCACTCTGGGCTCTCCCATGCATT | | | Somatic |
| | CAAAC | | | Cells. Science |
| | | | | (80-.). 318, |
| | | | | 1917-1920 |
| | | | | (2007). |
| RUNX1 | ATGGCTTCAGACAGCATATTTGAGT | 82 | Involved in | Woolf, E. et |
| | CATTTCCTTCGTACCCACAGTGCTT | | haematopoetic | al. Runx3 and |
| | CATGAGAGAATGCATACTTGGAAT | | cell | Runx1 are |
| | GAATCCTTCTAGAGACGTCCACGAT | | development | required for |
| | GCCAGCACGAGCCGCCGCTTCACG | | | CD8 T cell |
| | CCGCCTTCCACCGCGCTGAGCCCAG | | | development |
| | GCAAGATGAGCGAGGCGTTGCCGC | | | during |
| | TGGGCGCCCCGGACGCCGGCGCTG | | | thymopoiesis. |
| | CCCTGGCCGGCAAGCTGAGGAGCG | | | Proc. Natl. |
| | GCGACCGCAGCATGGTGGAGGTGC | | | Acad. Sci. U. |
| | TGGCCGACCACCCGGGCGAGCTGG | | | S. A. 100, |
| | TGCGCACCGACAGCCCCAACTTCCT | | | 7731-6 |
| | CTGCTCCGTGCTGCCTACGCACTGG | | | (2003). |
| | CGCTGCAACAAGACCCTGCCCATC | | | Lacaud, G. et |
| | GCTTTCAAGGTGGTGGCCCTAGGG | | | al. Runx1 is |
| | GATGTTCCAGATGGCACTCTGGTCA | | | essential for |
| | CTGTGATGGCTGGCAATGATGAAA | | | hematopoietic |
| | ACTACTCGGCTGAGCTGAGAAATG | | | commitment |
| | CTACCGCAGCCATGAAGAACCAGG | | | at the |
| | TTGCAAGATTTAATGACCTCAGGTT | | | hemangioblast |
| | TGTCGGTCGAAGTGGAAGAGGGAA | | | stage of |
| | AAGCTTCACTCTGACCATCACTGTC | | | development |
| | TTCACAAACCCACCGCAAGTCGCC | | | in vitro. |
| | ACCTACCACAGAGCCATCAAAATC | | | Blood 100, |
| | ACAGTGGATGGGCCCCGAGAACCT | | | 458-66 |
| | CGAAGACATCGGCAGAAACTAGAT | | | (2002). |
| | GATCAGACCAAGCCCGGGAGCTTG | | | |
| | TCCTTTTCCGAGCGGCTCAGTGAAC | | | |
| | TGGAGCAGCTGCGGCGCACAGCCA | | | |
| | TGAGGGTCAGCCCACACCACCCAG | | | |
| | CCCCCACGCCCAACCCTCGTGCCTC | | | |
| | CCTGAACCACTCCACTGCCTTTAAC | | | |
| | CCTCAGCCTCAGAGTCAGATGCAG | | | |
| | GATACAAGGCAGATCCAACCATCC | | | |
| | CCACCGTGGTCCTACGATCAGTCCT | | | |
| | ACCAATACCTGGGATCCATTGCCTC | | | |
| | TCCTTCTGTGCACCCAGCAACGCCC | | | |
| | ATTTCACCTGGACGTGCCAGCGGCA | | | |
| | TGACAACCCTCTCTGCAGAACTTTC | | | |
| | CAGTCGACTCTCAACGGCACCCGA | | | |
| | CCTGACAGCGTTCAGCGACCCGCG | | | |
| | CCAGTTCCCCGCGCTGCCCTCCATC | | | |
| | TCCGACCCCCGCATGCACTATCCAG | | | |
| | GCGCCTTCACCTACTCCCCGACGCC | | | |
| | GGTCACCTCGGGCATCGGCATCGG | | | |
| | CATGTCGGCCATGGGCTCGGCCAC | | | |
| | GCGCTACCACACCTACCTGCCGCCG | | | |
| | CCCTACCCCGGCTCGTCGCAAGCGC | | | |
| | AGGGAGGCCCGTTCCAAGCCAGCT | | | |
| | CGCCCTCCTACCACCTGTACTACGG | | | |
| | CGCCTCGGCCGGCTCCTACCAGTTC | | | |
| | TCCATGGTGGGCGGCGAGCGCTCG | | | |
| | CCGCCGCGCATCCTGCCGCCCTGCA | | | |
| | CCAACGCCTCCACCGGCTCCGCGCT | | | |
| | GCTCAACCCCAGCCTCCCGAACCA | | | |
| | GAGCGACGTGGTGGAGGCCGAGGG | | | |
| | CAGCCACAGCAACTCCCCCACCAA | | | |
| | CATGGCGCCCTCCGCGCGCCTGGA | | | |
| | GGAGGCCGTGTGGAGGCCCTAC | | | |
| SIX1 | ATGTCGATGCTGCCGTCGTTTGGCT | 83 | Involved in | Zheng, W. et |
| | TTACGCAGGAGCAAGTGGCGTGCG | | kidney, ear and | al. The role of |
| | TGTGCGAGGTTCTGCAGCAAGGCG | | olfactory | Six1 in |
| | GAAACCTGGAGCGCCTGGGCAGGT | | epithelium | mammalian |
| | TCCTGTGGTCACTGCCCGCCTGCGA | | development | auditory |
| | CCACCTGCACAAGAACGAGAGCGT | | | system |
| | ACTCAAGGCCAAGGCGGTGGTCGC | | | development. |
| | CTTCCACCGCGGCAACTTCCGTGAG | | | Development |
| | CTCTACAAGATCCTGGAGAGCCAC | | | 130, 3989- |
| | CAGTTCTCGCCTCACAACCACCCCA | | | 4000 (2003). |
| | AACTGCAGCAACTGTGGCTGAAGG | | | Xu, P. et al. |
| | CGCATTACGTGGAGGCCGAGAAGC | | | Six1 is |
| | TGTGCGGCCGACCCCTGGGCGCCGT | | | required for |
| | GGGCAAATATCGGGTGCGCCGAAA | | | the early |
| | ATTTCCACTGCCGCGCACCATCTGG | | | organogenesis |
| | GACGGCGAGGAGACCAGCTACTGC | | | of mammalian |
| | TTCAAGGAGAAGTCGAGGGGTGTC | | | kidney. |
| | CTGCGGGAGTGGTACGCGCACAAT | | | Development |
| | CCCTACCCATCGCCGCGTGAGAAG | | | 130, 3085- |
| | CGGGAGCTGGCCGAGGCCACCGGC | | | 3094 (2003). |
| | CTCACCACCACCCAGGTCAGCAACT | | | Ikeda, K. et |
| | GGTTTAAGAACCGGAGGCAAAGAG | | | al. Six1 is |
| | ACCGGGCCGCGGAGGCCAAGGAAA | | | essential for |
| | GGGAGAACACCGAAAACAATAACT | | | early |
| | CCTCCTCCAACAAGCAGAACCAAC | | | neurogenesis |
| | TCTCTCCTCTGGAAGGGGGCAAGCC | | | in the |
| | GCTCATGTCCAGCTCAGAAGAGGA | | | development |
| | ATTCTCACCTCCCCAAAGTCCAGAC | | | of olfactory |
| | CAGAACTCGGTCCTTCTGCTGCAGG | | | epithelium. |
| | GCAATATGGGCCACGCCAGGAGCT | | | Dev. Biol. |
| | CAAACTATTCTCTCCCGGGCTTAAC | | | 311, 53-68 |
| | AGCCTCGCAGCCCAGTCACGGCCT | | | (2007). |
| | GCAGACCCACCAGCATCAGCTCCA | | | |
| | AGACTCTCTGCTCGGCCCCCTCACC | | | |
| | TCCAGTCTGGTGGACTTGGGGTCC | | | |
| SIX2 | ATGTCCATGCTGCCCACCTTCGGCT | 84 | Involved in | Kobayashi, A. |
| | TCACGCAGGAGCAAGTGGCGTGCG | | kidney | et al. Six2 |
| | TGTGCGAGGTGCTGCAGCAGGGCG | | development | Defines and |
| | GCAACATCGAGCGGCTGGGCCGCT | | | Regulates a |
| | TCCTGTGGTCGCTGCCCGCCTGCGA | | | Multipotent |
| | GCACCTTCACAAGAATGAAAGCGT | | | Self- |
| | GCTCAAGGCCAAGGCCGTGGTGGC | | | Renewing |
| | CTTCCACCGCGGCAACTTCCGCGAG | | | Nephron |
| | CTCTACAAGATCCTGGAGAGCCAC | | | Progenitor |
| | CAGTTCTCGCCGCACAACCACGCCA | | | Population |
| | AGCTGCAGCAGCTGTGGCTCAAGG | | | throughout |
| | CACACTACATCGAGGCGGAGAAGC | | | Mammalian |
| | TGCGCGGCCGACCCCTGGGCGCCG | | | Kidney |
| | TGGGCAAATACCGCGTGCGCCGCA | | | Development. |
| | AATTCCCGCTGCCGCGCTCCATCTG | | | Cell Stem |
| | GGACGGCGAGGAGACCAGCTACTG | | | Cell 3, 169- |
| | CTTCAAGGAAAAGAGTCGCAGCGT | | | 181 (2008). |
| | GCTGCGCGAGTGGTACGCGCACAA | | | |
| | CCCCTACCCTTCACCCCGCGAGAAG | | | |
| | CGTGAGCTGACGGAGGCCACGGGC | | | |
| | CTCACCACCACACAGGTCAGCAAC | | | |
| | TGGTTCAAGAACCGGCGGCAGCGC | | | |
| | GACCGGGCGGCCGAGGCCAAGGAA | | | |
| | AGGGAGAACAACGAGAACTCCAAT | | | |
| | TCTAACAGCCACAACCCGCTGAAT | | | |
| | GGCAGCGGCAAGTCGGTGTTAGGC | | | |
| | AGCTCGGAGGATGAGAAGACTCCA | | | |
| | TCGGGGACGCCAGACCACTCATCA | | | |
| | TCCAGCCCCGCACTGCTCCTCAGCC | | | |
| | CGCCGCCCCCTGGGCTGCCGTCCCT | | | |
| | GCACAGCCTGGGCCACCCTCCGGG | | | |
| | CCCCAGCGCAGTGCCAGTGCCGGT | | | |
| | GCCAGGCGGAGGTGGAGCGGACCC | | | |
| | ACTGCAACACCACCATGGCCTGCA | | | |
| | GGACTCCATCCTCAACCCCATGTCA | | | |
| | GCCAACCTCGTGGACCTGGGCTCC | | | |
| SNAI2 | ATGCCGCGCTCCTTCCTGGTCAAGA | 85 | Involved in | Cobaleda, C., |
| | AGCATTTCAACGCCTCCAAAAAGC | | neural crest | Pérez-Caro, |
| | CAAACTACAGCGAACTGGACACAC | | development, | M., Vicente- |
| | ATACAGTGATTATTTCCCCGTATCT | | epithelial- | Dueñas, C. & |
| | CTATGAGAGTTACTCCATGCCTGTC | | mesenchymal | Sánchez- |
| | ATACCACAACCAGAGATCCTCAGC | | transition, and | García, I. |
| | TCAGGAGCATACAGCCCCATCACT | | melanocyte | Function of |
| | GTGTGGACTACCGCTGCTCCATTCC | | stem cell | the Zinc- |
| | ACGCCCAGCTACCCAATGGCCTCTC | | development | Finger |
| | TCCTCTTTCCGGATACTCCTCATCTT | | | Transcription |
| | TGGGGCGAGTGAGTCCCCCTCCTCC | | | Factor SNAI2 |
| | ATCTGACACCTCCTCCAAGGACCAC | | | in Cancer and |
| | AGTGGCTCAGAAAGCCCCATTAGT | | | Development. |
| | GATGAAGAGGAAAGACTACAGTCC | | | Annu. Rev. |
| | AAGCTTTCAGACCCCCATGCCATTG | | | Genet. 41, |
| | AAGCTGAAAAGTTTCAGTGCAATTT | | | 41-61 (2007). |
| | ATGCAATAAGACCTATTCAACTTTT | | | |
| | TCTGGGCTGGCCAAACATAAGCAG | | | |
| | CTGCACTGCGATGCCCAGTCTAGAA | | | |
| | AATCTTTCAGCTGTAAATACTGTGA | | | |
| | CAAGGAATATGTGAGCCTGGGCGC | | | |
| | CCTGAAGATGCATATTCGGACCCAC | | | |
| | ACATTACCTTGTGTTTGCAAGATCT | | | |
| | GCGGCAAGGCGTTTTCCAGACCCTG | | | |
| | GTTGCTTCAAGGACACATTAGAACT | | | |
| | CACACGGGGGAGAAGCCTTTTTCTT | | | |
| | GCCCTCACTGCAACAGAGCATTTGC | | | |
| | AGACAGGTCAAATCTGAGGGCTCA | | | |
| | TCTGCAGACCCATTCTGATGTAAAG | | | |
| | AAATACCAGTGCAAAAACTGCTCC | | | |
| | AAAACCTTCTCCAGAATGTCTCTCC | | | |
| | TGCACAAACATGAGGAATCTGGCT | | | |
| | GCTGTGTAGCACAC | | | |
| SOX10 | ATGGCGGAGGAGCAGGACCTATCG | 86 | Involved in | Southard- |
| | GAGGTGGAGCTGAGCCCCGTGGGC | | neural crest and | Smith, E. M., |
| | TCGGAGGAGCCCCGCTGCCTGTCCC | | neuronal | Kos, L. & |
| | CGGGGAGCGCGCCCTCGCTAGGGC | | development | Pavan, W. J. |
| | CCGACGGCGGCGGCGGCGGATCGG | | | SOX10 |
| | GCCTGCGAGCCAGCCCGGGGCCAG | | | mutation |
| | GCGAGCTGGGCAAGGTCAAGAAGG | | | disrupts |
| | AGCAGCAGGACGGCGAGGCGGACG | | | neural crest |
| | ATGACAAGTTCCCCGTGTGCATCCG | | | development |
| | CGAGGCCGTCAGCCAGGTGCTCAG | | | in Dom |
| | CGGCTACGACTGGACGCTGGTGCC | | | Hirschsprung |
| | CATGCCCGTGCGCGTCAACGGCGC | | | mouse model. |
| | CAGCAAAAGCAAGCCGCACGTCAA | | | Nat. Genet. |
| | GCGGCCCATGAACGCCTTCATGGTG | | | 18, 60-64 |
| | TGGGCTCAGGCAGCGCGCAGGAAG | | | (1998). |
| | CTCGCGGACCAGTACCCGCACCTGC | | | Britsch, S. et |
| | ACAACGCTGAGCTCAGCAAGACGC | | | al. The |
| | TGGGCAAGCTCTGGAGGCTGCTGA | | | transcription |
| | ACGAAAGTGACAAGCGCCCCTTCA | | | factor Sox10 |
| | TCGAGGAGGCTGAGCGGCTCCGTA | | | is a key |
| | TGCAGCACAAGAAAGACCACCCGG | | | regulator of |
| | ACTACAAGTACCAGCCCAGGCGGC | | | peripheral |
| | GGAAGAACGGGAAGGCCGCCCAGG | | | glial |
| | GCGAGGCGGAGTGCCCCGGTGGGG | | | development. |
| | AGGCCGAGCAAGGTGGGACCGCCG | | | Genes Dev. |
| | CCATCCAGGCCCACTACAAGAGCG | | | 15, 66-78 |
| | CCCACTTGGACCACCGGCACCCAG | | | (2001). |
| | GAGAGGGCTCCCCCATGTCAGATG | | | |
| | GGAACCCCGAGCACCCCTCAGGCC | | | |
| | AGAGCCATGGCCCACCCACCCCTC | | | |
| | CAACCACCCCGAAGACAGAGCTGC | | | |
| | AGTCGGGCAAGGCAGACCCGAAGC | | | |
| | GGGACGGGCGCTCCATGGGGGAGG | | | |
| | GCGGGAAGCCTCACATCGACTTCG | | | |
| | GCAACGTGGACATTGGTGAGATCA | | | |
| | GCCACGAGGTAATGTCCAACATGG | | | |
| | AGACCTTTGATGTGGCTGAGTTGGA | | | |
| | CCAGTACCTGCCGCCCAATGGGCA | | | |
| | CCCAGGCCATGTGAGCAGCTACTC | | | |
| | AGCAGCCGGCTATGGGCTGGGCAG | | | |
| | TGCCCTGGCCGTGGCCAGTGGACA | | | |
| | CTCCGCCTGGATCTCCAAGCCACCA | | | |
| | GGCGTGGCTCTGCCCACGGTCTCAC | | | |
| | CACCTGGTGTGGATGCCAAAGCCC | | | |
| | AGGTGAAGACAGAGACCGCGGGGC | | | |
| | CCCAGGGGCCCCCACACTACACCG | | | |
| | ACCAGCCATCCACCTCACAGATCGC | | | |
| | CTACACCTCCCTCAGCCTGCCCCAC | | | |
| | TATGGCTCAGCCTTCCCCTCCATCT | | | |
| | CCCGCCCCCAGTTTGACTACTCTGA | | | |
| | CCATCAGCCCTCAGGACCCTATTAT | | | |
| | GGCCACTCGGGCCAGGCCTCTGGC | | | |
| | CTCTACTCGGCCTTCTCCTATATGG | | | |
| | GGCCCTCGCAGCGGCCCCTCTACAC | | | |
| | GGCCATCTCTGACCCCAGCCCCTCA | | | |
| | GGGCCCCAGTCCCACAGCCCCACA | | | |
| | CACTGGGAGCAGCCAGTATATACG | | | |
| | ACACTGTCCCGGCCC | | | |
| SOX2 | ATGTACAACATGATGGAGACGGAG | 87 | Involved in | Boyer, L. A., |
| | CTGAAGCCGCCGGGCCCGCAGCAA | | regulation of | et al. Core |
| | ACTTCGGGGGGCGGCGGCGGCAAC | | pluripotency | Transcriptional |
| | TCCACCGCGGCGGCGGCCGGCGGC | | and | Regulatory |
| | AACCAGAAAAACAGCCCGGACCGC | | embryogenesis, | Circuitry in |
| | GTCAAGCGGCCCATGAATGCCTTCA | | and in neuronal | Human |
| | TGGTGTGGTCCCGCGGGCAGCGGC | | development. | Embryonic |
| | GCAAGATGGCCCAGGAGAACCCCA | | Reprogramming | Stem Cells. |
| | AGATGCACAACTCGGAGATCAGCA | | factor for | Cell 122, |
| | AGCGCCTGGGCGCCGAGTGGAAAC | | induction of | 947-956 |
| | TTTTGTCGGAGACGGAGAAGCGGC | | pluripotency. | (2005). |
| | CGTTCATCGACGAGGCTAAGCGGC | | | Graham, V. et |
| | TGCGAGCGCTGCACATGAAGGAGC | | | al. SOX2 |
| | ACCCGGATTATAAATACCGGCCCC | | | Functions to |
| | GGCGGAAAACCAAGACGCTCATGA | | | Maintain |
| | AGAAGGATAAGTACACGCTGCCCG | | | Neural |
| | GCGGGCTGCTGGCCCCCGGCGGCA | | | Progenitor |
| | ATAGCATGGCGAGCGGGGTCGGGG | | | Identity. |
| | TGGGCGCCGGCCTGGGCGCGGGCG | | | Neuron 39, |
| | TGAACCAGCGCATGGACAGTTACG | | | 749-765 |
| | CGCACATGAACGGCTGGAGCAACG | | | (2003). |
| | GCAGCTACAGCATGATGCAGGACC | | | Wang, Z., |
| | AGCTGGGCTACCCGCAGCACCCGG | | | Oron, E., |
| | GCCTCAATGCGCACGGCGCAGCGC | | | Nelson, B., |
| | AGATGCAGCCCATGCACCGCTACG | | | Razis, S. & |
| | ACGTGAGCGCCCTGCAGTACAACT | | | Ivanova, N. |
| | CCATGACCAGCTCGCAGACCTACAT | | | Distinct |
| | GAACGGCTCGCCCACCTACAGCAT | | | Lineage |
| | GTCCTACTCGCAGCAGGGCACCCCT | | | Specification |
| | GGCATGGCTCTTGGCTCCATGGGTT | | | Roles for |
| | CGGTGGTCAAGTCCGAGGCCAGCT | | | NANOG, |
| | CCAGCCCCCCTGTGGTTACCTCTTC | | | OCT4, and |
| | CTCCCACTCCAGGGCGCCCTGCCAG | | | SOX2 in |
| | GCCGGGGACCTCCGGGACATGATC | | | Human |
| | AGCATGTATCTCCCCGGCGCCGAG | | | Embryonic |
| | GTGCCGGAACCCGCCGCCCCCAGC | | | Stem Cells. |
| | AGACTTCACATGTCCCAGCACTACC | | | Cell Stem |
| | AGAGCGGCCCGGTGCCCGGCACGG | | | Cell 10, 440- |
| | CCATTAACGGCACACTGCCCCTCTC | | | 454 (2012). |
| | ACACATG | | | Takahashi, K. |
| | | | | & Yamanaka, |
| | | | | S. Induction |
| | | | | of pluripotent |
| | | | | stem cells |
| | | | | from mouse |
| | | | | embryonic |
| | | | | and adult |
| | | | | fibroblast |
| | | | | cultures by |
| | | | | defined |
| | | | | factors. Cell |
| | | | | 126, 663-76 |
| | | | | (2006). |
| | | | | Takahashi, K. |
| | | | | et al. |
| | | | | Induction of |
| | | | | pluripotent |
| | | | | stem cells |
| | | | | from adult |
| | | | | human |
| | | | | fibroblasts by |
| | | | | defined |
| | | | | factors. Cell |
| | | | | 131, 861-72 |
| | | | | (2007). |
| | | | | Yu, J. et al. |
| | | | | Induced |
| | | | | Pluripotent |
| | | | | Stem Cell |
| | | | | Lines Derived |
| | | | | from Human |
| | | | | Somatic |
| | | | | Cells. Science |
| | | | | (80-.). 318, |
| | | | | 1917-1920 |
| | | | | (2007). |
| SOX3 | ATGCGACCTGTTCGAGAGAACTCAT | 88 | Involved in | Rizzoti, K. et |
| | CAGGTGCGAGAAGCCCGCGGGTTC | | neuronal and | al. SOX3 is |
| | CTGCTGATTTGGCGCGGAGCATTTT | | pituitary | required |
| | GATAAGCCTACCCTTCCCGCCGGAC | | development | during the |
| | TCGCTGGCCCACAGGCCCCCAAGCT | | | formation of |
| | CCGCTCCGACGGAGTCCCAGGGCC | | | the |
| | TTTTCACCGTGGCCGCTCCAGCCCC | | | hypothalamo- |
| | GGGAGCGCCTTCTCCTCCCGCCACG | | | pituitary axis. |
| | CTGGCGCACCTTCTTCCCGCCCCGG | | | Nat. Genet. |
| | CAATGTACAGCCTTCTGGAGACTGA | | | 36, 247-255 |
| | ACTCAAGAACCCCGTAGGGACACC | | | (2004). |
| | CACACAAGCGGCGGGCACCGGCGG | | | |
| | CCCCGCAGCCCCGGGAGGCGCAGG | | | |
| | CAAGAGTAGTGCGAACGCAGCCGG | | | |
| | CGGCGCGAACTCGGGCGGCGGCAG | | | |
| | CAGCGGTGGTGCGAGCGGAGGTGG | | | |
| | CGGGGGTACAGACCAGGACCGTGT | | | |
| | GAAACGGCCCATGAACGCCTTCAT | | | |
| | GGTATGGTCCCGCGGGCAGCGGCG | | | |
| | CAAAATGGCCCTGGAGAACCCCAA | | | |
| | GATGCACAATTCTGAGATCAGCAA | | | |
| | GCGCTTGGGCGCCGACTGGAAACT | | | |
| | GCTGACCGACGCCGAGAAGCGACC | | | |
| | ATTCATCGACGAGGCCAAGCGACT | | | |
| | TCGCGCCGTGCACATGAAGGAGTA | | | |
| | TCCGGACTACAAGTACCGACCGCG | | | |
| | CCGCAAGACCAAGACGCTGCTCAA | | | |
| | GAAAGATAAGTACTCCCTGCCCAG | | | |
| | CGGCCTCCTGCCTCCCGGTGCCGCG | | | |
| | GCCGCCGCCGCCGCTGCCGCGGCC | | | |
| | GCAGCCGCTGCCGCCAGCAGTCCG | | | |
| | GTGGGCGTGGGCCAGCGCCTGGAC | | | |
| | ACGTACACGCACGTGAACGGCTGG | | | |
| | GCCAACGGCGCGTACTCGCTGGTG | | | |
| | CAGGAGCAGCTGGGCTACGCGCAG | | | |
| | CCCCCGAGCATGAGCAGCCCGCCG | | | |
| | CCGCCGCCCGCGCTGCCGCCGATG | | | |
| | CACCGCTACGACATGGCCGGCCTG | | | |
| | CAGTACAGCCCAATGATGCCGCCC | | | |
| | GGCGCTCAGAGCTACATGAACGTC | | | |
| | GCTGCCGCGGCCGCCGCCGCCTCG | | | |
| | GGCTACGGGGGCATGGCGCCCTCA | | | |
| | GCCACAGCAGCCGCGGCCGCCGCC | | | |
| | TACGGGCAGCAGCCCGCCACCGCC | | | |
| | GCGGCCGCAGCTGCGGCCGCAGCC | | | |
| | GCCATGAGCCTGGGCCCCATGGGC | | | |
| | TCGGTAGTGAAGTCTGAGCCCAGCT | | | |
| | CGCCGCCGCCCGCCATCGCATCGC | | | |
| | ACTCTCAGCGCGCGTGCCTCGGCGA | | | |
| | CCTGCGCGACATGATCAGCATGTAC | | | |
| | CTGCCACCCGGCGGGGACGCGGCC | | | |
| | GACGCCGCCTCTCCGCTGCCCGGCG | | | |
| | GTCGCCTGCACGGCGTGCACCAGC | | | |
| | ACTACCAGGGCGCCGGGACTGCAG | | | |
| | TCAACGGAACGGTGCCGCTGACCC | | | |
| | ACATC | | | |
| SPI1 | ATGTTACAGGCGTGCAAAATGGAA | 89 | Involved in | Scott, E. W. |
| | GGGTTTCCCCTCGTCCCCCCTCAGC | | haematopoetic | et al. |
| | CATCAGAAGACCTGGTGCCCTATG | | cell | Requirement |
| | ACACGGATCTATACCAACGCCAAA | | development | of |
| | CGCACGAGTATTACCCCTATCTCAG | | | transcription |
| | CAGTGATGGGGAGAGCCATAGCGA | | | factor PU.1 in |
| | CCATTACTGGGACTTCCACCCCCAC | | | the |
| | CACGTGCACAGCGAGTTCGAGAGC | | | development |
| | TTCGCCGAGAACAACTTCACGGAG | | | of multiple |
| | CTCCAGAGCGTGCAGCCCCCGCAG | | | hematopoietic |
| | CTGCAGCAGCTCTACCGCCACATGG | | | lineages. |
| | AGCTGGAGCAGATGCACGTCCTCG | | | Science 265, |
| | ATACCCCCATGGTGCCACCCCATCC | | | 1573-1577 |
| | CAGTCTTGGCCACCAGGTCTCCTAC | | | (1994). |
| | CTGCCCCGGATGTGCCTCCAGTACC | | | Rosenbauer, |
| | CATCCCTGTCCCCAGCCCAGCCCAG | | | F. & Tenen, |
| | CTCAGATGAGGAGGAGGGCGAGCG | | | D. G. |
| | GCAGAGCCCCCCACTGGAGGTGTC | | | Transcription |
| | TGACGGCGAGGCGGATGGCCTGGA | | | factors in |
| | GCCCGGGCCTGGGCTCCTGCCTGGG | | | myeloid |
| | GAGACAGGCAGCAAGAAGAAGATC | | | development: |
| | CGCCTGTACCAGTTCCTGTTGGACC | | | balancing |
| | TGCTCCGCAGCGGCGACATGAAGG | | | differentiation |
| | ACAGCATCTGGTGGGTGGACAAGG | | | with |
| | ACAAGGGCACCTTCCAGTTCTCGTC | | | transformation. |
| | CAAGCACAAGGAGGCGCTGGCGCA | | | Nat. Rev. |
| | CCGCTGGGGCATCCAGAAGGGCAA | | | Immunol. 7, |
| | CCGCAAGAAGATGACCTACCAGAA | | | 105-117 |
| | GATGGCGCGCGCGCTGCGCAACTA | | | (2007). |
| | CGGCAAGACGGGCGAGGTCAAGAA | | | |
| | GGTGAAGAAGAAGCTCACCTACCA | | | |
| | GTTCAGCGGCGAAGTGCTGGGCCG | | | |
| | CGGGGGCCTGGCCGAGCGGCGCCA | | | |
| | CCCGCCCCAC | | | |
| SPIB | ATGCTCGCCCTGGAGGCTGCACAG | 90 | Involved in | Maroulakou, |
| | CTCGACGGGCCACACTTCAGCTGTC | | differentiation | I. G. & Bowe, |
| | TGTACCCAGATGGCGTCTTCTATGA | | of lymphoid | D. B. |
| | CCTGGACAGCTGCAAGCATTCCAG | | cells | Expression |
| | CTACCCTGATTCAGAGGGGGCTCCT | | | and function |
| | GACTCCCTGTGGGACTGGACTGTGG | | | of Ets |
| | CCCCACCTGTCCCAGCCACCCCCTA | | | transcription |
| | TGAAGCCTTCGACCCGGCAGCAGC | | | factors in |
| | CGCTTTTAGCCACCCCCAGGCTGCC | | | mammalian |
| | CAGCTCTGCTACGAACCCCCCACCT | | | development: |
| | ACAGCCCTGCAGGGAACCTCGAAC | | | a regulatory |
| | TGGCCCCCAGCCTGGAGGCCCCGG | | | network. |
| | GGCCTGGCCTCCCCGCATACCCCAC | | | Oncogene 19, |
| | GGAGAACTTCGCTAGCCAGACCCT | | | 6432-6442 |
| | GGTTCCCCCGGCATATGCCCCGTAC | | | (2000). |
| | CCCAGCCCTGTGCTATCAGAGGAG | | | |
| | GAAGACTTACCGTTGGACAGCCCT | | | |
| | GCCCTGGAGGTCTCGGACAGCGAG | | | |
| | TCGGATGAGGCCCTCGTGGCTGGCC | | | |
| | CCGAGGGGAAGGGATCCGAGGCAG | | | |
| | GGACTCGCAAGAAGCTGCGCCTGT | | | |
| | ACCAGTTCCTGCTGGGGCTACTGAC | | | |
| | GCGCGGGGACATGCGTGAGTGCGT | | | |
| | GTGGTGGGTGGAGCCAGGCGCCGG | | | |
| | CGTCTTCCAGTTCTCCTCCAAGCAC | | | |
| | AAGGAACTCCTGGCGCGCCGCTGG | | | |
| | GGCCAGCAGAAGGGGAACCGCAAG | | | |
| | CGCATGACCTACCAGAAGCTGGCG | | | |
| | CGCGCCCTCCGAAACTACGCCAAG | | | |
| | ACCGGCGAGATCCGCAAGGTCAAG | | | |
| | CGCAAGCTCACCTACCAGTTCGACA | | | |
| | GCGCGCTGCTGCCTGCAGTCCGCCG | | | |
| | GGCCTTG | | | |
| SPIC | ATGACGTGTGTTGAACAAGACAAG | 91 | Involved in | Kohyama, M. |
| | CTGGGTCAAGCATTTGAAGATGCTT | | macrophage | et al. Role for |
| | TTGAGGTTCTGAGGCAACATTCAAC | | development | Spi-C in the |
| | TGGAGATCTTCAGTACTCGCCAGAT | | | development |
| | TACAGAAATTACCTGGCTTTAATCA | | | of red pulp |
| | ACCATCGTCCTCATGTCAAAGGAA | | | macrophages |
| | ATTCCAGCTGCTATGGAGTGTTGCC | | | and splenic |
| | TACAGAGGAGCCTGTCTATAATTGG | | | iron |
| | AGAACGGTAATTAACAGTGCTGCG | | | homeostasis. |
| | GACTTCTATTTTGAAGGAAATATTC | | | Nature 457, |
| | ATCAATCTCTGCAGAACATAACTGA | | | 318-321 |
| | AAACCAGCTGGTACAACCCACTCTT | | | (2009). |
| | CTCCAGCAAAAGGGGGGAAAAGGC | | | |
| | AGGAAGAAGCTCCGACTGTTTGAA | | | |
| | TACCTTCACGAATCCCTGTATAATC | | | |
| | CGGAGATGGCATCTTGTATTCAGTG | | | |
| | GGTAGATAAAACCAAAGGCATCTT | | | |
| | TCAGTTTGTATCAAAAAACAAAGA | | | |
| | AAAACTTGCCGAGCTTTGGGGGAA | | | |
| | AAGAAAAGGCAACAGGAAGACCAT | | | |
| | GACTTACCAGAAAATGGCCAGGGC | | | |
| | ACTCAGAAATTACGGAAGAAGTGG | | | |
| | GGAAATTACCAAAATCCGGAGGAA | | | |
| | GCTGACTTACCAGTTCAGTGAGGCC | | | |
| | ATTCTCCAAAGACTCTCTCCATCCT | | | |
| | ATTTCCTGGGGAAAGAGATCTTCTA | | | |
| | TTCACAGTGTGTTCAACCTGATCAA | | | |
| | GAATATCTCAGTTTAAATAACTGGA | | | |
| | ATGCAAATTATAATTATACATATGC | | | |
| | CAATTACCATGAGCTAAATCACCAT | | | |
| | GATTGC | | | |
| SRY | ATGCAATCATATGCTTCTGCTATGT | 92 | Involved in sex | Polanco, J. C. |
| | TAAGCGTATTCAACAGCGATGATTA | | determination | & Koopman, |
| | CAGTCCAGCTGTGCAAGAGAATAT | | and | P. Sry and the |
| | TCCCGCTCTCCGGAGAAGCTCTTCC | | spermatogenesis | hesitant |
| | TTCCTTTGCACTGAAAGCTGTAACT | | | beginnings of |
| | CTAAGTATCAGTGTGAAACGGGAG | | | male |
| | AAAACAGTAAAGGCAACGTCCAGG | | | development. |
| | ATAGAGTGAAGCGACCCATGAACG | | | Dev. Biol. |
| | CATTCATCGTGTGGTCTCGCGATCA | | | 302,13-24 |
| | GAGGCGCAAGATGGCTCTAGAGAA | | | (2007). |
| | TCCCAGAATGCGAAACTCAGAGAT | | | Koopman, P. |
| | CAGCAAGCAGCTGGGATACCAGTG | | | et al. Male |
| | GAAAATGCTTACTGAAGCCGAAAA | | | development |
| | ATGGCCATTCTTCCAGGAGGCACA | | | of |
| | GAAATTACAGGCCATGCACAGAGA | | | chromosomally |
| | GAAATACCCGAATTATAAGTATCG | | | female mice |
| | ACCTCGTCGGAAGGCGAAGATGCT | | | transgenic for |
| | GCCGAAGAATTGCAGTTTGCTTCCC | | | Sry. Nature |
| | GCAGATCCCGCTTCGGTACTCTGCA | | | 351,117-121 |
| | GCGAAGTGCAACTGGACAACAGGT | | | (1991). |
| | TGTACAGGGATGACTGTACGAAAG | | | |
| | CCACACACTCAAGAATGGAGCACC | | | |
| | AGCTAGGCCACTTACCGCCCATCAA | | | |
| | CGCAGCCAGCTCACCGCAGCAACG | | | |
| | GGACCGCTACAGCCACTGGACAAA | | | |
| | GCTG | | | |
| TBX5 | ATGGCCGACGCAGACGAGGGCTTT | 93 | Involved in | Bruneau, B. |
| | GGCCTGGCGCACACGCCTCTGGAG | | cardiac | G. et al. A |
| | CCTGACGCAAAAGACCTGCCCTGC | | development | Murine Model |
| | GATTCGAAACCCGAGAGCGCGCTC | | | of Holt-Oram |
| | GGGGCCCCCAGCAAGTCCCCGTCG | | | Syndrome |
| | TCCCCGCAGGCCGCCTTCACCCAGC | | | Defines Roles |
| | AGGGCATGGAGGGAATCAAAGTGT | | | of the T-Box |
| | TTCTCCATGAAAGAGAACTGTGGCT | | | Transcription |
| | AAAATTCCACGAAGTGGGCACGGA | | | Factor Tbx5 |
| | AATGATCATAACCAAGGCTGGAAG | | | in |
| | GCGGATGTTTCCCAGTTACAAAGTG | | | Cardiogenesis |
| | AAGGTGACGGGCCTTAATCCCAAA | | | and Disease. |
| | ACGAAGTACATTCTTCTCATGGACA | | | Cell 106, |
| | TTGTACCTGCCGACGATCACAGATA | | | 709-721 |
| | CAAATTCGCAGATAATAAATGGTCT | | | (2001). |
| | GTGACGGGCAAAGCTGAGCCCGCC | | | |
| | ATGCCTGGCCGCCTGTACGTGCACC | | | |
| | CAGACTCCCCCGCCACCGGGGCGC | | | |
| | ATTGGATGAGGCAGCTCGTCTCCTT | | | |
| | CCAGAAACTCAAGCTCACCAACAA | | | |
| | CCACCTGGACCCATTTGGGCATATT | | | |
| | ATTCTAAATTCCATGCACAAATACC | | | |
| | AGCCTAGATTACACATCGTGAAAG | | | |
| | CGGATGAAAATAATGGATTTGGCT | | | |
| | CAAAAAATACAGCGTTCTGCACTC | | | |
| | ACGTCTTTCCTGAGACTGCGTTTAT | | | |
| | AGCAGTGACTTCCTACCAGAACCA | | | |
| | CAAGATCACGCAATTAAAGATTGA | | | |
| | GAATAATCCCTTTGCCAAAGGATTT | | | |
| | CGGGGCAGTGATGACATGGAGCTG | | | |
| | CACAGAATGTCAAGAATGCAAAGT | | | |
| | AAAGAATATCCCGTGGTCCCCAGG | | | |
| | AGCACCGTGAGGCAAAAAGTGGCC | | | |
| | TCCAACCACAGTCCTTTCAGCAGCG | | | |
| | AGTCTCGAGCTCTCTCCACCTCATC | | | |
| | CAATTTGGGGTCCCAATACCAGTGT | | | |
| | GAGAATGGTGTTTCCGGCCCCTCCC | | | |
| | AGGACCTCCTGCCTCCACCCAACCC | | | |
| | ATACCCACTGCCCCAGGAGCATAG | | | |
| | CCAAATTTACCATTGTACCAAGAGG | | | |
| | AAAGAGGAAGAATGTTCCACCACA | | | |
| | GACCATCCCTATAAGAAGCCCTAC | | | |
| | ATGGAGACATCACCCAGTGAAGAA | | | |
| | GATTCCTTCTACCGCTCTAGCTATC | | | |
| | CACAGCAGCAGGGCCTGGGTGCCT | | | |
| | CCTACAGGACAGAGTCGGCACAGC | | | |
| | GGCAAGCTTGCATGTATGCCAGCTC | | | |
| | TGCGCCCCCCAGCGAGCCTGTGCCC | | | |
| | AGCCTAGAGGACATCAGCTGCAAC | | | |
| | ACGTGGCCAAGCATGCCTTCCTACA | | | |
| | GCAGCTGCACCGTCACCACCGTGC | | | |
| | AGCCCATGGACAGGCTACCCTACC | | | |
| | AGCACTTCTCCGCTCACTTCACCTC | | | |
| | GGGGCCCCTGGTCCCTCGGCTGGCT | | | |
| | GGCATGGCCAACCATGGCTCCCCA | | | |
| | CAGCTGGGAGAGGGAATGTTCCAG | | | |
| | CACCAGACCTCCGTGGCCCACCAG | | | |
| | CCTGTGGTCAGGCAGTGTGGGCCTC | | | |
| | AGACTGGCCTGCAGTCCCCTGGCAC | | | |
| | CCTTCAGCCCCCTGAGTTCCTCTAC | | | |
| | TCTCATGGCGTGCCAAGGACTCTAT | | | |
| | CCCCTCATCAGTACCACTCTGTGCA | | | |
| | CGGAGTTGGCATGGTGCCAGAGTG | | | |
| | GAGCGACAATAGCTTG | | | |
| TFAP2 | ATGTTGTGGAAAATAACCGATAAT | 94 | Involved in | Cao, Z. et al. |
| C | GTCAAGTACGAAGAGGACTGCGAG | | trophectoderm | Transcription |
| | GATCGCCACGACGGGAGCAGCAAT | | development | factor AP-2γ |
| | GGGAATCCGCGGGTCCCCCACCTCT | | | induces early |
| | CCTCCGCCGGGCAGCACCTCTACAG | | | Cdx2 |
| | CCCCGCGCCACCCCTCTCCCACACT | | | expression |
| | GGAGTCGCCGAATATCAGCCGCCA | | | and represses |
| | CCCTACTTTCCCCCTCCCTACCAGC | | | HIPPO |
| | AGCTGGCCTACTCCCAGTCGGCCGA | | | signaling to |
| | CCCCTACTCGCATCTGGGGGAAGC | | | specify the |
| | GTACGCCGCCGCCATCAACCCCCTG | | | trophectoderm |
| | CACCAGCCGGCGCCCACAGGCAGC | | | lineage. |
| | CAGCAGCAGGCCTGGCCCGGCCGC | | | Development |
| | CAGAGCCAGGAGGGAGCGGGGCTG | | | 142, 1606-15 |
| | CCCTCGCACCACGGGCGCCCGGCC | | | (2015). |
| | GGCCTACTGCCCCACCTCTCCGGGC | | | |
| | TGGAGGCGGGCGCGGTGAGCGCCC | | | |
| | GCAGGGATGCCTACCGCCGCTCCG | | | |
| | ACCTGCTGCTGCCCCACGCACACGC | | | |
| | CCTGGATGCCGCGGGCCTGGCCGA | | | |
| | GAACCTGGGGCTCCACGACATGCC | | | |
| | TCACCAGATGGACGAGGTGCAGAA | | | |
| | TGTCGACGACCAGCACCTGTTGCTG | | | |
| | CACGATCAGACAGTCATTCGCAAA | | | |
| | GGTCCCATTTCCATGACCAAGAACC | | | |
| | CTCTGAACCTCCCCTGTCAGAAGGA | | | |
| | GCTGGTGGGGGCCGTAATGAACCC | | | |
| | CACTGAGGTCTTCTGCTCAGTCCCT | | | |
| | GGAAGATTGTCGCTCCTCAGCTCTA | | | |
| | CGTCTAAATACAAAGTGACAGTGG | | | |
| | CTGAAGTACAGAGGCGACTGTCCC | | | |
| | CACCTGAATGCTTAAATGCCTCGTT | | | |
| | ACTGGGAGGTGTTCTCAGAAGAGC | | | |
| | CAAATCGAAAAATGGAGGCCGGTC | | | |
| | CTTGCGGGAGAAGTTGGACAAGAT | | | |
| | TGGGTTGAATCTTCCGGCCGGGAG | | | |
| | GCGGAAAGCCGCTCATGTGACTCTC | | | |
| | CTGACATCCTTAGTAGAAGGTGAA | | | |
| | GCTGTTCATTTGGCTAGGGACTTTG | | | |
| | CCTATGTCTGTGAAGCCGAATTTCC | | | |
| | TAGTAAACCAGTGGCAGAATATTT | | | |
| | AACCAGACCTCATCTTGGAGGACG | | | |
| | AAATGAGATGGCAGCTAGGAAGAA | | | |
| | CATGCTATTGGCGGCCCAGCAACTG | | | |
| | TGTAAAGAATTCACAGAACTTCTCA | | | |
| | GCCAAGACCGGACACCCCATGGGA | | | |
| | CCAGCAGGCTCGCCCCAGTCTTGGA | | | |
| | GACGAACATACAGAACTGCTTGTCT | | | |
| | CATTTCAGCCTGATTACCCACGGGT | | | |
| | TTGGCAGCCAGGCCATCTGTGCCGC | | | |
| | GGTGTCTGCCCTGCAGAACTACATC | | | |
| | AAAGAAGCCCTGATTGTCATAGAC | | | |
| | AAATCCTACATGAACCCTGGAGAC | | | |
| | CAGAGTCCAGCTGATTCTAACAAA | | | |
| | ACCCTGGAGAAAATGGAGAAACAC | | | |
| | AGGAAA | | | |

TABLE 2

| | | | Estimated | | Median |
|---|---|---|---|---|---|
| | | Media | Number of | Mean Reads | Genes per |
| Sample_ID | Description | Condition | Cells | per Cell | Cell |
| UP_TF_1 | HighMOI, (−) | Pluripotent | 3,640 | 45,983 | 3,317 |
| | TRA-1-60 | stem cell | | | |
| | MACS sorted | medium | | | |
| UP_TF_2 | HighMOI, | Pluripotent | 3,505 | 49,750 | 3,843 |
| | Unsorted | stem cell | | | |
| | | medium | | | |
| UP_TF_3 | HighMOI, | Pluripotent | 4,223 | 45,403 | 3,972 |
| | Unsorted | stem cell | | | |
| | | medium | | | |
| UP_TF_4 | HighMOI, (−) | Pluripotent | 3,461 | 56,290 | 4,475 |
| | TRA-1-60 | stem cell | | | |
| | MACS sorted | medium | | | |
| UP_TF_5 | LowMOI, (−) | Pluripotent | 3,748 | 46,895 | 4,165 |
| | TRA-1-60 | stem cell | | | |
| | MACS sorted | medium | | | |
| UP_TF_8 | Library, | Endothelial | 3,563 | 41,056 | 3,698 |
| | Endothelial | growth | | | |
| | | medium | | | |
| UP_TF_10 | Library, | Multilineage | 2,129 | 70,519 | 5,605 |
| | Multilineage | differentiation | | | |
| | | medium | | | |
| UP_TF_11 | Library, | Endothelial | 6,574 | 23,250 | 3,105 |
| | Endothelial | growth | | | |
| | | medium | | | |
| UP_TF_12 | Library, | Multilineage | 4,678 | 30,340 | 3,882 |
| | Multilineage | differentiation | | | |
| | | medium | | | |
| UP_TF_13 | KLF Family, | Pluripotent | 5,590 | 35,913 | 3,620 |
| | cMYC Mutants | stem cell | | | |
| | | medium | | | |

| | | | Reads | | | |
|---|---|---|---|---|---|---|
| | | | Mapped | | | Median |
| | | | Confidently | | Fraction | UMI |
| | Number of | Valid | to Exonic | Sequencing | Reads in | Counts |
| Sample_ID | Reads | Barcodes | Regions | Saturation | Cells | per Cell |
| UP_TF_1 | 167,381,505 | 97.90% | 65.60% | 17.00% | 55.40% | 11,785 |
| UP_TF_2 | 174,376,238 | 98.40% | 70.30% | 20.80% | 63.90% | 15,985 |
| UP_TF_3 | 191,740,141 | 98.10% | 63.10% | 18.90% | 77.20% | 16,090 |
| UP_TF_4 | 194,819,799 | 98.20% | 66.80% | 25.00% | 78.60% | 19,132 |
| UP_TF_5 | 175,765,276 | 98.10% | 65.70% | 17.70% | 76.90% | 17,349 |
| UP_TF_8 | 146,283,407 | 98.20% | 65.20% | 16.60% | 80.90% | 15,049 |
| UP_TF_10 | 150,135,344 | 98.20% | 68.60% | 20.20% | 83.00% | 27,785 |
| UP_TF_11 | 152,847,871 | 98.20% | 69.40% | 11.20% | 86.80% | 10,681 |
| UP_TF_12 | 141,934,669 | 98.20% | 70.00% | 11.00% | 88.10% | 14,526 |
| UP_TF_13 | 200,756,922 | 98.00% | 66.20% | 15.50% | 78.70% | 14,286 |

TABLE 3

| | Number of Genotyped Cells | | |
|---|---|---|---|
| | Stem cell | Endothelial | Multilineage |
| Genotype | media | media | media |
| ASCL1 | 186 | 78 | 21 |
| ASCL3 | 471 | 150 | 89 |
| ASCL4 | 286 | 90 | 75 |
| ASCL5 | 140 | 64 | 51 |
| ATF7 | 97 | 49 | 45 |
| CDX2 | 267 | 192 | 103 |
| CRX | 292 | 107 | 54 |
| ERG | 62 | 30 | 7 |
| ESRRG | 169 | 98 | 64 |
| ETV2 | 60 | 22 | 21 |
| FLI1 | 55 | 27 | 18 |
| FOXA1 | 53 | 27 | 14 |
| FOXA2 | 89 | 46 | 37 |
| FOXA3 | 255 | 90 | 61 |
| FOXP1 | 413 | 112 | 94 |
| GATA1 | 288 | 111 | 72 |
| GATA2 | 62 | 81 | 60 |
| GATA4 | 71 | 101 | 58 |
| GATA6 | 44 | 44 | 35 |
| GLI1 | 27 | 11 | 16 |
| HAND2 | 310 | 113 | 81 |
| HNF1A | 88 | 45 | 39 |
| HNF1B | 53 | 30 | 41 |
| HOXA1 | 166 | 67 | 57 |
| HOXA10 | 344 | 111 | 66 |
| HOXA11 | 237 | 82 | 47 |
| HOXB6 | 166 | 95 | 44 |
| KLF4 | 298 | 259 | 145 |
| LHX3 | 175 | 76 | 45 |
| LMX1A | 458 | 155 | 82 |
| mCherry | 1689 | 689 | 495 |
| MEF2C | 87 | 49 | 51 |
| MESP1 | 227 | 70 | 55 |
| MITF | 73 | 63 | 45 |
| MYC | 291 | 113 | 36 |
| MYCL | 356 | 112 | 75 |
| MYCN | 50 | 33 | 12 |
| MYODI | 197 | 68 | 40 |
| MYOG | 284 | 122 | 81 |
| NEUROD1 | 83 | 46 | 10 |
| NEUROG1 | 154 | 103 | 23 |
| NEUROG3 | 158 | 138 | 41 |
| NRL | 249 | 75 | 49 |
| ONECUT1 | 159 | 109 | 58 |
| OTX2 | 293 | 95 | 47 |
| PAX7 | 86 | 56 | 28 |
| POU1F1 | 126 | 61 | 50 |
| POU5F1 | 78 | 30 | 24 |
| RUNX1 | 139 | 47 | 43 |
| SIX1 | 260 | 119 | 66 |
| SIX2 | 295 | 103 | 84 |
| SNAI2 | 485 | 96 | 50 |
| SOX10 | 83 | 54 | 30 |
| SOX2 | 137 | 53 | 27 |
| SOX3 | 137 | 56 | 31 |
| SPI1 | 264 | 142 | 67 |
| SPIB | 199 | 70 | 47 |
| SPIC | 147 | 80 | 35 |
| SRY | 166 | 61 | 65 |
| TBX5 | 149 | 112 | 35 |
| TFAP2C | 90 | 58 | 34 |

TABLE 4

| | Enrichment p-value for each genotype in clusters using Fisher's exact test | | | | | | |
|---|---|---|---|---|---|---|---|
| | C6 | C2 | C5 | C3 | C1 | C7 | C4 |
| CDX2 | 0.999581 | 0.502321 | 1 | 1 | 1 | 3.42E−58 | 1 |
| KLF4 | 0.688329 | 1.12E−27 | 1 | 1 | 1 | 1 | 3.82E−21 |
| FOXA1 | 0.848222 | 1 | 1 | 8.00E−08 | 1 | 1 | 1 |
| FOXA2 | 0.559116 | 1 | 1 | 2.56E−15 | 1 | 0.788874 | 1 |
| GATA2 | 0.002284 | 1 | 1.57E−10 | 1 | 1 | 0.91906 | 0.832613 |
| GATA4 | 0.009787 | 0.781098 | 1.13E−09 | 1 | 0.553072 | 1 | 0.822422 |
| GATA6 | 0.03266 | 0.23167 | 0.000147 | 1 | 1 | 1 | 1 |
| SOX10 | 0.017774 | 0.043271 | 1 | 1 | 1 | 0.12661 | 1 |
| NEUROD1 | 0.280233 | 1 | 1 | 1 | 1 | 0.34423 | 1 |
| ETV2 | 0.016254 | 1 | 1 | 1 | 1 | 0.054486 | 1 |
| SPIB | 9.93E−07 | 1 | 0.29024 | 0.190193 | 1 | 1 | 1 |
| SOX3 | 1.53E−05 | 1 | 1 | 1 | 1 | 1 | 0.063768 |
| NEUROG3 | 6.23E−06 | 1 | 1 | 0.502271 | 1 | 0.50894 | 1 |
| TBX5 | 1.71E−07 | 1 | 1 | 0.449045 | 1 | 1 | 1 |
| MYOD1 | 3.73E−07 | 1 | 1 | 1 | 1 | 1 | 0.115324 |
| MYC | 9.91E−05 | 0.611641 | 1 | 1 | 0.394338 | 0.779857 | 1 |
| ESRRG | 5.02E−12 | 0.233929 | 1 | 1 | 0.58849 | 1 | 1 |
| TFAP2C | 6.90E−05 | 1 | 0.541387 | 1 | 1 | 1 | 0.638171 |
| GLI1 | 0.017877 | 1 | 1 | 1 | 1 | 1 | 0.380973 |
| NEUROG1 | 0.00162 | 1 | 1 | 1 | 1 | 0.620425 | 1 |
| ASCL5 | 9.82E−08 | 0.737393 | 1 | 1 | 1 | 0.353463 | 1 |
| FOXA3 | 3.08E−15 | 1 | 1 | 0.644816 | 1 | 1 | 1 |
| ATF7 | 2.03E−09 | 1 | 1 | 0.534822 | 1 | 1 | 1 |
| HOXA10 | 2.36E−09 | 1 | 0.4436 | 0.673452 | 0.599648 | 1 | 0.85978 |
| SOX2 | 4.01E−06 | 1 | 0.461875 | 1 | 1 | 1 | 1 |
| ONECUT1 | 2.98E−11 | 1 | 1 | 0.626421 | 1 | 1 | 0.822422 |
| RUNX1 | 3.65E−07 | 1 | 1 | 1 | 0.450277 | 1 | 0.364314 |
| SIX2 | 8.69E−16 | 0.888323 | 1 | 1 | 1 | 0.677188 | 0.710842 |
| HOXA11 | 4.51E−09 | 1 | 1 | 1 | 1 | 0.860947 | 0.406197 |
| SPIC | 1.28E−06 | 1 | 1 | 1 | 1 | 1 | 0.648778 |
| MYCL | 2.52E−22 | 1 | 1 | 1 | 1 | 1 | 1 |
| FOXP1 | 9.41E−17 | 0.702249 | 1 | 0.795614 | 0.374912 | 0.980162 | 1 |
| SNAI2 | 4.89E−09 | 1 | 0.681398 | 1 | 1 | 0.616212 | 1 |
| HNF1A | 7.52E−11 | 1 | 1 | 1 | 1 | 1 | 1 |
| LMX1A | 2.74E−19 | 1 | 0.845485 | 1 | 1 | 1 | 0.912434 |
| ERG | 0.164469 | 1 | 1 | 1 | 1 | 1 | 1 |
| HAND2 | 7.41E−17 | 1 | 1 | 1 | 1 | 0.653393 | 1 |
| MITF | 2.07E−10 | 1 | 0.643049 | 1 | 1 | 1 | 1 |
| PAX7 | 1.57E−05 | 1 | 1 | 1 | 1 | 0.692249 | 1 |
| SIX1 | 1.58E−14 | 0.822135 | 1 | 1 | 0.599648 | 1 | 1 |
| OTX2 | 3.17E−08 | 0.708559 | 1 | 1 | 1 | 1 | 0.754072 |
| SPI1 | 5.65E−12 | 0.826686 | 1 | 1 | 1 | 0.767724 | 1 |
| GATA1 | 2.36E−13 | 0.847734 | 1 | 1 | 1 | 1 | 0.629688 |
| MYOG | 7.41E−17 | 1 | 1 | 0.746058 | 1 | 0.966092 | 1 |
| HNF1B | 1.21E−06 | 1 | 1 | 1 | 0.434855 | 1 | 1 |
| POU1F1 | 2.52E−14 | 1 | 1 | 1 | 1 | 1 | 1 |
| FLI1 | 0.000193 | 1 | 1 | 1 | 1 | 1 | 1 |
| HOXA1 | 3.20E−15 | 1 | 1 | 1 | 1 | 1 | 1 |
| SRY | 1.01E−17 | 1 | 1 | 1 | 1 | 1 | 1 |
| CRX | 4.15E−13 | 1 | 1 | 1 | 1 | 0.896121 | 1 |
| ASCL1 | 0.000199 | 1 | 1 | 1 | 1 | 1 | 1 |
| NRL | 9.14E−09 | 1 | 1 | 1 | 0.494018 | 0.872071 | 1 |
| LHX3 | 1.65E−11 | 1 | 1 | 1 | 1 | 1 | 1 |
| MESP1 | 2.47E−11 | 1 | 1 | 1 | 0.534212 | 1 | 0.805949 |
| HOXB6 | 3.05E−08 | 1 | 1 | 1 | 1 | 1 | 1 |
| ASCL4 | 3.41E−17 | 1 | 1 | 1 | 0.646165 | 0.956545 | 1 |
| MYCN | 0.00932 | 1 | 1 | 1 | 1 | 1 | 1 |
| MEF2C | 3.40E−10 | 1 | 1 | 1 | 1 | 1 | 0.78156 |
| POU5F1 | 3.21E−06 | 1 | 1 | 1 | 1 | 1 | 1 |
| ASCL3 | 3.49E−19 | 1 | 1 | 1 | 0.707836 | 1 | 1 |
| mCherry | 1.64E−91 | 0.99443 | 0.961129 | 0.996934 | 0.263601 | 0.994961 | 0.947099 |

TABLE 5

| Module | Description | n_genes |
|---|---|---|
| GM1 | Cytoskeleton and polarity | 444 |
| GM2 | Ion transport | 973 |
| GM3 | Chromatin accessibility | 1568 |
| GM4 | Signaling pathways | 873 |
| GM5 | Neuron differentiation | 444 |
| GM6 | Notch pathway | 859 |
| GM7 | Embryonic development | 509 |
| GM8 | Mitochondrial metabolism | 2242 |
| | and translation | |
| GM9 | Ribosome biogenesis | 190 |
| GM10 | Growth factor response | 492 |
| GM11 | Pluripotent state | 234 |

TABLE 6

| | | SEQ | | SEQ |
|---|---|---|---|---|
| | | ID | | ID |
| Gene | Forward Primer (5′→3′) | NO: | Reverse Primer (5′→3′) | NO: |
| CDH5 | AGACCACGCCTCTGTCATGTACCAAATC | 95 | CACGATCTCATACCTGGCCTGCTTC | 113 |
| PECAM1 | GGTCAGCAGCATCGTGGTCAACATAAC | 96 | TGGAGCAGGACAGGTTCAGTCTTTCA | 114 |
| VWF | TCTCCGTGGTCCTGAAGCAGACATA | 97 | AGGTTGCTGCTGGTGAGGTCATT | 115 |
| KDR | AGCCATGTGGTCTCTCTGGTTGTGTATG | 98 | GTTTGAGTGGTGCCGTACTGGTAGGA | 116 |
| NANOG | TTTGTGGGCCTGAAGAAAACT | 99 | AGGGCTGTCCTGAATAAGCAG | 117 |
| POU5F1 | CTTGAATCCCGAATGGAAAGGG | 100 | GTGTATATCCCAGGGTGATCCTC | 118 |
| SOX2 | TACAGCATGTCCTACTCGCAG | 101 | GAGGAAGAGGTAACCACAGGG | 119 |
| DNMT3B | GAGTCCATTGCTGTTGGAACCG | 102 | ATGTCCCTCTTGTCGCCAACCT | 120 |
| SALL2 | CAGCGGAAACCCCAACAGTTA | 103 | GAGGGTCAGTAGAACATGCGT | 121 |
| DPPA4 | GACCTCCACAGAGAAGTCGAG | 104 | TGCCTTTTTCTTAGGGCAGAG | 122 |
| VIM | AGTCCACTGAGTACCGGAGAC | 105 | CATTTCACGCATCTGGCGTTC | 123 |
| CDH1 | CGAGAGCTACACGTTCACGG | 106 | GGGTGTCGAGGGAAAAATAGG | 124 |
| CDH2 | AGCCAACCTTAACTGAGGAGT | 107 | GGCAAGTTGATTGGAGGGATG | 125 |
| EPCAM | TGATCCTGACTGCGATGAGAG | 108 | CTTGTCTGTTCTTCTGACCCC | 126 |
| LAMC1 | GGCAACGTGGCCTTTTCTAC | 109 | AGTGGCAGTTACCCATTCCTG | 127 |
| SPP1 | GAAGTTTCGCAGACCTGACAT | 110 | GTATGCACCATTCAACTCCTCG | 128 |
| THY1 | ATCGCTCTCCTGCTAACAGTC | 111 | CTCGTACTGGATGGGTGAACT | 129 |
| TPM2 | CTGAGACCCGAGCAGAGTTTG | 112 | TGAATCTCGACGTTCTCCTCC | 130 |

### REFERENCES

1. Xu, J., Du, Y. & Deng, H. Direct lineage reprogramming: strategies, mechanisms, and applications. Cell Stem Cell 16, 119-34 (2015).
2. Davis, Robert L; Weintraub, Harold; Lassar, A. B. Expression of a single transfected cDNA converts fibroblasts to myoblasts. Cell 51, 987-1000 (1987).
3. Takahashi, K. & Yamanaka, S. Induction of pluripotent stem cells from mouse embryonic and adult fibroblast cultures by defined factors. Cell 126, 663-76 (2006).
4. Takahashi, K. et al. Induction of pluripotent stem cells from adult human fibroblasts by defined factors. Cell 131, 861-72 (2007).
5. Yu, J. et al. Induced Pluripotent Stem Cell Lines Derived from Human Somatic Cells. Science 318, 1917-1920 (2007).
6. Wernig, M. et al. In vitro reprogramming of fibroblasts into a pluripotent ES-cell-like state. Nature 448, 318-324 (2007).
7. Maherali, N. et al. Directly Reprogrammed Fibroblasts Show Global Epigenetic Remodeling and Widespread Tissue Contribution. Cell Stem Cell 1, 55-70 (2007).
8. Park, I.-H. et al. Reprogramming of human somatic cells to pluripotency with defined factors. Nature 451, 141-146 (2008).
9. Pang, Z. P. et al. Induction of human neuronal cells by defined transcription factors. Nature 476, 220-223 (2011).
10. Sugimura, R. et al. Haematopoietic stem and progenitor cells from human pluripotent stem cells. Nature 545, 432-438 (2017).
11. Yang, N. et al. Generation of pure GABAergic neurons by transcription factor programming. Nat. Methods 14, 621-628 (2017).
12. Sugimura, R. et al. Haematopoietic stem and progenitor cells from human pluripotent stem cells. Nature 545, 432-438 (2017).
13. Zhang, Y. et al. Rapid single-step induction of functional neurons from human pluripotent stem cells. Neuron 78, 785-98 (2013).
14. Abujarour, R. et al. Myogenic differentiation of muscular dystrophy-specific induced pluripotent stem cells for use in drug discovery. Stem Cells Transl. Med. 3, 149-60 (2014).
15. Chanda, S. et al. Generation of induced neuronal cells by the single reprogramming factor ASCL1. Stem Cell Reports 3, 282-96 (2014).
16. Kolodziejczyk, A. A., Kim, J. K., Svensson, V., Marioni, J. C. & Teichmann, S. A. The technology and biology of single-cell RNA sequencing. Mol. Cell 58, 610-20 (2015).
17. Mohr, S., Bakal, C. & Perrimon, N. Genomic screening with RNAi: results and challenges. Annu. Rev. Biochem. 79, 37-64 (2010).
18. Shalem, O., Sanjana, N. E. & Zhang, F. High-throughput functional genomics using CRISPR-Cas9. Nat. Rev. Genet. 16, 299-311 (2015).
19. Adamson, B. et al. A Multiplexed Single-Cell CRISPR Screening Platform Enables Systematic Dissection of the Unfolded Protein Response. Cell 167, 1867-1882.e21 (2016).
20. Dixit, A. et al. Perturb-Seq: Dissecting Molecular Circuits with Scalable Single-Cell RNA Profiling of Pooled Genetic Screens. Cell 167, 1853-1866.e17 (2016).
21. Jaitin, D. A. et al. Dissecting Immune Circuits by Linking CRISPR-Pooled Screens with Single-Cell RNA-Seq. Cell 167, 1883-1896.e15 (2016).
22. Xie, S., Duan, J., Li, B., Zhou, P. & Hon, G. C. Multiplexed Engineering and Analysis of Combinatorial Enhancer Activity in Single Cells. Mol. Cell 66, 285-299.e5 (2017).
23. Datlinger, P. et al. Pooled CRISPR screening with single-cell transcriptome readout. Nat. Methods 14, 297-301 (2017).
24. Macosko, E. Z. et al. Highly Parallel Genome-wide Expression Profiling of Individual Cells Using Nanoliter Droplets. Cell 161, 1202-1214 (2015).
25. Nishiyama, A. et al. Uncovering Early Response of Gene Regulatory Networks in ESCs by Systematic Induction of Transcription Factors. Cell Stem Cell 5, 420-433
26. Blondel, V. D., Guillaume, J.-L., Lambiotte, R. & Lefebvre, E. Fast unfolding of communities in large networks. arXiv 1-12 (2008). doi: 10.1088/1742-5468/2008/10/P10008
27. Orkin, S. H. & Hochedlinger, K. Chromatin connections to pluripotency and cellular reprogramming. Cell 145, 835 (2011).
28. Busskamp, V. et al. Rapid neurogenesis through transcriptional activation in human stem cells. Mol Syst Biol 10, (2014).
29. Velkey, J. M. & O'Shea, K. S. Expression of Neurogenin 1 in mouse embryonic stem cells directs the differentiation of neuronal precursors and identifies unique patterns of downstream gene expression. Dev. Dyn. 242, 230-53 (2013).
30. Castro, D. S. et al. A novel function of the proneural factor Ascl1 in progenitor proliferation identified by genome-wide characterization of its targets. Genes Dev. 25, 930 45 (2011).
31. Tapscott, S. J. The circuitry of a master switch: Myod and the regulation of skeletal muscle gene transcription. Development 132, 2685-2695 (2005).
32. Treutlein, B. et al. Dissecting direct reprogramming from fibroblast to neuron using single-cell RNA-seq. Nature 534, 391-5 (2016).
33. Niwa, H. et al. Interaction between Oct3/4 and Cdx2 Determines Trophectoderm Differentiation. Cell 123, 917-929 (2005).
34. Pelengaris, S., Khan, M. & Evan, G. c-MYC: more than just a matter of life and death. Nat. Rev. Cancer 2, 764-776 (2002).
35. McConnell, B. B. & Yang, V. W. Mammalian Krüppel-like factors in health and diseases. Physiol. Rev. 90, 1337-81 (2010).
36. Tiwari, N. et al. Klf4 Is a Transcriptional Regulator of Genes Critical for EMT, Including Jnk1 (Mapk8). PLOS One 8, e57329 (2013).
37. Zhang, B. et al. KLF5 activates microRNA 200 transcription to maintain epithelial characteristics and prevent induced epithelial-mesenchymal transition in epithelial cells. Mol. Cell. Biol. 33, 4919-35 (2013).
38. Gumireddy, K. et al. KLF17 is a negative regulator of epithelial-mesenchymal transition and metastasis in breast cancer. Nat. Cell Biol. 11, 1297-304 (2009).
39. Liu, Y.-N. et al. Critical and reciprocal regulation of KLF4 and SLUG in transforming growth factor β-initiated prostate cancer epithelial-mesenchymal transition. Mol. Cell. Biol. 32, 941-53 (2012).
40. Li, R. et al. A Mesenchymal-to-Epithelial Transition Initiates and Is Required for the Nuclear Reprogramming of Mouse Fibroblasts. Cell Stem Cell 7, 51-63 (2010).
41. Barrallo-Gimeno, A., Nieto, M. A. & Ip, Y. T. The Snail genes as inducers of cell movement and survival: implications in development and cancer. Development 132, 3151-61 (2005).
42. Subramanian, A. et al. Gene set enrichment analysis: a knowledge-based approach for interpreting genome-wide expression profiles. Proc. Natl. Acad. Sci. 102, 15545-15550 (2005).
43. Morita, R. et al. ETS transcription factor ETV2 directly converts human fibroblasts into functional endothelial cells. Proc. Natl. Acad. Sci. 112, 160-165 (2015).
44. Li. W. et al. MAGcCK enables robust identification of essential genes from genome-scale CRISPR/Cas9 knockout screens. Genome Biol. 15, 554 (2014)

1-11. (canceled)

12. A kit for performing a high throughput gene overexpression screen in a transduced target cell comprising:
(a) a library of polynucleotides, wherein each polynucleotide comprises:
(i) a nucleic acid encoding a Transcription Factor (TF) gene Open Reading Frame (ORF); and
(ii) a nucleic acid encoding a selectable marker;
(b) a library of barcode nucleic acids, wherein each barcode nucleic acid encodes a TF barcode; and
(c) optionally instructions for use;
wherein when incorporated into a vector, each barcode nucleic acid is introduced 3′ to the nucleic acid encoding the TF ORF; and
wherein the TF gene is a wild-type TF gene, an engineered TF gene, or a mutated TF gene.

13-22. (canceled)

23. The kit of claim 12, wherein the nucleic acid encoding the TF ORF is operably linked to the nucleic acid encoding the selectable marker by a nucleic acid encoding a 2A peptide.

24. The kit of claim 12, wherein the TF gene drives differential expression of more than 100 genes.

25. The kit of claim 12, wherein the wild-type TF gene encodes a developmentally critical TF selected from ASCL1, ASCL3, ASCL4, ASCL5, ATF7, CDX2, CRX, ERG, ESRRG, ETV2, FLI1, FOXA1, FOXA2, FOXA3, FOXP1, GATA1, GATA2, GATA4, GATA6, GLI1, HAND2, HNF1A, HNF1B, HNF4A, HOXA1, HOXA10, HOXA11, HOXB6, KLF4, LHX3, LMXIA, MEF2C, MESP1, MITF, MYC, MYCL, MYCN, MYOD1, MYOG, NEUROD1, NEUROG1, NEUROG3, NRL, ONECUT1, OTX2, PAX7, POU1F1, POU5F1, RUNX1, SIX1, SIX2, SNAI2, SOX10, SOX2, SOX3, SPI1, SPIB, SPIC, SRY, TBX5, or TFAP2C.

26. The kit of claim 12, wherein the library of polynucleotides comprises a nucleic acid sequence selected from the group consisting of SEQ ID NO: 2-28, and 34-94.

27. The kit of claim 12, wherein the library of polynucleotides comprises:
(a) the nucleic acid sequence of SEQ ID NOs: 2-12;
(b) the nucleic acid sequence of SEQ ID NOs: 13-28; or
(c) the nucleic acid sequence of SEQ ID NO: 34-94.

28. The kit of claim 12, wherein the library of polynucleotides comprises at least 10, at least 20, at least 30, at least 40, at least 50, at least 60, at least 70, at least 80, at least 90, or at least 100 polynucleotides.

29. The kit of claim 12, wherein the vector comprises:
(a) a nucleic acid encoding an expression control element; and/or
(b) a 3′-long terminal repeat (LTR) region.

30. The kit of claim 12, wherein the vector is a retroviral viral vector or a lentiviral vector.

31. The kit of claim 12, wherein the vector is a viral particle.

32. The kit of claim 29, wherein the expression control element comprises a promoter or a 5′-long terminal repeat (LTR) region.

33. The kit of claim 29, wherein the expression control element comprises a translation elongation factor 1A (EF1A) promoter.

34. The kit of claim 12, wherein when assembled on the vector, the TF barcode nucleic acid is located 3′ to the nucleic acid encoding the selectable marker.

35. The kit of claim 29, wherein when assembled on the vector, the TF barcode nucleic acid is located about 200 base pairs upstream of the 3′-LTR region.

36. The kit of claim 12, further comprising a target cell, wherein the target cell is in the same or a separate kit.

37. The kit of claim 36, wherein the target cell is:
(a) a mammalian cell selected from equine cell, bovine cell, canine cell, murine cell, porcine cell, feline cell, or human cell; and/or
(b) a stem cell; or
(c) an embryonic stem cell (ESC); or
(d) an induced pluripotent stem cell (iPSC).

38. The kit of claim 12, wherein the instructions for use comprise:
(a) determining a fitness effect of a TF ORF overexpression in the transduced target cell;
(b) identifying the transduced target cell comprising a significant TF ORF in conjunction with single cell RNA sequencing;
(c) identifying the effect of a TF ORF overexpression on a gene-to-gene co-perturbation network in the transduced target cell; and/or
(d) segmenting a co-perturbation network into functional gene modules.

39. The kit of claim 38, wherein determining the fitness effect comprises determining the effect of the TF ORF expression on the transduced target cell proliferation, viability, rate of senescence, apoptosis, DNA repair mechanism, genome stability, gene transcription, or stress response.

40. The kit of claim 38, wherein the significant TF ORF exhibits a cluster enrichment with a false discovery rate (FDR) of less than 10⁻⁶; and a cluster enrichment profile different from a non-TF control with a FDR less than 10⁻⁶ based on a Fisher's exact test.

41. A kit for performing a high throughput gene overexpression screen in a transduced target cell comprising:
(a) a barcoded open reading frame (ORF) screening library of transcription factor (TF) genes, wherein each TF ORF in the library is expressed by a lentiviral vector, wherein each lentiviral vector comprises:
(i) a polynucleotide encoding the TF gene ORF;
(ii) a nucleic acid encoding a selectable marker; and
(iii) a nucleic acid barcode located downstream of the selectable marker; and
(b) optionally instructions for use,
wherein the library of polynucleotides comprises a nucleic acid sequence selected from the group consisting of SEQ ID NO: 2-28, and 34-94.
